(19) United States
(12) Patent Application Publication
(10) Pub. No.: US 2024/0392335 A1
(43) Pub. Date: Nov. 28, 2024

(54) CHONDROITIN-PRODUCING RECOMBINANT CELL

(71) Applicant: GIVAUDAN SA, Vernier (CH)

(72) Inventors: Dominique LOUIS, Orsay (FR); Karine JAILLARDON, Orsay (FR); Muriel MERKAMM, Orsay (FR); Dominique THOMAS, Argenteuil (FR)

(21) Appl. No.: 18/553,310

(22) PCT Filed: Mar. 31, 2022
(86) PCT No.: PCT/EP2022/058583

§ 371 Date: Sep. 29, 2023

(30) Foreign Application Priority Data
Apr. 1, 2021 (EP) 21166740.7

(51) Int. Cl. C12P 19/26; C12N 9/04; C12N 9/10; C12N 9/12; C12N 9/88; C12N 9/90; C12N 15/81

(52) U.S. Cl. CPC C12P 19/26; C12N 9/0006; C12N 9/1029; C12N 9/1051; C12N 9/1096; C12N 9/1241; C12N 9/88; C12N 9/90; C12N 15/81; C12Y 101/01022; C12Y 203/01004; C12Y 206/01016; C12Y 207/07009; C12Y 207/07023; C12Y 402/02004; C12Y 501/03002; C12Y 504/02002; C12Y 504/02003

(57) ABSTRACT

The present invention relates to the field of bio-production of chondroitin. There is a need in the art for chondroitin production methods allowing its highly efficient synthesis and secretion. The solution proposed in the present invention is the use of a recombinant cell, in particular a recombinant yeast, comprising many modifications as described in the present text. The present invention further proposes methods allowing the bio-production of chondroitin using the recombinant cell, in particular a recombinant yeast, of the invention.

### FIELD OF THE INVENTION

The present invention relates to the field of bio-production of chondroitin.

### REFERENCE TO SEQUENCE LISTING

This application includes as the Sequence Listing the complete contents of the accompanying text file “13390124us_corrected_seq_listing” created Jun. 20, 2024, containing 349,183 bytes, hereby incorporated by reference.

### BACKGROUND OF THE INVENTION

Chondroitin is a high molecular weight polysaccharide that occurs naturally in the connective tissues of people and animals. Chondroitin belongs to the family of heteropolysaccharides called glycosaminoglycans.

Glycosaminoglycans (GAGs) or mucopolysaccharides are unbranched, negatively charged polysaccharide chains composed of repeating disaccharide units including an acidic sugar and an amino sugar (N-acetylglucosamine or N-acetylgalactosamine). Because of their inflexible nature and high negative charge, GAGs are used in the body as a lubricant or shock absorber. The cartilage matrix lining the knee joint, for example, which is rich in GAGs, can support pressures of hundreds of atmospheres by this mechanism.

Chondroitin, which is formed of D-glucuronic acid and of N-acetyl-D-galactosamine, may be sulfated. Chondroitin sulfate is important for maintenance of cartilage strength and resilience and is marketed as a nutritional supplement to reduce joint pain and promote healthy cartilage and joint function. Clinical studies support the use of chondroitin and chondroitin sulfate for treatment of osteoarthritis.

Chondroitin sulfate or chondroitin being naturally present in the extracellular matrix of conjunctive tissues of the skin, it is also used for cosmetic application, in particular to moisturize, heal and soothe the skin or as a component of hair conditioner.

Currently, chondroitin is mainly produced by extraction from cartilage of animals using chemical and enzymatic treatments to dissociate the polysaccharide from the protein and produce a polysaccharide product of varying quality. However, these methods can be laborious and expensive.

As such, microbial production of chondroitin has been suggested. Indeed, certain bacteria produce chondroitin and chondroitin-like polysaccharide polymers as a component of their capsule. However, these known bacteria, such as Pasteurella multocida or Escherichia coli, are pathogens to many mammals and produce low quantities of the polysaccharides.

As a consequence, research has turned towards recombinant microorganisms, and in particular recombinant bacteria, for the production of chondroitin. Mention may be made for example of WO2011109438, of EP2142643, or of US20090263867.

Contrary to other microorganisms that are commonly used for the production of biological molecules, yeasts are generally recognised as safe. They can grow rapidly and be cultivated at higher density, compared to bacteria, and do not require an aseptic environment. Furthermore, yeast cells can be more easily separated from the culture medium than bacteria, greatly simplifying the process for product extraction and purification. Finally, yeasts present the advantage of being more resistant to pH changes in the culture medium, and as such represent stronger fermentation systems.

However, among yeasts, distinguishing features between species may also present certain challenges. This is mainly due to the difference in metabolisms between the species and their culture conditions.

However, the inventors have found that Saccharomyces cerevisiae is particularly useful as a tool for the production of molecules of interest since it has a long safe history when it comes to being used by humans (such as in wine, beer, or bread) and as such is a well established model whose genetic information is well known in the art. S. cerevisiae further presents the advantage of being generally recognised as safe for humans and animals. What's more, the acidification of the medium which occurs when cultivating this yeast reduces the possibility of contamination of the bio fermenters, and therefore reduces the need to add antibiotics to the medium. Finally, many genetic tools have been developed allowing for stable modifications of its genome (integration within the chromosome).

Accordingly, there is still a need in the art for further chondroitin production methods allowing its highly efficient synthesis and secretion. In particular, there is still a need to provide production methods that are cost-efficient and supply chondroitin which is safe for human application.

In a particular case, there is still a need in the art for chondroitin production methods allowing to obtain large amounts of chondroitin of a particular and controlled size.

### SUMMARY OF THE INVENTION

The present invention accordingly relates to the following items.

Item 1: A recombinant yeast cell producing chondroitin wherein the recombinant yeast cell comprises:
(a) one or more recombinant nucleic acids encoding a polypeptide having a chondroitin synthase (HCOS) activity; and
(b) one or more recombinant nucleic acids encoding a polypeptide having UDP-Glucose dehydrogenase (UDP-GlcDH or HASB) activity; and
(c) one or more recombinant nucleic acids encoding a polypeptide having a UDP-glucose-4-epimerase (kfoA or GNE1) activity.

As illustrated in the examples, the recombinant yeasts of the invention allow for the production of chondroitin in a yeast cell which is not naturally able to produce this glycosaminoglycan. It is further demonstrated in the examples that the size of the chondroitin produced by the recombinant yeast may be controlled.

Said advantageous properties can be further increased by recombining the yeast with additional modifications described here-after.

Item 2: The recombinant according to item 1, wherein the recombinant cell comprises one or more recombinant nucleic acids encoding a polypeptide having a chondroitinase activity wherein the polypeptide having chondroitinase activity comprises a secretion signal, and optionally an anchoring signal.

Item 3: A recombinant host cell producing chondroitin wherein the recombinant host cell comprises:
(a) one or more recombinant nucleic acids encoding a polypeptide having a chondroitin synthase (HCOS) activity; and
(b) one or more recombinant nucleic acids encoding a polypeptide having UDP-Glucose dehydrogenase (UDP-GlcDH or HASB) activity;
(c) one or more recombinant nucleic acids encoding a polypeptide having a UDP-glucose-4-epimerase (kfoA or GNE1) activity; and
(d) one or more recombinant nucleic acids encoding a polypeptide having a chondroitinase activity wherein the polypeptide having chondroitinase activity comprises a secretion signal or comprises a secretion signal, and optionally an anchoring signal, so that chondroitin, in particular of a desired molecular weight is produced by the host cell.

Item 4: The recombinant host cell according to item 2 or 3, wherein the recombinant nucleic acid encoding a polypeptide having a chondroitinase activity is obtained or derived from at least one of Cupiennius salei, Tityus serrulatus, Bos taurus, Vespa magnifica, Macaca mulatta or Apis mellifera, and preferably from Tityus serrulatus.

Item 5: The recombinant cell according to any one of items 2 to 4, wherein the molecular weight of the chondroitin is in the range of less than 50 kDa, preferably is in the range of about 20 kDa to about 50 kDa.

Item 6: The recombinant cell according to any one of items 1 to 4, wherein the molecular weight of the chondroitin is in the range of greater than 50 kDa, preferably is in the range of about 50 kDa to about 250 kDa.

Item 7: The recombinant cell according to any one of items 1 to 4, wherein the molecular weight of the chondroitin is in the range of greater than 100 kDa, preferably is in the range of about 100 kDa to about 1500 kDa.

Item 8: The recombinant cell according to any one of items 1 to 7, wherein the nucleic acid encoding a polypeptide having a UDP-Glucose dehydrogenase (UDP-GlcDH or HASB) activity is obtained or derived from at least one of Arabidopsis thaliana, Chlorella virus PBCV1 or Streptococcus zooepidemicus, and in particular from at least one of Arabidopsis thaliana or Chlorella virus PBCV1.

Item 9: The recombinant cell according to any one of items 1 to 8, wherein the nucleic acid encoding a polypeptide having a chondroitin synthase (HCOS) activity is
(i) a nucleic acid encoding a chondroitin synthase; or
(ii) a nucleic acid encoding a chimeric polypeptide having a chondroitin synthase activity.

Item 10: The recombinant cell according to item 9, wherein the nucleic acids are, obtained or derived from at least one of Pasteurella multocida, Chlorella virus PBCV1, Mycobacterium tuberculosis, Homo sapiens, Escherichia coli, Saccharomyces cerevisiae or Penicillium oxalicum.

Item 11: The recombinant cell according to any one of items 1 to 10, wherein the nucleic acid encoding a polypeptide having a UDP-glucose-4-epimerase (kfoA or GNE1) activity is obtained or derived from a bacteria, and in particular from a bacteria selected from the group consisting of Pseudomonas aeruginosa, Pasteurella multocida, and Escherichia coli.

Item 12: The recombinant cell according to any one of items 1 to 11, wherein the recombinant cell further comprises at least one recombinant nucleic acid encoding one or more of:
(i) a polypeptide having glutamine-fructose-6-phosphate amidotransferase (GFA1) activity; and/or
(ii) a polypeptide having UDP-N-acetylglucosamine pyrophosphorylase (QRI1) activity.

Item 13: The recombinant cell according to any one of items 1 to 12, wherein the recombinant cell further comprises at least one recombinant nucleic acid encoding one or more of:
(i) a polypeptide having Phosphoglucomutase-1 (PGM1) activity; and/or
(ii) a polypeptide having UTP-glucose-1-phosphate uridylyltransferase (UGP1) activity; and/or
(iii) a polypeptide having Glucosamine-6-phosphate N-acetyltransferase (GNA1) activity; and/or
(iv) a polypeptide having phosphoacetylglucosamine mutase (PCM1) activity.

Item 14: The recombinant cell according to item 12 or 13, wherein:
the nucleic acid encoding the polypeptide having a glutamine-fructose-6-phosphate amidotransferase (GFA1) activity;
the nucleic acid encoding the polypeptide having a UDP-N-acetylglucosamine pyrophosphorylase (QRI1) activity;
the nucleic acid encoding the polypeptide having a Phosphoglucomutase-1 (PGM1) activity;
the nucleic acid encoding the polypeptide having a UTP-glucose-1-phosphate uridylyltransferase (UGP1) activity;
the nucleic acid encoding the polypeptide having a Glucosamine-6-phosphate N-acetyltransferase (GNA1) activity; and/or
the nucleic acid encoding the polypeptide having phosphoacetylglucosamine mutase (PCM1);
are obtained or derived from Saccharomyces cerevisiae.

Item 15: The recombinant host cell according to any one of items 3 to 14, wherein the recombinant host cell is a yeast.

Item 16: The recombinant cell according to any one of items 1 to 15, wherein the recombinant cell belongs to the Saccharomyces genus, or to the Candida genus, or to the Kluyveromyces genus, or to the Ogataea genus, or to the Yarrowia genus, or to the Debaryomyces genus, or to the Ashbya genus, and in particular belongs to the Saccharomyces genus.

Item 17: The recombinant cell according to item 16, wherein the recombinant cell is selected from the group consisting of Saccharomyces cerevisiae, Saccharomyces boulardii, Saccharomyces bayanus, Saccharomyces paradoxus, Saccharomyces mikatae, Saccharomyces castelli, Candida albicans, Candida glabrata, Candida tropicalis, Kluyveromyces lactis, Kluyveromyces marxianus, Kluyveromyces polysporus, Kluyveromyces thermotolerens, Ogataea polymorpha, Yarrowia lypolytica, Debaryomyces hansenii, and Ashbya gossypii, and is preferably Saccharomyces cerevisiae.

Item 18: A method of producing chondroitin of a desired molecular weight comprising:
(a) cultivating a recombinant cell as defined in any one of items 1 to 17 in a cultivation medium for a time sufficient to produce chondroitin of the desired molecular weight; and
(b) optionally isolating or recovering the chondroitin from the recombinant cell and/or from the cultivation medium.

Item 19: The method of item 18, wherein the chondroitin has a molecular weight of from about 20 kDa to about 50 kDa.

Item 20: The method of item 18, wherein chondroitin has a molecular weight of from about 50 kDa to about 150 kDa.

Item 21: The method of item 18, wherein chondroitin has a molecular weight of from about 150 kDa to about 1500 kDa.

Item 22: The method according to any one of items 18 to 21, wherein the recombinant cell is a yeast belonging to the Saccharomyces genus, and in particular is Saccharomyces cerevisiae.

Item 23: The method according to any one of items 18 to 22, wherein the time sufficient to produce chondroitin of a desired molecule weight is a period of from about 35 hours to about 50 hours, preferably from about 40 hours to about 50 hours, preferably is about 48 hours.

Item 24: The method according to any one of items 18 to 23, wherein the molecular weight of the produced chondroitin is controlled by regulating the pH of the cultivation medium during step (a).

Item 25: The method according to any one of items 18 to 24, wherein the method is carried out on an industrial scale, preferably where the cultivation medium is at least about 100 L, more preferably is in the range of about 1,000 L to about 3,000 L, even more preferably is about 10,000 L or even more preferably is about 100,000 L, or even is about 250,000 L.

Item 26: Chondroitin obtainable from a recombinant cell according to any one of items 1 to 17, or from the method of according to any one of items 18 to 25.

Item 27: A cultivation medium comprising the chondroitin according to item 26.

Item 28: A composition comprising the chondroitin according to item 26.

Item 29: An industrial product or a consumer product or a consumable comprising (i) the chondroitin having a molecular weight as defined in any one of items 5 to 7, (ii) the cultivation medium of item 27 or (iii) the composition of item 28.

Item 30: The industrial product or the consumer product or the consumable of item 29, wherein the industrial product or the consumer product or the consumable is a cosmetic product, a flavour product, a fragrance product, a food product, a food, a beverage, a texturant, a pharmaceutical composition, a dietary supplement, a nutraceutical, a cleaning product, a dental and/or an oral hygiene composition.

Item 31: Use of a recombinant cell as defined in any one of items 2 to 17 for the production of chondroitin having a molecular weight in the range of from about 20 kDa to about 50 k Da or from about 50 kDa to about 1000 kDa.

Item 32: A method for producing chondroitin, comprising the steps of:
(a) culturing a recombinant yeast as defined in any one of items 1 to 17 in a cultivation medium; and
(b) recovering the chondroitin from said cultivation medium,
wherein the chondroitin recovered in step (b) has a molecular weight controlled through the selection of:
   the nature and origin of the nucleic acid encoding a polypeptide having a chondroitinase activity of the recombinant yeast,
   the nature and origin of the promoter controlling the expression of the recombinant nucleic acid encoding a polypeptide having a chondroitinase activity of the recombinant yeast,
   the presence of an anchoring and/or of a secretion signal associated to the polypeptide having a chondroitinase activity of the recombinant yeast,
   the pH of the cultivation medium during the step of culturing the recombinant yeast, and/or
   the duration of the culturing of the recombinant yeast.

Certain embodiments of the present invention may provide one or more of the following advantages:
a process for producing chondroitin of controlled molecular weight;
a process for producing chondroitin of controlled molecular weight in a Saccharomyces yeast cell; and
a process for producing chondroitin of controlled molecular weight by varying genetic parameters (eg regulatory sequences) and/or process parameters (pH or fermentation time

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### BRIEF DESCRIPTION OF THE FIGURES

FIG. 1 shows a schematic pathway for the production of chondroitin

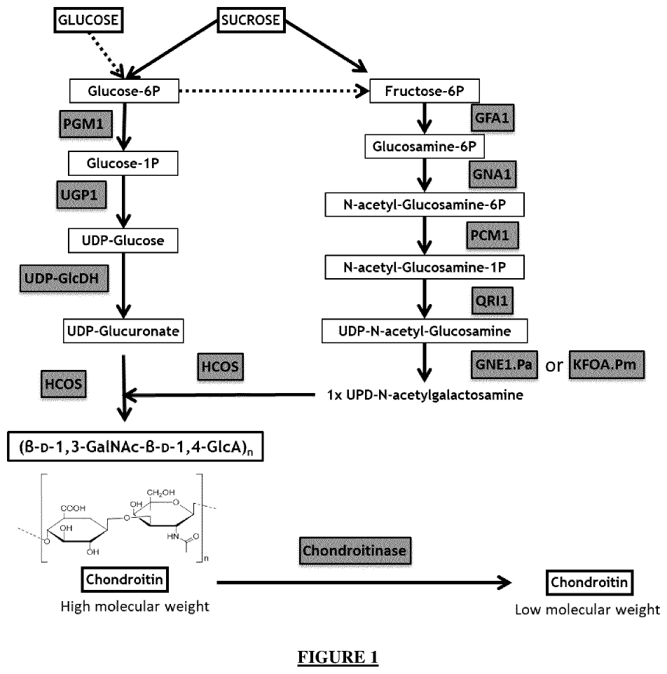

### SUMMARY OF THE SEQUENCES

SEQ ID NO: 1 is a reencoded nucleic acid sequence of a polypeptide having a chondroitin synthase activity:chimeric nucleic acid comprising a fragment from a nucleic acid encoding hyaluronan synthase originating from Pasteurella multocida and a fragment from a nucleic acid encoding a chondroitin synthase from Pasteurella multocida (HCOS1-1)
SEQ ID NO: 2 is a reencoded nucleic acid sequence of a polypeptide having a chondroitin synthase activity:chimeric nucleic acid comprising a fragment from a nucleic acid encoding a hyaluronan synthase originating from Pasteurella multocida and a fragment from a nucleic acid encoding a chondroitin synthase from Pasteurella multocida (HCOS1-2)
SEQ ID NO: 3 is a reencoded nucleic acid sequence of a polypeptide having a chondroitin synthase activity:chimeric nucleic acid comprising a fragment from a nucleic acid encoding a hyaluronan synthase originating from Pasteurella multocida and a fragment from a nucleic acid encoding a chondroitin synthase from Pasteurella multocida (HCOS1-3)
SEQ ID NO: 4: is a nucleic acid sequence of a polypeptide having chondroitin synthase activity:chimeric nucleic acid comprising a fragment from a nucleic acid encoding a hyaluronan synthase originating from Pasteurella multocida, a fragment from a nucleic acid encoding a chondroitin synthase from Pasteurella multocida and a fragment from a nucleic acid encoding a chitin synthase 2 from Saccharomyces cerevisiae (HCOS.Sc)
SEQ ID NO: 5: is a nucleic acid sequence of a polypeptide having chondroitin synthase activity:chimeric nucleic acid comprising a fragment from a nucleic acid encoding a hyaluronan synthase originating from Pasteurella multocida and a fragment from a nucleic acid encoding a chitin synthase 2 from Saccharomyces cerevisiae (HHASA.Sc)
SEQ ID NO: 6: is a nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin synthase from Escherichia coli (HCOS1-Vir)
SEQ ID NO: 7: is a reencoded nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin synthase from Escherichia coli (HCOS2-Vir)
SEQ ID NO: 8: is a reencoded nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a Galactofuranosyltransferase from Mycobacterium tuberculosis (HCOS3-Vir)
SEQ ID NO: 9: is a reencoded nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin synthase from Escherichia coli (HCOS4-Vir)
SEQ ID NO: 10: is a nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin synthase from Penicillium oxalicum (HCOS5-Vir)
SEQ ID NO: 11: is a nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin synthase from Pasteurella multocida (HCOS6-Vir)
SEQ ID NO: 12: is a reencoded nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin synthase from Pasteurella multocida (HCOS7-Vir)
SEQ ID NO: 13: is a reencoded nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin synthase from Pasteurella multocida (HCOS8-Vir)
SEQ ID NO: 14: is a nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin sulfate synthase from Homo sapiens (HCOS9-Vir)
SEQ ID NO: 15: is a reencoded nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin sulfate synthase from Homo sapiens (HCOS10-Vir)
SEQ ID NO: 16: is a reencoded nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin sulfate synthase from Homo sapiens (HCOS11-Vir)
SEQ ID NO: 17: is a reencoded nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin sulfate synthase from Homo sapiens (HCOS12-Vir)
SEQ ID NO: 18 is an amino acid sequence of a polypeptide having a chondroitin synthase activity:chimeric amino acid comprising a fragment from an amino acid encoding a hyaluronan synthase originating from Pasteurella multocida and a fragment from an amino acid encoding a chondroitin synthase from Pasteurella multocida (HCOS1-1)
SEQ ID NO: 19 is an amino acid sequence of a polypeptide having a chondroitin synthase activity:chimeric amino acid comprising a fragment from an amino acid encoding a hyaluronan synthase originating from Pasteurella multocida and a fragment from an amino acid encoding a chondroitin synthase from Pasteurella multocida (HCOS1-2)
SEQ ID NO: 20 is an amino acid sequence of a polypeptide having a chondroitin synthase activity:chimeric amino acid comprising a fragment from an amino acid encoding a hyaluronan synthase originating from Pasteurella multocida and a fragment from an amino acid encoding a chondroitin synthase from Pasteurella multocida (HCOS1-3)
SEQ ID NO: 21: is an amino acid sequence of a polypeptide having chondroitin synthase activity:chimeric amino acid comprising a fragment from an amino acid encoding a hyaluronan synthase originating from Pasteurella multocida, a fragment from an amino acid encoding a chondroitin synthase from Pasteurella multocida and a fragment from an amino acid encoding a chitin synthase 2 from Saccharomyces cerevisiae (HCOS.Sc)
SEQ ID NO: 22: is a nucleic acid sequence of a polypeptide having chondroitin synthase activity:chimeric amino acid comprising a fragment from an amino acid encoding a hyaluronan synthase originating from Pasteurella multocida and a fragment from an amino acid encoding a chitin synthase 2 from Saccharomyces cerevisiae (HHASA.Sc)
SEQ ID NO: 23: is an amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin synthase from Escherichia coli (HCOS1-Vir)
SEQ ID NO: 24: is a reencoded amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin synthase from Escherichia coli (HCOS2-Vir)
SEQ ID NO: 25: is a reencoded amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin synthase from Escherichia coli (HCOS3-Vir)
SEQ ID NO: 26: is a reencoded amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin synthase from Escherichia coli (HCOS4-Vir)
SEQ ID NO: 27: is an amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin synthase from Penicillium oxalicum (HCOS5-Vir)
SEQ ID NO: 28: is an amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin synthase from Pasteurella multocida (HCOS6-Vir)
SEQ ID NO: 29: is a reencoded amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin synthase from Pasteurella multocida (HCOS7-Vir)
SEQ ID NO: 30: is a reencoded amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin synthase from Pasteurella multocida (HCOS8-Vir)
SEQ ID NO: 31: is an amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin sulfate synthase from Homo sapiens (HCOS9-Vir)
SEQ ID NO: 32: is a reencoded amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin sulfate synthase from Homo sapiens (HCOS10-Vir)
SEQ ID NO: 33: is a reencoded amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin sulfate synthase from Homo sapiens (HCOS11-Vir)
SEQ ID NO: 34: is a reencoded amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin sulfate synthase from Homo sapiens (HCOS12-Vir)
SEQ ID NO: 35 is a nucleic acid sequence of UDP-Glucose dehydrogenase (HASB) originating from Arabidopsis thaliana
SEQ ID NO: 36 is a reencoded nucleic acid sequence of UDP-Glucose dehydrogenase (HASB) originating from Chlorella virus PBCV-1
SEQ ID NO: 37 is a reencoded nucleic acid sequence of UDP-Glucose dehydrogenase (HASB-A) originating from Chlorella virus PBCV-1
SEQ ID NO: 38 is a nucleic acid sequence of UDP-Glucose dehydrogenase (HASB) originating from Streptococcus zooepidemicus
SEQ ID NO: 39 is an amino acid sequence of UDP-Glucose dehydrogenase (HASB) originating from Arabidopsis thaliana
SEQ ID NO: 40 is an amino acid sequence of UDP-Glucose dehydrogenase (HASB) originating from Chlorella virus PBCV-1
SEQ ID NO: 41 is an amino acid sequence of UDP-Glucose dehydrogenase (HASB) originating from Streptococcus zooepidemicus
SEQ ID NO: 42 is a nucleic acid sequence of UDP-glucose-4-epimerase (GNE1) originating from Pseudomonas aeruginosa
SEQ ID NO: 43 is a nucleic acid sequence of UDP-glucose-4-epimerase (KFOA) originating from Pasteurella multocida
SEQ ID NO: 44 is a nucleic acid sequence of UDP-glucose-4-epimerase (KFOA) originating from Escherichia coli
SEQ ID NO: 45 is an amino acid sequence of UDP-glucose-4-epimerase (GNE1) originating from Pseudomonas aeruginosa
SEQ ID NO: 46 is an amino acid sequence of UDP-glucose-4-epimerase (KFOA) originating from Pasteurella multocida
SEQ ID NO: 47 is an amino acid sequence of UDP-glucose-4-epimerase (KFOA) originating from Escherichia coli
SEQ ID NO: 48 is a reencoded nucleic acid sequence of hyaluronidase (HYAL) originating from Cupiennius salei with a N-terminal secretion signal
SEQ ID NO: 49 is a reencoded nucleic acid sequence of hyaluronidase (HYAL) originating from Tityus serrulatus with a N-terminal secretion signal
SEQ ID NO: 50 is a reencoded nucleic acid sequence of hyaluronidase (HYAL) originating from Bos Taurus with a N-terminal secretion signal
SEQ ID NO: 51 is a reencoded nucleic acid sequence of hyaluronidase (HYAL) originating from Apis mellifera with a N-terminal secretion signal
SEQ ID NO: 52 is a reencoded nucleic acid sequence of hyaluronidase (HYAL) originating from Macaca mulatta with a N-terminal secretion signal
SEQ ID NO: 53 is a reencoded nucleic acid sequence of hyaluronidase (HYAL) originating from Vespa magnifica with a N-terminal secretion signal
SEQ ID NO: 54 is a reencoded nucleic acid sequence of hyaluronidase (HYAL) originating from Cupiennius salei with a N-terminal secretion signal and a C-terminal anchoring signal
SEQ ID NO: 55 is a reencoded nucleic acid sequence of hyaluronidase (HYAL) originating from Tityus serrulatus with a N-terminal secretion signal and a C-terminal anchoring signal
SEQ ID NO: 56 is a reencoded nucleic acid sequence of hyaluronidase (HYAL) originating from Bos taurus with a N-terminal secretion signal and a C-terminal anchoring signal
SEQ ID NO: 57 is a reencoded nucleic acid sequence of hyaluronidase (HYAL) originating from Apis mellifera with a N-terminal secretion signal and a C-terminal anchoring signal
SEQ ID NO: 58 is a reencoded nucleic acid sequence of hyaluronidase (HYAL) originating from Macaca mulatta with a N-terminal secretion signal and a C-terminal anchoring signal
SEQ ID NO: 59 is a reencoded nucleic acid sequence of hyaluronidase (HYAL) originating from Vespa magnifica with a N-terminal secretion signal and a C-terminal anchoring signal
SEQ ID NO: 60 is an amino acid sequence of hyaluronidase (HYAL) originating from Cupiennius salei with a N-terminal secretion signal
SEQ ID NO: 61 is an amino acid sequence of hyaluronidase (HYAL) originating from Tityus serrulatus with a N-terminal secretion signal
SEQ ID NO: 62 is an amino acid sequence of hyaluronidase (HYAL) originating from Bos taurus with a N-terminal secretion signal
SEQ ID NO: 63 is an amino acid sequence of hyaluronidase (HYAL) originating from Apis mellifera with a N-terminal secretion signal
SEQ ID NO: 64 is an amino acid sequence of hyaluronidase (HYAL) originating from Macaca mulatta with a N-terminal secretion signal
SEQ ID NO: 65 is an amino acid sequence of hyaluronidase (HYAL) originating from Vespa magnifica with a N-terminal secretion signal
SEQ ID NO: 66 is an amino acid sequence of hyaluronidase (HYAL) originating from Cupiennius salei with a N-terminal secretion signal and a C-terminal anchoring signal
SEQ ID NO: 67 is an amino acid sequence of hyaluronidase (HYAL) originating from Tityus serrulatus with a N-terminal secretion signal and a C-terminal anchoring signal
SEQ ID NO: 68 is an amino acid sequence of hyaluronidase (HYAL) originating from Bos taurus with a N-terminal secretion signal and a C-terminal anchoring signal
SEQ ID NO: 69 is an amino acid sequence of hyaluronidase (HYAL) originating from Apis mellifera with a N-terminal secretion signal and a C-terminal anchoring signal
SEQ ID NO: 70 is an amino acid sequence of hyaluronidase (HYAL) originating from Macaca mulatta with a N-terminal secretion signal and a C-terminal anchoring signal
SEQ ID NO: 71 is an amino acid sequence of hyaluronidase (HYAL) originating from Vespa magnifica with a N-terminal secretion signal and a C-terminal anchoring signal
SEQ ID NO: 72 is a nucleic acid sequence of glutamine-fructose-6-phosphate amidotransferase (GFA1) originating from Saccharomyces cerevisiae
SEQ ID NO: 73 is a reencoded nucleic acid sequence of glutamine-fructose-6-phosphate amidotransferase (GFA1) originating from Chlorella virus 1 (PBCV-1)
SEQ ID NO: 74 is a reencoded nucleic acid sequence of glutamine-fructose-6-phosphate amidotransferase (GFA1) originating from Chlorella virus 1 (PBCV-1)
SEQ ID NO: 75 is an amino acid sequence of glutamine-fructose-6-phosphate amidotransferase (GFA1) originating from Saccharomyces cerevisiae
SEQ ID NO: 76 is an amino acid sequence of glutamine-fructose-6-phosphate amidotransferase (GFA1) originating from Chlorella virus 1 (PBCV-1)
SEQ ID NO: 77 is a nucleic acid sequence of UDP-N-acetylglucosamine pyrophosphorylase (QRI1) originating from Saccharomyces cerevisiae
SEQ ID NO: 78 is an amino acid sequence of UDP-N-acetylglucosamine pyrophosphorylase (QRI1) originating from Saccharomyces cerevisiae
SEQ ID NO: 79 is a nucleic acid sequence of Phosphoglucomutase-1 (PGM1) originating from Saccharomyces cerevisiae
SEQ ID NO: 80 is an amino acid sequence of Phosphoglucomutase-1 (PGM1) originating from Saccharomyces cerevisiae
SEQ ID NO: 81 is a nucleic acid sequence of UTP-glucose-1-phosphate uridylyltransferase (UGP1) originating from Saccharomyces cerevisiae
SEQ ID NO: 82 is an amino acid sequence of UTP-glucose-1-phosphate uridylyltransferase (UGP1) originating from Saccharomyces cerevisiae
SEQ ID NO: 83 is a nucleic acid sequence of Glucosamine 6-phosphate N-acetyltransferase (GNA1) originating from Saccharomyces cerevisiae
SEQ ID NO: 84 is an amino acid sequence of Glucosamine 6-phosphate N-acetyltransferase (GNA1) originating from Saccharomyces cerevisiae
SEQ ID NO: 85 is a nucleic acid sequence of phosphoacetylglucosamine mutase (PCM1) originating from Saccharomyces cerevisiae
SEQ ID NO: 86 is an amino acid sequence of phosphoacetylglucosamine mutase (PCM1) originating from Saccharomyces cerevisiae
SEQ ID NO: 87 is the nucleic acid sequence of promoter pTDH3
SEQ ID NO: 88 is the nucleic acid sequence of promoter pTDH3.Sk
SEQ ID NO: 89 is the nucleic acid sequence of promoter pTDH3-1.sba
SEQ ID NO: 90 is the nucleic acid sequence of promoter pTDH3-1.Sar
SEQ ID NO: 91 is the nucleic acid sequence of promoter pENO2
SEQ ID NO: 92 is the nucleic acid sequence of promoter pTEF3
SEQ ID NO: 93 is the nucleic acid sequence of promoter pTEF1
SEQ ID NO: 94 is the nucleic acid sequence of promoter pTEF1.Ago
SEQ ID NO: 95 is the nucleic acid sequence of promoter pTEF1.Sba
SEQ ID NO: 96 is the nucleic acid sequence of promoter pPDC1
SEQ ID NO: 97 is the nucleic acid sequence of promoter pCCW12
SEQ ID NO: 98 is the nucleic acid sequence of promoter pCCW12.Sm
SEQ ID NO: 99 is the nucleic acid sequence of promoter pCCW12.Sk
SEQ ID NO: 100 is the nucleic acid sequence of promoter pCCW12.Sba
SEQ ID NO: 101 is the nucleic acid sequence of promoter pCCW12.Sar
SEQ ID NO: 102 is the nucleic acid sequence of promoter pNUP57
SEQ ID NO: 103 is the nucleic acid sequence of promoter pCCW10.Ago
SEQ ID NO: 104 is the nucleic acid sequence of promoter pCWP2
SEQ ID NO: 105 is the nucleic acid sequence of promoter pFBA1
SEQ ID NO: 106 is the nucleic acid sequence of promoter pCCW120.Sm
SEQ ID NO: 107 is the nucleic acid sequence of promoter pCUP1
SEQ ID NO: 108 is the nucleic acid sequence of promoter pMET6
SEQ ID NO: 109 is the nucleic acid sequence of promoter pMET25
SEQ ID NO: 110 is the nucleic acid sequence of promoter pSAM1
SEQ ID NO: 111 is the nucleic acid sequence of terminator tTPI1
SEQ ID NO: 112 is the nucleic acid sequence of terminator tMET25
SEQ ID NO: 113 is the nucleic acid sequence of terminator tDIT1
SEQ ID NO: 114 is the nucleic acid sequence of terminator tRPL3
SEQ ID NO: 115 is the nucleic acid sequence of terminator tRPL3.Sm
SEQ ID NO: 116 is the nucleic acid sequence of terminator tRPL3.sba
SEQ ID NO: 117 is the nucleic acid sequence of terminator tRPL41B
SEQ ID NO: 118 is the nucleic acid sequence of terminator tRPL41B.Sba
SEQ ID NO: 1119 is the nucleic acid sequence of terminator tRPL15A
SEQ ID NO: 120 is the nucleic acid sequence of terminator tRPL15A.Sm
SEQ ID NO: 121 is the nucleic acid sequence of terminator tRPL15A.sba
SEQ ID NO: 122 is the nucleic acid sequence of terminator tIDP1
SEQ ID NO: 123 is the nucleic acid sequence of terminator tIDP1.Sba
SEQ ID NO: 124 is the nucleic acid sequence of terminator tTEF1.sba
SEQ ID NO: 125 is the nucleic acid sequence of the secretion sequence added in 5′
SEQ ID NO: 126 is the amino acid sequence of the secretion sequence added in N-ter
SEQ ID NO: 127 is the nucleic acid sequence of the anchoring sequence added in 3′
SEQ ID NO: 128 is the amino acid sequence of the anchoring sequence added in C-ter

### DETAILED DESCRIPTION OF THE INVENTION

The inventors have conceived genetically modified cells, and especially genetically modified yeasts, having the ability to produce chondroitin, as compared to the parent cells, and especially as compared to the parent yeasts which are not naturally capable of doing so.

These genetically modified cells, are described throughout the present specification.

### Definitions

The term “chondroitin” is a polysaccharide of N-acetylgalactosamine and glucuronique acid, linked via alternating β-(1→4) and β-(1→3) glycosidic bonds (alternating polymer of β-glucuronic acid-(1→3)-N-acetyl-β-galactosamine-4-sulfate-(1→4)) and having the chemical formula (C₁₄H₂₁NO₁₁)n.

The term “Chondroitin”, as used herein, is intended to cover chondroitin and its derivatives, which include but are not limited to chondroitin sulfates, useful for applications in a cosmetic product, flavor product a fragrance product, a food product, a food, a beverage, a texturant, a pharmaceutical composition, a dietary supplement, a nutraceutical, a cleaning product and/or a dental and/or an oral hygiene composition or combinations thereof.

Chondroitin sulfates include but are not limited to Chondroitin-4-sulfate, Chondroitin-6-sulfate, Chondroitin-2,6-sulfate, Chondroitin-4,6-sulfate and/or mixtures thereof.

The site of sulfation of Chondroitin-4-sulfate is carbon 4 of the N-acetylgalactosamine (GalNAc) sugar.

The site of sulfation of Chondroitin-6-sulfate is carbon 6 of the GalNAc sugar.

The site of sulfation of Chondroitin-2,6-sulfate is carbon 2 of the glucuronic acid and 6 of the GalNAc sugar.

The site of sulfation of Chondroitin-4,6-sulfate is carbons 4 and 6 of the GalNAc sugar.

Chondroitin derivatives, such as Chondroitin sulfate, may be useful in alleviating the risk of, reducing, preventing and/or treating osteoarthritis, cartilage deterioration, and osteoarthritis-related joint pain, tenderness, and/or swelling.

In particular, Chondroitin derivatives, such as Chondroitin sulfate, may be useful in alleviating the risk of, reducing, preventing and/or treating osteoarthritis; osteoarthritis-related joint pain, joint tenderness, and joint swelling; joint degeneration; and/or cartilage deterioration.

Chondroitin, in particular Chondroitin sulfate, may be used alone or with glucosamine as a dietary supplement for treatment of osteoarthritis; osteoarthritis-related joint pain, joint tenderness, and joint swelling; joint degeneration; and/or cartilage deterioration.

Chondroitin, in particular Chondroitin sulfate, may be prepared as a composition.

Chondroitin derivatives may be prepared from chondroitin through an appropriate chemical or enzymatic step.

Chondroitin may be produced in a recombinant cell.

As used herein, the term “recombinant” when used in reference to a cell indicates that the cell has been modified by the introduction of an endogenous and/or heterologous nucleic acid or protein into the cell or the alteration of a native cell, or that the cell is derived from a cell so modified. Thus, for example, recombinant cells express genes or nucleic acid that are not found within the native (non-recombinant) form of the cell or express native (eg endogenous) genes at a different level than their native level or express additional or supplementary copies of native (eg endogenous) at a different level than their native level.

As used herein, the term “recombinant”, when used in reference to a nucleic acid or vector, are sequences formed/obtained by technics of genetic engineering well known to the man skilled in the art. The guidelines of the US National Institute for Health (NIH) accordingly indicate that:
“recombinant [ . . . ] nucleic acids are defined as:
i. molecules that (a) are constructed by joining nucleic acid molecules and (b) that can replicate in a living cell, i.e., recombinant nucleic acids;”,
which reflects the conventional use of the word “recombinant” attached to nucleic acid sequences to mean recombined following the insertion of, or joining together with, another nucleic acid.

Proteins that result from the expression of recombinant DNA or recombinant vector within living cells are also termed recombinant proteins.

The term “recombinant” is accordingly synonymous with the term “genetically modified”. The term “gene” is synonymous with the term “nucleic acid” or “nucleotide sequence”.

A recombinant nucleic acid sequence for use in a recombinant cell, and in particular a recombinant yeast, of the invention may be provided in the form of a nucleic acid construct. The term “nucleic acid construct” refers to as a nucleic acid molecule, either single- or double-stranded, which is isolated or derived from a native (eg endogeneous) naturally occurring gene or is a heterologous nucleic acid or which has been modified to contain segments of nucleic acids which are combined and juxtaposed in a manner which would not otherwise exist in nature. The term “nucleic acid construct” is synonymous with the term “expression cassette” or “heterologous nucleic acid expression cassette” when the nucleic acid construct contains one or more regulatory elements required for expression of a coding sequence, wherein said control sequences are operably linked to said coding sequence. Non-limiting examples of regulatory elements include promoters, enhancers, silencers, terminators, and poly-A signals.

A recombinant nucleic acid sequence for use in a recombinant cell of the invention may be provided in the form of an expression vector, wherein the polynucleotide sequence is operably linked to at least one control sequence for the expression of the polynucleotide sequence in a recombinant cell.

The terms “obtained from” or “originate from” or “originating from” a microorganism or animal generally means that a substance (e.g., a nucleic acid molecule or polypeptide) originating from the microorganism or animal is native to that microorganism or animal.

The terms “derived from” a microorganism or animal generally means that a substance (e.g., a nucleic acid molecule or polypeptide) derived from the microorganism or animal is the result of modifications brought to the substance native to (i.e. present as such) in that microorganism or animal. For example, regarding a nucleic acid sequence derived from a microorganism or animal, said nucleic acid sequence can correspond to a reencoded and/or truncated version of the native nucleic acid sequence from this microorganism or animal. Other modifications well known to the man skilled in the art can be brought to the native substance of a microorganism or animal leading to the substance used being “derived from” said microorganism or animal.

As used herein, the term “polypeptide” refers to a molecule comprising amino acid residues linked by peptide bonds and containing more than five amino acid residues. The amino acids are identified by either the single-letter or three-letter designations. The term “protein” as used herein is synonymous with the term “polypeptide” and may also refer to two or more polypeptides. Thus, the terms “protein”, “peptide” and “polypeptide” can be used interchangeably. Polypeptides may optionally be modified (e.g., glycosylated, phosphorylated, acylated, farnesylated, prenylated, sulfonated, and the like) to add functionality. Polypeptides exhibiting activity may be referred to as enzymes. It will be understood that, as a result of the degeneracy of the genetic code, a multitude of nucleotide sequences encoding a given polypeptide may be produced.

A polypeptide encoded by a recombinant nucleic acid for use in a recombinant cell, and in particular in a recombinant yeast, of the invention may comprise a signal peptide and/or a propeptide sequence. In the event that a polypeptide expressed by a recombinant cell, and in particular a recombinant yeast, of the invention comprises a signal peptide and/or a propeptide, sequence identity may be calculated over the mature polypeptide sequence.

The term “operably linked” as used herein refers to two or more nucleic acid sequence elements that are physically linked and are in a functional relationship with each other. For instance, a promoter is operably linked to a coding sequence if the promoter is able to initiate or regulate the transcription or expression of the coding sequence, in which case the coding sequence should be understood as being “under the control of” the promoter. Generally, when two nucleic acid sequences are operably linked, they will be in the same orientation and usually also in the same reading frame. They usually will be essentially contiguous, although this may not be required.

The term “native” or “endogenous” as used herein with reference to molecules, and in particular enzymes and nucleic acids, indicates molecules that are expressed in the organism in which they originated or are found in nature.

The term “endogenous gene” means that the gene was present in the cell before any genetic modification, in the wild-type strain. Endogenous genes may be overexpressed by introducing heterologous sequences in addition to, or to replace endogenous regulatory elements, or by introducing one or more additional or supplementary copies of the gene into the chromosome or a plasmid (said additional or supplementary copies being designated “exogenous or heterologous genes” or “heterologous nucleotide sequences” or “heterologous nucleic acids” as defined herein). Endogenous genes may also be modified to modulate their expression and/or activity. For example, mutations may be introduced into the coding sequence to modify the gene product or heterologous sequences may be introduced in addition to or to replace endogenous regulatory elements. Modulation of an endogenous gene may result in the up-regulation and/or enhancement of the activity of the gene product, or alternatively, in the down-regulation and/or attenuation of the activity of the endogenous gene product. Another way to enhance expression of endogenous genes is to introduce one or more additional or supplementary copies of the gene onto the chromosome or a plasmid (said supplementary copies being “exogenous or heterologous genes” or “heterologous nucleotide sequences” or heterologous nucleic acids” as defined here-after).

By “one or more additional or supplementary copies of a gene” according to the invention, it is for example understood in the present invention from 1 to 50 copies, in particular from 1 to 30 copies, more particularly from 1 to 20 copies, and preferably from 1 to 10 copies. Said copies may be inserted into the same locus or into different loci of a recombinant cell of the invention.

The term “exogenous gene” means that the gene was introduced into a cell, by means well known to the man skilled in the art, whereas this gene is or is not naturally occurring in the wild-type cell. Cells can express exogenous genes if these genes are introduced into the cell with all the elements allowing their expression in the cell. Transforming cells with exogenous DNA is a routine task for the man skilled in the art. Exogenous genes may be integrated into the host chromosome, or be expressed extra-chromosomally from plasmids or vectors. A variety of plasmids, which differ with respect to their origin of replication and their copy number in the cell, are all known in the art. The sequence of exogenous genes may be adapted for its expression in the cell. Indeed, the man skilled in the art knows the notion of codon usage bias and how to adapt nucleic sequences for a particular codon usage bias without modifying the deduced protein. In particular embodiments, codon optimized genes express native enzymes.

The term “heterologous gene” or “heterologous nucleic acid sequence” refers to a gene or nucleic acid sequence not normally found in a given cell in nature. As such, a heterologous nucleic acid sequence may be: (a) foreign to its host cell (i.e. is“exogenous” to the cell); (b) naturally found in the host cell (i.e. “endogenous”) but present at an unnatural quantity in the cell (i.e., greater or lesser quantity than naturally found in the host cell); or (c) be naturally found in the host cell but positioned outside of its natural locus.

In the present application, all genes are referenced with their common names and with references to their nucleotide sequences and, the case arising, to their amino acid sequences. Using the references given in accession number for known genes, those skilled in the art are able to determine the equivalent genes in other organisms, bacterial strains, yeast, fungi, mammals, plants, etc. This routine work is advantageously done using consensus sequences that can be determined by carrying out sequence alignments with genes derived from other cells and designing degenerated probes to clone the corresponding gene in another organism.

The man skilled in the art knows different means to modulate, and in particular up-regulate or down-regulate, the expression of endogenous genes. For example, a way to enhance expression of, or overexpress, endogenous genes is to introduce one or more additional or supplementary copies of the gene onto the chromosome or a plasmid.

Another way is to replace the endogenous promoter of a gene with a stronger promoter. These promoters may be homologous or heterologous. Promoters particularly interesting in the present invention are described in more detail elsewhere in the present specification.

The nucleic acid expression construct may further comprise 5′ and/or 3′ recognition sequences and/or selection markers.

The term “inducible promoter” is used to qualify a promoter whose activity is induced, i.e. increased:
in the presence of one or more particular metabolite(s). The higher the metabolite concentration in the medium, the stronger the promoter activity; or
in the presence of a low concentration, or in the absence, of one or more metabolite(s). These metabolites are different from those whose increasing presence induces the activity of the promoter. The lower the metabolite concentration in the medium, the stronger the promoter activity.

The term “repressible promoter” is used to qualify a promoter whose activity is repressed, i.e. reduced:
in the presence of one or more particular metabolite(s). The higher the metabolite concentration in the medium, the weaker the promoter activity; or
in the presence of a low concentration, or in the absence, of one or more metabolite(s). These metabolites are different from those whose increasing presence represses the activity of the promoter. The lower the metabolite concentration in the medium, the weaker the promoter activity.

As used herein, the term “anchoring signal” when used in conjunction with a protein or polypeptide such as an enzyme (such as, for example hyaluronidase) means for example a first nucleic acid encoding a protein that is operably linked to a second nucleic acid encoding a protein or a polypeptide, or a first protein or polypeptide that is operably linked to a second protein or polypeptide, such as an enzyme (such as, for example hyaluronidase to form, for example a fusion protein), and that enables the cellular transport machinery of a cell, in particular of a S. cerevisiae cell, to correctly anchor and/or position in the membrane of the cell the second protein operably linked to the first protein.

As used herein, the term “secretion signal” when used in conjunction with a protein or polypeptide such as an enzyme (such as, for example hyaluronidase) means for example a first nucleic acid encoding a peptide or protein that is operably linked to a second nucleic acid encoding a protein, or a first protein that is linked to a second protein, such as an enzyme (such as, for example hyaluronidase to form, for example a fusion protein), and that enables the cellular transport machinery of a cell, in particular of a S. cerevisiae cell, to locate at least the second protein to the membrane of the cell and to secrete the second protein outside of the cell, after the first protein has, for example, been cleaved from the second protein.

As used herein the terms “secretion signal” and “anchoring signal” when used in conjunction with a protein or polypeptide such as an enzyme (such as, for example, hyaluronidase), mean for example a first nucleic acid encoding a peptide or protein that is operably linked to a second nucleic acid encoding a protein, or a first protein that is operably linked to a second protein, such as an enzyme (such as, for example hyaluronidase), and that enables the cellular transport machinery of a cell, in particular of a S. cerevisiae cell, to locate at least the second protein to the membrane of the cell wherein if the second protein is also operably linked to an “anchoring signal” the second protein is not secreted but remains attached to the membrane of the cell. In some cases, a secretion-anchoring signal may provide a dual secretion signal and anchoring signal function.

Sequences of secretion and anchoring signals, methods for the expression, anchoring and/or secretion of heterologous proteins, such as enzymes (such as, for example, hyaluronidase) on the surface of a cell (such, as for example, a yeast cell) are well known in the art (see for example, Ast et al (2013) Cell 152: 1134-1145, Ast and Schuldiner (2013) Crit Rev Biochem Mol Biol 48(3) 273-288, Van der Vaart et al (1997) Applied Environmental Microbiology 63(2) 615-620 and the entire contents of each of these publications are incorporated herein by reference). The “activity” of an enzyme is used interchangeably with the term “function” and designates, in the context of the invention, the capacity of an enzyme to catalyze a desired reaction. The amount of an enzyme in a host cell may be altered by modifying the transcription of the gene that encodes the enzyme. This can be achieved for example by modifying the copy number of the nucleotide sequence encoding the enzyme (e.g., by using a higher or lower copy number expression vector comprising the nucleotide sequence, or by introducing additional copies of the nucleotide sequence into the genome of the host cell or by deleting or disrupting the nucleotide sequence in the genome of the host cell), by changing the order of coding sequences on a polycistronic mRNA of an operon or breaking up an operon into individual genes each with its own control elements, or by increasing the strength of the promoter or operator to which the nucleotide sequence is operably linked.

Alternatively or in addition, the copy number of an enzyme in a host cell may be altered by modifying the level of translation of an mRNA that encodes the enzyme. This can be achieved for example by modifying the stability of the mRNA, modifying the sequence of the ribosome binding site, modifying the distance or sequence between the ribosome binding site and the start codon of the enzyme coding sequence, modifying the entire intercistronic region located “upstream of” or adjacent to the 5′ side of the start codon of the enzyme coding region, stabilizing the 3′-end of the mRNA transcript using hairpins and specialized sequences, modifying the codon usage of enzyme, altering expression of rare codon tRNAs used in the biosynthesis of the enzyme, and/or increasing the stability of the enzyme, as, for example, via mutation of its coding sequence.

The activity of an enzyme in a host cell can be altered in a number of ways, including, but not limited to, expressing a modified form of the enzyme that exhibits increased or decreased solubility in the host cell, expressing an altered form of the enzyme that lacks a domain through which the activity of the enzyme is inhibited, expressing a modified form of the enzyme that has a higher or lower Kcat or a lower or higher Km for the substrate, or expressing an altered form of the enzyme that is more or less affected by feed back or feed-forward regulation by another molecule in the pathway. The terms “encoding” or “coding for” refer to the process by which a polynucleotide, through the mechanisms of transcription and translation, produces an amino-acid sequence.

The gene(s) encoding the enzyme(s) considered in the present invention can be exogenous or endogenous.

The methods implemented in the present invention preferably require the use of one or more chromosomal integration constructs for the stable introduction of a heterologous nucleotide sequence into a specific location on a chromosome or for the functional disruption of one or more target genes in a genetically modified cell. In some embodiments, disruption of a target gene prevents the expression of the related functional protein. In some embodiments, disruption of a target gene results in the expression of a non-functional protein from the disrupted gene.

Parameters of chromosomal integration constructs that may be varied in the practice of the present invention include, but are not limited to, the lengths of the homologous sequences; the nucleotide sequence of the homologous sequences; the length of the integrating sequence; the nucleotide sequence of the integrating sequence; and the nucleotide sequence of the target locus. In some embodiments, an effective range for the length of each homologous sequence is 20 to 5,000 base pairs, preferentially 50 to 100 base pairs. In particular embodiments, the length of each homologous sequence is about 50 base pairs. For more information on the length of homology required for gene targeting, see D. Burke et al., Methods in yeast Genetics—A cold spring harbor laboratory course Manual (2000).

In some embodiments, (a) disrupted gene(s) in which the above-mentioned DNA construct(s) is/are intended to be inserted may advantageously comprises one or more selectable markers useful for the selection of transformed cells. Preferably, said selectable marker(s) are comprised in the DNA construct(s) according to the present invention.

In some embodiments, the selectable marker is an antibiotic resistance marker. Illustrative examples of antibiotic resistance markers include, but are not limited to the, NAT1, AUR1-C, HPH, DSDA, KAN<R>, and SH BLE gene products. The NAT 1 gene product from S. noursei confers resistance to nourseothricin; the AUR1-C gene product from Saccharomyces cerevisiae confers resistance to Auerobasidin A (AbA); the HPH gene product of Klebsiella pneumoniae confers resistance to Hygromycin B; the DSDA gene product of E. coli allows cells to grow on plates with D-serine as the sole nitrogen source; the KAN<R> gene of the Tn903 transposon confers resistance to G418; and the SH BLE gene product from Streptoalloteichus hindustanus confers resistance to Zeocin (bleomycin).

In some embodiments, the antibiotic resistance marker is deleted after the genetically modified cell of the invention is isolated. The man skilled in the art is able to choose suitable marker in specific genetic context.

In a particular embodiment, a recombinant cell according to the invention is devoid of any antibiotic resistance marker. This advantageously prevents the necessity to add antibiotics in the selection medium.

In some embodiments, the selectable marker rescues an auxotrophy (e.g., a nutritional auxotrophy) in the genetically modified cell. In such embodiments, a parent cell, and in particular a parent yeast, comprises a functional disruption in one or more gene products that function in an amino acid or nucleotide biosynthetic pathway, such as, for example, the HIS3, LEU2, LYS1, LYS2, MET15, TRP1, ADE2, and URA3 gene products in yeast, which renders the parent cell incapable of growing in media without supplementation with one or more nutrients (auxotrophic phenotype). The auxotrophic phenotype can then be rescued by transforming the parent cell with a chromosomal integration encoding a functional copy of the disrupted gene product (in some embodiments, the functional copy of the gene may originate from close species, such as Kluyveromyces, Candida etc.), and the genetically modified cell generated can be selected based on the loss of the auxotrophic phenotype of the parent cell.

For each of the nucleic acid sequences comprising a promoter sequence, a coding sequence (e.g. an enzyme coding sequence), or a terminator sequence, reference sequences are described herein. The present description also encompasses nucleic acid sequences having specific percentages of nucleic acid identity with a reference nucleic acid sequence.

For each or the amino acid sequences of interest, reference sequences are described herein. The present description also encompasses amino acid sequences (e.g. enzyme amino acid sequences), having specific percentages of amino acid identity with a reference amino acid sequence.

For obvious reasons, in all the present description, a specific nucleic acid sequence or a specific amino acid sequence which complies with, respectively, the considered nucleotide or amino acid identity, should further lead to obtaining a protein (or enzyme) which displays the desired biological activity. As used herein, the “percentage of identity” between two nucleic acid sequences or between two amino acid sequences is determined by comparing both optimally aligned sequences through a comparison window.

The portion of the nucleotide or amino-acid sequence in the comparison window may thus include additions or deletions (for example “gaps”) as compared to the reference sequence (which does not include these additions or these deletions) so as to obtain an optimal alignment between both sequences.

The terms “sequence homology” or “sequence identity” or “homology” or “identity” are used interchangeably herein. For the purpose of the invention, it is defined here that in order to determine the percentage of sequence homology or sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/based or amino acids. The sequence identity is the percentage of identical matches between the two sequences over the reported aligned region. A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley).

The percent sequence identity between two amino acid sequences or between two nucleotide sequences may be determined using the Needleman and Wunsch algorithm for the alignment of two sequences. (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE.

For the purpose of the invention, the NEEDLE program from the EMBOSS package was used (version 2.8.0 or higher, EMBOSS: The European Molecular Biology Open Software Suite (2000) Rice, P. Longden J. and Bleasby, A. Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap opening penalty of 10 and a gap extension penalty of 0.5. No end gap penalty is added. In the Output section, Yes has been indicated in response to the question “Brief identity and similarity” and “SRS pairwise” indicated as Output alignment format.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment. The identity defined as herein can be obtained from NEEDLE by using the NOBRIEF option and is labeled in the output of the program as “longest-identity”.

The similarity of nucleotide and amino acid sequences, i.e. the percentage of sequence identity, can be determined via sequence alignments using several other art-known algorithms, preferably with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) available e.g. on https://www.ebi.ac.uk/Tools/msa/clustalo/ or the GAP program (mathematical algorithm of the University of Iowa) or the mathematical algorithm of Myers and Miller (1989—Cabios 4: 11-17) or Clone Manager 9. Preferred parameters used are the default parameters as they are set on https://www.ebi.ac.uk/Tools/msa/clustalo/.

The grade of sequence identity (sequence matching) may be calculated using e.g. BLAST, BLAT or BlastZ (or BlastX). A similar algorithm is incorporated into the BLASTN and BLASTP programs of Altschul et al (1990) J. Mol. Biol. 215, 403-410. BLAST polynucleotide searches are performed with the BLASTN program, score=100, word length=12, to obtain polynucleotide sequences that are homologous to those nucleic acids which encode the relevant protein.

BLAST protein searches are performed with the BLASTP program, score=50, word length=3, to obtain amino acid sequences homologous to the SHC polypeptide. To obtain gapped alignments for comparative purposes, Gapped BLAST is utilized as described in Altschul et al (1997) Nucleic Acids Res. 25, 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1: 154-162) or Markov random fields. When percentages of sequence identity are referred to in the present application, these percentages are calculated in relation to the full length of the longer sequence, if not specifically indicated otherwise.

In particular embodiments, % identity between two sequences is determined using CLUSTAL O (version 1.2.4).

The “fermentation” or “culture” is generally conducted in fermenters with an appropriate culture medium adapted to the cell being cultivated, containing at least one simple carbon source, and if necessary co-substrates.

The term “fermentation composition” refers to a composition which comprises genetically modified host cells and products or metabolites produced by the genetically modified host cells. An example of a fermentation composition is a whole cell broth, which can be the entire contents of a vessel (e.g., a flasks, plate, or fermentor), including cells, aqueous phase, and compounds produced from the genetically modified host cells.

The term “medium” refers to a culture medium or cultivation medium or fermentation medium.

For maximal production of chondroitin, the recombinant cells used as production hosts preferably have a high rate of carbohydrate utilization. These characteristics may be conferred by mutagenesis and selection, genetic engineering, or may be natural. Fermentation media, or “culture medium” or “cultivation medium”, for the present cells may contain at least about 10 g/L of glucose and/or sucrose. Additional carbon substrates may include but are not limited to monosaccharides such as fructose, mannose, xylose and arabinose; oligosaccharides such as lactose maltose, galactose, or sucrose; polysaccharides such as starch or cellulose or mixtures thereof and unpurified mixtures from renewable feedstocks such as cheese whey permeate cornsteep liquor, sugar beet molasses, and barley malt. Other carbon substrates may include glycerol, acetate and/or ethanol.

Hence, it is contemplated that the source of carbon utilized in the present invention may encompass a wide variety of carbon containing substrates and will only be limited by the choice of cell, and in particular of yeast.

Although it is contemplated that all of the above-mentioned carbon substrates and mixtures thereof are suitable in the present invention, preferred carbon substrates are glucose, fructose, and sucrose, or mixtures of these with C5 sugars such as xylose and/or arabinose for cells, and in particular yeasts, modified to use C5 sugars, and more particularly glucose.

Preferred carbon substrates are glucose or sucrose.

In addition to an appropriate carbon source, fermentation media may contain suitable minerals, salts, cofactors, buffers and other components, known to those skilled in the art, suitable for the growth of the cultures and promotion of the enzymatic pathway necessary for the production of the desired product.

Besides, additional genetic modifications suitable for the growth of recombinant cells according to the invention may be considered.

The terms “Aerobic conditions” refers to concentrations of oxygen in the culture medium that are sufficient for an aerobic or facultative anaerobic cell, and in particular yeast, to use di-oxygene as a terminal electron acceptor.

“Microaerobic condition” refers to a culture medium in which the concentration of oxygen is less than that in air, i.e. oxygen concentration up to 6% O₂.

An “appropriate culture medium” designates a medium (e.g. a sterile, liquid medium) comprising nutrients essential or beneficial to the maintenance and/or growth of the cell such as carbon sources or carbon substrate, nitrogen sources, for example, peptone, yeast extracts, meat extracts, malt extracts, urea, ammonium sulfate, ammonium chloride, ammonium nitrate and ammonium phosphate; phosphorus sources, for example, monopotassium phosphate or dipotassium phosphate; trace elements (e.g., metal salts), for example magnesium salts, cobalt salts and/or manganese salts; as well as growth factors such as amino acids, vitamins, growth promoters, and the like. The term “carbon source” or “carbon substrate” or “source of carbon” according to the present invention denotes any source of carbon that can be used by those skilled in the art to support the normal growth of a cell, including hexoses (such as glucose, galactose or lactose), pentoses, monosaccharides, oligosaccharides, disaccharides (such as sucrose, cellobiose or maltose), molasses, starch or its derivatives, cellulose, hemicelluloses and combinations thereof.

Culture mediums that are particularly appropriate to produce recombinant cells of the invention, and in particular recombinant yeasts of the invention, are described further below in the text. By “controlled” molecular weight of a chondroitin of the invention is intended to mean that at least 80%, in particular at least 85%, of the chondroitin produced by a method of the invention has a molecular weight comprised within a certain pre-determined molecular weight's range through the regulation of at least one parameter of the method and/or of the recombinant cell of the invention, such as for example:
the nature and origin of the nucleic acid encoding the polypeptide having chondroitinase activity of the recombinant cell,
the nature and origin of the promoter controlling the expression of the nucleic acid encoding the polypeptide having chondroitinase activity of the recombinant cell,
the presence of an anchoring and/or of a secretion signal associated to the the polypeptide having chondroitinase activity of the recombinant cell,
the pH of the culture medium during the step of culturing the recombinant cell, and/or
the duration of the culturing of the recombinant cell.

As used herein, the term “about” refers to a reasonable range about a value as determined by the practitioner of skill. In certain embodiments, the term about refers to ±one, two, or three standard deviations. In certain embodiments, the term about refers to ±5%, 10%, 20%, or 25%. In certain embodiments, the term about refers to ±0.1, 0.2, or 0.3 logarithmic units, e.g. pH units.

### General Features of Genetic Modifications Introduced According to the Invention

All the genome modifications are inserted in recombinant cells, and in particular recombinant yeasts, according to known genetic engineering techniques:
The successive nucleic acid sequences included in a gene construct that is introduced in the recombinant cell genome according to the invention are of the following structure:

Prom₁-ORF₁-term₁-ORF₂-gene₂-term₂- . . . / . . . -Promₙ-ORFₙ-termₙ, wherein:
Prom1 is a sequence regulating the expression of the coding sequence ORF1,
ORF1 is a nucleic acid sequence encoding a desired protein PROT1, and especially a desired enzyme PROT1,
Term1 is a transcription terminator sequence that mediates transcriptional termination by providing signals in the newly synthesized mRNA that trigger processes which release the mRNA from the transcriptional complex, and
“1”, “2”, . . . / . . . “n” may or may not describe the same ORF (Open Reading Frame), promoter or terminator. The order of the nucleic acid sequences does not matter. “n” is an integer usually ranging from 5 and 20. These constructs are inserted in one of the recombinant cell chromosome at a controlled location. In some embodiments, the insertion site is neither essential for the functionality of the inserted construct, nor for the viability of the resulting genetically modified cell.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or“controlling for species codon bias.” Codon optimization for other host cells can be readily determined using codon usage tables or can be performed using commercially available software, such as CodonOp (www.idtdna.com/CodonOptfrom) from Integrated DNA Technologies. Optimized coding sequences containing codons preferred by a particular prokaryotic or eukaryotic host (Murray et al, 1989, Nucl Acids Res. 17: 477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al, 1996, Nucl Acids Res. 24: 216-8).
When the recombinant cell is a yeast cell, and in particular is Saccharomyces cerevisiae yeast cell, nucleic acid sequences introduced in the yeast genome and originating from other organisms than Saccharomyces cerevisiae are generally “transcoded” (generally codon-optimized”), meaning that these nucleic acid sequences are synthesized with an optimal codon usage for expression in S. cerevisiae. The nucleotide sequence (and not the protein sequence) of some nucleic acid sequences from S. cerevisiae has also been modified (“transcoded”) to minimize recombination with an endogenous copy of the said gene.
Genes may be deleted through standard procedures used in cell genetic engineering. In some embodiments, the genes targeted for deletion may be interrupted by insertion of one of the above described gene constructs, or alternatively the genes targeted for deletion are replaced by a short stretch of nucleotide.
A nucleic acid sequence may be rendered “inducible or repressible” by deleting an endogenous copy of the nucleic acid sequences (if necessary) and placing a new copy of the ORF under the control of an inducible or repressible promoter. An inducible or repressible promoter is a promoter which activity is modulated or controlled, i.e. either increased or decreased upon a change in the environmental conditions or external stimuli. Induction or repression may be artificially controlled, which encompasses induction or repression by abiotic factors such as chemical compounds not found naturally in the cell, and in particular yeast, of interest, light, oxygen levels, heat or cold. A list and sequences of inducible or repressible promoters are described elsewhere in the present specification.

### Recombinant Cells According to the Invention

The inventors have conceived recombinant cells, in particular recombinant yeasts, having an ability of producing chondroitin.

The present invention relates to recombinant cells, and in particular recombinant yeasts having the ability to produce chondroitin, and wherein this ability to produce chondroitin is obtained through a plurality of alterations that have been introduced in the genome thereof, by genetic engineering methods.

This present invention pertains to a recombinant yeast cell producing chondroitin wherein the recombinant cell comprises:
(a) one or more recombinant nucleic acids encoding a polypeptide having a chondroitin synthase (HCOS) activity;
(b) one or more recombinant nucleic acids encoding a polypeptide having UDP-Glucose dehydrogenase (UDP-GlcDH or HASB) activity; and
(c) one or more recombinant nucleic acids encoding a polypeptide having a UDP-glucose-4-epimerase (kfoA or GNE1) activity.

The present invention further relates to a recombinant host cell producing chondroitin wherein the recombinant host cell comprises:
(a) one or more recombinant nucleic acids encoding a polypeptide having a chondroitin synthase (HCOS) activity;
(b) one or more recombinant nucleic acids encoding a polypeptide having UDP-Glucose dehydrogenase (UDP-GlcDH or HASB) activity;
(c) one or more recombinant nucleic acids encoding a polypeptide having a UDP-glucose-4-epimerase (kfoA or GNE1) activity; and
(d) one or more recombinant nucleic acids encoding a polypeptide having a chondroitinase activity wherein the polypeptide having chondroitinase activity comprises a secretion signal, and optionally an anchoring signal, so that chondroitin, in particular of a desired molecular weight is produced by the host cell.

The inventors have found that an ability to produce chondroitin by cells, and in particular by yeast cells, may be reached by introducing in the genome of these cells a plurality of genetic alterations.

The production of chondroitin by cells of the invention, and in particular by yeast cells of the invention, has been achieved by optimizing the endogenous metabolism of UDP-Glucose and UDP-N-Acetyl-glucosamine and direct the subsequent artificially modified metabolic pathway mainly towards chondroitin production whereas in the same time maintaining an optimal viability of the resulting genetically modified cells.

It has been determined that a chondroitin production by recombinant cells according to the invention may be increased by increasing the conversion of glucose-6-phosphate into the successive intermediate metabolites (i) glucose-1-phosphate, UDP-glucose, UDP-glucuronate and chondroitin or (ii) fructose-6-phosphate, glucosamine-6-phosphate, N-acetyl-glucosamine-6-phosphate, N-acetyl-glucosamine-1-phosphate, UDP-N-acetyl-glucosamine, UDP-N-acetyl-galactosamine and chondroitin, while maintaining a metabolic balance allowing a good viability of the resulting recombinant cells.

Indeed, in order to obtain a viable recombinant cell of the invention, many different constructs were tested in order to obtain a viable and efficient recombinant cell, and in particular a viable recombinant yeast. In particular such recombinant yeasts were difficult to obtain because the temporary accumulation of some intermediates appeared to be toxic for yeasts.

Unexpected technical difficulties were encountered in establishing the conditions suitable for the preparation of a recombinant cell able to produce chondroitin, and in particular to produce chondroitin with a controlled molecular weight.

Indeed, after much research and experimental trial, the inventors discovered that it was possible to cultivate a recombinant cell, and in particular a recombinant yeast cell, more particularly a recombinant Saccharomyces cerevisiae yeast cell, able to produce chondroitin having a controlled molecular weight controlled using the following parameters:
the selection of the nature and origin of the nucleic acid sequences encoding the polypeptide having chondroitinase activity of the recombinant cell, in particular the recombinant yeast, of the invention; and/or
the nature and origin of the promoter controlling the expression of the nucleic acid sequences encoding the polypeptide having chondroitinase activity of the recombinant cell, in particular the recombinant yeast, of the invention; and/or
the optional presence of an anchoring signal, in addition to a secretion signal, associated to the encoded polypeptide having chondroitinase activity of the recombinant cell, in particular the recombinant yeast, according to the invention; and/or
the pH of the cultivation medium during the step of culturing the recombinant cell, in particular the recombinant yeast, according to the invention; and/or
the duration of the culturing of the recombinant cell, in particular the recombinant yeast, according to the invention.

To the inventors' knowledge, this has never been achieved before.

The nucleic acid encoding a polypeptide having a chondroitinase activity in a recombinant cell of the invention may be obtained or derived from at least one of Cupiennius salei, Tityus serrulatus, Bos taurus, Vespa magnifica, Macaca mulatta or Apis mellifera, and preferably from Tityus serrulatus.

The molecular weight of the chondroitin can be in the range of less than 50 kDa, preferably in the range of about 20 kDa to about 50 kDa.

Alternatively, the molecular weight of the chondroitin can be in the range of greater than 50 kDa, preferably in the range of about 50 kDa to about 250 kDa.

In another alternative, the molecular weight of the chondroitin can be in the range of greater than 100 kDa, preferably in the range of about 100 kDa to about 1500 kDa.

The nucleic acid encoding a polypeptide having a UDP-Glucose dehydrogenase (UDP-GlcDH or HASB) activity may be obtained or derived from at least one of Arabidopsis thaliana, Chlorella virus PBCV1 or Streptococcus zooepidemicus, and is in particular obtained or derived from Arabidopsis thaliana or Chlorella virus PBCV.

The nucleic acid encoding a polypeptide having a chondroitin synthase (HCOS) activity may be
(i) a nucleic acid encoding a chondroitin synthase; or
(ii) a nucleic acid encoding a chimeric polypeptide having a chondroitin synthase activity

These nucleic acids may be obtained or derived from at least one of Pasteurella multocida, Chlorella virus PBCV1, Mycobacterium tuberculosis, Homo sapiens, Escherichia coli, Saccharomyces cerevisiae or Penicillium oxalicum.

The nucleic acid encoding a polypeptide having a UDP-glucose-4-epimerase (kfoA or GNE1) activity may be obtained or derived from a bacteria, and in particular from a bacteria selected from the group consisting of Pseudomonas aeruginosa, Pasteurella multocida, and Escherichia coli.

The recombinant cell may further comprise at least one recombinant nucleic acid encoding one or more of:
(i) a polypeptide having glutamine-fructose-6-phosphate amidotransferase (GFA1) activity; and/or
(ii) a polypeptide having UDP-N-acetylglucosamine pyrophosphorylase (QRI1) activity.

The recombinant cell may further comprise at least one recombinant nucleic acid encoding one or more of:
(i) a polypeptide having Phosphoglucomutase-1 (PGM1) activity; and/or
(ii) a polypeptide having UTP-glucose-1-phosphate uridylyltransferase (UGP1) activity; and/or
(iii) a polypeptide having Glucosamine-6-phosphate N-acetyltransferase (GNA1) activity; and/or
(iv) a polypeptide having phosphoacetylglucosamine mutase (PCM1) activity.

In a particular embodiment of the recombinant cell,
the nucleic acid encoding the polypeptide having a glutamine-fructose-6-phosphate amidotransferase (GFA1) activity;
the nucleic acid encoding the polypeptide having UDP-N-acetylglucosamine pyrophosphorylase (QRI1) activity;
the nucleic acid encoding the polypeptide having a Phosphoglucomutase-1 (PGM1) activity;
the nucleic acid encoding the polypeptide having a UTP-glucose-1-phosphate uridylyltransferase (UGP1) activity;
the nucleic acid encoding the polypeptide having Glucosamine-6-phosphate N-acetyltransferase (GNA1) activity; and/or
the nucleic acid encoding the polypeptide having phosphoacetylglucosamine mutase (PCM1);
are obtained or derived from Saccharomyces cerevisiae.

In a particular embodiment, the recombinant host cell is a yeast.

In particular, the recombinant cell belongs to the Saccharomyces genus, or to the Candida genus, or to the Kluyveromyces genus, or to the Ogataea genus, or to the Yarrowia genus, or to the Debaryomyces genus, or to the Ashbya genus, and in particular belongs to the Saccharomyces genus.

In particular, the recombinant cell is selected from the group consisting of Saccharomyces cerevisiae, Saccharomyces boulardii, Saccharomyces bayanus, Saccharomyces paradoxus, Saccharomyces mikatae, Saccharomyces castelli, Candida albicans, Candida glabrata, Candida tropicalis, Kluyveromyces lactis, Kluyveromyces marxianus, Kluyveromyces polysporus, Kluyveromyces thermotolerens, Ogataea polymorpha, Yarrowia lypolytica, Debaryomyces hansenii, and Ashbya gossypii, and is preferably Saccharomyces cerevisiae.

Another object of the invention relates to a method of producing chondroitin of a desired molecular weight comprising:
(a) cultivating a recombinant cell as defined herein in a cultivation medium for a time sufficient to produce chondroitin of the desired molecular weight; and
(b) optionally isolating or recovering the chondroitin from the recombinant cell and/or from the cultivation medium.

In the method, the chondroitin may have a molecular weight of from about 20 kDa to about 50 kDa.

In another embodiment, the chondroitin may have a molecular weight of from about 50 kDa to about 150 kDa.

In another embodiment, the chondroitin may have a molecular weight of from about 150 kDa to about 1500 kDa.

In the method of the invention, the recombinant cell may be a yeast belonging to the Saccharomyces genus, and in particular may be Saccharomyces cerevisiae.

The time sufficient to produce chondroitin of a desired molecular weight may be a period of from about 35 hours to about 50 hours, preferably from about 40 hours to about 50 hours, preferably may be about 48 hours.

In a particular embodiment, the molecular weight of the produced chondroitin may be controlled by regulating the pH of the cultivation medium during step (a).

In an embodiment, the method is carried out on an industrial scale, preferably where the cultivation medium is at least about 100 L, more preferably is in the range of about 1,000 L to about 3,000 L, even more preferably is about 10,000 L or even more preferably is about 100,000 L, or even is about 250,000 L.

Another object of the invention relates to a chondroitin obtained from a recombinant cell according to the invention, or from a method according to the invention.

The invention also relates to a cultivation medium comprising chondroitin of the invention.

Another object of the invention is a composition comprising chondroitin of the invention.

The invention further relates to an industrial product or a consumer product or a consumable comprising (i) the chondroitin having a molecular weight according to the invention, (ii) the cultivation medium according to the invention or (iii) the composition according to the invention.

In particular, the industrial product or the consumer product or the consumable may be a cosmetic product, a flavour product, a fragrance product, a food product, a food, a beverage, a texturant, a pharmaceutical composition, a dietary supplement, a nutraceutical, a cleaning product, a dental and/or an oral hygiene composition

The invention also relates to the use of a recombinant cell of the invention for the production of chondroitin having a molecular weight in the range of from about 20 kDa to about 50 k Da or from about 50 kDa to about 1000 kDa.

Another object of the invention relates to a method for producing chondroitin, comprising the steps of:
(a) culturing a recombinant yeast as defined herein in a cultivation medium; and
(b) recovering the chondroitin from said cultivation medium,
wherein the chondroitin recovered in step (b) has a molecular weight controlled through the selection of:
   the nature and origin of the recombinant nucleic acid encoding a polypeptide having a chondroitinase activity of the recombinant yeast,
   the nature and origin of the promoter controlling the expression of the recombinant nucleic acid encoding a polypeptide having a chondroitinase activity of the recombinant yeast,
   the presence of an anchoring and/or of a secretion signal associated to the polypeptide having a chondroitinase activity of the recombinant yeast,
   the pH of the cultivation medium during the step of culturing the recombinant yeast, and/or
   the duration of the culturing of the recombinant yeast.

Moreover, the invention pertains to a chondroitin-producing recombinant yeast, wherein the recombinant yeast comprises:
(A) one or more recombinant nucleic acids encoding a polypeptide having a glutamine-fructose-6-phosphate amidotransferase (GFA1) activity,
(B) one or more recombinant nucleic acids encoding a polypeptide having a UDP-N-acetylglucosamine pyrophosphorylase (QRI1) activity,
(C) one or more recombinant nucleic acids encoding a polypeptide having a UDP-Glucose dehydrogenase (UDP-GlcDH or HASB) activity,
(D) one or more recombinant nucleic acids encoding a polypeptide having a chondroitin synthase (HCOS) activity, and
(E) one or more recombinant nucleic acids encoding a polypeptide having a UDP-glucose-4-epimerase (kfoA or GNE1) activity said recombinant yeast being Saccharomyces cerevisiae.

In a particular embodiment, the recombinant cell according to the invention, and in particular the recombinant yeast according to the invention, further comprises (F) one nucleic acids encoding a polypeptide having a chondroitinase activity, wherein the nucleic acid encoding a polypeptide having a chondroitinase activity comprises an anchoring signal and/or a secretion signal so that chondroitin, in particular of a desired molecular weight, is produced by the host cell.

In a particular embodiment, the recombinant cell according to the invention, and in particular the recombinant yeast, comprises only one recombinant nucleic acid encoding a polypeptide having a glutamine-fructose-6-phosphate amidotransferase (GFA1) activity.

In a particular embodiment, the recombinant cell according to the invention, and in particular the recombinant yeast comprises between 1 and 10, preferably between 2 and 8 recombinant nucleic acids encoding a polypeptide having a UDP-N-acetylglucosamine pyrophosphorylase (QRI1) activity.

In a particular embodiment, the recombinant cell according to the invention, and in particular the recombinant yeast comprises between 1 and 5 recombinant nucleic acids encoding a polypeptide having a UDP-Glucose dehydrogenase (UDP-GlcDH or HASB) activity.

In another embodiment, the one or more recombinant nucleic acids encoding a polypeptide having a UDP-Glucose dehydrogenase (UDP-GlcDH or HASB) are obtained or derived from at least one of Arabidopsis thaliana, Chlorella virus PBCV1 or Streptococcus zooepidemicus, and in particular are obtained or derived from Chlorella virus PBCV1 or from Arabidopsis thaliana.

In another embodiment, the recombinant cell according to the invention, and in particular the recombinant yeast comprises between 1 and 5 recombinant nucleic acids encoding a polypeptide having a chondroitin synthase (HCOS) activity.

In a particular embodiment, the one or more nucleic acids encoding polypeptide having a chondroitin synthase (HCOS) activity is obtained or derived from at least one of Pasteurella multocida, Chlorella virus PBCV1, Mycobacterium tuberculosis, Homo sapiens, Escherichia coli, Saccharomyces cerevisiae or Penicillium oxalicum.

In another embodiment, the recombinant cell according to the invention, and in particular the recombinant yeast comprises between 1 and 5 recombinant nucleic acids encoding a polypeptide having a UDP-glucose-4-epimerase (kfoA or GNE1) activity.

In a particular embodiment, the one or more nucleic acids encoding a polypeptide having a UDP-glucose-4-epimerase (kfoA or GNE1) activity is obtained or derived from at least one of Pseudomonas aeruginosa, Pasteurella multocida, and Escherichia coli.

In a particular embodiment, the recombinant cell according to the invention, and in particular the recombinant yeast comprises only one recombinant nucleic acid encoding a polypeptide having a chondroitinase activity.

In another embodiment, the one or more recombinant nucleic acids encoding a polypeptide having a chondroitinase activity are obtained or derived from at least one of Cupiennius salei, Tityus serrulatus, Bos taurus, Apis mellifera, Macaca mulatta, or Vespa magnifica.

In another embodiment, the recombinant cell according to the invention, and in particular the recombinant yeast cell may comprise at least one recombinant nucleic acid encoding one or more:
(A) a polypeptide having a Phosphoglucomutase-1 (PGM1) activity, and/or
(B) a polypeptide having a UTP-glucose-1-phosphate uridylyltransferase (UGP1) activity, and/or
(C) a polypeptide having a Glucosamine-6-phosphate N-acetyltransferase (GNA1) activity, and/or
(D) a polypeptide having a phosphoacetylglucosamine mutase (PCM1) activity.

In particular, the recombinant cell according to the invention, and in particular the recombinant yeast cell of the invention comprises at least two, in particular at least three, and more particularly all of the modifications indicated above.

In a particular embodiment, the recombinant nucleic acid encoding polypeptide having a Phosphoglucomutase-1 (PGM1) activity, the recombinant nucleic acid encoding a polypeptide having a UTP-glucose-1-phosphate uridylyltransferase (UGP1) activity, the recombinant nucleic acid encoding a polypeptide having a glutamine-fructose-6-phosphate amidotransferase (GFA1) activity, the recombinant nucleic acid encoding polypeptide having a Glucosamine-6-phosphate N-acetyltransferase (GNA1) activity, the recombinant nucleic acid encoding a polypeptide having a phosphoacetylglucosamine mutase (PCM1) activity and the recombinant nucleic acid encoding a polypeptide having a UDP-N-acetylglucosamine pyrophosphorylase (QRI1) activity are recombinant nucleic acids obtained or derived from a yeast, preferably from Saccharomyces cerevisiae.

In a particular embodiment, the one or more recombinant nucleic acids encoding polypeptides defined above and comprised in a recombinant cell according to the invention, and in particular the recombinant yeast of the invention, are under the the control of a promoter selected from the group consisting of pPDC1, pTDH3, pTDH3.Sk, pTDH3-1.Sba, pCCW12, pCCW120.Sm, pTEF1, pENO2 pTEF1.Ago, and pTEF1.Sba.

In a particular embodiment, the inducible or repressible promoters mentioned in the present specification are selected from the group consisting of promoters inducible or repressible with copper or promoters inducible or repressible with methionine, in particular pCUP1.

In a particular embodiment, the recombinant nucleic acids are under the control of the promoter pCWP2.

Another object of the invention relates to a method for producing chondroitin, said method comprising the steps of:
(a) culturing a recombinant cell according to the invention, and in particular a recombinant yeast, as defined herein in a culture medium; and
(b) recovering the chondroitin from said culture medium.

In a particular embodiment, the chondroitin recovered in step (b) has a pre-determined molecular weight controlled through the selection of:
the nature and origin of the recombinant nucleic acid encoding a polypeptide having a chondroitinase activity, of the recombinant cell, and in particular the recombinant yeast,
the nature and origin of the promoter controlling the expression of the recombinant nucleic acid encoding a polypeptide having a chondroitinase activity of the recombinant cell and in particular of the recombinant yeast,
the presence of an anchoring or of a secretion signal associated to the recombinant nucleic acid encoding a polypeptide having a chondroitinase activity of the recombinant cell and in particular the recombinant yeast,
the pH of the culture medium during the step of culturing the recombinant cell and in particular the combinant yeast, and/or
the duration of the culturing of the recombinant cell and in particular the recombinant yeast.

In a particular embodiment, the method of the invention is for producing chondroitin having a molecular weight strictly inferior to 50 kDa, and in particular strictly inferior to 50 kDa and superior or equal to 20 kDa, wherein (c) the step of recovering the chondroitin from the culture medium is performed 48 hours after the beginning of the culturing of the recombinant cell and in particular the recombinant yeast.

In another embodiment, the method of the invention is for producing chondroitin having a molecular weight superior or equal to 50 kDa and inferior or equal to 1000 kDa. wherein (c) the step of recovering the chondroitin from the culture medium is performed 48 hours after the beginning of the culturing of the recombinant yeast.

In another embodiment, the method of the invention is for producing chondroitin having a molecular weight strictly superior to 1000 kDa, and in particular having a molecular weight comprised between strictly superior to 1000 kDa and 1.5 Million kDa, wherein (c) the step of recovering the chondroitin from the culture medium is performed 48 hours after the beginning of the culturing of the recombinant cell and in particular the recombinant yeast.

In particular, the culture medium comprises at least a carbon source, preferably a carbon source selected from the group consisting of glucose and sucrose.

Another object of the invention relates to the use of a recombinant cell and in particular the recombinant yeast of the invention as defined herein, for the production of chondroitin, in particular of chondroitin of controlled molecular weight.

### Recombinant Nucleic Acids Encoding a Polypeptide Having a Chondroitin Synthase Activity (HCOS)

A recombinant cell according to the invention, and in particular a recombinant yeast of the invention, comprises one or more recombinant nucleic acids encoding a polypeptide having a chondroitin synthase activity.

A polypeptide having a chondroitin synthase activity according to the invention means a polypeptide that converts intermediate metabolites UDP-Glucoronate and UDP-N-actyl-galactosamine into chondroitin ((β-D-1,3-GalNAc-β-D-1,4-GlcA)n).

In some embodiments, the nucleic acid encoding a polypeptide having chondroitin synthase activity may be under the control of an inducible or repressible promoter.

One or more of the recombinant nucleic acids encoding a polypeptide having chondroitin synthase activity may be under the control of a pTDH3-1.Sba or a pTDH3.Sar promoter.

The said one or more recombinant nucleic acids encoding a polypeptide having chondroitin synthase (HCOS) activity may be:
(i) a nucleic acid encoding a chondroitin synthase; or
(ii) a nucleic acid encoding a chimeric polypeptide having a chondroitin synthase activity.

The nucleic acids encoding a polypeptide having a chondroitin synthase activity may be obtained or derived from at least one of Pasteurella multocida, Chlorella virus PBCV1, Mycobacterium tuberculosis, Homo sapiens, Escherichia coli, Saccharomyces cerevisiae or Penicillium oxalicum;

A recombinant cell according to the invention, and in particular a recombinant yeast according to the invention, may comprise one recombinant nucleic acid encoding a polypeptide having chondroitin synthase activity.

Illustratively, the chondroitin synthase gene may be inserted within the JLP1 gene and/or within the SAM3 gene, as it is shown in the examples herein.

In an embodiment of the invention, a recombinant cell, and in particular a recombinant yeast, of the invention comprises:
one recombinant nucleic acid encoding a polypeptide having chondroitin synthase activity;
said one recombinant nucleic acid encoding a polypeptide having chondroitin synthase activity being obtained or derived from at least one of Pasteurella multocida, Chlorella virus PBCV1, Mycobacterium tuberculosis, Homo sapiens, Escherichia coli, Saccharomyces cerevisiae or Penicillium oxalicum;
said one recombinant nucleic acid encoding a polypeptide having chondroitin synthase activity being under the control of an inducible or repressible promoter and/or under the control of a pTDH3-1.Sba or a pTDH3.Sar promoter.

### Recombinant Nucleic Acids Encoding a Polypeptide Having a UDP-Glucose Dehydrogenase Activity (HASB)

A recombinant cell according to the invention, and in particular a recombinant yeast according to the invention, comprises one or more recombinant nucleic acids encoding a polypeptide having UDP-Glucose dehydrogenase (UDP-GlcDH or HASB) activity.

A polypeptide having UDP-Glucose dehydrogenase (UDP-GlcDH or HASB) activity according to the invention means a polypeptide that converts the intermediate metabolite Uridine-Diphosphate-Glucose (UDP-Glucose) into UDP-Glucuronate.

One or more recombinant nucleic acids encoding a polypeptide having a UDP-Glucose dehydrogenase activity may be under the control of a promoter selected from the group consisting of pTEF1.sba, pCCW12 and pTDH3.Sk.

The one or more recombinant nucleic acids encoding a polypeptide having a UDP-Glucose dehydrogenase activity may be obtained or derived from at least one of Arabidopsis thaliana, Chlorella virus PBCV1 or Streptococcus zooepidemicus, and in particular from at least one of Arabidopsis thaliana or Chlorella virus PBCV1.

A recombinant cell according to the invention, and in particular a recombinant yeast according to the invention, may comprise between 1 and 5 recombinant nucleic acids encoding a polypeptide having UDP-Glucose dehydrogenase activity.

Illustratively, the one or more recombinant nucleic acids encoding a polypeptide having UDP-Glucose dehydrogenase activity may be inserted within the JLP1 gene and/or within the SAM3 gene of the recombinant cell, and in particular of the recombinant yeast, as it is shown in the examples herein.

In an embodiment of the invention, a recombinant cell, and in particular a recombinant yeast, of the invention comprises:
between 1 and 5 recombinant nucleic acids encoding a polypeptide having UDP-Glucose dehydrogenase activity;
said recombinant nucleic acid encoding a polypeptide having UDP-Glucose dehydrogenase activity being obtained or derived from at least one of Arabidopsis thaliana, Chlorella virus PBCV1 or Streptococcus zooepidemicus, and in particular from at least one of Arabidopsis thaliana or Chlorella virus PBCV1;
said recombinant nucleic acid encoding a polypeptide having UDP-Glucose dehydrogenase activity being under the control of an inducible or repressible promoter that is functional in the recombinant cells of the invention and/or under the control of a promoter selected from the group consisting of pTEF1.sba, pCCW12 and pTDH3.Sk.
   Recombinant Nucleic Acids Encoding a Polypeptide Having a UDP-Glucose-4-Epimerase Activity (kfoA or GNE1)

A recombinant cell according to the invention, and in particular a recombinant yeast according to the invention, comprises one or more recombinant nucleic acids encoding a polypeptide having UDP-glucose-4-epimerase activity.

A polypeptide having UDP-glucose-4-epimerase activity according to the invention means a polypeptide that converts the intermediate metabolite UDP-N-acetyl-glucosamine into UDP-N-acetyl-galactosamine.

In an embodiment, one or more of the recombinant nucleic acids encoding a polypeptide having a UDP-glucose-4-epimerase activity may be under the control of a promoter selected from the group consisting of pCCW12 or pCCW120.Sm.

The one or more recombinant nucleic acids encoding a polypeptide having a UDP-glucose-4-epimerase activity may be obtained or derived from a bacteria, and in particular from a bacteria selected from the group consisting of Pseudomonas aeruginosa, Pasteurella multocida, and Escherichia coli.

A recombinant cell according to the invention, and in particular a recombinant yeast according to the invention, may comprise between 1 and 5 recombinant nucleic acids encoding a polypeptide having a UDP-glucose-4-epimerase activity.

Illustratively, the one or more recombinant nucleic acids encoding a polypeptide having a UDP-glucose-4-epimerase activity may be inserted within the JLP1 gene and/or within the SAM3 gene of the recombinant cell, and in particular of the recombinant yeast, as it is shown in the examples herein.

In an embodiment of the invention, a recombinant cell, and in particular a recombinant yeast cell, of the invention comprises:
between 1 and 5 recombinant nucleic acids encoding a polypeptide having a UDP-glucose-4-epimerase activity;
said recombinant nucleic acid encoding a polypeptide having a UDP-glucose-4-epimerase activity being derived or obtained from the group consisting of Pseudomonas aeruginosa, Pasteurella multocida, and Escherichia coli;
said recombinant nucleic acid encoding a polypeptide having a UDP-glucose-4-epimerase activity being under the control of an inducible or repressible promoter that is functional in the recombinant cells of the invention and/or under the control of a promoter selected from the group consisting of pCCW12 or pCCW120.Sm.

### Recombinant Nucleic Acids Encoding a Polypeptide Having a Chondroitinase Activity

A recombinant cell according to the invention, and in particular a recombinant yeast according to the invention, comprises one or more recombinant nucleic acids encoding a polypeptide having chondroitinase activity.

A polypeptide having chondroitinase activity according to the invention means a polypeptide that degrades chondroitin, i.e. that converts a chondroitin of a given molecular weight into a chondroitin of a lower molecular weight.

Polypeptides having a chondroitinase activity according to the invention may include hyaluronoglucosaminidases, such as chondroitinases or hyaluronidases.

As previously indicated, polypeptides having chondroitinase activity of the present invention comprise a secretion signal.

In an embodiment, polypeptides having chondroitinase activity comprise both a secretion signal and an anchoring signal.

In an embodiment, the said one or more recombinant nucleic acids encoding a polypeptide having chondroitinase activity are under the control of an inducible or repressible promoter that is functional in the recombinant cells of the invention.

One or more of the recombinant nucleic acids encoding a polypeptide having chondroitinase activity may be under the control of a promoter selected from the group consisting of pTEF1, pCCW12, pCCW12.sba, pCCW12.Sar, pPDC1, pTEF3, pTDH3, pNUP57 and pCCW10.ago, and in particular of a pCCW12.Sba promoter.

The said one or more recombinant nucleic acids encoding a polypeptide having a chondroitinase activity may be obtained or derived from at least one of Cupiennius salei (Csa), Tityus serrulatus (Ts), Bos taurus (Bt), Apis mellifera (Am), Macaca mulatta (Mm), or Vespa magnifica (Vm) and in particular from Tityus serrulatus (Ts).

A recombinant cell according to the invention, and in particular a recombinant yeast according to the invention, may comprise only one recombinant nucleic acid encoding a polypeptide having a chondroitinase activity.

Illustratively, the one or more recombinant nucleic acids encoding a polypeptide having chondroitinase activity may be inserted within the JLP1 gene and/or within the LYP1 gene and/or within the LEU2 gene, and in particular within the LEU2 gene.

In a preferred embodiment, the recombinant nucleic acid encoding a polypeptide having chondroitinase activity is a recombinant nucleic acid encoding a hyaluronidase. In particular, the polypeptide having chondroitinase activity is a hyaluronidase.

All the characteristics above regarding a recombinant nucleic acid encoding a polypeptide having chondroitinase activity apply to a recombinant nucleic acid encoding a hyaluronidase.

In an embodiment of the invention, a recombinant cell, and in particular a recombinant yeast, of the invention comprises:
only one recombinant nucleic acid encoding a polypeptide having a chondroitinase activity;
said recombinant nucleic acid encoding a polypeptide having a chondroitinase activity being derived or obtained from at least one of Cupiennius salei (Csa), Tityus serrulatus (Ts), Bos taurus (Bt), Apis mellifera (Am), Macaca mulatta (Mm), or Vespa magnifica (Vm) and in particular from Tityus serrulatus (Ts);
said recombinant nucleic acid encoding a polypeptide having chondroitinase activity comprising either (i) a secretion signal and no anchoring signal or (ii) a secretion signal and an anchoring signal;
said polypeptide having a chondroitinase is a hyaluronidase,
said recombinant nucleic acid encoding a polypeptide having chondroitinase activity being under the control of an inducible or repressible promoter that is functional in the recombinant cells of the invention and/or under the control of a promoter selected from the group consisting of pTEF1, pCCW12, pCCW12.sba, pCCW12.Sar, pPDC1, pTEF3, pTDH3, pNUP57 and pCCW10.ago, and in particular of a pCCW12.Sba promoter.

### Recombinant Nucleic Acids Encoding a Polypeptide Having Glutamine-Fructose-6-Phosphate Amidotransferase Activity (GFA1)

A recombinant cell according to the invention, and in particular a recombinant yeast cell according to the invention, may comprise only one recombinant nucleic acid encoding a polypeptide having glutamine-fructose-6-phosphate amidotransferase activity.

A polypeptide having glutamine-fructose-6-phosphate amidotransferase activity according to the invention means a polypeptide that converts fructose-6-phosphate into glucosamine-6-phosphate.

In an embodiment, one or more of the recombinant nucleic acids encoding a polypeptide having glutamine-fructose-6-phosphate amidotransferase activity are under the control of an inducible or repressible promoter that is functional in the recombinant cells of the invention.

One or more of the recombinant nucleic acids encoding a polypeptide having glutamine-fructose-6-phosphate amidotransferase activity may be under the control of a pTEF1.Ago promoter.

The said one or more recombinant nucleic acids encoding a polypeptide having glutamine-fructose-6-phosphate amidotransferase activity may be obtained or derived from Saccharomyces cerevisiae, as shown in the examples herein.

A recombinant cell according to the invention, and in particular a recombinant yeast cell according to the invention, may comprise only one recombinant nucleic acid encoding a polypeptide having glutamine-fructose-6-phosphate amidotransferase activity.

Illustratively, the one or more recombinant nucleic acids encoding a polypeptide having glutamine-fructose-6-phosphate amidotransferase activity may be inserted within the JLP1 gene of the recombinant cell, and in particular of the recombinant yeast cell, as it is shown in the examples herein.

In an embodiment of the invention, a recombinant cell, and in particular a recombinant yeast cell, of the invention comprises:
only one recombinant nucleic acid encoding a polypeptide having glutamine-fructose-6-phosphate amidotransferase activity;
said recombinant nucleic acid encoding a polypeptide having glutamine-fructose-6-phosphate amidotransferase activity originating or being derived from Saccharomyces cerevisiae; and
said recombinant nucleic acid encoding a polypeptide having glutamine-fructose-6-phosphate amidotransferase activity being under the control of a pTEF1.Ago promoter.

### Recombinant Nucleic Acids Encoding a Polypeptide Having a UDP-N-Acetylglucosamine Pyrophosphorylase Activity (ORI1)

A recombinant cell according to the invention, and in particular a recombinant yeast cell according to the invention, may comprise one or more recombinant nucleic acids encoding a polypeptide having UDP-N-acetylglucosamine pyrophosphorylase activity.

A polypeptide having UDP-N-acetylglucosamine pyrophosphorylase activity according to the invention means a polypeptide that converts N-acetyl-glucosamine into UDP-N-acetyl-glucosamine.

In an embodiment, one or more of the recombinant nucleic acids encoding a polypeptide having UDP-N-acetylglucosamine pyrophosphorylase activity are under the control of an inducible or repressible promoter that is functional in the recombinant cells of the invention, such as for example the inducible or repressible promoters pMET6 or pCUP1.

One or more of the recombinant nucleic acids encoding a polypeptide having UDP-N-acetylglucosamine pyrophosphorylase activity may be under the control of the promoters pTDH3-1.Sba or pTDH3.

The said one or more recombinant nucleic acids encoding a polypeptide having UDP-N-acetylglucosamine pyrophosphorylase activity may be obtained or derived from Saccharomyces cerevisiae, as shown in the examples herein.

A recombinant cell according to the invention, and in particular a recombinant yeast according to the invention, may comprise between 1 and 10 recombinant nucleic acids encoding a polypeptide having UDP-N-acetylglucosamine pyrophosphorylase activity, in particular may comprise between 2 and 8 recombinant nucleic acids encoding a polypeptide having UDP-N-acetylglucosamine pyrophosphorylase activity, and for example may comprise 2, 6 or 7 recombinant nucleic acids encoding a polypeptide having UDP-N-acetylglucosamine pyrophosphorylase activity.

Illustratively, the one or more recombinant nucleic acids encoding a polypeptide having UDP-N-acetylglucosamine pyrophosphorylase activity may be inserted within the HIS3 gene and/or within the MET14 gene and/or within the JLP1 gene of the recombinant cell, and in particular of the recombinant yeast, as it is shown in the examples herein.

In an embodiment of the invention, a recombinant cell, and in particular a recombinant yeast cell, of the invention comprises:
between 2 and 8 recombinant nucleic acids encoding a polypeptide having UDP-N-acetylglucosamine pyrophosphorylase activity;
said one or more recombinant nucleic acids encoding a polypeptide having UDP-N-acetylglucosamine pyrophosphorylase activity originating or being derived from Saccharomyces cerevisiae;
said one or more recombinant nucleic acids encoding a polypeptide having UDP-N-acetylglucosamine pyrophosphorylase activity being under the control of an inducible or repressible promoter that is functional in the recombinant cells of the invention, such as for example the inducible or repressible promoters pMET6 or pCUP1 and/or under the control of the promoters pTDH3-1.Sba or pTDH3.

### Recombinant Nucleic Acids Encoding a Polypeptide Having a Phosphoglucomutase-1 Activity (PGM1)

A recombinant cell according to the invention, and in particular a recombinant yeast cell according to the invention, may comprise one or more recombinant nucleic acids encoding a polypeptide having Phosphoglucomutase-1 (PGM1) activity.

A polypeptide having Phosphoglucomutase-1 (PGM1) activity according to the invention means a polypeptide that converts Glucose-6-phosphate into the intermediate metabolite Glucose-1-phosphate.

In an embodiment, one or more of the recombinant nucleic acids encoding a polypeptide having Phosphoglucomutase-1 (PGM1) activity are under the control of an inducible or repressible promoter that is functional in recombinant cells of the invention.

One or more of the recombinant nucleic acids encoding a polypeptide having Phosphoglucomutase-1 (PGM1) activity may be under the control of the promoter pTDH3.

The one or more recombinant nucleic acids encoding a polypeptide having Phosphoglucomutase-1 (PGM1) activity may be obtained or derived from Saccharomyces cerevisiae, as shown in the examples herein.

A recombinant cell according to the invention, and in particular a recombinant yeast cell according to the invention, may comprise only one recombinant nucleic acid encoding a polypeptide having Phosphoglucomutase-1 (PGM1) activity.

Illustratively, the one or more recombinant nucleic acids encoding a polypeptide having Phosphoglucomutase-1 activity may be inserted within the MET14 gene of the recombinant cell, and in particular of the recombinant yeast.

In an embodiment of the invention, a recombinant cell, and in particular a recombinant yeast, of the invention comprises:
only one recombinant nucleic acid encoding a polypeptide having Phosphoglucomutase-1 activity;
said recombinant nucleic acid encoding a polypeptide having Phosphoglucomutase-1 activity originating or being derived from Saccharomyces cerevisiae; and
said recombinant nucleic acid encoding a polypeptide having Phosphoglucomutase-1 activity being under the control of an inducible or repressible promoter that is functional in recombinant cells of the invention and/or under the promoter pTDH3.

### Recombinant Nucleic Acids Encoding a Polypeptide Having a UTP-Glucose-1-Phosphate Uridylyltransferase Activity (UGP1)

A recombinant cell according to the invention, and in particular a recombinant yeast cell according to the invention, comprises one or more recombinant nucleic acids encoding a polypeptide having UTP-glucose-1-phosphate uridylyltransferase (UGP1) activity.

A polypeptide having UTP-glucose-1-phosphate uridylyltransferase (UGP1) activity according to the invention means a polypeptide that converts the intermediate metabolite Glucose-1-Phosphate into the intermediate metabolite UDP-glucose.

In an embodiment, one or more of the recombinant nucleic acids encoding a polypeptide having a UTP-glucose-1-phosphate uridylyltransferase activity are under the control of an inducible or repressible promoter that is functional in recombinant cells of the invention, such as for example the inducible or repressible promoters pSAM1 or pCUP1.

One or more of the recombinant nucleic acids encoding a polypeptide having UTP-glucose-1-phosphate uridylyltransferase activity may be under the control of a pFBA1, a pENO2 or a pPDC1 promoter.

The one or more recombinant nucleic acids encoding a polypeptide having UTP-glucose-1-phosphate uridylyltransferase activity may be obtained or derived from Saccharomyces cerevisiae, as shown in the examples herein.

A recombinant cell according to the invention, and in particular a recombinant yeast cell according to the invention, may comprise between 1 and 10 recombinant nucleic acids encoding a polypeptide having UTP-glucose-1-phosphate uridylyltransferase activity, in particular may comprise between 2 and 8 recombinant nucleic acids encoding a polypeptide having UTP-glucose-1-phosphate uridylyltransferase activity, and for example may comprise 2, 6 or 8 recombinant nucleic acids encoding a polypeptide having UTP-glucose-1-phosphate uridylyltransferase activity.

Illustratively, the one or more recombinant nucleic acids encoding a polypeptide having UTP-glucose-1-phosphate uridylyltransferase activity gene may be inserted within the HIS3 gene and/or within the MET14 gene and/or within the JLP1 gene and/or within the SAM3 gene of the recombinant cell, and in particular of the recombinant yeast, as it is shown in the examples herein.

In an embodiment of the invention, a recombinant cell, and in particular a recombinant yeast cell, of the invention comprises:
between 2 and 8 recombinant nucleic acids encoding a polypeptide having UTP-glucose-1-phosphate uridylyltransferase activity;
said one or more recombinant nucleic acids encoding a polypeptide having UTP-glucose-1-phosphate uridylyltransferase activity originating or being derived from Saccharomyces cerevisiae; and
said one or more recombinant nucleic acids encoding a polypeptide having being under the control of an inducible or repressible promoter that is functional in the recombinant cells of the invention, such as for example the inducible or repressible promoters pSAM1 or pCUP1 and/or under the control of a promoter selected from the group consisting of pFBA1, pENO2 and pPDC1.

### Recombinant Nucleic Acids Encoding a Polypeptide Having a Glucosamine-6-Phosphate N-Acetyltransferase (GNA1) Activity

A recombinant cell according to the invention, and in particular a recombinant yeast cell according to the invention, may comprise only one recombinant nucleic acid encoding a polypeptide having Glucosamine-6-phosphate N-acetyltransferase (GNA1) activity.

A polypeptide having Glucosamine-6-phosphate N-acetyltransferase (GNA1) activity according to the invention means a polypeptide that converts Glucosamine-6-phosphate into N-acetyl-glucosamine-6-phosphate.

In an embodiment, one or more of the recombinant nucleic acids encoding a polypeptide having Glucosamine-6-phosphate N-acetyltransferase activity are under the control of an inducible or repressible promoter that is functional in the recombinant cells of the invention.

One or more of the recombinant nucleic acids encoding a polypeptide having Glucosamine-6-phosphate N-acetyltransferase activity may be under the control of the promoter pCWP2.

The said one or more recombinant nucleic acids encoding a polypeptide having Glucosamine-6-phosphate N-acetyltransferase activity may be obtained or derived from Saccharomyces cerevisiae, as shown in the examples herein.

A recombinant cell according to the invention, and in particular a recombinant yeast cell according to the invention, may comprise only one recombinant nucleic acid encoding a polypeptide having Glucosamine-6-phosphate N-acetyltransferase activity.

Illustratively, the one or more recombinant nucleic acids encoding a polypeptide having Glucosamine-6-phosphate N-acetyltransferase activity may be inserted within the MET14 gene and/or within the SAM3 gene of the recombinant cell, and in particular of the recombinant yeast, as it is shown in the examples herein.

In an embodiment of the invention, a recombinant cell, and in particular a recombinant yeast, of the invention comprises:
only one recombinant nucleic acid encoding a polypeptide having Glucosamine-6-phosphate N-acetyltransferase activity;
said recombinant nucleic acid encoding a polypeptide having Glucosamine-6-phosphate N-acetyltransferase activity originating or being derived from Saccharomyces cerevisiae; and
said recombinant nucleic acid encoding a polypeptide having Glucosamine-6-phosphate N-acetyltransferase activity being under the control of an inducible or repressible promoter that is functional in the recombinant cells of the invention and/or under the control of the promoter pCWP2.

### Recombinant Nucleic Acids Encoding a Polypeptide Having Phosphoacetylglucosamine Mutase (PCM1) Activity

A recombinant cell according to the invention, and in particular a recombinant yeast cell according to the invention, may comprise one or more recombinant nucleic acids encoding a polypeptide having phosphoacetylglucosamine mutase (PCM1) activity.

A polypeptide having phosphoacetylglucosamine mutase activity according to the invention means a polypeptide that converts N-acetyl-glucosamine-6-phosphate N-acetyl-glucosamine-1-phosphate.

In an embodiment, one or more of the recombinant nucleic acids encoding a polypeptide having phosphoacetylglucosamine mutase activity are under the control of an inducible or repressible promoter that is functional in the recombinant cells of the invention.

One or more of the recombinant nucleic acids encoding a polypeptide having phosphoacetylglucosamine mutase activity may be under the control a pTEF1 promoter.

The said one or more recombinant nucleic acids encoding a polypeptide having phosphoacetylglucosamine mutase activity may be obtained or be derived from Saccharomyces cerevisiae, as shown in the examples herein.

A recombinant cell according to the invention, and in particular a recombinant yeast according to the invention, may comprise only one recombinant nucleic acid encoding a polypeptide having phosphoacetylglucosamine mutase activity.

Illustratively, the one or more recombinant nucleic acids encoding a polypeptide having phosphoacetylglucosamine mutase activity may be inserted within the MET14 gene and/or within the SAM3 gene of the recombinant cell, and in particular of the recombinant yeast, as it is shown in the examples herein.

In an embodiment of the invention, a recombinant cell, and in particular a recombinant yeast cell, of the invention comprises:
only one recombinant nucleic acid encoding a polypeptide having phosphoacetylglucosamine mutase activity;
said recombinant nucleic acid encoding a polypeptide having phosphoacetylglucosamine mutase activity originating or being derived from Saccharomyces cerevisiae; and
said recombinant nucleic acid encoding a polypeptide having phosphoacetylglucosamine mutase activity being under the control of an inducible or repressible promoter that is functional in the recombinant cells of the invention and/or under the control of a pTEF1 promoter.

### Chondroitin Synthase (HCOS)

The chondroitin synthase enzyme is a protein which is described in the art for catalyzing the conversion of UDP-glucuronate and UDP-N-acetyl-galactosamine into chondroitin.

In a particular embodiment, the nucleic acid encoding a polypeptide having a chondroitin synthase (HCOS) activity is:
(i) a nucleic acid encoding a chondroitin synthase; or
(ii) a nucleic acid encoding a chimeric polypeptide having a chondroitin synthase activity.

A chimeric polypeptide, also known as a fusion polypeptide, is a polypeptide consisting of at least two domains or fragments, preferably from two distinct polypeptides, that are encoded by separate nucleic acids that have been joined so that they are transcribed and translated as a single unit, producing a single polypeptide. Chimeric polypeptides may be created in vivo, or in vitro using recombinant DNA techniques. Such techniques known in the art may notably include but not only DNA solid phase synthesis, recombinant PCR, routine molecular techniques involving the use of restriction enzymes, ligase, recombinase, using techniques such as Gibson assembly (Gibson D G, Young L, Chuang R Y, Venter J C, Hutchison C A 3rd, Smith H O (2009). Nature Methods. 6 (5): 343-345) or taking advantage of linear DNA recombination in vivo preferably in yeast.

A chimeric polypeptide having a chondroitin synthase activity that is suitable according to the invention may be a hybride protein which is known in the art (the concept of chimaeric chondroitine synthase is described in Tracy et al. (2006) Journal of biological chemistry, 282, 337-344 and Jing and DeAngelis (2003) Glycobiology 13, 661-71) encoded by a nucleic acid comprising a nucleic acid encoding a polypeptide having a hyaluronan synthase activity and a nucleic acid encoding a polypeptide having a chondroitin synthase activity. The resulting polypeptide is a chimeric polypeptide between a hyaluronan synthase and a chondroitin synthase.

Another chimeric polypeptide having a chondroitin synthase activity that is suitable according to the invention is encoded by a nucleic acid comprising a nucleic acid encoding a polypeptide having hyaluronan synthase activity and a nucleic acid encoding a polypeptide having Galactofuranosyltransferase activity. The resulting polypeptide is a fusion protein between a hyaluronan synthase and a galactofuranosyltransferase (Galactofuranosyltransferase GlfT2 Mycobacterium tuberculosis UNIPROT 053585 EC 2.1.4.288).

Another chimeric polypeptide having a chondroitin synthase activity that is suitable according to the invention is encoded by a nucleic acid comprising a nucleic acid encoding a polypeptide having hyaluronan synthase activity and a nucleic acid encoding a polypeptide having chitin synthase activity. The resulting polypeptide is a fusion protein between a hyaluronan synthase and a chitin synthase (chitin synthase CHS2 Saccharomyces cerevisiae UNIPROT P14180 EC 2.1.4.16).

Examples of chimeric polypeptides having a chondroitin synthase activity that are suitable according to the invention may be encoded by a nucleic acid sequence comprising:
a fragment from a nucleic acid encoding a hyaluronan synthase originating from Pasteurella multocida and a fragment from a nucleic acid encoding a chondroitin synthase from Pasteurella multocida (such as HCOS1-1, HCOS1-2 or HCOS1-3);
a fragment from a nucleic acid encoding a hyaluronan synthase originating from Pasteurella multocida, a fragment from a nucleic acid encoding a chondroitin synthase from Pasteurella multocida and a fragment from a nucleic acid encoding a chitin synthase 2 from Saccharomyces cerevisiae (such as HCOS.Sc);
a fragment from a nucleic acid encoding a hyaluronan synthase originating from Pasteurella multocida and a fragment from a nucleic acid encoding a chitin synthase 2 from Saccharomyces cerevisiae (such as HHASA.Sc)
a fragment from a nucleic acid encoding a hyaluronan synthase originating from Chlorella virus PBCV-1 and a fragment from a nucleic acid encoding a chondroitin synthase from Escherichia coli (such as HCOS1-Vir, HCOS2-Vir, or HCOS4-Vir);
a fragment from a nucleic acid encoding a hyaluronan synthase originating from Chlorella virus PBCV-1 and a fragment from a nucleic acid encoding a Galactofuranosyltransferase from Mycobacterium tuberculosis (such as HCOS3-Vir);
a fragment from a nucleic acid encoding a hyaluronan synthase originating from Chlorella virus PBCV-1 and a fragment from a nucleic acid encoding a chondroitin synthase from Penicillium oxalicum (such as HCOS5-Vir, HCOS6-Vir, HCOS7-Vir or HCOS8-Vir);
a fragment from a nucleic acid encoding a hyaluronan synthase originating from Chlorella virus PBCV-1 and a fragment from a nucleic acid encoding a chondroitin sulfate synthase from Homo sapiens (such as HCOS9-Vir, HCOS10-Vir, HCOS11-Vir, or HCOS12-Vir).

A method implemented to measure the activity level of a polypeptide having a chondroitin synthase activity belongs to the general knowledge of the one skilled in the art.

In this regard, the one skilled in the art may advantageously refer to the method wherein the enzyme's capacity of producing chondroitin is evaluated by the presence of chondroitin in the supernatant assayed using the carbazole method (Bitter and Muir (1962) analytical biochemistry 4, 330-334).

Preferred polypeptide having a chondroitin synthase activity in the present specification is an enzyme carrying two activities EC 2.4.1.175 and 2.4.1.226.

According to a preferred embodiment, one or more recombinant nucleic acids encoding a polypeptide having a chondroitin synthase activity may be obtained or derived from organisms preferably selected in a group consisting of prokaryotic organisms and eukaryotic organisms. In some embodiments, the one or more recombinant nucleic acids encoding a polypeptide having a chondroitin synthase activity be obtained or derived from archaebacteria.

In some preferred embodiments, the one or more recombinant nucleic acids encoding a polypeptide having a chondroitin synthase activity may be obtained or derived from the group consisting of Pasteurella multocida (Pm), Chlorella virus PBCV1 (Vir), Mycobacterium tuberculosis (Mt), Homo sapiens (Hs), Escherichia coli (Ec), Saccharomyces cerevisiae (Sc) or Penicillium oxalicum (Po).

According to a yet preferred embodiment, the one or more recombinant nucleic acids encoding a polypeptide having a chondroitin synthase activity may be selected from the group consisting of nucleic acid sequences having (i) at least 65%, advantageously at least 70%, preferably at least 80%, nucleic acid identity with a nucleic acid sequence as set forth as sequence SEQ ID NO: 1 (HCOS1-1), SEQ ID NO: 2 (HCOS1-2), SEQ ID NO: 3 (HCOS-1-3), SEQ ID NO: 4 (HCOS.Sc), SEQ ID NO: 5 (HHASA.Sc), SEQ ID NO: 6 (HCOS1-Vir), SEQ ID NO: 7 (HCOS2-Vir), SEQ ID NO: 8 (HCOS3-Vir), SEQ ID NO: 9 (HCOS4-Vir), SEQ ID NO: 10 (HCOS5-Vir), SEQ ID NO: 11 (HCOS6-Vir), SEQ ID NO: 12 (HCOS7-Vir), SEQ ID NO: 13 (HCOS8-Vir), SEQ ID NO: 14 (HCOS9-Vir), SEQ ID NO: 15 (HCOS10-Vir), SEQ ID NO: 16 (HCOS11-Vir), or SEQ ID NO: 17 (HCOS12-Vir), and (ii) a biological activity of the same nature as the nucleic acid sequence having, respectively, the nucleic sequence as set forth as sequence SEQ ID NO: 1 (HCOS1-1), SEQ ID NO: 2 (HCOS1-2), SEQ ID NO: 3 (HCOS-1-3), SEQ ID NO: 4 (HCOS.Sc), SEQ ID NO: 5 (HHASA.Sc), SEQ ID NO: 6 (HCOS1-Vir), SEQ ID NO: 7 (HCOS2-Vir), SEQ ID NO: 8 (HCOS3-Vir), SEQ ID NO: 9 (HCOS4-Vir), SEQ ID NO: 10 (HCOS5-Vir), SEQ ID NO: 11 (HCOS6-Vir), SEQ ID NO: 12 (HCOS7-Vir), SEQ ID NO: 13 (HCOS8-Vir), SEQ ID NO: 14 (HCOS9-Vir), SEQ ID NO: 15 (HCOS10-Vir), SEQ ID NO: 16 (HCOS11-Vir), or SEQ ID NO: 17 (HCOS12-Vir).

A biological activity of the same nature regarding this sequence is, as previously explained, the capacity to code for a polypeptide that converts UDP-glucuronate and UDP-N-acetyl-galactosamine into chondroitin.

As described herein, a nucleic acid sequence having at least 65% nucleotide identity with a reference nucleic acid sequence encompasses nucleic acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequences, and also a biological activity of the same nature as the said reference nucleic acid sequence.

As described herein, a nucleic acid sequence having at least 70% nucleotide identity with a reference nucleic acid sequence encompasses nucleic acid sequences having at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequences, and also a biological activity of the same nature as the said reference nucleic acid sequence.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

For the amino acid sequence of the chimaeric polypeptide having a chondroitin synthase activity comprising an amino acid comprising a fragment from an amino acid encoding a hyaluronan synthase originating from Pasteurella multocida and a fragment from an amino acid encoding a chondroitin synthase from Pasteurella multocida the one skilled in the art may refer to the accession numbers Q7BLV3 and Q9CMP0, respectively, in the UniProt database, or to the sequences as set forth in SEQ ID NO: 18 (HCOS1-1), SEQ ID NO: 19 (HCOS1-2) and SEQ ID NO: 20 (HCOS-1-3) described herein.

For the amino acid sequence of the chimaeric polypeptide having a chondroitin synthase activity comprising a fragment from an amino acid encoding a hyaluronan synthase originating from Pasteurella multocida, a fragment from an amino acid encoding a chondroitin synthase from Pasteurella multocida and a fragment from an amino acid encoding a chitin synthase 2 from Saccharomyces cerevisiae the one skilled in the art may refer to the accession numbers Q7BLV3, Q9CMP0 and P14180, respectively, in the UniProt database, or to the sequence as set forth in SEQ ID NO: 21 (HCOS.Sc) as described herein.

For the amino acid sequence of the chimaeric polypeptide having a chondroitin synthase activity comprising a fragment from an amino acid encoding a hyaluronan synthase originating from Pasteurella multocida and a fragment from an amino acid encoding a chitin synthase 2 from Saccharomyces cerevisiae the one skilled in the art may refer to the accession numbers Q7BLV3 and P14180, respectively, in the UniProt database, or to the sequence as set forth in SEQ ID NO: 22 (HHASA.Sc) as described herein.

For the amino acid sequence of the chimaeric polypeptide having a chondroitin synthase activity comprising a fragment from an amino acid encoding a hyaluronan synthase originating from Chlorella virus PBCV-1 and a fragment from an amino acid encoding a chondroitin synthase from Escherichia coli the one skilled in the art may refer to the accession numbers Q84419 and Q8L0V4, respectively, in the UniProt database, or to the sequences as set forth in SEQ ID NO: 23 (HCOS1-Vir), SEQ ID NO: 24 (HCOS2-Vir), or SEQ ID NO: 26 (HCOS4-Vir) as described herein.

For the amino acid sequence of the chimaeric polypeptide having a chondroitin synthase activity comprising a fragment from an amino acid encoding a hyaluronan synthase originating from Chlorella virus PBCV-1 and a fragment from an amino acid encoding a Galactofuranosyltransferase from Mycobacterium tuberculosis the one skilled in the art may refer to the accession numbers Q84419 and O53585, respectively, in the UniProt database, or to the sequence as set forth in SEQ ID NO: 25 (HCOS3-Vir) as described herein.

For the amino acid sequence of the chimaeric polypeptide having a chondroitin synthase activity comprising a fragment from an amino acid encoding a hyaluronan synthase originating from Chlorella virus PBCV-1 and a fragment from an amino acid encoding a chondroitin synthase from Penicillium oxalicum the one skilled in the art may refer to the accession numbers Q84419 and S7Z8F8, respectively, in the UniProt database, or to the sequences as set forth in SEQ ID NO: 27 (HCOS5-Vir).

For the amino acid sequence of the chimaeric polypeptide having a chondroitin synthase activity comprising a fragment from an amino acid encoding a hyaluronan synthase originating from Chlorella virus PBCV-1 and a fragment from an amino acid encoding a chondroitin synthase from Pasteurella multocida the one skilled in the art may refer to the accession numbers Q84419 and Q9CMP0, respectively, in the UniProt database, or to the sequences as set forth in SEQ ID NO: 28 (HCOS6-Vir), SEQ ID NO: 29 (HCOS7-Vir), or SEQ ID NO: 30 (HCOS8-Vir) as described herein.

For the amino acid sequence of the chimaeric polypeptide having a chondroitin synthase activity comprising a fragment from an amino acid encoding a hyaluronan synthase originating from Chlorella virus PBCV-1 and a fragment from an amino acid encoding a chondroitin sulfate synthase from Homo sapiens the one skilled in the art may refer to the accession numbers Q84419 and Q86X52, respectively, in the UniProt database, or to the sequences as set forth in SEQ ID NO: 31 (HCOS9-Vir), SEQ ID NO: 32 (HCOS10-Vir), SEQ ID NO: 33 (HCOS11-Vir) or SEQ ID NO: 34 (HCOS12-Vir) as described herein.

According to another particular embodiment, the one or more recombinant nucleic acids encoding a polypeptide having a chondroitin synthase activity may be nucleic acid(s) encoding a polypeptide having an amino acid sequence selected from the group consisting of sequences having at least 55%, advantageously at least 65%, preferably at least 80%, amino acid identity with the amino acid sequence of SEQ ID NO: 18 (HCOS1-1), SEQ ID NO: 19 (HCOS1-2), SEQ ID NO: 20 (HCOS-1-3), SEQ ID NO: 21 (HCOS.Sc), SEQ ID NO: 22 (HHASA.Sc), SEQ ID NO: 23 (HCOS1-Vir), SEQ ID NO: 24 (HCOS2-Vir), SEQ ID NO: 25 (HCOS3-Vir), SEQ ID NO: 26 (HCOS4-Vir), SEQ ID NO: 27 (HCOS5-Vir), SEQ ID NO: 28 (HCOS6-Vir), SEQ ID NO: 29 (HCOS7-Vir), SEQ ID NO: 30 (HCOS8-Vir), SEQ ID NO: 31 (HCOS9-Vir), SEQ ID NO: 32 (HCOS10-Vir), SEQ ID NO: 33 (HCOS11-Vir), and SEQ ID NO: 34 (HCOS12-Vir) and also a biological activity of the same nature.

A biological activity of the same nature regarding this sequence is as described previously, i.e. the capacity to catalyze the conversion of UDP-glucuronate and UDP-N-acetyl-galactosamine into UDP-N-acetyl-galactosamine.

As described herein, an amino acid sequence having at least 55% amino acid identity with a reference amino acid sequence encompasses amino acid sequences having at least 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 65% amino acid identity with a reference amino acid sequence encompasses amino acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the one or more recombinant nucleic acids encoding a polypeptide having chondroitin synthase activity in the present invention is regulated by at least one promoter and at least one terminator, such as herein after defined more in details, which are present in 5′ and 3′ position respectively of the one or more recombinant nucleic acids encoding a polypeptide having a chondroitin synthase activity.

### UDP-Glucose Dehydrogenase (UDP-GlcDH or HASB)

The UDP-Glucose dehydrogenase is a protein which is known in the art to catalyze the conversion of UDP-glucose into UDP-glucuronate. The UDP-Glucose dehydrogenase originating from the genome of Arabidopsis thaliana, Chlorella virus PBCV1 or Streptococcus zooepidemicus may be termed HASB.

A method implemented to measure the activity level of a polypeptide having a UDP-Glucose dehydrogenase activity belongs to the general knowledge of the one skilled in the art.

In this regard, the one skilled in the art may advantageously refer to the method described by Oka and Jigami (FEBS Journal 273, 2645-2657, 2006).

Preferred polypeptide having a UDP-Glucose dehydrogenase activity in the present specification is an enzyme having an EC number 1.1.1.22.

According to a preferred embodiment, the one or more recombinant nucleic acids encoding a polypeptide having UDP-Glucose dehydrogenase activity may be obtained or derived from organisms preferably selected in a group comprising prokaryotic organisms and eukaryotic organisms. In some embodiments, the one or more recombinant nucleic acids encoding a polypeptide having UDP-Glucose dehydrogenase activity obtained or derived from archaebacteria. In some preferred embodiments, the one or more recombinant nucleic acids encoding a polypeptide having UDP-Glucose dehydrogenase activity may obtained or derived from yeast, and especially from Arabidopsis thaliana, Chlorella virus PBCV1 or Streptococcus zooepidemicus, preferably from Arabidopsis thaliana, or Chlorella virus PBCV1.

According to a yet preferred embodiment, the one or more recombinant nucleic acids encoding a polypeptide having UDP-Glucose dehydrogenase activity may be selected from the group consisting of nucleic acid sequences having (i) at least 65%, advantageously at least 70%, preferably at least 80%, nucleic acid identity with a nucleic acid sequence as set forth as sequence SEQ ID NO: 35 (At), SEQ ID NO: 36 (Vir), SEQ ID NO: 37 (Vir) and SEQ ID NO: 38 (Sz), and (ii) a biological activity of the same nature as set forth as sequence SEQ ID NO: 35 (At), SEQ ID NO: 36 (Vir), SEQ ID NO: 37 (Vir) and SEQ ID NO: 38 (Sz). The nucleic acid sequences set forth as SEQ ID NO: 35 (At), SEQ ID NO: 36 (Vir), SEQ ID NO: 37 (Vir) and SEQ ID NO: 38 (Sz) encode a polypeptide having a UDP-Glucose dehydrogenase activity obtained or derived, respectively, from Arabidopsis thaliana (At), Chlorella virus PBCV1 (Vir) or Streptococcus zooepidemicus (Sz), that may herein also be collectively termed HASB herein.

A biological activity of the same nature regarding this sequence is, as previously explained, the capacity to code for a polypeptide that converts UDP-glucose into UDP-glucuronate.

As described herein, a nucleic acid sequence having at least 65% nucleotide identity with a reference nucleic acid sequence encompasses nucleic acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequences, and also a biological activity of the same nature as the said reference nucleic acid sequence.

As described herein, a nucleic acid sequence having at least 70% nucleotide identity with a reference nucleic acid sequence encompasses nucleic acid sequences having at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequences, and also a biological activity of the same nature as the said reference nucleic acid sequence.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

For the amino acid sequence of the polypeptide having a UDP-Glucose dehydrogenase activity from Arabidopsis thaliana, Chlorella virus PBCV1 or Streptococcus zooepidemicus the one skilled in the art may refer to the accession numbers NP_173979.1, NP_048965 or KIS19289, respectively, in the UniProt database, or to the sequences as set forth in SEQ ID NO: 39 (At), SEQ ID NO: 40 (Vir) and SEQ ID NO: 41 (Sz) described herein.

According to another particular embodiment, the nucleic acid(s) encoding a UDP-Glucose dehydrogenase may be nucleic acid(s) encoding an amino acid sequence selected from the group consisting of sequences having at least 55%, advantageously at least 65%, preferably at least 80%, amino acid identity with the amino acid sequence of SEQ ID NO: 39 (At), SEQ ID NO: 40 (Cv) and SEQ ID NO: 41 (Sz), and also a biological activity of the same nature as the amino acid sequences of SEQ ID NO: 39 (At), SEQ ID NO: 40 (Vir) and SEQ ID NO: 41 (Sz) and also a biological activity of the same nature.

A biological activity of the same nature regarding this sequence is as described previously, i.e. the capacity to catalyze the conversion of UDP-glucose into UDP-glucuronate.

As described herein, an amino acid sequence having at least 55% amino acid identity with a reference amino acid sequence encompasses amino acid sequences having at least 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 65% amino acid identity with a reference amino acid sequence encompasses amino acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the polypeptide having a UDP-Glucose dehydrogenase activity in the present invention is regulated by at least one promoter and at least one terminator, such as herein after defined more in detail, which are present in 5′ and 3′ position respectively of the nucleic acids encoding the said polypeptide having a UDP-Glucose dehydrogenase activity.

UDP-Glucose-4-Epimerase (kfoA or GNE1)

The UDP-glucose-4-epimerase is a protein which is known in the art to catalyze the conversion of UDP-N-acetyl-glucosamine into UDP-N-acetyl-galactosamine. The UDP-glucose-4-epimerase obtained or derived from Pseudomonas aeruginosa, Pasteurella multocida, or Escherichia coli may be termed kfoA or GNE1.

A method implemented to measure the activity level of a polypeptide having a UDP-glucose-4-epimerase activity belongs to the general knowledge of the one skilled in the art.

In this regard, the one skilled in the art may advantageously refer to the method described in Creuzenet et al. (2000, Journal of Biological Chemistry 275, 19060-67).

Preferred polypeptide having a UDP-glucose-4-epimerase activity in the present specification is an enzyme having an EC number 5.1.3.7.

According to a preferred embodiment, the one or more recombinant nucleic acids encoding a polypeptide having a UDP-glucose-4-epimerase activity may be obtained or derived from organisms preferably selected in a group consisting of prokaryotic organisms and eukaryotic organisms. In some embodiments, the one or more recombinant nucleic acids encoding a polypeptide having a UDP-glucose-4-epimerase activity may be obtained or derived from archaebacteria. In some preferred embodiments, the one or more recombinant nucleic acids encoding a polypeptide having a UDP-glucose-4-epimerase activitymay be obtained or derived from Pseudomonas aeruginosa, Pasteurella multocida (Pm), or Escherichia coli (Ec).

According to a yet preferred embodiment, the one or more recombinant nucleic acids encoding a polypeptide having a UDP-glucose-4-epimerase activitymay be selected from the group consisting of nucleic acid sequences having (i) at least 65%, advantageously at least 70%, preferably at least 80%, nucleic acid identity with a nucleic acid sequence as set forth as sequence SEQ ID NO: 42 (Pa), SEQ ID NO: 43 (Pm), and SEQ ID NO: 44 (Ec), and (ii) a biological activity of the same nature as set forth as sequence SEQ ID NO: 42 (Pa), SEQ ID NO: 43 (Pm), and SEQ ID NO: 44 (Ec).

A biological activity of the same nature regarding this sequence is, as previously explained, the capacity to code for a polypeptide that converts UDP-N-acetyl-glucosamine into UDP-N-acetyl-galactosamine.

As described herein, a nucleic acid sequence having at least 65% nucleotide identity with a reference nucleic acid sequence encompasses nucleic acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequences, and also a biological activity of the same nature as the said reference nucleic acid sequences.

As described herein, a nucleic acid sequence having at least 70% nucleotide identity with a reference nucleic acid sequence encompasses nucleic acid sequences having at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequences, and also a biological activity of the same nature as the said reference nucleic acid sequences.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequences.

For the amino acid sequence of the polypeptide having a UDP-glucose-4-epimerase activity from Pseudomonas aeruginosa, Pasteurella multocida, and Escherichia coli, the one skilled in the art may refer to the accession numbers Q8KN66, AAK02370, and Q8L0V2, respectively, in the UniProt database, or to the sequences as set forth as SEQ ID NO: 45 (Pa), SEQ ID NO: 46 (Pm), and SEQ ID NO: 47 (Ec) described herein.

According to another particular embodiment, the one or more recombinant nucleic acids encoding a polypeptide having a UDP-glucose-4-epimerase activity may be nucleic acid(s) encoding a polypeptide having an amino acid sequence selected from the group consisting of sequences having at least 55%, advantageously at least 65%, preferably at least 80%, amino acid identity with the amino acid sequence set forth as SEQ ID NO: 45 (Pa), SEQ ID NO: 46 (Pm), or SEQ ID NO: 47 (Ec) and also a biological activity of the same nature as the amino acid sequences of SEQ ID NO: 45 (Pa), SEQ ID NO: 46 (Pm), or SEQ ID NO: 47 (Ec).

A biological activity of the same nature regarding this sequence is as described previously, i.e. the capacity to catalyze the conversion of UDP-N-acetyl-glucosamine into UDP-N-acetyl-galactosamine.

As described herein, an amino acid sequence having at least 55% amino acid identity with a reference amino acid sequence encompasses amino acid sequences having at least 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 65% amino acid identity with a reference amino acid sequence encompasses amino acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the one or more recombinant nucleic acids encoding a polypeptide having a UDP-glucose-4-epimerase activity in the present invention is regulated by at least one promoter and at least one terminator, such as herein after defined more in details, which are present in 5′ and 3′ position respectively of the one or more recombinant nucleic acids encoding a polypeptide having a UDP-glucose-4-epimerase activity.

### Chondroitinase (HYAL)

The chondroitinase enzyme is a protein which is described in the art for catalyzing the degradation of chondroitin molecules into smaller chondroitin molecules. As mentioned above, a polypeptide having a chondroitinase activity may be a hyaluronoglucosaminidase, such as a chondroitinase or a hyaluronidase.

In a particular embodiment, a polypeptide having a chondroitinase activity is a hyaluronidase.

The polypeptide having a chondroitinase activity may encoded by the genome of Cupiennius salei, Tityus serrulatus, Bos taurus, Apis mellifera, Macaca mulatta, or Vespa magnifica and may be termed HYAL.

The polypeptide having a chondroitinase activity may possess both a secretion signal and an anchoring signal or a secretion signal and no anchoring signal signal or a secretion-anchor signal with a dual secretion and anchoring function. When the encoded polypeptide having chondroitinase activity possesses both a secretion signal and an anchoring signal, it may be termed HYAL-31.

When the encoded polypeptide having the chondroitinase activity possesses a secretion signal and no anchoring signal, it may be termed HYAL-3.

A method implemented to measure the activity level of a polypeptide having a chondroitinase activity belongs to the general knowledge of the one skilled in the art.

In this regard, the one skilled in the art may advantageously monitor the molecular weight of the obtained chondroitin on an agarose gel.

Preferred polypeptide having a chondroitinase activity in the present specification is an enzyme having an EC number of n^{o} EC 3.2.1.35.

According to a preferred embodiment, the one or more recombinant nucleic acids encoding a polypeptide having a chondroitinase activity may be obtained or derived from organisms preferably selected in a group comprising prokaryotic organisms and eukaryotic organisms. In some embodiments, one or more recombinant nucleic acids encoding a polypeptide having a chondroitinase activity may be obtained or derived from archaebacteria. In some embodiments, one or more recombinant nucleic acids encoding a polypeptide having a chondroitinase activity may be obtained or derived from organisms preferably selected from yeasts. In some other preferred embodiments, the one or more recombinant nucleic acids encoding a polypeptide having a chondroitinase activity may be obtained or derived from Cupiennius salei (Csa), Tityus serrulatus (Ts), Bos taurus (Bt), Apis mellifera (Am), Macaca mulatta (Mm), or Vespa magnifica (Vm) and in particular from Tityus serrulatus (Ts).

According to a yet preferred embodiment, the one or more recombinant nucleic acids encoding a polypeptide having a chondroitinase activity may be selected from the group consisting of nucleic acid sequences having (i) at least 65%, advantageously at least 70%, preferably at least 80%, nucleic acid identity with a nucleic acid of SEQ ID NO: 48 (Csa), SEQ ID NO: 49 (Ts), SEQ ID NO: 50 (Bt), SEQ ID NO: 51 (Am), SEQ ID NO: 52 (Mm) or SEQ ID NO: 53 (Vm), (ii) encoding a polypeptide having a chondroitinase activity, comprising a secretion signal and no anchoring signal, obtained or derived from Cupiennius salei, Tityus serrulatus, Bos taurus, Apis mellifera, Macaca mulatta, or Vespa magnifica, respectively, and (iii) having a biological activity of the same nature as the sequences of SEQ ID NO: 48 (Csa), SEQ ID NO: 49 (Ts), SEQ ID NO: 50 (Bt), SEQ ID NO: 51 (Am), SEQ ID NO: 52 (Mm) or SEQ ID NO: 53 (Vm).

According to another preferred embodiment, the one or more recombinant nucleic acids encoding a polypeptide having a chondroitinase activity may be selected from the group consisting of nucleic acid sequences having at least 65%, advantageously at least 70%, preferably at least 80%, nucleic acid identity with a nucleic acid of SEQ ID NO: 54 (Csa), SEQ ID NO: 55 (Ts), SEQ ID NO: 56 (Bt), SEQ ID NO: 57 (Am), SEQ ID NO: 58 (Mm) or SEQ ID NO: 59 (Vm), (ii) encoding a polypeptide having a chondroitinase activity comprising a secretion signal and an anchoring signal obtained or derived from Cupiennius salei, Tityus serrulatus, Bos taurus, Apis mellifera, Macaca mulatta, or Vespa magnifica, respectively, and (iii) having a biological activity of the same nature as the sequences of of SEQ ID NO: 54 (Csa), SEQ ID NO: 55 (Ts), SEQ ID NO: 56 (Bt), SEQ ID NO: 57 (Am), SEQ ID NO: 58 (Mm) or SEQ ID NO: 59 (Vm).

A biological activity of the same nature regarding this sequence is, as previously explained, the capacity to code for a polypeptide that catalyzes the degradation of chondroitin molecules into smaller chondroitin molecules.

As described herein, a nucleic acid sequence having at least 65% nucleotide identity with a reference nucleic acid sequence encompasses nucleic acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature, as the said reference nucleic acid sequence.

As described herein, a nucleic acid sequence having at least 70% nucleotide identity with a reference nucleic acid sequence encompasses nucleic acid sequences having at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature, as the said reference nucleic acid sequence.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature, as the said reference nucleic acid sequence.

For the amino acid sequence of the hyaluronidase from Cupiennius salei, Tityus serrulatus, Bos taurus, Apis mellifera, Macaca mulatta, or Vespa magnifica, the one skilled in the art may refer to the accession numbers A0A0S4JYH2, P85841, Q7YS45, Q08169, G7ML68 or P86875, respectively in the UniProt database or to SEQ ID NO: 60 (Csa), SEQ ID NO: 61 (Ts), SEQ ID NO: 62 (Bt), SEQ ID NO: 63 (Am), SEQ ID NO: 64 (Mm) or SEQ ID NO: 65 (Vm), described herein.

According to another particular embodiment, the one or more recombinant nucleic acids encoding a polypeptide having a chondroitinase activity may be nucleic acid(s) encoding a polypeptide having an amino acid sequence selected from the group consisting of sequences having at least 55%, advantageously at least 65%, preferably at least 80%, amino acid identity with the amino acid sequence of SEQ ID NO: 60 (Csa), SEQ ID NO: 61 (Ts), SEQ ID NO: 62 (Bt), SEQ ID NO: 63 (Am), SEQ ID NO: 64 (Mm) or SEQ ID NO: 65 (Vm) which comprise a secretion signal and no anchoring signal and also a biological activity of the same nature as the sequences SEQ ID NO: 60 (Csa), SEQ ID NO: 61 (Ts), SEQ ID NO: 62 (Bt), SEQ ID NO: 63 (Am), SEQ ID NO: 64 (Mm) or SEQ ID NO: 65 (Vm).

According to another particular embodiment, the one or more recombinant nucleic acids encoding a polypeptide having a chondroitinase activity may be nucleic acid(s) encoding a polypeptide having an amino acid sequence selected from the group consisting of sequences having at least 55%, advantageously at least 65%, preferably at least 80%, amino acid identity with the amino acid sequence of SEQ ID NO: 66 (Csa), SEQ ID NO: 67 (Ts), SEQ ID NO: 68 (Bt), SEQ ID NO: 69 (Am), SEQ ID NO: 70 (Mm) or SEQ ID NO: 71 (Vm) which comprise a secretion signal and an anchoring signal and also a biological activity of the same nature as the amino acid sequences of SEQ ID NO: 66 (Csa), SEQ ID NO: 67 (Ts), SEQ ID NO: 68 (Bt), SEQ ID NO: 69 (Am), SEQ ID NO: 70 (Mm) or SEQ ID NO: 71 (Vm).

A biological activity of the same nature regarding this sequence is as described previously, i.e. the capacity to catalyze the degradation of chondroitin molecules into smaller chondroitin molecules.

As described herein, an amino acid sequence having at least 55% amino acid identity with a reference amino acid sequence encompasses amino acid sequences having at least 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 65% amino acid identity with a reference amino acid sequence encompasses amino acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the one or more recombinant nucleic acids encoding a polypeptide having a chondroitinase activity in the present invention is regulated by at least one promoter and at least one terminator, such as herein after defined more in details, which are present in 5′ and 3′ position respectively of the one or more recombinant nucleic acids encoding a polypeptide having chondroitinase activity.

### Glutamine-Fructose-6-Phosphate Amidotransferase (GFA1)

The glutamine-fructose-6-phosphate amidotransferase enzyme is a protein which is described in the art for catalyzing the conversion of fructose-6-phosphate into glucosamine-6-phosphate. The glutamine-fructose-6-phosphate amidotransferase originating from Saccharomyces cerevisiae may be termed GFA1.

A method implemented to measure the activity level of a polypeptide having glutamine-fructose-6-phosphate amidotransferase activity belongs to the general knowledge of the one skilled in the art.

In this regard, the one skilled in the art may advantageously refer to the method described by Shiga Shibatan and Hiroaki Kitazawa (Plant Biotechnology 26, 149-152, 2009).

Preferred polypeptide having glutamine-fructose-6-phosphate amidotransferase activity in the present invention is an enzyme having an EC number of n^{o} EC 2.6.1.16.

According to a preferred embodiment, the one or more recombinant nucleic acids encoding a polypeptide having glutamine-fructose-6-phosphate amidotransferase activity may originate or be derived from organisms preferably selected in a group comprising prokaryotic organisms and eukaryotic organisms. In some embodiments, the one or more recombinant nucleic acids encoding a polypeptide having glutamine-fructose-6-phosphate amidotransferase activity may originate or be derived from archaebacteria. In some embodiments, the one or more recombinant nucleic acids encoding a polypeptide having glutamine-fructose-6-phosphate amidotransferase activity may originate or be derived from organisms preferably selected from Bacillus subtilis, and yeasts. In some other preferred embodiments, the one or more recombinant nucleic acids encoding a polypeptide having glutamine-fructose-6-phosphate amidotransferase activity may originate or be derived from a yeast, and especially from Saccharomyces cerevisiae.

According to a yet preferred embodiment, the one or more recombinant nucleic acids encoding a polypeptide having glutamine-fructose-6-phosphate amidotransferase activity may be selected from the group consisting of nucleic acid sequences having (i) at least 65%, advantageously at least 70%, preferably at least 80%, nucleic acid identity with the nucleic acid sequence as set forth as sequence SEQ ID NO: 72 (Sc), and (ii) a biological activity of the same nature as the nucleic acid sequence as set forth as sequence SEQ ID NO: 72. The nucleic acid as set forth as sequence SEQ ID NO: 72 encodes a polypeptide having glutamine-fructose-6-phosphate amidotransferase activity obtained or derived from Saccharomyces cerevisiae, that may also be termed GFA1.

According to yet another embodiment, the one or more recombinant nucleic acids encoding a polypeptide having glutamine-fructose-6-phosphate amidotransferase activity may be selected from the group consisting of nucleic acid sequences having (i) at least 65%, advantageously at least 70%, preferably at least 80%, nucleic acid identity with the nucleic acid sequence as set forth as sequence SEQ ID NO: 73 or SEQ ID NO: 74, and (ii) a biological activity of the same nature as the nucleic acid sequence as set forth as sequence SEQ ID NO: 73 or SEQ ID NO: 74. The nucleic acid sequence as set forth as sequence SEQ ID NO: 73 or SEQ ID NO: 74 encodes a polypeptide having glutamine-fructose-6-phosphate amidotransferase activity originating from Chlorella virus PBCV1.

A biological activity of the same nature regarding this sequence is, as previously explained, the capacity to code for a polypeptide that converts fructose-6-phosphate into glucosamine-6-phosphate.

As described herein, a nucleic acid sequence having at least 65% nucleotide identity with a reference nucleic acid sequence encompasses nucleic acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

As described herein, a nucleic acid sequence having at least 70% nucleotide identity with a reference nucleic acid sequence encompasses nucleic acid sequences having at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

For the amino acid sequence of the polypeptide having glutamine-fructose-6-phosphate amidotransferase activity originating from Saccharomyces cerevisiae, the one skilled in the art may refer to the accession number NP8012818 in the UniProt database, or to the sequence SEQ ID NO: 75 described herein.

For the amino acid sequence of the polypeptide having glutamine-fructose-6-phosphate amidotransferase activity originating from Chlorella virus PBCV1, the one skilled in the art may also refer to the accession number NP_048448 in the UniProt database, or to the sequence SEQ ID NO: 76 described herein.

According to another particular embodiment, the one or more recombinant nucleic acids encoding a polypeptide having glutamine-fructose-6-phosphate amidotransferase activity may be nucleic acid(s) encoding polypeptide having an amino acid sequence selected from the group consisting of sequences having at least 35%, advantageously at least 65%, preferably at least 80%, amino acid identity with the amino acid sequence of SEQ ID NO: 75 or with SEQ ID NO: 76, and also a biological activity of the same nature as the amino acid sequence of SEQ ID NO: 75 or with SEQ ID NO: 76.

A biological activity of the same nature regarding this sequence is as described previously, i.e. the capacity to catalyze the conversion of fructose-6-phosphate into glucosamine-6-phosphate.

As described herein, an amino acid sequence having at least 35% amino acid identity with a reference amino acid sequence encompasses amino acid sequences having at least 36%, 37%, 38%, 39%, 40% 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 65% amino acid identity with a reference amino acid sequence encompasses amino acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the polypeptide having glutamine-fructose-6-phosphate amidotransferase activity in the present invention is regulated by at least one promoter and at least one terminator, such as herein after defined more in details, which are present in 5′ and 3′ position respectively of one or more recombinant nucleic acids encoding a polypeptide having glutamine-fructose-6-phosphate amidotransferase activity.

### UDP-N-Acetylglucosamine Pyrophosphorylase (QRI1)

The UDP-N-acetylglucosamine pyrophosphorylase enzyme is a protein which is described in the art for catalyzing the conversion of N-acetyl-glucosmine-6-phosphate into UDP-N-acetyl-glucose. The UDP-N-acetylglucosamine pyrophosphorylase originating from Saccharomyces cerevisiae may be termed QRI1.

A method implemented to measure the activity level of a polypeptide having UDP-N-acetylglucosamine pyrophosphorylase activity belongs to the general knowledge of the one skilled in the art.

In this regard, the one skilled in the art may advantageously refer to the method described by Mio et al. (The Journal of Biological Chemistry, Col. 273, No 23, Jun. 5, 1998, 14392-14397) except that UDP-N-acetyl-glucosamine is detected by LC MS/MS using a Synergi RP Fusion column.

Preferred polypeptide having UDP-N-acetylglucosamine pyrophosphorylase activity in the present invention is an enzyme having an EC number of n^{o} EC 2.7.7.23 According to a preferred embodiment, the one or more recombinant nucleic acids encoding a polypeptide having UDP-N-acetylglucosamine pyrophosphorylase activity may originate or be derived from organisms preferably selected in a group consisting of prokaryotic organisms and eukaryotic organisms. In some embodiments, the one or more recombinant nucleic acids encoding a polypeptide having UDP-N-acetylglucosamine pyrophosphorylase activity may originate or be derived from archaebacteria. In some embodiments, the one or more recombinant nucleic acids encoding a polypeptide having UDP-N-acetylglucosamine pyrophosphorylase activity may originate or be derived from organisms preferably selected from Bacillus subtilis, and yeasts. In some other preferred embodiments, the one or more recombinant nucleic acids encoding a polypeptide having UDP-N-acetylglucosamine pyrophosphorylase activity may originate or be derived from yeasts, and especially from Saccharomyces cerevisiae.

According to a yet preferred embodiment, the one or more recombinant nucleic acids encoding a polypeptide having UDP-N-acetylglucosamine pyrophosphorylase activity may be selected from the group consisting of nucleic acid sequences having (i) at least 65%, advantageously at least 70%, preferably at least 80%, nucleic acid identity with a nucleic acid sequence as set forth as SEQ ID NO: 77, and (ii) a biological activity of the same nature as the nucleic acid sequence as set forth as SEQ ID NO: 77. The nucleic acid sequence set forth as SEQ ID NO: 77 encodes a polypeptide having UDP-N-acetylglucosamine pyrophosphorylase activity originating from Saccharomyces cerevisiae, that may also be termed QRIL.

A biological activity of the same nature regarding this sequence is, as previously explained, the capacity to code for a polypeptide that converts N-acetyl-glucosmine-6-phosphate into UDP-N-acetyl-glucose.

As described herein, a nucleic acid sequence having at least 65% nucleotide identity with a reference nucleic acid sequence encompasses nucleic acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

As described herein, a nucleic acid sequence having at least 70% nucleotide identity with a reference nucleic acid sequence encompasses nucleic acid sequences having at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

For the amino acid sequence of the polypeptide having UDP-N-acetylglucosamine pyrophosphorylase activity originating from Saccharomyces cerevisiae, the one skilled in the art may refer to the accession number NP_010180 in the UniProt database, or to SEQ ID NO. 78 described herein.

According to another particular embodiment, the one or more recombinant nucleic acids encoding a polypeptide having UDP-N-acetylglucosamine pyrophosphorylase activity may be nucleic acid(s) encoding polypeptide having an amino acid sequence selected from the group consisting of sequences having at least 35%, advantageously at least 45%, preferably at least 80%, amino acid identity with the amino acid sequence set forth as sequence SEQ ID NO. 78, and also a biological activity of the same nature as the amino acid sequence set forth as sequence SEQ ID NO. 78.

A biological activity of the same nature regarding this sequence is as described previously, i.e. the capacity to catalyze the conversion of of N-acetyl-glucosmine-6-phosphate into UDP-N-acetyl-glucose.

As described herein, an amino acid sequence having at least 35% amino acid identity with a reference amino acid sequence encompasses amino acid sequences having at least 36%, 37%, 38%, 39%, 40% 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 45% amino acid identity with a reference amino acid sequence encompasses amino acid sequences having at least 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the one or more recombinant nucleic acids encoding a polypeptide having UDP-N-acetylglucosamine pyrophosphorylase activity in the present invention is regulated by at least one promoter and at least one terminator, such as herein after defined more in details, which are present in 5′ and 3′ position respectively of the one or more recombinant nucleic acids encoding a polypeptide having UDP-N-acetylglucosamine pyrophosphorylase activity.

### Phosphoglucomutase-1 (PGM1)

The Phosphoglucomutase-1 enzyme is a protein which is described in the art for catalyzing the conversion of Glucose-6-phosphate into Glucose-1-phosphate. The Phosphoglucomutase-1 originating from Saccharomyces cerevisiae may be termed PGM1.

A method implemented to measure the activity level of a polypeptide having Phosphoglucomutase-1 activity belongs to the general knowledge of the one skilled in the art.

In this regard, the one skilled in the art may advantageously refer to the method described by Tiwari and Bhat (Biochemical and Biophysical Research Communications 366, 340-345, 2008).

Preferred polypeptide having a Phosphoglucomutase-1 activity in the present invention is an enzyme having an EC number of n^{o} 5.4.2.2.

According to a preferred embodiment, the one or more recombinant nucleic acids encoding a polypeptide having Phosphoglucomutase-1 activity may originate from organisms preferably selected in a group consisting of prokaryotic organisms and eukaryotic organisms. In some embodiments, the one or more recombinant nucleic acids encoding a polypeptide having Phosphoglucomutase-1 activity may originate or be derived from archaebacteria. In some embodiments, the one or more recombinant nucleic acids encoding a polypeptide having Phosphoglucomutase-1 activity may originate or be derived from organisms preferably selected from bacteria. In a preferred embodiment, the one or more recombinant nucleic acids encoding a polypeptide having Phosphoglucomutase-1 activity may originate or be derived from Saccharomyces cerevisiae.

According to an embodiment, the one or more recombinant nucleic acids encoding a polypeptide having Phosphoglucomutase-1 activity may be selected from the group consisting of nucleic acid sequences having (i) at least 80% nucleic acid identity with the nucleic acid sequence set forth as sequence SEQ ID NO: 79, which originates from Saccharomyces cerevisiae, and (ii) a biological activity of the same nature as the nucleic acid sequence set forth as sequence SEQ ID NO: 79.

A biological activity of the same nature regarding this sequence is, as previously explained, the capacity to code for a polypeptide that converts Glucose-6-phosphate into Glucose-1-phosphate.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

For the amino acid sequence of the peptide having a Phosphoglucomutase-1 activity from Saccharomyces cerevisiae, the one skilled in the art may refer to the accession number NP33401 in the UniProt database, or to SEQ ID NO: 80 described herein.

According to another particular embodiment, the one or more recombinant nucleic acids encoding a polypeptide having Phosphoglucomutase-1 activity may be nucleic acid(s) encoding polypeptide having an amino acid sequence selected from the group consisting of sequences having at least 80% amino acid identity with the amino acid sequence set forth as sequence SEQ ID NO: 80, and also a biological activity of the same nature as the amino acid sequence set forth as sequence SEQ ID NO: 80.

A biological activity of the same nature regarding this sequence is as described previously, i.e. the capacity to catalyze the conversion of Glucose-6-phosphate into Glucose-1-phosphate.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the one or more recombinant nucleic acids encoding a polypeptide having a Phosphoglucomutase-1 activity in the present invention is regulated by at least one promoter and at least one terminator, such as herein after defined more in details, which are present in 5′ and 3′ position respectively of the one or more recombinant nucleic acids encoding a polypeptide having a Phosphoglucomutase-1 activity.

### UTP-Glucose-1-Phosphate Uridylyltransferase (UGP1)

The UTP-glucose-1-phosphate uridylyltransferase enzyme is a protein which is described in the art for catalyzing the conversion of Glucose-1-Phosphate into UDP-glucose.

The UTP-glucose-1-phosphate uridylyltransferase originating from Saccharomyces cerevisiae may be termed UGP1.

A method implemented to measure the activity level of a polypeptide having a UTP-glucose-1-phosphate uridylyltransferase activity belongs to the general knowledge of the one skilled in the art.

In this regard, the one skilled in the art may advantageously refer to the method described by Roeben (J. Mol. Biol 364, 551-560, 2006).

Preferred polypeptide having a UTP-glucose-1-phosphate uridylyltransferase activity in the present invention is an enzyme having an EC number of n^{o} 2.7.7.9.

According to a preferred embodiment, the one or more recombinant nucleic acids encoding a polypeptide having UTP-glucose-1-phosphate uridylyltransferase activity may originate or be derived from organisms preferably selected in a group consisting of prokaryotic organisms and eukaryotic organisms. In some embodiments, the one or more recombinant nucleic acids encoding a polypeptide having UTP-glucose-1-phosphate uridylyltransferase activity may originate or be derived from archaebacteria. In some embodiments, the one or more recombinant nucleic acids encoding a polypeptide having UTP-glucose-1-phosphate uridylyltransferase activity may originate or be derived from organisms preferably selected from bacteria. In a preferred embodiment, the one or more recombinant nucleic acids encoding a polypeptide having UTP-glucose-1-phosphate uridylyltransferase activity may originate or be derived from Saccharomyces cerevisiae.

According to a particular embodiment, the one or more recombinant nucleic acids encoding a polypeptide having a UTP-glucose-1-phosphate uridylyltransferase activity may be selected from the group consisting of nucleic acid sequences having (i) at least 80% nucleic acid identity with a nucleic acid as set forth in SEQ ID NO: 81, originating from Saccharomyces cerevisiae, and (ii) a biological activity of the same nature as nucleic acid as set forth in SEQ ID NO: 81.

A biological activity of the same nature regarding this sequence is, as previously explained, the capacity to code for a polypeptide that converts Glucose-1-phosphate into UDP-glucose.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

For the amino acid sequence of the polypeptide having a UTP-glucose-1-phosphate uridylyltransferase activity originating from Saccharomyces cerevisiae, the one skilled in the art may refer to the accession number NP_32861 in the UniProt database, or to the sequence set forth as sequence SEQ ID NO: 82 described herein.

According to another particular embodiment, the one or more recombinant nucleic acids encoding a polypeptide having UTP-glucose-1-phosphate uridylyltransferase activity may be nucleic acid(s) encoding polypeptide having an amino acid sequence selected from the group consisting of sequences having at least 80% amino acid identity with the amino acid sequence set forth as SEQ ID NO: 82, and also a biological activity of the same nature as the amino acid sequence set forth as SEQ ID NO: 82.

A biological activity of the same nature regarding this sequence is as described previously, i.e. the capacity to catalyze the conversion of Glucose-1-phosphate into UDP-glucose.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the one or more recombinant nucleic acids encoding a polypeptide having a UTP-glucose-1-phosphate uridylyltransferase activity in the present invention is regulated by at least one promoter and at least one terminator, such as herein after defined more in details, which are present in 5′ and 3′ position respectively of the one or more recombinant nucleic acids encoding a polypeptide having a UTP-glucose-1-phosphate uridylyltransferase activity.

### Glucosamine-6-Phosphate N-Acetyltransferase (GNA1)

The Glucosamine-6-phosphate N-acetyltransferase enzyme is a protein which is described in the art for catalyzing the conversion of Glucosamine-6-phosphate into N-acetyl-glucosamine-6-phosphate. The Glucosamine-6-phosphate N-acetyltransferase originating from Saccharomyces cerevisiae may be termed GNA1.

A method implemented to measure the activity level of a polypeptide having a Glucosamine-6-phosphate N-acetyltransferase activity belongs to the general knowledge of the one skilled in the art.

In this regard, the one skilled in the art may advantageously refer to the method described by Li et al. (Anal. Biochem. 370, 142-146, 2007).

Preferred polypeptide having a Glucosamine-6-phosphate N-acetyltransferase activity in the present invention is an enzyme having an EC number of n^{o} 2.3.1.4.

According to a preferred embodiment, the one or more recombinant nucleic acids encoding a polypeptide having Glucosamine-6-phosphate N-acetyltransferase activity may originate or be derived from organisms preferably selected in a group consisting of prokaryotic organisms and eukaryotic organisms. In some preferred embodiments, the one or more recombinant nucleic acids encoding a polypeptide having Glucosamine-6-phosphate N-acetyltransferase activity may originate or be derived from a yeast, and especially from Saccharomyces cerevisiae.

According to a particular embodiment, the one or more recombinant nucleic acids encoding a polypeptide having a Glucosamine-6-phosphate N-acetyltransferase activity may be selected from the group consisting of nucleic acid sequences having (i) at least 80% nucleic acid identity with a nucleic acid sequence as set forth as sequence SEQ ID NO: 83, and (ii) a biological activity of the same nature as the nucleic acid sequence as set forth as sequence SEQ ID NO: 83. The nucleic acid sequence as set forth as sequence SEQ ID NO: 83 encodes a polypeptide having a Glucosamine-6-phosphate N-acetyltransferase activity originating from Saccharomyces cerevisiae, that may also be termed GNA1.

A biological activity of the same nature regarding this sequence is, as previously explained, the capacity to code for a polypeptide that converts Glucosamine-6-phosphate into N-acetyl-glucosamine-6-phosphate.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

For the amino acid sequence of the polypeptide having a Glucosamine-6-phosphate N-acetyltransferase activity obtained or derived from Saccharomyces cerevisiae, the one skilled in the art may refer to the accession number NP_116637 in the UniProt database, or to SEQ ID NO. 84 described herein.

According to another particular embodiment, the one or more recombinant nucleic acids encoding a polypeptide having Glucosamine-6-phosphate N-acetyltransferase activity may be nucleic acid(s) encoding polypeptide having an amino acid sequence selected from the group consisting of sequences having at least 80% amino acid identity with the amino acid sequence set forth as SEQ ID NO. 84, and also a biological activity of the same nature as the amino acid sequence set forth as SEQ ID NO: 84.

A biological activity of the same nature regarding this sequence is as described previously, i.e. the capacity to catalyze the conversion of Glucosamine-6-phosphate into N-acetyl-glucosamine-6-phosphate.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the one or more recombinant nucleic acids encoding a polypeptide having a Glucosamine-6-phosphate N-acetyltransferase activity in the present invention is regulated by at least one promoter and at least one terminator, such as herein after defined more in details, which are present in 5′ and 3′ position respectively of the one or more recombinant nucleic acids encoding a polypeptide having a Glucosamine-6-phosphate N-acetyltransferase activity.

### Phosphoacetylglucosamine Mutase (PCM1)

The phosphoacetylglucosamine mutase enzyme is a protein which is described in the art for catalyzing the conversion of N-acetyl-glucosamine-6-phosphate into N-acetyl-glucosamine-1-phosphate. The phosphoacetylglucosamine mutase originating from Saccharomyces cerevisiae may be termed PCM1.

A method implemented to measure the activity level of a polypeptide having phosphoacetylglucosamine mutase activity belongs to the general knowledge of the one skilled in the art.

In this regard, the one skilled in the art may advantageously refer to the method described by Bandini et al. (Molecular Microbiology 85(3), 513-534, 2012).

Preferred polypeptide having a phosphoacetylglucosamine mutase activity in the present invention is an enzyme having an EC number of n^{o} 5.4.2.3.

According to a preferred embodiment, the one or more recombinant nucleic acids encoding a polypeptide having phosphoacetylglucosamine mutase activity may originate or be derived from organisms preferably selected in a group consisting of prokaryotic organisms and eukaryotic organisms. In some preferred embodiments, the one or more recombinant nucleic acids encoding a polypeptide having phosphoacetylglucosamine mutase activity may originate or be derived from a yeast, and especially from Saccharomyces cerevisiae.

According to a particular embodiment, the one or more recombinant nucleic acids encoding a polypeptide having a phosphoacetylglucosamine mutase activity may be selected from the group consisting of nucleic acid sequences having (i) at least 80% nucleic acid identity with a nucleic acid sequence set forth as SEQ ID NO: 85, and (ii) a biological activity of the same nature as the nucleic acid sequence set forth as SEQ ID NO: 85. The nucleic acid set forth as SEQ ID NO: 85 encodes a polypeptide having phosphoacetylglucosamine mutase activity originating from Saccharomyces, that may also be termed PCM1.

A biological activity of the same nature regarding this sequence is, as previously explained, the capacity to code for a polypeptide that converts N-acetyl-glucosamine-6-phosphate into N-acetyl-glucosamine-1-phosphate.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

For the amino acid sequence of the polypeptide having a phosphoacetylglucosamine mutase activity originating from Saccharomyces cerevisiae, the one skilled in the art may refer to the accession number NP_010856 in the UniProt database, or to SEQ ID NO: 86 described herein.

According to another particular embodiment, the one or more recombinant nucleic acids encoding a polypeptide having phosphoacetylglucosamine mutase activity may be nucleic acid(s) encoding polypeptide having an amino acid sequence selected from the group consisting of sequences having at least 80% amino acid identity with the amino acid sequence set forth as SEQ ID NO: 86, and also a biological activity of the same nature as the amino acid sequence set forth as SEQ ID NO: 86.

A biological activity of the same nature regarding this sequence is as described previously, i.e. the capacity to catalyze the conversion of N-acetyl-glucosamine-6-phosphate into N-acetyl-glucosamine-1-phosphate.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the one or more recombinant nucleic acids encoding a polypeptide having a phosphoacetylglucosamine mutase activity in the present invention is regulated by at least one promoter and at least one terminator, such as herein after defined more in detail, which are present in 5′ and 3′ position respectively of the one or more recombinant nucleic acids encoding a polypeptide having a phosphoacetylglucosamine mutase activity.

### Promoters

As disclosed herein, the expression of the genes of interest that have been genetically engineered for obtaining a recombinant cell according to the invention comprise appropriate regulatory sequences that are functional in the recombinant cells of the invention, and in particular in recombinant yeast cells of the invention, including in particular Saccharomyces cerevisiae.

Various promoters may be used for the desired expression of the coding sequences of interest.

Promoters according to the invention may be selected from the group consisting of the following promoters:
pTDH3 (SEQ ID NO: 87),
pTDH3.Sk (SEQ ID NO: 88)
pTDH3-1.Sba (SEQ ID NO: 89),
pTDH3.Sar (SEQ ID NO: 90),
pENO2 (SEQ ID NO: 91),
pTEF3 (SEQ ID NO: 92),
pTEF1 (SEQ ID NO: 93),
pTEF1.Ago (SEQ ID NO: 94),
pTEF1.sba (SEQ ID NO: 95),
pPDC1 (SEQ ID NO: 96),
pCCW12 (SEQ ID NO: 97),
pCCW12.Sm (SEQ ID NO: 98),
pCCW12.sk (SEQ ID NO: 99),
pCCW12.sba (SEQ ID NO: 100),
pCCW12.sar (SEQ ID NO: 101),
pNUP57 (SEQ ID NO: 102),
pCCW10.ago (SEQ ID NO: 103),
pCWP2 (SEQ ID NO: 104),
pFBA1 (SEQ ID NO: 105),
pCCW120.Sm (SEQ ID NO: 106).

Promoters more particularly interesting in the present invention may be selected from the group consisting of:
pTDH3 (SEQ ID NO: 87)
pTDH3.Sk (SEQ ID NO: 88)
pTDH3-1.Sba (SEQ ID NO: 89)
pTDH3.Sar (SEQ ID NO: 90),
pENO2 (SEQ ID NO: 91),
pTEF3 (SEQ ID NO: 92),
pTEF1 (SEQ ID NO: 93),
pTEF1.Ago (SEQ ID NO: 94),
pTEF1.sba (SEQ ID NO: 95),
pPDC1 (SEQ ID NO: 96),
pCCW12 (SEQ ID NO: 97),
pCCW12.sba (SEQ ID NO: 100)
pCCW120.Sm (SEQ ID NO: 106),
pNUP57 (SEQ ID NO: 102),
pCCW10.ago (SEQ ID NO: 103),
pCWP2 (SEQ ID NO: 104), and
pFBA1 (SEQ ID NO: 105)

Said promoters can in particular be selected from the group consisting of pTDH3, pTDH3.Sk, pTDH3-1.Sba, pTEF1, pTEF1.Ago, pTEF1.sba, pCCW12, pCCW120.Sm, pCWP2 and pFBA1.

Alternatively, promoters interesting in the present invention may be selected from the group consisting of:
pNUP57 (SEQ ID NO: 102), and
pCCW10.ago (SEQ ID NO: 103).

Another promoter of interest in the present invention may be:
pCWP2 (SEQ ID NO: 104).

As previously mentioned, inducible or repressible promoters are promoters whose activity is controlled by the presence or absence of biotic or abiotic factors and also by the quantity of said factor. Accordingly, for some promoters, their activity will in particular be induced and thus increased when the quantity of a given factor increases or is increased, and, accordingly, the activity of these same promoters can be repressed and thus reduced when the quantity of said factor diminishes or is reduced. The quantity of said factor(s) in the culture medium of a recombinant yeast cell of the invention comprising inducible or repressible promoters can be decided and thus controlled by the man skilled in the art.

For example, increasing the quantity of copper in a culture medium of a recombinant yeast cell according to the invention comprising a pCUP1 promoter will induce and thus increase transcription of the gene under the control of this promoter. On the contrary, reducing the quantity of copper in said culture medium will lead to a repression, and thus a reduced, transcription of the gene under the control of this promoter.

In another example, increasing the quantity of methionine in a culture medium of a recombinant yeast cell according to the invention comprising a pMET6 promoter will repress and thus decrease transcription of the gene under the control of this promoter. On the contrary, reducing the quantity of methionine in said culture medium will lead to an induced, and thus an increased, transcription of the gene under the control of this promoter.

For this reason, the following promoters are referred to in the present text as being inducible or repressible promoters.

According to a first embodiment, inducible or repressible promoters according to the invention may be selected from the group comprising promoters inducible or repressible with copper, promoters inducible or repressible with methionine and promoters inducible or repressible with threonine, and are in particular CUP-1—copper inducible or repressible.

According to this embodiment, the inducible or repressible promoter according to the invention can in particular be pCUP1 (SEQ ID NO: 107).

The activity of these promoters is thus induced by the increasing presence of methionine, copper or threonine as indicated above, and their activity diminishes, i.e. is repressed, when the quantity of methionine, copper or threonine is reduced.

According to a second embodiment, inducible or repressible promoters according to the invention may be selected from the group comprising promoters inducible or repressible with copper, promoters inducible or repressible with lysine and promoters inducible or repressible with methionine, and in particular selected from the group consisting of:
pMET6—methionine inducible or repressible (SEQ ID NO: 108),
pMET25—methionine inducible or repressible (SEQ ID NO: 109), and
pSAM1—methionine inducible or repressible (SEQ ID NO: 110).

According to this particular embodiment, the inducible or repressible promoter according to the invention may, be selected from the group consisting of pMET6 and pSAM1.

The activity of these promoters is thus repressed by the increasing presence of methionine, copper, lysine or glucose as indicated above, and their activity increases, i.e. is induced, when the quantity of methionine, copper, lysine or glucose is reduced.

In a particular embodiment, inducible or repressible promoters according to the invention may be selected from the group comprising promoters inducible or repressible with copper, promoters inducible or repressible with glucose, promoters inducible or repressible with lysine, promoters inducible or repressible with methionine and promoters inducible or repressible with threonine.

Synthetic promoters as described in Blazeck & Alper (2013) Biotechnol. J. 8 46-58 can also be used.

The promoters of the invention can originate from any organism from the Saccharomycetes class and can in particular originate from an organism selected from the group consisting of at least one of Saccharomyces cerevisiae, Saccharomyces boulardii, Saccharomyces castelii, Saccharomyces bayanus, Saccharomyces arboricola, Saccharomyces kudriavzevii, Saccharomyces mikatae, Ashbya gossypii, Kluveromyces lactis, Pichia pastoris, Candida glabrata, Candida tropicalis, Debaryomyces castelii, Yarrowia lipolitica, and Cyberlindnera jadinii.

The promoters of the invention can preferably originate from an organism selected from the group consisting of Saccharomyces cerevisiae (sc), Saccharomyces mikatae (Sm), Saccharomyces kudriavzevii (sk), Saccharomyces bayanus (sba), Saccharomyces arboricola (Sar), and Ashbya gossypii (Ago).

### Terminators

As it is disclosed herein, the expression of the genes of interest that have been genetically engineered for obtaining a recombinant cell according to the invention, and in particular a recombinant yeast according to the invention comprise appropriate transcription terminator sequences that are functional in recombinant cells of the invention, and in particular in recombinant yeast cells of the invention, in particular in Saccharomyces cerevisiae.

Said transcription terminators, identical or different, may be found in literature Yamanishi et al., (2013) ACS synthetic biology 2, 337-347.

Terminators more particularly interesting in the present invention may be selected from the group comprising:
tTPI1 from the gene encoding for the Triose Phosphate Isomerase (SEQ ID NO: 111),
tMET25 from the gene encoding for the O-acetyl homoserine-O-acetyl serine sulfhydrylase (SEQ ID NO: 112),
tDIT1 (SEQ ID NO: 113),
tRPL3 (SEQ ID NO: 114),
tRPL3.sm (SEQ ID NO: 115),
tRPL3.sba (SEQ ID NO: 116),
tRPL41B (SEQ ID NO: 117),
tRPL41B.Sba (SEQ ID NO: 118)
tRPL15A (SEQ ID NO: 119),
tRPL15A.Sm (SEQ ID NO: 120),
tRPL15A.sba (SEQ ID NO: 121),
tIDP1 (SEQ ID NO: 122),
tIDP1.Sba (SEQ ID NO: 123), and
tTEF1.sba (SEQ ID NO: 124).

Terminators more particularly interesting in the present invention may be selected from the group consisting in:
tTPI1 from the gene encoding for the Triose Phosphate Isomerase (SEQ ID NO: 111),
tMET25 from the gene encoding for the O-acetyl homoserine-O-acetyl serine sulfhydrylase (SEQ ID NO: 112)
tDIT1 (SEQ ID NO: 113)
tRPL3 (SEQ ID NO: 114)
tRPL3.Sm (SEQ ID NO: 115)
tRPL3.sba (SEQ ID NO: 116)
tRPL41B (SEQ ID NO: 117)
tRPL41B.Sba (SEQ ID NO: 118)
tRPL15A (SEQ ID NO: 119)
tRPL15A.Sm (SEQ ID NO: 120)
tIDP1 (SEQ ID NO: 122)
tIDP1.Sba (SEQ ID NO: 123), and
tTEF1.sba (SEQ ID NO: 124).

In particular, said terminator may be selected from the group consisting of tTPI1, tDIT11, tRPL3, tRPL3.Sm, tRPL41B, tRPL41B.Sba, tRPL15A, tRPL15A.Sm, tIDP1, tIDP1.Sba and tTEF1.sba.

The terminators of the invention can originate from any organism from the Saccharomycetes class and can in particular originate from an organism selected from the group consisting of Saccharomyces cerevisiae (sc), Saccharomyces mikatae (Sm), and Saccharomyces Bayanus (sba).

### Recombinant Cells

Recombinant cells of the invention can be selected from the group consisting of yeasts and bacteria.

Recombinant cells of the invention, such as a recombinant host cell of the invention, are preferably recombinant yeast cells.

Generally, yeast can grow rapidly and can be cultivated at higher density as compared with bacteria, and does not require an aseptic environment in the industrial setting.

Furthermore, yeast cells can be more easily separated from the culture medium compared to bacterial cells, greatly simplifying the process for product extraction and purification.

A recombinant cell of the invention, and in particular a recombinant yeast cell of the invention is preferably a Saccharomycetales cell.

A recombinant cell of the invention, and in particular a recombinant yeast of the invention can in particular belong to the Saccharomyces genus, or to the Candida genus, or to the Kluyveromyces genus, or to the Ogataea genus, or to the Yarrowia genus, or to the Debaryomyces genus, or to the Ashbya genus.

A recombinant cell of the invention belonging to the Saccharomyces genus can be selected from the group consisting of Saccharomyces cerevisiae, Saccharomyces boulardii, Saccharomyces bayanus, Saccharomyces paradoxus, Saccharomyces mikatae, Saccharomyces castelli, Saccharomyces cariocanus, Saccharomyces kudriavzevii, Saccharomyces arboricolus, Saccharomyces pastorianus, Saccharomyces uvarum and Saccharomyces delbrueckii.

A recombinant cell of the invention belonging to the Candida genus can be selected from the group consisting of Candida albicans, Candida glabrata, Candida tropicalis, Candida dubliniensis, Candida parapsilosis, Candida lusitaniae and Candida guilliermondii.

A recombinant cell of the invention belonging to the Kluyveromyces genus can be selected from the group consisting of Kluyveromyces lactis, Kluyveromyces marxianus, Kluyveromyces polysporus, Kluyveromyces thermotoleren, Kluyveromyces dobzhanskii and Kluyveromyces wickerhamii.

A recombinant cell of the invention belonging to the Ogataea genus can be selected from the group consisting of Ogataea polymorpha, Ogataea histrianica, Ogataea deakii, Ogataea kolombanensis, Ogataea philodendra, Ogataea siamensis, Ogataea angusta, Ogataea parapolymorpha, Ogataea minuta, Ogataea nonfermentans and Ogataea kodamae.

A recombinant cell of the invention belonging to the Yarrowia genus can be selected from the group consisting of Yarrowia lypolytica, Yarrowia parophonii, Yarrowia galli, Yarrowia oslonensis, Yarrowia alimentaria, Yarrowia hollandica and Yarrowia yakushimensis.

A recombinant cell of the invention belonging to the Debaryomyces genus can be selected from the group consisting of Debaryomyces hansenii, Debaryomyces carsonii, Debaryomyces castellii, Debaryomyces marama, Debaryomyces occidentalis, Debaryomyces oviformis, Debaryomyces nepalensis, Debaryomyces coudertii, Debaryomyces udenii, Debaryomyces psychrosporus and Debaryomyces yamadae.

A recombinant cell of the invention belonging to the Ashbya genus can be selected from the group consisting of Ashbya gossypii and Ashbya aceri.

A recombinant cell of the invention, and in particular a recombinant yeast cell of the invention, can in particular be selected from the group consisting of Saccharomyces cerevisiae, Saccharomyces boulardii, Saccharomyces bayanus, Saccharomyces paradoxus, Saccharomyces mikatae, Saccharomyces castelli, Candida albicans, Candida glabrata, Candida tropicalis, Kluyveromyces lactis, Kluyveromyces marxianus, Kluyveromyces polysporus, Kluyveromyces thermotolerens, Ogataea polymorpha, Yarrowia lypolytica, Debaryomyces hansenii, and Ashbya gossypii, and is preferably Saccharomyces cerevisiae.

In a particular embodiment, the recombinant yeast cell according to the invention is of the genus Saccharomyces, Kluyveromyces, or Eremothecium, more particularly is of the species selected from the group consisting of Saccharomyces cerevisiae, Kluyveromyces Marxianus, Ogataea polymorpha and Ashbya gossypii.

In an embodiment, a recombinant host cell of the invention is a yeast selected belonging to the Saccharomycetales order, in particular to the Saccharomycetaceae family, and is in particular selected from the group consisting of Yarrowia lipolitica, Kluyveromyces Marxianus, Ogataea polymorpha, Ashbya gossypii and Saccharomyces cerevisiae.

A recombinant cell of the invention can most preferably be a Saccharomyces cerevisiae cell.

As above-mentioned, a recombinant cell according to the invention has the ability to produce chondroitin by insertion of the one or more recombinant nucleic acids according to the present invention. In certain embodiments, a recombinant yeast according to the invention has the ability to produce chondroitin having a controlled size (controlled molecular weight) by insertion of the one or more recombinant nucleic acids according to the present invention.

Methods implemented to insert a specific DNA construct within a gene belong to the general knowledge of a man skilled in the art. A related method is described in more details in the herein after examples.

However, unexpected technical difficulties were encountered because the consequences of the insertion of DNA constructs within the genome of a cell, and in particular within the genome of a yeast, such as for example in the genome of Saccharomyces cerevisiae, are unpredictable. In particular, the survival rate of the cells, and in particular of the yeasts, and their ability to grow and produce the desired chondroitin is also unpredictable.

In order to obtain a recombinant cell, and in particular a recombinant yeast of the invention, many different constructs were tested by the inventors in order to obtain a viable and efficient recombinant cell, and in particular yeast.

### Culture Conditions

The present invention also relates to the use of a recombinant cell of the invention, for the production of chondroitin, in particular of chondroitin of controlled molecular weight.

The present invention further relates to a method of producing chondroitin of a desired molecular weight, comprising:
(a) cultivating a recombinant cell as defined herein in a cultivation medium for a time sufficient to produce chondroitin of the desired molecular weight; and
(b) optionally isolating or recovering the chondroitin from the recombinant cell and/or from the cultivation medium. Typically, cells of the invention, and in particular yeasts of the invention, are grown at a temperature in the range of about 20° C. to about 37° C., preferably at a temperature ranging from 27 to 34° C., in an appropriate culture medium.

Suitable growth media for cells of the invention, and in particular for yeasts of the invention, are common commercially prepared media such as broth that includes yeast nitrogen base, ammonium sulfate, and dextrose as the carbon/energy source or YPD Medium, a blend of peptone, yeast extract, and dextrose in optimal proportions for growing most. Other defined or synthetic growth media may also be used and the appropriate medium for growth of the particular cell, and in particular yeast, will be known by one skilled in the art of microbiology or fermentation science.

A particular medium that is suitable herein is the SY medium which comprises the following elements:
KH2PO4: 100 mM; MgSO4 7H2O: 2.8 mM; K2SO4: 11.5 mM; Na2SO4:
1.1 mM; NaCl: 2.6 mM; CaCl₂) 2H2O: 0.7 mM; CuSO4 5H2O: 15 μM; KI: 6 μM; FeCl3: 30 μM; ZnSO4 7H2O: 61 μM; MnSO4 H2O: 25 μM; H2SO4: 110 μM; Panthotenic Acids hemicalcium salt: 42 μM; Thiamin hydrochloride: 59 μM; Pyridoxin hydrochloride: 49 μM; Myo-Inositol (C6H1206): 555 μM; Nicotinic acid (C6H5NO2): 29 μM; D-Biotine:
0.82 μM; Ammonium citrate tribasic: 33 mM; and glucose or sucrose 2-30%.

Carbon sources that may be used in the culture medium include fructose, mannose, xylose and arabinose; oligosaccharides such as lactose, maltose, galactose, or sucrose; polysaccharides such as starch or cellulose or mixtures thereof; and unpurified mixtures from renewable feedstocks such as cheese whey permeate cornsteep liquor, sugar beet molasses, and barley malt.

Nitrogen sources that may be included in the culture medium include peptone, yeast extracts, meat extracts, malt extracts, urea, ammonium sulfate, ammonium chloride, ammonium nitrate, ammonium phosphate, ammonium citrate and combinations thereof.

The culture medium may further comprise trace elements (e.g., metal salts), for example magnesium salts, cobalt salts and/or manganese salts; as well as growth factors such as amino acids, vitamins, growth promoters, and the like.

Examples of vitamins that may be included are Panthotenic Acids hemicalcium, Thiamin hydrochloride; Pyridoxin hydrochloride; Myo-Inositol; Nicotinic acid; D-Biotine, folic acid, p-aminobenzoique acid, riboflavin.

A culture medium of the invention may further include rare elements, such as CuSO₄·5H₂O, KI, FeCl₃, ZnSO₄·7H₂O, MnSO₄·H₂O, or H₂SO₄, MgCl2, CaCl2, NaCl, K2HPO4, KH2PO4, ZnCl, H3BO3, MnSO4, Na2MoO4.

The term “appropriate culture medium” is above-defined.

Examples of known culture media for a recombinant cell according to the present invention are known to the person skilled in the art, and are presented in the following publication D. Burke et al., Methods in yeast Genetics—A cold spring harbor laboratory course Manual (2000).

Suitable pH ranges for the fermentation may be between pH 3.0 to pH 7.5, where pH 4 to pH 6 is preferred as the initial condition.

As mentioned elsewhere in this specification, the pH value of the culture medium can be regulated during the cultivation step of a method of the invention in order to modulate the activity of the polypeptide having chondroitin activity, which will impact the molecular weight of the chondroitin molecules produced by the recombinant cell of the invention, and in particular by the recombinant yeast of the invention.

In particular, the pH of the culture medium can be modified according to the chondroitin that is meant to be produced by the recombinant yeast. For example, the pH of the culture medium may remain at a pH of 4, of 5.5 or of 6 during the culture time.

In a particular embodiment, the pH of the culture medium may change or be changed during the time length of the culture of the recombinant cell of the invention, in particular of the recombinant yeast of the invention. As previously mentioned, Saccharomyces cerevisiae acidifies the medium in which it is cultivated and thus reduces the pH of its culture medium. For example, the pH of the culture medium may begin at 6, may be taken down to 4 and then brought back up to 6. In another example, the pH of the culture medium may begin at 6, remain or be maintained at 6, and then may be lowered to 4.

In a particular embodiment, the pH of the culture medium may be modulated during the cultivating step (a) of a method of the invention so that at the end of the cultivating step of a method of the invention, the pH of the cultivation medium is the same as the pH at the beginning of said cultivation step (a).

In another embodiment, the pH of the culture medium may remain the same during the time length of the culture of the recombinant cell of the invention, and in particular the recombinant yeast cell of the invention.

Said time length of the culture of a recombinant cell according to the invention, and in particular of the recombinant yeast of the invention, can vary depending on the molecular weight of the chondroitin of interest. The longer said time length, the lower the molecular weight of chondroitin in a given culture medium of a recombinant cell of the invention, and in particular a recombinant yeast cell of the invention.

The time length of the culture time of a recombinant cell of the invention, and in particular a recombinant yeast cell in the present invention can be a period of from about 35 hours to about 50 hours, preferably from about 40 hours to about 50 hours, and is in particular about 48 hours.

Fermentations may be performed under aerobic conditions or micro-aerobic conditions.

The amount of chondroitin product in the fermentation medium can be determined using a number of methods known in the art, for example, high performance liquid chromatography (HPLC) or gas chromatography (GC).

The present process may employ a batch method of fermentation. A classical batch fermentation is a closed system where the composition of the medium is set at the beginning of the fermentation and not subject to artificial alterations during the fermentation. Thus, at the beginning of the fermentation, the medium is inoculated with the desired organism or organisms, and fermentation is permitted to occur without adding anything to the system. Typically, however, a “batch” fermentation method or system is batch with respect to the addition of carbon source and attempts are often made at controlling factors such as temperature, pH and oxygen concentration. In batch systems, the metabolite and biomass compositions of the system change constantly up to the time when the fermentation is stopped. Within batch cultures cells progress through a static lag phase to a high growth log phase and finally to a stationary phase where growth rate is diminished or halted. If untreated, cells in the stationary phase will eventually die. Cells in log phase generally are responsible for the bulk of production of end product or intermediate.

A Fed-Batch system may also be used in the present invention. A Fed-Batch system is similar to a typical batch system with the exception that the carbon source substrate is added in increments as the fermentation progresses. Fed-Batch systems are useful when catabolite repression (e.g. glucose repression) is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO₂.

Batch and Fed-Batch culturing methods are common and well known in the art and examples may be found in Biotechnology: A Textbook of Industrial Microbiology, Crueger, Crueger, and Brock, Second Edition (1989) Sinauer Associates, Inc., Sunderland, MA, or Deshpande, Mukund V., Appl. Biochem. Biotechnol., 36, 227, (1992). Although the present invention is performed in batch mode it is contemplated that the method would be adaptable to continuous fermentation.

Continuous fermentation is an open system where a defined fermentation medium is added continuously to a bioreactor and an equal amount of conditioned media is removed simultaneously for processing. Continuous fermentation generally maintains the cultures at a constant high density where cells are primarily in log phase growth.

Continuous fermentation allows for the modulation of one factor or any number of factors that affect cell growth or end product concentration. For example, one method will maintain a limiting nutrient such as the carbon source or nitrogen level at a fixed rate and allow all other parameters to vary. In other systems a number of factors affecting growth can be altered continuously while the cell concentration, measured by media turbidity, is kept constant. Continuous systems strive to maintain steady state growth conditions and thus the cell loss due to the medium being drawn off must be balanced against the cell growth rate in the fermentation. Methods of modulating nutrients and growth factors for continuous fermentation processes as well as techniques for maximizing the rate of product formation are well known in the art of industrial microbiology.

It is contemplated that the present invention may be practiced using either batch, fed-batch or continuous processes and that any known mode of fermentation would be suitable. Additionally, it is contemplated that cells may be immobilized on a substrate as whole cell catalysts and subjected to fermentation conditions for production.

In order to still improve the chondroitin production, a particular embodiment may consist of culturing the recombinant cells of the invention, in particular the recombinant yeast cells of the invention, in an appropriate culture medium, such as above-mentioned, wherein the said culture medium comprises an optimal amount of carbon source, especially glucose or sucrose.

In preferred embodiments, the carbon source comprised in said optimal culture medium consists of glucose and/or sucrose. In preferred embodiments, the said optimal culture medium comprises 1% w/w or more glucose and/or sucrose, in particular comprises 5% w/w or more glucose and/or sucrose, in particular comprises 10% w/w or more glucose and/or sucrose, in particular comprises 15% w/w or more glucose and/or sucrose. In preferred embodiments, the said optimal culture medium comprises at most 40% w/w glucose, which includes at most 35% w/w glucose.

In a preferred embodiment, the method of the invention is carried out on an industrial scale.

More particularly, the cultivation medium of the method according to the invention can be at least about 100 L, more preferably in the range of about 1,000 L to about 3,000 L, even more preferably about 10,000 L, even more preferably 100,000 L, or even about 250,000 L.

The invention further relates to a method for producing chondroitin as previously described, and comprising the steps of:
(a) culturing a recombinant cell of the invention in a culture medium; and
(b) recovering the chondroitin from said culture medium,
wherein the chondroitin recovered in step (b) has a molecular weight controlled through the selection of:
   the nature and origin of the one or more recombinant nucleic acids encoding a polypeptide having a chondroitinase activity of the recombinant cell of the invention, and in particular of the recombinant yeast of the invention,
   the nature and origin of the promoter controlling the expression of the one or more recombinant nucleic acids encoding a polypeptide having a chondroitin activity of the recombinant cell of the invention, and in particular of the recombinant yeast of the invention,
   the presence or the absence of an anchoring signal associated to one or more recombinant nucleic acids encoding a polypeptide having chondroitin activity of the recombinant cell of the invention, and in particular of the recombinant yeast of the invention,
   the pH of the culture medium during the step of culturing the recombinant cell of the invention, and in particular of the recombinant yeast of the invention, and/or
   the duration of the culturing of the recombinant cell of the invention, and in particular of the recombinant yeast of the invention.

The molecular weight of the chondroitin can be a particular molecular weight or more preferably a specific range of molecular weights such as, for example, less than 50 kDa, in the range of about 20 kDa to about 50 kDa, greater than or equal to 50 kDa, in the range of about 50 kDa to about 150 kDa, in the range of about 50 kDa to about 250 kDa, greater than or equal to 100 kDa, in the range of about 100 kDa to about 1500 kDa, in the range of about 150 kDa to about 1500 kDa, greater than 1000 kDa or greater than 1500 kDa.

The present invention also relates to the use of a recombinant cell according to the invention, and in particular a recombinant yeast of the invention, for the production of chondroitin having a molecular weight in the range of from about 20 kDa to about 50 kDa or from about 50 kDa to about 1000 kDa.

The one or more recombinant nucleic acids encoding a polypeptide having chondroitinase activity of a recombinant cell of the invention can for example be selected from those obtained or derived from at least one of Cupiennius salei, Tityus serrulatus, Bos taurus, Apis mellifera, Macaca mulatta, or Vespa magnifica, in particular from those of_sequences set forth as sequences SEQ ID NO: 48 (Csa), SEQ ID NO: 49 (Ts), SEQ ID NO: 50 (Bt), SEQ ID NO: 51 (Am), SEQ ID NO: 52 (Mm) or SEQ ID NO: 53 (Vm) when they are secreted (i.e. in the presence of a secretion signal and in the absence of an anchoring signal).

The one or more recombinant nucleic acids encoding a polypeptide having chondroitinase activity of a recombinant cell of the invention can for example be selected from those obtained or derived from at least one of Cupiennius salei, Tityus serrulatus, Bos taurus, Apis mellifera, Macaca mulatta, or Vespa magnifica, in particular from those of sequences set forth as sequences SEQ ID NO: 54 (Csa), SEQ ID NO: 55 (Ts), SEQ ID NO: 56 (Bt), SEQ ID NO: 57 (Am), SEQ ID NO: 58 (Mm) or SEQ ID NO: 59 (Vm) when they are anchored (i.e. in the presence of both a secretion signal and an anchoring signal).

The one or more recombinant nucleic acids encoding a polypeptide having chondroitinase activity of a recombinant cell of the invention can for example be placed under the control of a promoter selected from the group consisting of pTDH3, pTDH3.Sk, pTDH3-1.Sba, pTEF1, pTEF1.Ago, pTEF1.sba, pCCW12, pCCW12.Sba, pCCW120.Sm, pCWP2, pCW10.Ago, pNUP57 and pFBA1, and in particular from the group consisting of pCCW12.Sba, pCW10.Ago and pNUP57.

In particular, the promoters of the one or more recombinant nucleic acids encoding a polypeptide having chondroitinase activity of a recombinant cell of the invention, and in particular of a recombinant yeast of the invention, may be obtained or derived from Saccharomyces Bayanus, Saccharomyces kudriavzevii, Saccharomyces mikatae, Saccharomyces arboricola or other saccharomycetales, or Abishia gossypii.

The secretion signal of the present invention may for example have:
the nucleic acid sequence set forth as SEQ ID NO: 125; and/or
the amino acid sequence set forth as SEQ ID NO: 126.

The anchoring signal of the present invention may for example have:
the nucleic acid sequence set forth as SEQ ID NO: 127; and/or
the amino acid sequence set forth SEQ ID NO: 128.

The secretion signal may be fused to the polypeptide having a chondroitinase activity by creating a chimeric nucleic acid starting by a nucleic acid sequence encoding the signal peptide followed by a recombinant nucleic acid encoding a polypeptide having chondroitinase activity as previously defined.

The secretion signal and anchoring signal may be fused to the polypeptide having chondroitinase activity by creating a chimeric nucleic acid starting by a nucleic acid sequence encoding the signal peptide followed by a recombinant nucleic acid encoding a polypeptide having chondroitinase activity as previously defined followed by a nucleic acid sequence encoding for the anchoring signal.

Such chimeric nucleic acid sequences may be obtained by techniques known by the man skilled in the art such as chemical synthesis of nucleic acids or any recombination techniques such as cloning or PCR.

The invention further relates to a method as previously described, for producing chondroitin having a molecular weight less than about 10 kDa, wherein:
(a) the pH of the medium is superior to 4, and is in particular in the range of from about 5 to about 7;
(b) the one or more recombinant nucleic acids encoding a polypeptide having chondroitinase activity integrated in the genome of the recombinant cell of the invention originates or is derived from Tityus serrulatus, possesses a secretion signal but is devoid of an anchoring signal, and is under the control of a pCCW12.sba promoter;
(c) the polypeptide having chondroitinase activity is a hyaluronidase;
(d) the step of recovering the chondroitin from the culture medium is performed about 48 hours after the beginning of the culturing of the recombinant cell of the invention, in particular the recombinant yeast cell of the invention;
the recombinant cell being in particular a recombinant yeast cell, and more particularly a Saccharomyces cerevisiae cell.

The invention further relates to a method as previously described, for producing chondroitin having a molecular from about 10 kDa to about 50 kDa, wherein:
(a) the pH of the medium is superior to 4, and is in particular in the range of from about 5 to about 7;
(b) the one or more recombinant nucleic acids encoding a polypeptide having chondroitinase activity integrated in the genome of the recombinant cell of the invention originates or is derived from Tityus serrulatus, possesses a secretion signal but is devoid of an anchoring signal, and is under the control of a pCCW10.Ago promoter;
(c) the polypeptide having chondroitinase activity is a hyaluronidase;
(d) the step of recovering the chondroitin from the culture medium is performed about 48 hours after the beginning of the culturing of the recombinant cell of the invention, in particular the recombinant yeast cell of the invention;
the recombinant cell being in particular a recombinant yeast cell, and more particularly a Saccharomyces cerevisiae cell.

The invention further relates to a method as previously described, for producing chondroitin having a molecular from about 50 kDa to about 500 kDa, wherein:
(a) the pH of the medium is superior to 4, and is in particular in the range of from about 5 to about 7;
(b) the one or more recombinant nucleic acids encoding a polypeptide having chondroitinase activity integrated in the genome of the recombinant cell of the invention originates or is derived from Tityus serrulatus, possesses a secretion signal but is devoid of an anchoring signal, and is under the control of a pNUP57 promoter;
(c) the polypeptide having chondroitinase activity is a hyaluronidase;
(d) the step of recovering the chondroitin from the culture medium is performed about 48 hours after the beginning of the culturing of the recombinant cell of the invention, in particular the recombinant yeast cell of the invention;
the recombinant cell being in particular a recombinant yeast cell, and more particularly a Saccharomyces cerevisiae cell.

Another aspect of the present invention relates to chondroitin obtained or obtainable from a recombinant cell of the invention or from a method according to the invention.

A further aspect of the invention is a cultivation medium comprising the chondroitin of the invention.

The invention further relates to a composition comprising the chondroitin according to the invention.

The invention also relates to an industrial product or a consumer product or a consumable comprising (i) chondroitin of the invention (ii) the cultivation medium comprising chondroitin of the invention or (iii) the composition comprising chondroitin of the invention.

In particular, said industrial product or consumer product or consumable according to the invention can be a cosmetic product, a flavour product, a fragrance product, a food product, a food, a beverage, a texturant, a pharmaceutical composition, a dietary supplement, a nutraceutical, a cleaning product and/or a dental and/or an oral hygiene composition.

### Purification of Chondroitin

According to a specific aspect of the invention, the fermentative production of chondroitin preferably comprises a step of isolation of the chondroitin produced from the culture medium. Recovering the chondroitin from the culture medium is a routine task for a man skilled in the art. It may be achieved by a number of techniques well known in the art including but not limiting to pervaporation, selective precipitation, filtration, centrifugation, spray drying, lyophylisation or liquid extraction. The expert in the field knows how to adapt parameters of each technique dependent on the characteristics of the material to be separated.

The yeast as model of a cell in the present invention is preferred in that the synthesized chondroitin is/are entirely exported outside the cells, thus simplifying the purification process.

Gas stripping is achieved with a stripping gas chosen among helium, argon, carbon dioxide, hydrogen, nitrogen or mixture thereof.

Liquid extraction is achieved with organic solvent as the hydrophobic phase such as pentane, hexane, heptane or dodecane. Renewal solvents may also be used.

### Formulations and Products

A composition of the invention may be incorporated into a formulation/product, such as a nutraceutical, pharmaceutical, veterinary, oenological or cosmetic formulation/product.

Thus, the present invention provides a formulation comprising a composition of the invention. For example, the present invention may provide a cosmetic formulation comprising the composition of the invention.

The present invention also provides a product comprising a composition of the invention. For example, the present invention may provide a cosmetic product comprising the composition of the invention.

A “cosmetic product” is intended to mean any substance or mixture intended to be placed in contact with the external parts of the human body (epidermis, hair system, nails, lips and external genital organs) or with the teeth and the mucous membranes of the oral cavity with a view exclusively or mainly to cleaning them, perfuming them, changing their appearance, protecting them, keeping them in good condition, correcting body odours and/or combinations thereof.

A “substance” is intended to mean a chemical element and its compounds in the natural state or obtained by any manufacturing process, including any additive necessary to preserve its stability and any impurity deriving from the process used but excluding any solvent which may be separated without affecting the stability of the substance or changing its composition.

A “mixture” is intended to mean a mixture or solution composed of two or more substances.

The present invention also provides the use of a composition of the invention in a nutraceutical, pharmaceutical, veterinary, oenological or cosmetic formulation/product.

Such formulations or products are hereinafter referred to as the “formulations or products of the invention”.

The nutraceutical, pharmaceutical, veterinary, oenological or cosmetic formulation/product may optionally further comprise pharmaceutically/veterinary/cosmetic (including cosmetic active) ingredients, such as excipients, carriers and mixtures thereof as appropriate.

“Cosmetic” or “Cosmetic Active Ingredients” means any and all natural, naturally occurring, nature identical, synthetic synthetically produced, biosynthetically produced, sustainable, renewable and/or biodegradable compounds, ingredients, intermediates, molecules, substances, raw materials or products individually or as part of a mixture of compounds, ingredients, intermediates, molecules, substances, raw materials or products, blends, compositions, formulations (including but not limited to skin moisturizers, creams, balms, serums, oils, eye, facial makeup, wash off hair products, leave-on hair products, hair colorants (including but not limited to natural hair colorants) and/or combinations thereof), finished products and related technologies including, but not limited to components, incorporated, for example, into a cosmetic formulation (such as but not limited to natural colorants, preservatives, emulsifiers, anti-oxidants and the like which do not, for example, have an activity on the skin, hair, scalp and the like but play a role in the formulation of the finished product), delivery systems, marketing aids (such as, for example, coloured unispheres applied to translucent formulations) and methods of making anything related thereto useful in/used for/intended for:
application by rubbing, pouring, sprinkling, spraying or otherwise directly on or to a human or animal body and/or by placing in contact with the various external and/or on surface parts of a human or animal body (including but not limited to the skin, hair, body hair, the hair system, scalp, nails, lips, external genitalia, teeth, oral and/or nasal mucosa and the like); and/or application indirectly on or to a human or animal body such as for example, application as part of a textile or application to a textile as part of a delivery device (such as a capsule) or a delivery system (such as blends or formulations) applied to a textile; and/or
cleansing, caring, cooling, beautifying, conditioning, treating, soothing, texturizing, promoting attractiveness, protecting, maintaining, improving, enhancing, altering and/or changing an external part and/or surface of a human or animal body (such as, but not limited to the scalp) or the aesthetic appearance of a human or animal body; and/or with a view to mainly cleaning or perfuming, or protecting or maintaining in good condition or combating body odour or changing the appearance of or correcting or repairing a state of imbalance in skin, oral mucosa, scalp or hair by providing a calming, healing, repairing, or revitalization, hydration of the skin or in order to provide relief to, lubricate, moisten, tone, heal, sterilize, relieve, correct and/or remedy states of dryness, irritation, injury or fatigue, and/or with a view to correcting pigmentation disorders or providing a non-pharmaceutical prevention and/or treatment of dandruff, acne, irritation and/or inflammation and the like and/or rebalancing the bacterial flora (such as, for example, the microbiome) on the surface of the skin (such as, for example, by promoting the level of beneficial bacterial flora on the skin surface) and/or for the purposes of keeping a human or animal body in good condition for health and/or wellbeing purposes and/or for improving the appearance of a human or animal body by, for example, improving the appearance of a product applied to a human or body; and/or
providing a cosmetic and/or dermatological function and/or benefit with a biological activity benefit (but without affecting a body's structure or function. For the avoidance of doubt, a Cosmetic or Cosmetic Active Ingredient or any part thereof may also qualify as a Functional Ingredient and/or Nutraceutical.

“Functional Ingredient” means a food ingredient or part of a food that provides medicinal or health benefits, including any of the following: a carotenoid, dietary fiber, fatty acid, saponin, antioxidant, flavonoid, isothiocyanate, phenol, polyphenol (such as resveratrol), plant sterol or stanol (phytosterols and phytostanols), a polyol, a prebiotic, a phytoestrogen, soy protein, sulfides/thiol, a vitamin, glucosamine, preservatives, hydration agents, edible gelling ingredients, edible gel mixes and gel compositions, long chain primary aliphatic saturated alcohols, colour agents, texturizing agents, emulsifiers and combinations thereof.

“Nutraceutical” means any and all natural, naturally occurring, sustainable, synthetically-produced and bio-synthetically-produced compounds, mixtures of compounds, Functional Ingredients, molecules, compositions, raw materials, and intermediates (including components and delivery devices (such as capsules) related thereto, delivery systems thereof (such as blends or formulations) and methods of making the foregoing) that are associated with health and/or cosmetic benefits, as well as improving or maintaining the appearance of the human body. For the avoidance of doubt, Nutraceuticals includes compounds that can be used as supplements to food or beverage, whether a solid formulation, capsule, tablet, liquid formulation, solution or suspension.

Alternatively, the nutraceutical, pharmaceutical, veterinary, oenological or cosmetic formulation/product may consist or consist essentially of the composition of the invention.

The cosmetic formulation/product may be an anti-aging formulation.

As used herein, references to pharmaceutically, veterinary or cosmetically acceptable excipients may refer to pharmaceutically, veterinary or cosmetically acceptable adjuvants, diluents and/or carriers as known to those skilled in the art.

By “pharmaceutically/veterinary/cosmetically acceptable” we mean that the additional components of the composition are generally safe, non-toxic, and neither biologically nor otherwise undesirable. For example, the additional components may be generally sterile and pyrogen free. Such components must be “acceptable” in the sense of being compatible with the composition of the invention and not deleterious to the recipients thereof. Thus, “pharmaceutically acceptable excipients” includes any compound(s) used in forming a part of the formulation that is intended to act merely as an excipient, i.e. not intended to have biological activity itself.

The nutraceutical, pharmaceutical, veterinary, oenological or cosmetic formulation/product may be in the form of a liquid or a solid.

Liquid dosage formulations/products for oral administration include solutions, emulsions, aqueous or oily suspensions, syrups and elixirs.

Formulations and products (e.g. pharmaceutical, veterinary or cosmetic formulations/products) described herein, such as those intended for oral administration, may be prepared according to methods known to those skilled in the art, such as by mixing the components of the formulation/product together.

The formulation or product (e.g. pharmaceutical, veterinary or cosmetic formulation/product) may contain one or more additional ingredients, such as pharmaceutical ingredients and excipients, such as sweetening agents, flavouring agents, colouring agents and preserving agents.

The formulation or product (e.g. pharmaceutical, veterinary or cosmetic formulation/product) may also contain one or more additional active ingredients, such as cosmetic or pharmaceutical active ingredients, such as hyaluronic acid, Centella asiatica extract, peptides such as Matrixyl® and Argireline®, and mixtures thereof.

The formulation or product of the invention may contain the active ingredient(s) in admixture with non-toxic pharmaceutically acceptable excipients (or ingredients). These excipients (or ingredients) may, for example, be: inert diluents, such as calcium carbonate, sodium carbonate, lactose, calcium phosphate or sodium phosphate; granulating and disintegrating agents, for example, corn starch, maltodextrin or alginic acid; binding agents, for example, starch, gelatine or acacia; or lubricating agents, for example magnesium stearate, stearic acid, talc and mixtures thereof.

Liquid formulations or products (e.g. pharmaceutical, veterinary or cosmetic formulations/products) may be contained within a capsule, which may be uncoated or coated as defined above.

Suitable pharmaceutical or veterinary carriers include inert solid diluents or fillers, sterile aqueous solutions and various organic solvents. Examples of liquid carriers are syrup, peanut oil, olive oil, phospholipids, fatty acids, fatty acid amines, polyoxyethylene and water.

Moreover, the carrier or diluent may include any sustained release material known in the art, such as glyceryl monostearate or glyceryl distearate, alone or mixed with a wax.

Suitable pharmaceutical carriers include inert sterile aqueous solutions and various organic solvents. Examples of liquid carriers are syrup, vegetables oils, phospholipids, fatty acids, fatty acid amines, polyoxyethylene and water. Moreover, the carrier or diluent may include any sustained release material known in the art, such as glyceryl monostearate or glyceryl distearate, alone or mixed with a wax.

Suitable cosmetic carriers are typically those that are suitable for topical administration to the outer surface of the human body, such as the skin and/or hair and/or scalp.

Typically, such carriers are dermatologically acceptable.

The phrase “dermatologically acceptable carrier” means that the carrier is suitable for topical application to the keratinous tissue, has good aesthetic properties, is compatible with the actives in the composition, and will not cause any unreasonable safety or toxicity concerns.

The carrier can be in a wide variety of forms. In some instances, the solubility or dispersibility of the components (e.g. extracts, sunscreen active, additional components) may dictate the form and character of the carrier. Non-limiting examples include simple solutions (e.g. aqueous or anhydrous), dispersions, emulsions, and solid forms (e.g. gels, sticks, flowable solids, or amorphous materials).

The dermatologically acceptable carrier may be in the form of an emulsion. An emulsion may be generally classified as having a continuous aqueous phase (e.g. oil-in-water and water-in-oil-in-water) or a continuous oil phase (e.g. water-in-oil or oil-in-water). The oil phase of the present invention may comprise silicone oils, non-silicone oils such as hydrocarbon oils, esters, ethers, and the like, and mixtures thereof. The aqueous phase typically comprises water and water-soluble ingredients (e.g. water-soluble moisturizing agents, conditioning agents, anti-microbials, humectants and/or other skin care actives). However, in some instances, the aqueous phase may comprise components other than water, including but not limited to watersoluble moisturizing agents, conditioning agents, antimicrobials, humectants and/or other watersoluble skin care actives. In some instances, the non-water component of the composition comprises a humectant, such as glycerin and/or other polyol(s). Emulsions may also contain an emulsifier. Emulsifiers may be non-ionic, anionic or cationic.

The carrier may contain one or more dermatologically acceptable, hydrophilic diluents. As used herein, “diluent” includes materials in which the composition of the invention can be dispersed, dissolved, or otherwise incorporated. Hydrophilic diluents include water, organic hydrophilic diluents, such as lower monovalent alcohols (e.g., C1-C4), and low molecular weight glycols and polyols, including propylene glycol, polyethylene glycol, polypropylene glycol, glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

The cosmetic formulation/product may optionally include one or more additional ingredients commonly used in cosmetic compositions (e.g., colorants, skin tone agents, skin anti-aging agents, anti-inflammatory agents, sunscreen agents, combinations of these and the like), provided that the additional ingredients do not undesirably alter the anti-glycation benefits provided by the composition.

In some instances, it may be desirable to select skin tone agents that function via different biological pathways so that the actives do not interfere with one another, which could reduce the efficacy of both agents. The additional ingredients, when incorporated into the composition, should be suitable for use in contact with human skin tissue without undue toxicity, incompatibility, instability, allergic response, and the like.

The term “carrier” as used herein, may also refer to a natural product or a product originating from nature that has been transformed or modified so that it is distinct from the natural product from which it originated, such as maltodextrin.

The amount of the composition of the invention present in nutraceutical, pharmaceutical, veterinary, oenological or cosmetic formulations or products will vary depending on the application.

Typically, the amount of composition of the invention that may be present in nutraceutical, pharmaceutical, veterinary, oenological or cosmetic formulations or products will be from about 0.001 to about 50% by weight, such as from about 0.01% to about 30% or from about 1% to about 20% of the nutraceutical, pharmaceutical, veterinary, oenological or cosmetic formulations or products, such as from about 0.01 to about 20%, or from about 0.1 to 10% or from about 1 to about 5% by weight of the formulation or product.

Those of skill in the art will recognize that, due to the degenerate nature of the genetic code, a variety of DNA molecules differing in their nucleotide sequences can be used to encode a given enzyme of the disclosure. The native DNA sequence encoding the biosynthetic enzymes described above are referenced herein merely to illustrate an embodiment of the disclosure, and the disclosure includes DNA molecules of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

Described herein are specific genes and proteins useful in the methods, compositions and organisms of the disclosure; however it will be recognized that absolute identity to such genes is not necessary. For example, changes in a particular gene or polynucleotide comprising a sequence encoding a polypeptide or enzyme can be performed and screened for activity. Typically such changes comprise conservative mutations and silent mutations. Such modified or mutated polynucleotides and polypeptides can be screened for expression of a functional enzyme using methods known in the art.

Due to the inherent degeneracy of the genetic code, other polynucleotides which encode substantially the same or functionally equivalent polypeptides can also be used to clone and express the polynucleotides encoding such enzymes

Techniques known to those skilled in the art may be suitable to identify additional homologous genes and homologous enzymes. Generally, analogous genes and/or analogous enzymes can be identified by functional analysis and will have functional similarities.

Techniques known to those skilled in the art may be suitable to identify analogous genes and analogous enzymes or any biosynthetic pathway genes, proteins, or enzymes, techniques may include, but are not limited to, cloning a gene by PCR using primers based on a published sequence of a gene/enzyme of interest, or by degenerate PCR using degenerate primers designed to amplify a conserved region among a gene of interest. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity (e.g. as described herein or in Kiritani, K., Branched-Chain Amino Acids Methods Enzymology, 1970), then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, or MetaCYC. The candidate gene or enzyme may be identified within the above mentioned databases in accordance with the teachings herein.

The terms “between . . . and . . . ” and “ranging from . . . to . . . ” should be understood as being inclusive of the limits, unless otherwise specified. Throughout this specification and the claims which follow, unless the context requires otherwise, the word “comprise”, and variations such as “comprises”, “comprising” and “possesses”, will be understood to imply the inclusion of a stated integer or step or group of integers or steps but not the exclusion of any other integer or step or group of integer or step. The term “comprising” also means “including” as well as “consisting” e.g. a composition “comprising” X may consist exclusively of X or may include something additional e.g. X+Y. It must be noted also that, as used in this specification and the appended claims, the singular forms “a”, “an” and “the” include plural referents unless the content clearly dictates otherwise. By way of example, a reference to “a gene” or “an enzyme” is a reference to “one or more genes” or “one or more enzymes”.

It is to be understood that this disclosure is not limited to the particular methodology, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by the person skilled in the art. In accordance with the present disclosure there may be conventional molecular biology, microbiology, and recombinant DNA techniques employed which are within the skill of the art.

This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. Preferably, the terms used herein are defined as described in “A multilingual glossary of biotechnological terms: (IUPAC Recommendations)”, Leuenberger, H. G. W, Nagel, B. and Kolbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety.

The examples and figures which follow are presented by way of illustration and without implied limitation of the invention.

### EXAMPLES

### Example 1: Protocol for Making a Recombinant Saccharomyces cerevisiae Strain According to the Invention

All the hereinafter implemented recombinant Saccharomyces cerevisiae strains were constructed from standard strains using standard yeast molecular genetics procedure (Methods in yeast Genetics—A cold spring harbor laboratory course Manual (2000) by D. Burke, D. Dawson, T. Stearns CSHL Press).

Cluster of the following-mentioned genes were integrated in recombinant yeast at once using the ability of yeast to efficiently recombine free DNA ends which have sequence homology.

In addition, for a better comprehension of following genotypes:
jlp1, his 3, leu2, sam3 and met14 are insertion sites.
Lowercase letters mean that the considered gene is inactive, uppercase letters reflect an active gene.
“::”: following a gene name means that the gene is interrupted by what follows (if more than one gene are inserted, they are noted in brackets [ ]). The interruption of the gene is concomitant with an entire deletion of the coding sequence but preserves the promoter. In consequence the gene followed by “::” is inactive and is noted in lowercase. If not specified the transcription of the gene inserted is controlled by the promoter of the disrupted gene.
“gene.Kl” means that the gene originates from Kluyveromyces lactis. When nothing is indicated after a gene in the present examples, this means that the gene is from Saccharomyces cerevisiae.

More particularly, the coding sequences to be cloned were artificially synthetized. For heterologous sequences (non-yeast), the nucleic sequences were modified in order to obtain a synonymous coding sequence using the yeast codon usage. Using restriction enzyme and classical cloning technology, each synthetic sequence was cloned in between a transcription promoter and a transcription terminator. Each promoter sequence is preceded by a 50 to 200 nucleotide sequence homologous to the sequence of the terminator of the upstream gene. Similarly, the terminator of each gene (a gene comprising the promoter-coding sequence-terminator) is followed by sequences homologous to the gene immediately following. So that each of the unit to be integrated have a 50-200 nucleotide overlap with both the unit upstream and the unit downstream. For the first unit, the promoter is preceded by 50-200 nucleotides homologous to the yeast chromosome nucleotide for the locus in which it will be integrated. Similarly, for the last unit, the terminator is followed by 50-200 nucleotides homologous to the yeast chromosome nucleotide for the locus in which it will be integrated.

Each unit is then PCR amplified from the plasmids constructs, yielding X unit of linear DNA having overlapping sequences. At least one of this gene is an auxotrophic marker, in order to select for recombination event. All the linear fragments are transformed in the yeast at once, and a recombinant yeast cell is selected for the auxotrophy related to the marker used.

The integrity of the sequence is then verified by PCR and sequencing.

### Example 2: Comparative Examples for the Production of Chondroitin

Firstly, three recombinant strains are obtained: YA5809, YA5810 and YA5571.

Accordingly, these three strains are as follows:
YA5809: MAT-α, his3::[tRPL3-UGP1-pSAM1, pMET6-QRI1-tIDP1, HIS3]x5, jlp1::[LEU2.Sba-loxP, pTEF1.Sba-HASB.Vir-tRPL3.Sm, pTDH3.Sk-HASB-A.Vir-tTEF1.Sba, pTDH3-1.Sba-HCOS1-3.Pm-tRPL15A.Sm, pCCW12-KFOA.Pm-tRPL41B.Sba, pCCW120.Sm-KFOA.Pm-tRPL15A.Sba, pTEF1.Ago-GFA1.Vir-tRPL15A], leu2, met14::[TRP1.Sba-RS, pTDH3-PGM1-tIDP1, pCWP2-GNA1-tTPI1, pTEF1-PCM1-tRPL41B, pFBA1-UGP1-tRPL3, pTDH3-1.Sba-QRI1-tIDP1.Sba, pTEF1.Ago-GFA1-tRPL15A], trp1, ura3
YA5810: MAT-α, his3::[tRPL3-UGP1-pSAM1, pMET6-QRI1-tIDP1, HIS3]x5, jlp1::[LEU2.Sba-loxP, pTEF1.Sba-HASB.Vir-tRPL3.Sm, pTDH3.Sk-HASB-A.Vir-tTEF1.Sba, pTDH3-1.Sba-HCOS1-3.Pm-tRPL15A.Sm, pCCW12-KFOA.Pm-tRPL41B.Sba, pCCW120.Sm-KFOA.Pm-tRPL15A.Sba, pTEF1.Ago-GFA1.Vir-tRPL15A], leu2, met14::[TRP1.Sba-RS, pTDH3-PGM1-tIDP1, pCWP2-GNA1-tTPI1, pTEF1-PCM1-tRPL41B, pFBA1-UGP1-tRPL3, pTDH3-1.Sba-QRI1-tIDP1.Sba], trp1, ura3
YA5571: MAT-α, his3::[tRPL3-UGP1-pSAM1, pMET6-QRI1-tIDP1, HIS3]x5, jlp1::[LEU2.Sba-loxP, pTEF1.Sba-HASB.Vir-tRPL3.Sm, pTDH3.Sk-HASB-A.Vir-tTEF1.Sba, pTDH3-1.Sba-HCOS1-3.Pm-tRPL15A.Sm, pCCW12-KFOA.Pm-tRPL41B.Sba, pCCW120.Sm-KFOA.Pm-tRPL15A.Sba, pTEF1.Ago-GFA1.Vir-tRPL15A], leu2, trp1, ura3

HASB and HASB-A represent a nucleic acid sequence encoding a polypeptide having UDP-glucose 6-dehydrogenase activity. They differ from one another in that they are different reencoded versions of a nucleic acid sequence encoding the enzyme. HASB has the sequence SEQ ID NO: 36 and HASB-A has the sequence SEQ ID NO: 37. HCOS1-3.Pm represents a nucleic acid sequence encoding a polypeptide having chondroitin synthase activity that is a nucleic acid comprising a nucleic acid encoding a polypeptide having a Hyaluronan synthase activity and fragments of a nucleic acid encoding a polypeptide having a chondroitin synthase activity. HCOS1-3.Pm has the sequence SEQ ID NO: 3.

These strains were incubated in 25 ml of SY medium supplemented with 2% glucose for 48 hours at 28° in Erlenmeyer flasks.

SY medium comprises the following elements:
KH2PO4: 100 mM; MgSO4 7H2O: 2.8 mM; K2SO4: 11.5 mM; Na2SO4:
1.1 mM; NaCl: 2.6 mM; CaCl₂) 2H2O: 0.7 mM; CuSO4 5H2O: 15 μM; KI: 6 μM; FeCl3: 30 μM; ZnSO4 7H2O: 61 μM; MnSO4 H2O: 25 μM; H2SO4: 110 μM; Panthotenic Acids hemicalcium salt: 42 μM; Thiamin hydrochloride: 59 μM; Pyridoxin hydrochloride: 49 μM; Myo-Inositol (C6H1206): 555 μM; Nicotinic acid (C6H5NO2): 29 μM; D-Biotine:
0.82 μM; Ammonium citrate tribasic: 33 mM; and glucose or sucrose 2-30%

Growth medium was recovered at 48 h hours and assayed for chondroitin content.

An aliquot of the supernatant was loaded and ran on a 0.5% agarose gel subsequently colored with bromophenol blue.

The amount of chondroitin present in the medium was evaluated by a colorimetric determination after treatment with concentrated sulfuric acid and carbazole (Bitter and Muir (1962) analytical biochemistry 4, 330-334).

The amounts obtained with these different strains are respectively:
YA5809: 200 mg·L⁻¹
YA5810: 200 mg·L⁻¹
YA5571: 200 mg·L⁻¹

In comparison, a native strain (Chérest et al. (2000) J Biol. Chem. 275: 14056-14063) from which the three other strains derive does not produce chondroitin.

It results from this experiment that a recombinant strain comprising the modifications according to the invention produces a greater amount of chondroitin when cultured in the same conditions as other strains not comprising all the genetic modifications according to the invention.

### Example 3: Examples for the Production of Chondroitin of Controlled Molecular Weight

1. Production of Chondroitin Having a Molecular Weight of Less than 10 kDa

Next, two recombinant strains are obtained: YA5887 and 5902.

Accordingly, these two strains are as follows:
YA5887: MAT-α, his3, jlp1::[LEU2.K1, pCUP1-UGP1-tRPL3, pCUP1-QRI1-tIDP1, pPDC1-UGP1-tTPI1, pTDH3-QRI1-tMET25, pCCW12-HASB.At-tRPL15A], leu2::[TRP1.Sba, pCCW12.Sba-HYAL-3.Ts-tRPL15A], sam3::[HASB.Vir-tTEF1.Sba, pTDH3-1.Sba-HCOS1-3.Pm-tRPL3.Sba, pTDH3.Sar-HCOS1-3.Pm-tRPL15A.Sm, pCCW12-KFOA.Pm-tRPL41B.Sba, pCCW120.Sm-KFOA.Pm-tRPL15A.Sba, HIS3.Sba], trp1
YA5902: MAT-α, his3 [pSAM1-UGP1-tRPL3, pMET6-QRI1-tIDP1, HIS3]x5, jlp1::[LEU2.K1, pCUP1-UGP1-tRPL3, pCUP1-QRI1-tIDP1, pPDC1-UGP1-tTPI1, pTDH3-QRI1-tMET25, pCCW12-HASB.At-tRPL15A], leu2::[TRP1.Sba, pCCW12.Sba-HYAL-3.Ts-tRPL15A], sam3::[LEU2.K1, pENO2-UGP1-tRPL3, pCWP2-GNA1-tTPI1, pTEF1-PCM1-tRPL41B, pTEF1.Sba-HASB.vir-tRPL3.Sm, pTDH3.Sk-HASB-A.Vir-tTEF1.Sba, pTDH3-1.Sba-HCOS1-3.Pm-tRPL15A.Sm, pCCW12-KFOA.Pm-tRPL15A.Sba, HIS3.Sba-loxP], trp1

HYAL-3.Ts represents a nucleic acid sequence encoding a polypeptide having chondroitinase activity associated with a secretion signal but no anchoring signal. HYAL-3.Ts has the amino acid sequence SEQ ID NO: 23.

HASB, HASB-A and HCOS-1.Pm are as defined above.

These strains were incubated in 25 ml of SY medium supplemented with 2% glucose for 48 hours at 28° in Erlenmeyer flasks.

Growth medium was recovered at 48 h hours and assayed for chondroitin content.

An aliquot of the supernatant was loaded and ran on a 0.5% agarose gel subsequently colored with bromophenol blue.

The amount of chondroitin present in the medium was evaluated by a colorimetric determination after treatment with concentrated sulfuric acid and carbazole (Bitter and Muir (1962) analytical biochemistry 4, 330-334).

The amounts obtained with these different strains are respectively:
YA5887: 600 mg·L⁻¹
YA5902: 600 mg·L⁻¹

In comparison, a native strain (Chérest et al. (2000) J Biol. Chem. 275: 14056-14063) from which the two other strains derive does not produce chondroitin.

It results from this experiment that a recombinant strain comprising the modifications according to the invention produces a greater amount of chondroitin when cultured in the same conditions as other strains not comprising all the genetic modifications according to the invention.

Moreover, these two strains led to the production, after 48 hours, of chondroitin having a molecular weight of less than 10 kDa.

2. Production of Chondroitin Having a Molecular Weight Comprised Between about 10 kDa and about 50 kDa

Next, two toher recombinant strains are obtained: YA5888 and 5903.

Accordingly, these two strains are as follows:
YA5888: MAT-α, his3, jlp1::[LEU2.K1, pCUP1-UGP1-tRPL3, pCUP1-QRI1-tIDP1, pPDC1-UGP1-tTPI1, pTDH3-QRI1-tMET25, pCCW12-HASB.At-tRPL15A], leu2::[TRP1.Sba, pCCW10.Ago-HYAL-3.Ts-tRPL15A], sam3::[HASB.Vir-tTEF1.Sba, pTDH3-1.Sba-HCOS1-3.Pm-tRPL3.Sba, pTDH3.Sar-HCOS1-3.Pm-tRPL15A.Sm, pCCW12-KFOA.Pm-tRPL41B.Sba, pCCW120.Sm-KFOA.Pm-tRPL15A.Sba, HIS3.Sba], trp1
YA5903: MAT-α, his3 [pSAM1-UGP1-tRPL3, pMET6-QRI1-tIDP1, HIS3]x5, jlp1::[LEU2.K1, pCUP1-UGP1-tRPL3, pCUP1-QRI1-tIDP1, pPDC1-UGP1-tTPI1, pTDH3-QRI1-tMET25, pCCW12-HASB.At-tRPL15A], leu2::[TRP1.Sba, pCCW10.Ago-HYAL-3.Ts-tRPL15A], sam3::[LEU2.K1, pENO2-UGP1-tRPL3, pCWP2-GNA1-tTPI1, pTEF1-PCM1-tRPL41B, pTEF1.Sba-HASB.vir-tRPL3.Sm, pTDH3.Sk-HASB-A.Vir-tTEF1.Sba, pTDH3-1.Sba-HCOS1-3.Pm-tRPL15A.Sm, pCCW12-KFOA.Pm-tRPL15A.Sba, HIS3.Sba-loxP], trp1

HYAL-3.Ts, HASB, HASB-A and HCOS-1.Pm are as defined above.

These strains were incubated in 25 ml of SY medium supplemented with 2% glucose for 48 hours at 28° in Erlenmeyer flasks.

Growth medium was recovered at 48 h hours and assayed for chondroitin content.

An aliquot of the supernatant was loaded and ran on a 0.5% agarose gel subsequently colored with bromophenol blue.

The amount of chondroitin present in the medium was evaluated by a colorimetric determination after treatment with concentrated sulfuric acid and carbazole (Bitter and Muir (1962) analytical biochemistry 4, 330-334).

The amounts obtained with these different strains are respectively:
YA5888: 600 mg·L⁻¹
YA5903: 600 mg·L⁻¹

In comparison, a native strain (Chérest et al. (2000) J Biol. Chem. 275: 14056-14063) from which the two other strains derive does not produce chondroitin.

It results from this experiment that a recombinant strain comprising the modifications according to the invention produces a greater amount of chondroitin when cultured in the same conditions as other strains not comprising all the genetic modifications according to the invention.

Moreover, these two strains led to the production, after 48 hours, of chondroitin having a molecular weight ranging from about 10 kDa to about 50 kDa.

3. Production of Chondroitin Having a Molecular Weight Comprised Between about 50 kDa and about 500 kDa

Next, two other recombinant strains are obtained: YA5889 and 5904.

Accordingly, these two strains are as follows:
YA5889: MAT-a, his3, jlp1::[LEU2.K1, pCUP1-UGP1-tRPL3, pCUP1-QRI1-tIDP1, pPDC1-UGP1-tTPI1, pTDH3-QRI1-tMET25, pCCW12-HASB.At-tRPL15A], leu2::[TRP1.Sba, pNUP57-HYAL-3.Ts-tRPL15A], sam3::[HASB.Vir-tTEF1.Sba, pTDH3-1.Sba-HCOS1-3.Pm-tRPL3.Sba, pTDH3.Sar-HCOS1-3.Pm-tRPL15A.Sm, pCCW12-KFOA.Pm-tRPL41B.Sba, pCCW120.Sm-KFOA.Pm-tRPL15A.Sba, HIS3.Sba], trp1
YA5904: MAT-α, his3 [pSAM1-UGP1-tRPL3, pMET6-QRI1-tIDP1, HIS3]x5, jlp1::[LEU2.K1, pCUP1-UGP1-tRPL3, pCUP1-QRI1-tIDP1, pPDC1-UGP1-tTPI1, pTDH3-QRI1-tMET25, pCCW12-HASB.At-tRPL15A], leu2::[TRP1.Sba, pNUP57-HYAL-3.Ts-tRPL15A], sam3::[LEU2.K1, pENO2-UGP1-tRPL3, pCWP2-GNA1-tTPI1, pTEF1-PCM1-tRPL41B, pTEF1.Sba-HASB.vir-tRPL3.Sm, pTDH3.Sk-HASB-A.Vir-tTEF1.Sba, pTDH3-1.Sba-HCOS1-3.Pm-tRPL15A.Sm, pCCW12-KFOA.Pm-tRPL15A.Sba, HIS3.Sba-loxP], trp1

HYAL-3.Ts, HASB, HASB-A and HCOS-1.Pm are as defined above.

These strains were incubated in 25 ml of SY medium supplemented with 2% glucose for 48 hours at 28° in Erlenmeyer flasks.

Growth medium was recovered at 48 h hours and assayed for chondroitin content.

An aliquot of the supernatant was loaded and ran on a 0.5% agarose gel subsequently colored with bromophenol blue.

The amount of chondroitin present in the medium was evaluated by a colorimetric determination after treatment with concentrated sulfuric acid and carbazole (Bitter and Muir (1962) analytical biochemistry 4, 330-334).

The amounts obtained with these different strains are respectively:
YA5889: 600 mg·L⁻¹
YA5904: 600 mg·L⁻¹

In comparison, a native strain (Chérest et al. (2000) J Biol. Chem. 275: 14056-14063) from which the two other strains derive does not produce chondroitin.

It results from this experiment that a recombinant strain comprising the modifications according to the invention produces a greater amount of chondroitin when cultured in the same conditions as other strains not comprising all the genetic modifications according to the invention.

Moreover, these two strains led to the production, after 48 hours, of chondroitin having a molecular weight ranging from about 10 kDa to about 50 kDa.

### Sequences

SEQ ID NO: 1 is a reencoded nucleic acid sequence of a polypeptide having a chondroitin synthase activity:chimeric nucleic acid comprising a fragment from a nucleic acid encoding a hyaluronan synthase originating from Pasteurella multocida and a fragment from a nucleic acid encoding a chondroitin synthase from Pasteurella multocida (HCOS1-1):

ATGAACACCCTTTCCCAGGCAATTAAGGCTTATAATAGTAATGATTATCAACTAGCCCTCAAGCTG
TTCGAAAAGTCGGCAGAAATCTACGGCAGAAAGATAGTTGAgTTCCAAATTACGAAGTGCAAGGA
GAAACTTTCTGCGCACCCATCCGTTAACAGTGCGCATCTCTCGGTTAACAAAGAAGAGAAGGTGAA
TGTTTGCGACAGTCCATTAGACATAGCCACTCAATTATTGTTATCGAATGTGAAAAAACTCGTCCTT
AGTGATAGCGAGAAAAATACACTAAAGAATAAATGGAAGTTGTTGACTGAAAAGAAGTCTGAGAA
CGCAGAGGTCCGGGCCGTAGCTTTAGTTCCCAAGGACTTTCCGAAAGATTTAGTGCTTGCGCCGTT
ACCAGACCATGTGAACGATTTTACGTGGTATAAGAAGCGGAAGAAACGCCTAGGAATCAAACCAG
AACACCAACACGTTGGCCTCTCCATTATTGTGACAACGTTCAACCGTCCTGCCATCCTCAGCATCAC
CCTGGCTTGCCTCGTCAATCAAAAGACCCATTATCCTTTTGAGGTGATCGTGACCGACGATGGTTCT
CAAGAGGATTTATCGCCTATAATCCGTCAATATGAGAACAAGCTAGACATCCGTTATGTTCGGCAA
AAGGACTATGGGTATCAGCTGTGCGCCGTTAGAAACCTTGGTCTAAGGACAGCGAAGTATGACTTT
GTCTCTATCTTGGATTGCGACATGGCCCCGCAGCAGCTATGGGTTCACTCTTACCTTACAGAACTAT
TAGAGGACGATGATTTGACCATAATAGGCCCCAGGAAGTACATTGACACGCAACACATAGATCCC
AAGGATTTCCTTAATAACGCCTCTCTGTTAGAGTCGTTGCCAGAGGTTAAGACCAATAATTCCGTCG
CGGCTAAGGGCGAGGGGACCGTATCTTTAGACTGGCGTTTGGAACAATTTGAGAAGACCGAGAAC
TTGAGGCTATCCGATAGCCCTTTCCGATTCTTCGCAGCTGGGAATGTGGCTTTCGCCAAGAAGTGGC
TTAACAAGTCAGGATTCTTCGACGAGGAATTCAATCACTGGGGAGGTGAAGATGTAGAGTTCGGTT
ATCGTCTGTTTCGGTACGGTTCGTTCTTCAAAACTATAGACGGCATCATGGCCTATCATCAGGAACC
GCCAGGTAAAGAAAACGAAACTGACAGAGAAGCGGGCAAGAACATTACCCTCGATATAATGAGG
GAGAAGGTGCCTTACATCTACCGTAAACTCCTGCCTATAGAAGACAGTCATATCAACCGAGTACCA
TTGGTATCAATTTACATCCCGGCCTACAACTGTGCTAACTATATTCAAAGATGTGTTGATTCAGCTT
TGAATCAAACGGTAGTTGATCTCGAAGTGTGCATTTGCAACGACGGTAGTACTGACAACACGCTGG
AAGTTATTAACAAGCTGTATGGTAATAATCCGCGTGTGCGTATAATGTCTAAACCCAATGGCGGCA
TTGCGAGTGCATCCAACGCAGCGGTCAGCTTCGCAAAGGGTTATTACATAGGACAGTTGGACAGCG
ACGATTACTTAGAACCCGACGCAGTGGAGTTATGTCTCAAGGAATTTCTTAAGGATAAGACCCTTG
CGTGCGTTTACACCACTAATCGTAACGTCAACCCAGATGGCTCTTTAATAGCCAATGGCTATAACT
GGCCAGAGTTCAGTCGTGAGAAGTTGACTACGGCCATGATTGCTCATCACTTCCGGATGTTTACCA
TTCGTGCTTGGCATCTGACGGATGGGTTCAATGAGAAGATTGAGAACGCTGTTGACTACGACATGT
TTCTCAAGCTCAGTGAAGTTGGTAAATTTAAGCATCTGAACAAAATATGTTATAATCGGGTGTTAC
ACGGCGATAACACCTCAATCAAGAAgCTTGGCATACAAAAGAAGAATCATTTCGTAGTTGTCAATC
AGTCTCTAAACCGCCAAGGTATAACTTATTATAACTACGATGAATTTGATGATCTCGATGAGAGTC
GGAAATACATTTTCAACAAGACTGCAGAGTATCAAGAAGAGATAGATATTCTTAAAGATATTAAG
ATCATTCAGAACAAAGACGCCAAAATAGCTGTCTCCATTTTCTATCCGAACACTCTAAACGGGTTG
GTGAAGAAGCTAAATAATATTATAGAGTATAATAAGAATATCTTCGTTATTGTACTTCATGTCGAT
AAGAATCACTTAACCCCAGACATCAAGAAGGAGATATTAGCCTTCTACCATAAGCATCAGGTGAAC
ATCTTATTGAACAATGACATCTCCTACTATACATCAAATCGTCTGATTAAGACAGAAGCCCATTTGA
GTAACATTAACAAGCTaAGTCAATTAAATCTTAACTGCGAATACATTATATTCGACAATCACGATTC
CTTATTTGTGAAGAACGATTCCTATGCATACATGAAGAAGTATGATGTTGGAATGAACTTCTCTGC
ATTGACTCATGATTGGATTGAAAAGATAAACGCTCACCCGCCATTTAAGAAGCTgATCAAAACTTA
CTTCAATGACAATGATCTTAAGTCGATGAATGTAAAGGGTGCCTCCCAGGGAATGTTTATGACATA
CGCATTAGCCCACGAGTTATTGACGATCATCAAGGAGGTGATAACCTCTTGTCAATCCATTGACTC
CGTCCCCGAATACAACACAGAAGATATTTGGTTTCAGTTTGCACTTTTAATTCTGGAAAAGAAGAC
CGGCCACGTATTCAACAAGACAAGCACTCTCACGTATATGCCATGGGAACGTAAACTGCAGTGGAC
GAATGAACAAATAGAGTCCGCAAAGAGGGGCGAAAACATTCCGGTAAACAAGTTCATCATTAACA
GCATTACCCTTTAA

SEQ ID NO: 2 is a reencoded nucleic acid sequence of a polypeptide having a chondroitin synthase activity:chimeric nucleic acid comprising a fragment from a nucleic acid encoding a hyaluronan synthase originating from Pasteurella multocida and a fragment from a nucleic acid encoding a chondroitin synthase from Pasteurella multocida (HCOS1-2):

ATGAACACCCTTTCCCAGGCAATTAAGGCTTATAATAGTAATGATTATCAACTAGCCCTCAAGCTG
TTCGAAAAGTCGGCAGAAATCTACGGCAGAAAGATAGTTGAgTTCCAAATTACGAAGTGCAAGGA
GAAACTTTCTGCGCACCCATCCGTTAACAGTGCGCATCTCTCGGTTAACAAAGAAGAGAAGGTGAA
TGTTTGCGACAGTCCATTAGACATAGCCACTCAATTATTGTTATCGAATGTGAAAAAACTCGTCCTT
AGTGATAGCGAGAAAAATACACTAAAGAATAAATGGAAGTTGTTGACTGAAAAGAAGTCTGAGAA
CGCAGAGGTCCGGGCCGTAGCTTTAGTTCCCAAGGACTTTCCGAAAGATTTAGTGCTTGCGCCGTT
ACCAGACCATGTGAACGATTTTACGTGGTATAAGAAGCGGAAGAAACGCCTAGGAATCAAACCAG
TGAACAAGAACATCGGGTTGTCAATCATAATCCCAACTTTCAATCGTAGTAGGATATTAGACATCA
CCCTGGCTTGCCTCGTCAATCAAAAGACCCATTATCCTTTTGAGGTGATCGTGACCGACGATGGTTC
TCAAGAGGATTTATCGCCTATAATCCGTCAATATGAGAACAAGCTAGACATCCGTTATGTTCGGCA
AAAGGACTATGGGTATCAGCTGTGCGCCGTTAGAAACCTTGGTCTAAGGACAGCGAAGTATGACTT
TGTCTCTATCTTGGATTGCGACATGGCCCCGCAGCAGCTATGGGTTCACTCTTACCTTACAGAACTA
TTAGAGGACGATGATTTGACCATAATAGGCCCCAGGAAGTACATTGACACGCAACACATAGATCCC
AAGGATTTCCTTAATAACGCCTCTCTGTTAGAGTCGTTGCCAGAGGTTAAGACCAATAATTCCGTCG
CGGCTAAGGGCGAGGGGACCGTATCTTTAGACTGGCGTTTGGAACAATTTGAGAAGACCGAGAAC
TTGAGGCTATCCGATAGCCCTTTCCGATTCTTCTCATGCGGAAATGTGGCCTTCTCCAAAGAGTGGC
TGAACAAAGTTGGCTGGTTCGACGAGGAATTCAATCACTGGGGAGGTGAAGATGTAGAGTTCGGTT
ATCGTCTGTTTCGGTACGGTTCGTTCTTCAAAACTATAGACGGCATCATGGCCTATCATCAGGAACC
GCCAGGTAAAGAAAACGAAACTGACAGAGAAGCGGGCAAGAACATTACCCTCGATATAATGAGG
GAGAAGGTGCCTTACATCTACCGTAAACTCCTGCCTATAGAAGACAGTCATATCAACCGAGTACCA
TTGGTATCAATTTACATCCCGGCCTACAACTGTGCTAACTATATTCAAAGATGTGTTGATTCAGCTT
TGAATCAAACGGTAGTTGATCTCGAAGTGTGCATTTGCAACGACGGTAGTACTGACAACACGCTGG
AAGTTATTAACAAGCTGTATGGTAATAATCCGCGTGTGCGTATAATGTCTAAACCCAATGGCGGCA
TTGCGAGTGCATCCAACGCAGCGGTCAGCTTCGCAAAGGGTTATTACATAGGACAGTTGGACAGCG
ACGATTACTTAGAACCCGACGCAGTGGAGTTATGTCTCAAGGAATTTCTTAAGGATAAGACCCTTG
CGTGCGTTTACACCACTAATCGTAACGTCAACCCAGATGGCTCTTTAATAGCCAATGGCTATAACT
GGCCAGAGTTCAGTCGTGAGAAGTTGACTACGGCCATGATTGCTCATCACTTCCGGATGTTTACCA
TTCGTGCTTGGCATCTGACGGATGGGTTCAATGAGAAGATTGAGAACGCTGTTGACTACGACATGT
TTCTCAAGCTCAGTGAAGTTGGTAAATTTAAGCATCTGAACAAAATATGTTATAATCGGGTGTTAC
ACGGCGATAACACCTCAATCAAGAAgCTTGGCATACAAAAGAAGAATCATTTCGTAGTTGTCAATC
AGTCTCTAAACCGCCAAGGTATAACTTATTATAACTACGATGAATTTGATGATCTCGATGAGAGTC
GGAAATACATTTTCAACAAGACTGCAGAGTATCAAGAAGAGATAGATATTCTTAAAGATATTAAG
ATCATTCAGAACAAAGACGCCAAAATAGCTGTCTCCATTTTCTATCCGAACACTCTAAACGGGTTG
GTGAAGAAGCTAAATAATATTATAGAGTATAATAAGAATATCTTCGTTATTGTACTTCATGTCGAT
AAGAATCACTTAACCCCAGACATCAAGAAGGAGATATTAGCCTTCTACCATAAGCATCAGGTGAAC
ATCTTATTGAACAATGACATCTCCTACTATACATCAAATCGTCTGATTAAGACAGAAGCCCATTTGA
GTAACATTAACAAGCTaAGTCAATTAAATCTTAACTGCGAATACATTATATTCGACAATCACGATTC
CTTATTTGTGAAGAACGATTCCTATGCATACATGAAGAAGTATGATGTTGGAATGAACTTCTCTGC
ATTGACTCATGATTGGATTGAAAAGATAAACGCTCACCCGCCATTTAAGAAGCTgATCAAAACTTA
CTTCAATGACAATGATCTTAAGTCGATGAATGTAAAGGGTGCCTCCCAGGGAATGTTTATGACATA
CGCATTAGCCCACGAGTTATTGACGATCATCAAGGAGGTGATAACCTCTTGTCAATCCATTGACTC
CGTCCCCGAATACAACACAGAAGATATTTGGTTTCAGTTTGCACTTTTAATTCTGGAAAAGAAGAC
CGGCCACGTATTCAACAAGACAAGCACTCTCACGTATATGCCATGGGAACGTAAACTGCAGTGGAC
GAATGAACAAATAGAGTCCGCAAAGAGGGGCGAAAACATTCCGGTAAACAAGTTCATCATTAACA
GCATTACCCTTTAA

SEQ ID NO: 3 is a reencoded nucleic acid sequence of a polypeptide having a chondroitin synthase activity:chimeric nucleic acid comprising a fragment from a nucleic acid encoding a hyaluronan synthase originating from Pasteurella multocida and a fragment from a nucleic acid encoding a chondroitin synthase from Pasteurella multocida (HCOS1-3):

ATGAACACCCTTTCCCAGGCAATTAAGGCTTATAATAGTAATGATTATCAACTAGCCCTCAAGCTG
TTCGAAAAGTCGGCAGAAATCTACGGCAGAAAGATAGTTGAGTTCCAAATTACGAAGTGCAAGGA
GAAACTTTCTGCGCACCCATCCGTTAACAGTGCGCATCTCTCGGTTAACAAAGAAGAGAAGGTGAA
TGTTTGCGACAGTCCATTAGACATAGCCACTCAATTATTGTTATCGAATGTGAAAAAACTCGTCCTT
AGTGATAGCGAGAAAAATACACTAAAGAATAAATGGAAGTTGTTGACTGAAAAGAAGTCTGAGAA
CGCAGAGGTCCGGGCCGTAGCTTTAGTCCCAAAGGATTTTCCCAAAGACTTAGTTTTAGCGCCACT
ACCAGACCATGTGAACGATTTTACCTGGTACAAGAATAGAAAGAAGAGCCTGGGCATAAAGCCTG
TGAACAAGAACATCGGGTTGTCAATCATAATCCCAACTTTCAATCGTAGTAGGATATTAGACATTA
CCCTGGCGTGTCTAGTTAATCAGAAAACTAACTATCCGTTTGAGGTGGTTGTTGCAGATGATGGGT
CGAAGGAAAATCTATTGACTATAGTTCAGAAATACGAGCAGAAACTAGACATTAAGTATGTGAGA
CAGAAGGACTATGGGTATCAGCTGTGCGCCGTTAGAAACCTTGGTCTAAGGACAGCGAAGTATGA
CTTTGTCTCTATCTTGGATTGCGACATGGCCCCGCAGCAGCTATGGGTTCACTCTTACCTTACAGAG
TTACTGGAAGACAACGACATCGTCTTGATCGGGCCAAGGAAATACGTTGATACCCACAACATCACG
GCTGAGCAGTTTCTGAACGACCCATATTTAATTGAGAGTTTACCGGAAACCGCCACAAACAATAAT
CCATCGATTACCAGTAAGGGTAACATCTCATTAGATTGGAGATTAGAACATTTTAAGAAGACAGAC
AACCTAAGACTATGCGACTCCCCGTTCAGGTATTTCTCATGCGGAAATGTGGCCTTCTCCAAAGAG
TGGCTGAACAAAGTTGGCTGGTTCGACGAAGAATTTAATCACTGGGGCGGCGAAGACGTCGAATTC
GGCTACAGGTTATTTGCAAAAGGGTGCTTCTTTAGGGTTATCGACGGTGGTATGGCCTACCATCAA
GAGCCACCAGGAAAGGAGAACGAAACGGATCGTGAGGCCGGTAAGTCTATCACTCTTAAAATAGT
AAAGGAGAAAGTGCCCTACATCTACAGGAAGTTGTTGCCGATTGAGGACTCCCACATAAACCGAG
TACCATTGGTATCAATTTACATCCCGGCCTACAACTGTGCTAACTATATTCAAAGATGTGTTGATTC
AGCTTTGAATCAAACGGTAGTTGATCTCGAAGTGTGCATTTGCAACGACGGTAGTACTGACAACAC
GCTGGAAGTTATTAACAAGCTGTATGGTAATAATCCGCGTGTGCGTATAATGTCTAAACCCAATGG
CGGCATTGCGAGTGCATCCAACGCAGCGGTCAGCTTCGCAAAGGGTTATTACATAGGACAGTTGGA
CAGCGACGATTACTTAGAACCCGACGCAGTGGAGTTATGTCTCAAGGAATTTCTTAAGGATAAGAC
CCTTGCGTGCGTTTACACCACTAATCGTAACGTCAACCCAGATGGCTCTTTAATAGCCAATGGCTAT
AACTGGCCAGAGTTCAGTCGTGAGAAGTTGACTACGGCCATGATTGCTCATCACTTCCGGATGTTT
ACCATTCGTGCTTGGCATCTGACGGATGGGTTCAATGAGAAGATTGAGAACGCTGTTGACTACGAC
ATGTTTCTCAAGCTCAGTGAAGTTGGTAAATTTAAGCATCTGAACAAAATATGTTATAATCGGGTG
TTACACGGCGATAACACCTCAATCAAGAAGCTTGGCATACAAAAGAAGAATCATTTCGTAGTTGTC
AATCAGTCTCTAAACCGCCAAGGTATAACTTATTATAACTACGATGAATTTGATGATCTCGATGAG
AGTCGGAAATACATTTTCAACAAGACTGCAGAGTATCAAGAAGAGATAGATATTCTTAAAGATATT
AAGATCATTCAGAACAAAGACGCCAAAATAGCTGTCTCCATTTTCTATCCGAACACTCTAAACGGG
TTGGTGAAGAAGCTAAATAATATTATAGAGTATAATAAGAATATCTTCGTTATTGTACTTCATGTCG
ATAAGAATCACTTAACCCCAGACATCAAGAAGGAGATATTAGCCTTCTACCATAAGCATCAGGTGA
ACATCTTATTGAACAATGACATCTCCTACTATACATCAAATCGTCTGATTAAGACAGAAGCCCATTT
GAGTAACATTAACAAGCTAAGTCAATTAAATCTTAACTGCGAATACATTATATTCGACAATCACGA
TTCCTTATTTGTGAAGAACGATTCCTATGCATACATGAAGAAGTATGATGTTGGAATGAACTTCTCT
GCATTGACTCATGATTGGATTGAAAAGATAAACGCTCACCCGCCATTTAAGAAGCTGATCAAAACT
TACTTCAATGACAATGATCTTAAGTCGATGAATGTAAAGGGTGCCTCCCAGGGAATGTTTATGACA
TACGCATTAGCCCACGAGTTATTGACGATCATCAAGGAGGTGATAACCTCTTGTCAATCCATTGAC
TCCGTCCCCGAATACAACACAGAAGATATTTGGTTTCAGTTTGCACTTTTAATTCTGGAAAAGAAG
ACCGGCCACGTATTCAACAAGACAAGCACTCTCACGTATATGCCATGGGAACGTAAACTGCAGTGG
ACGAATGAACAAATAGAGTCCGCAAAGAGGGGCGAAAACATTCCGGTAAACAAGTTCATCATTAA
CAGCATTACCCTTTAA

SEQ ID NO: 4 is a nucleic acid sequence of a polypeptide having chondroitin synthase activity: chimeric nucleic acid comprising a fragment from a nucleic acid encoding a hyaluronan synthase originating from Pasteurella multocida, a fragment from a nucleic acid encoding a chondroitin synthase from Pasteurella multocida and a fragment from a nucleic acid encoding a chitin synthase 2 from Saccharomyces cerevisiae (HCOS.Sc)

ATGAACACCCTTTCCCAGGCAATTAAGGCTTATAATAGTAATGATTATCAACTAGCCCTCAAGCTG
TTCGAAAAGTCGGCAGAAATCTACGGCAGAAAGATAGTTGAGTTCCAAATTACGAAGTGCAAGGA
GAAACTTTCTGCGCACCCATCCGTTAACAGTGCGCATCTCTCGGTTAACAAAGAAGAGAAGGTGAA
TGTTTGCGACAGTCCATTAGACATAGCCACTCAATTATTGTTATCGAATGTGAAAAAACTCGTCCTT
AGTGATAGCGAGAAAAATACACTAAAGAATAAATGGAAGTTGTTGACTGAAAAGAAGTCTGAGAA
CGCAGAGGTCCGGGCCGTAGCTTTAGTCCCAAAGGATTTTCCCAAAGACTTAGTTTTAGCGCCACT
ACCAGACCATGTGAACGATTTTACCTGGTACAAGAATAGAAAGAAGAGCCTGGGCATAAAGCCTG
TGAACAAGAACATCGGGTTGTCAATCATAATCCCAACTTTCAATCGTAGTAGGATATTAGACATTA
CCCTGGCGTGTCTAGTTAATCAGAAAACTAACTATCCGTTTGAGGTGGTTGTTGCAGATGATGGGT
CGAAGGAAAATCTATTGACTATAGTTCAGAAATACGAGCAGAAACTAGACATTAAGTATGTGAGA
CAGAAGGACTATGGGTATCAGCTGTGCGCCGTTAGAAACCTTGGTCTAAGGACAGCGAAGTATGA
CTTTGTCTCTATCTTGGATTGCGACATGGCCCCGCAGCAGCTATGGGTTCACTCTTACCTTACAGAG
TTACTGGAAGACAACGACATCGTCTTGATCGGGCCAAGGAAATACGTTGATACCCACAACATCACG
GCTGAGCAGTTTCTGAACGACCCATATTTAATTGAGAGTTTACCGGAAACCGCCACAAACAATAAT
CCATCGATTACCAGTAAGGGTAACATCTCATTAGATTGGAGATTAGAACATTTTAAGAAGACAGAC
AACCTAAGACTATGCGACTCCCCGTTCAGGTATTTCTCATGCGGAAATGTGGCCTTCTCCAAAGAG
TGGCTGAACAAAGTTGGCTGGTTCGACGAAGAATTTAATCACTGGGGCGGCGAAGACGTCGAATTC
GGCTACAGGTTATTTGCAAAAGGGTGCTTCTTTAGGGTTATCGACGGTGGTATGGCCTACCATCAA
GAGCCACCAGGAAAGGAGAACGAAACGGATCGTGAGGCCGGTAAGTCTATCACTCTTAAAATAGT
AAAGGAGAAAGTGCCCTACATCTACAGGAAGTTGTTGCCGATTGAGGACTCCCACATAAACCGAG
TACCATTGGTATCAATTTACATCCCGGCCTACAACTGTGCTAACTATATTCAAAGATGTGTTGATTC
AGCTTTGAATCAAACGGTAGTTGATCTCGAAGTGTGCATTTGCAACGACGGTAGTACTGACAACAC
GCTGGAAGTTATTAACAAGCTGTATGGTAATAATCCGCGTGTGCGTATAATGTCTAAACCCAATGG
CGGCATTGCGAGTGCATCCAACGCAGCGGTCAGCTTCGCAAAGGGTTATTACATAGGACAGTTGGA
CAGCGACGATTACTTAGAACCCGACGCAGTGGAGTTATGTCTCAAGGAATTTCTTAAGGATAAGAC
CCTTGCGTGCGTTTACACCACTAATCGTAACGTCAACCCAGATGGCTCTTTAATAGCCAATGGCTAT
AACTGGCCAGAGTTCAGTCGTGAGAAGTTGACTACGGCCATGATTGCTCATCACTTCCGGATGTTT
ACCATTCGTGCTTGGCATCTGACGGATGGGTTCAATGAGAAGATTGAGAACGCTGTTGACTACGAC
ATGTTTCTCAAGCTCAGTGAAGTTGGTAAATTTAAGCATCTGAACAAAATATGTTATAATCGGGTG
TTACACGGCGATAACACCTCAATCAAGAAGCTTGGCATACAAAAGAAGAATCATTTCGTAGTTGTC
AATCAGTCTCTAAACCGCCAAGGTATAACTTATTATAACTACGATGAATTTGATGATCTCGATGAG
AGTCGGAAATACATTTTCAACAAGACTGCAGAGTATCAAGAAGAGATAGATATTCTTAAAGATATT
TTTATTCAGATGCTTTTTTCCTGGTTTTCTATTGCAAATTTCGTTCTTACCTTTTATTATTTAGCAGGA
TCAATGAATTTAGTTATTAAACATGGTGAGGCCTTATTCATTTTTTTTAAATACCTGATCTTTTGTGA
CTTGGCAAGTTTATTCATTATTTCCATGGGTAATAGACCCCAGGGCGCGAAACATTTATTCATTACC
TCCATGGTTATACTGTCTATATGTGCCACATATTCTCTAATTTGTGGGTTTGTTTTTGCTTTCAAGTC
GTTAGCTTCTGGAACGGAATCCCACAAAATATTTGTCGACATCGTTATCTCATTGCTCTCCACCTAT
GGCCTATACTTTTTCTCATCACTGATGTACCTAGATCCTTGGCACATGTTTACATCATCCATACAAT
ACTTTTTGACACTTCCCGCCTTTACGTGTACTTTACAGATTTTTGCCTTCTGTAATACACACGACGTT
TCCTGGGGTACTAAAGGTTCCACACAGGAGTCCAAGCAATTGTCCAAGGCCATTGTCGTTCAAGGT
CCAGATGGGAAACAGATTGTGGAAACAGATTGGCCTCAGGAAGTTGATAAGAAGTTTTTGGAAAT
AAAAAGTCGTTTGAAAGAACCAGAATTTGAAGAATCAAGCGGCAATGAAAAACAATCCAAGAATG
ATTATTATAGAGATATAAGAACCAGAATTGTGATGATTTGGATGCTATCAAATCTAATACTGATCA
TGTCTATAATTCAAGTCTTTACACCACAAGATACTGACAATGGTTATTTGATATTCATTTTATGGTC
TGTGGCCGCTTTAGCTGCCTTTAGGGTGGTTGGTTCCATGGCCTTTTTGTTCATGAAATACTTGCGT
ATAATAGTGAGTTACAGAAATAAAGTTGAAGGTAGCGGCTCATGGGAAGTCTCTAAATTAGACTTA
CCAAATGTTTTCCACAAAAAGGGCTAA

SEQ ID NO: 5 is a nucleic acid sequence of a polypeptide having chondroitin synthase activity: chimeric nucleic acid comprising a fragment from a nucleic acid encoding a hyaluronan synthase originating from Pasteurella multocida and a fragment from a nucleic acid encoding a chitin synthase 2 from Saccharomyces cerevisiae (HHASA.Sc)

ATGAACACCCTTTCCCAGGCAATTAAGGCTTATAATAGTAATGATTATCAACTAGCCCTCAAGCTG
TTCGAAAAGTCGGCAGAAATCTACGGCAGAAAGATAGTTGAgTTCCAAATTACGAAGTGCAAGGA
GAAACTTTCTGCGCACCCATCCGTTAACAGTGCGCATCTCTCGGTTAACAAAGAAGAGAAGGTGAA
TGTTTGCGACAGTCCATTAGACATAGCCACTCAATTATTGTTATCGAATGTGAAAAAACTCGTCCTT
AGTGATAGCGAGAAAAATACACTAAAGAATAAATGGAAGTTGTTGACTGAAAAGAAGTCTGAGAA
CGCAGAGGTCCGGGCCGTAGCTTTAGTTCCCAAGGACTTTCCGAAAGATTTAGTGCTTGCGCCGTT
ACCAGACCATGTGAACGATTTTACGTGGTATAAGAAGCGGAAGAAACGCCTAGGAATCAAACCAG
AACACCAACACGTTGGCCTCTCCATTATTGTGACAACGTTCAACCGTCCTGCCATCCTCAGCATCAC
CCTGGCTTGCCTCGTCAATCAAAAGACCCATTATCCTTTTGAGGTGATCGTGACCGACGATGGTTCT
CAAGAGGATTTATCGCCTATAATCCGTCAATATGAGAACAAGCTAGACATCCGTTATGTTCGGCAA
AAGGACAACGGTTTTCAGGCATCGGCCGCACGTAATATGGGCCTACGTCTAGCCAAATATGACTTC
ATTGGGCTCCTTGATTGTGATATGGCTCCCAACCCTTTGTGGGTACACTCATACGTTGCGGAACTAT
TAGAGGACGATGATTTGACCATAATAGGCCCCAGGAAGTACATTGACACGCAACACATAGATCCC
AAGGATTTCCTTAATAACGCCTCTCTGTTAGAGTCGTTGCCAGAGGTTAAGACCAATAATTCCGTCG
CGGCTAAGGGCGAGGGGACCGTATCTTTAGACTGGCGTTTGGAACAATTTGAGAAGACCGAGAAC
TTGAGGCTATCCGATAGCCCTTTCCGATTCTTCGCAGCTGGGAATGTGGCTTTCGCCAAGAAGTGGC
TTAACAAGTCAGGATTCTTCGACGAGGAATTCAATCACTGGGGAGGTGAAGATGTAGAGTTCGGTT
ATCGTCTGTTTCGGTACGGTTCGTTCTTCAAAACTATAGACGGCATCATGGCCTATCATCAGGAACC
GCCAGGTAAAGAAAACGAAACTGACAGAGAAGCGGGCAAGAACATTACCCTCGATATAATGAGG
GAGAAGGTGCCTTACATCTACCGTAAACTCCTGCCTATAGAAGACAGTCATATCAACCGAGTACCA
TTGGTATCAATTTACATCCCGGCCTACAACTGTGCTAACTATATTCAAAGATGTGTTGATTCAGCTT
TGAATCAAACGGTAGTTGATCTCGAAGTGTGCATTTGCAACGACGGTAGTACTGACAACACGCTGG
AAGTTATTAACAAGCTGTATGGTAATAATCCGCGTGTGCGTATAATGTCTAAACCCAATGGCGGCA
TTGCGAGTGCATCCAACGCAGCGGTCAGCTTCGCAAAGGGTTATTACATAGGACAGTTGGACAGCG
ACGATTACTTAGAACCCGACGCAGTGGAGTTATGTCTCAAGGAATTTCTTAAGGATAAGACCCTTG
CGTGCGTTTACACCACTAATCGTAACGTCAACCCAGATGGCTCTTTAATAGCCAATGGCTATAACT
GGCCAGAGTTCAGTCGTGAGAAGTTGACTACGGCCATGATTGCTCATCACTTCCGGATGTTTACCA
TTCGTGCTTGGCATCTGACGGATGGGTTCAATGAGAAGATTGAGAACGCTGTTGACTACGACATGT
TTCTCAAGCTCAGTGAAGTTGGTAAATTTAAGCATCTGAACAAAATATGTTATAATCGGGTGTTAC
ACGGCGATAACACCTCAATCAAGAAgCTTGGCATACAAAAGAAGAATCATTTCGTAGTTGTCAATC
AGTCTCTAAACCGCCAAGGTATAACTTATTATAACTACGATGAATTTGATGATCTCGATGAGAGTC
GGAAATACATTTTCAACAAGACTGCAGAGTATCAAGAAGAGATAGATATTCTTAAAGATATTTTTA
TTCAGATGCTTTTTTCCTGGTTTTCTATTGCAAATTTCGTTCTTACCTTTTATTATTTAGCAGGATCA
ATGAATTTAGTTATTAAACATGGTGAGGCCTTATTCATTTTTTTTAAATACCTGATCTTTTGTGACTT
GGCAAGTTTATTCATTATTTCCATGGGTAATAGACCCCAGGGCGCGAAACATTTATTCATTACCTCC
ATGGTTATACTGTCTATATGTGCCACATATTCTCTAATTTGTGGGTTTGTTTTTGCTTTCAAGTCGTT
AGCTTCTGGAACGGAATCCCACAAAATATTTGTCGACATCGTTATCTCATTGCTCTCCACCTATGGC
CTATACTTTTTCTCATCACTGATGTACCTAGATCCTTGGCACATGTTTACATCATCCATACAATACTT
TTTGACACTTCCCGCCTTTACGTGTACTTTACAGATTTTTGCCTTCTGTAATACACACGACGTTTCCT
GGGGTACTAAAGGTTCCACACAGGAGTCCAAGCAATTGTCCAAGGCCATTGTCGTTCAAGGTCCAG
ATGGGAAACAGATTGTGGAAACAGATTGGCCTCAGGAAGTTGATAAGAAGTTTTTGGAAATAAAA
AGTCGTTTGAAAGAACCAGAATTTGAAGAATCAAGCGGCAATGAAAAACAATCCAAGAATGATTA
TTATAGAGATATAAGAACCAGAATTGTGATGATTTGGATGCTATCAAATCTAATACTGATCATGTC
TATAATTCAAGTCTTTACACCACAAGATACTGACAATGGTTATTTGATATTCATTTTATGGTCTGTG
GCCGCTTTAGCTGCCTTTAGGGTGGTTGGTTCCATGGCCTTTTTGTTCATGAAATACTTGCGTATAA
TAGTGAGTTACAGAAATAAAGTTGAAGGTAGCGGCTCATGGGAAGTCTCTAAATTAGACTTACCAA
ATGTTTTCCACAAAAAGGGCTAA

SEQ ID NO: 6 is a nucleic acid sequence of a polypeptide having chondroitin synthase activity: chimeric nucleic acid comprising a fragment from a nucleic acid encoding a hyaluronan synthase originating from Chlorella virus PBCV-1 and a fragment from a nucleic acid encoding a chondroitin synthase from Escherichia coli (HCOS1-Vir)

ATGGGTAAGAACATCATTATTATGGTTTCTTGGTACACTATTATTACATCCAATCTCATCGCAGTGG
GTGGCGCCTCACTCATACTAGCCCCAGCTATTACGGGCTATGTCCTTCACTGGAACATTGCCCTTTC
AACAATTTGGGGAGTGTCGGCCTACGGAATTTTCGTGTTTGGTTTCTTTCTTGCCCAGGTATTATTT
AGTGAACTCAACCGGAAAAGGCTCCGGAAGTGGATTTCCCTCCGACCCAAAGGGTGGAATGATGT
TAGGTTGGCTGTAATTATTGCCGGCTACCGTGAGGACCCGTATATGTTCCAAAAGTGTCTTGAAAG
TGTGCGTGACTCAGACTATGGGAATGTAGCTAGACTAATATGCGTTATTGACGGCGATGAAGACGA
CGACATGAGGATGGCTGCAGTGTACAAGGCTATCTATAACGACAACATCAAGAAACCTGAGTTTGT
CCTCTGTGAGAGTGACGATAAGGAGGGTGAGAGAATAGATAGCGATTTCAGCCGTGATATCTGCGT
GCTGCAACCGCATCGCGGAAAGCGTGAATGTTTGTACACAGGGTTCCAATTGGCAAAGATGGACCC
CTCAGTTAATGCCGTCGTCCTAATCGACAGTGACACTGTGTTAGAAAAGGACGCGATTCTCGAaGT
AGTATACCCGCTGGCATGCGATCCAGAAATACAGGCTGTAGCAGGCGAATGCAAAATATGGAATA
CTGACACACTGTTGAGTTTGCTGGTAGCCTGGCGATATTACAGCGCATTTTGCGTAGAGCGTAGCG
CCCAATCATTCTTCAGGACAGTACAATGCGTCGGAGGACCTCTCGGCGCCTACAAGATTGATATAA
TTAAGGAAATCAAGGACCCATGGATCAGCCAACGTTTCCTTGGCCAAAAGTGCACATACGGCGAC
GATAGACGACTCACTAATGAAATACTAATGAGGGGTAAGAAAGTAGTTTTCACCCCATTCGCTGTT
GGCTGGGGATGGTTCGACGAGGAGTTCACACACTGGGGTGGCCGTTACATTGTACAGCAAACACG
GTGGTCCAAGTCGTGGTGTAGGGAGATATGGTATACACTGTTTGCAGCATGGAAGCATGGACTTTC
GGGCATTTGGCTCGCATTCGAGTGCTTATACCAGATTACTTATTTCTTCCTGGTGATTTACCTATTCT
CCCGTTTGGCTGTTGAGGCTGACCCACGGGCGCAAACGGCCACCGTCATTGTTTCGACCACAGTTG
CGCTTATTAAGTGCGGTTACTTTAGCTTCAGAGCTAAAGACATTAGGGCCTTCTATTTCGTCCTCTA
CACGTTCGTTTACTTCTTCTGCATGATTCCGGCACGAATAACTGCGATGATGACCCTGTGGGACATA
GGTTGGGGAACCAGGGGAGGAAATGAGAAGCCTTCCGTAGGCACCAGAGTTGCTTTGTGGGCCAA
GCAATATTTGATTGCTTACATGTGGTGGGCCGCTGTCGTCGGTGCTGGCGTGTACTCCATCGTTCAT
AATTGGATGTTTGACTGGAATTCACTTTCCTACAGGTTCGCACTGGTAGGCATCTGTTCGTATATAG
TTTTCATTGTAATAGTGCTGGTAGTCTACTTTACAGGAAAGATCACGACCTGGAACTTCACGAAGCT
ACAGAAAGAATTGATCGAGGACCGCGTACTGTACGACGCAACGACCAATGCCCAGTCGGTATAA

SEQ ID NO: 7 is a reencoded nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin synthase from Escherichia coli (HCOS2-Vir)

ATGGGTAAGAACATCATTATTATGGTTTCTTGGTACACTATTATTACATCCAATCTCATCGCAGTGG
GTGGCGCCTCACTCATACTAGCCCCAGCTATTACGGGCTATGTCCTTCACTGGAACATTGCCCTTTC
AACAATTTGGGGAGTGTCGGCCTACGGAATTTTCGTGTTTGGTTTCTTTCTTGCCCAGGTATTATTT
AGTGAACTCAACCGGAAAAGGCTCCGGAAGTGGATTTCCCTCCGACCCAAAGGGTGGAATGATGT
TAGGTTGGCTGTAATTATTGCCGGCTACCGTGAGGACCCGTATATGTTCCAAAAGTGTCTTGAAAG
TGTGCGTGACTCAGACTATGGGAATGTAGCTAGACTAATATGCGTTATTGACGGCGATGAAGACGA
CGACATGAGGATGGCTGCAGTGTACAAGGCTATCTATAACGACAACATCAAGAAACCTGAGTTTGT
CCTCTGTGAGAGTGACGATAAGGAGGGTGAGAGAATAGATAGCGATTTCAGCCGTGATATCTGCGT
GCTGCAACCGCATCGCGGAAAGCGTGAATGTTTGTACACAGGGTTCCAATTGGCAAAGATGGACCC
CTCAGTTAATGCCGTCGTCCTAATCGACAGTGACACTGTGTTAGAAAAGGACGCGATTCTCGAaGT
AGTATACCCGCTGGCATGCGATCCAGAAATACAGGCTGTAGCAGGCGAATGCAAAATATGGAATA
CTGACACACTGTTGAGTTTGCTGGTAGCCTGGCGATATTACAGCGCATTTTGCGTAGAGCGTAGCG
CCCAATCATTCTTCAGGACAGTACAATGCGTCGGAGGACCTCTCGGCGCCTACAAGATTGATATAA
TTAAGGAAATCAAGGACCCATGGATCAGCCAACGTTTCCTTGGCCAAAAGTGCACATACGGCGAC
GATAGACGACTCACTAATGAAATACTAATGAGGGGTAAGAAAGTAGTTTTCACCCCATTCGCTGTT
GGCTGGCTACACGGTGAGAATACGTCTATCAAGAAGTTACGTTACATTGTACAGCAAACACGGTGG
TCCAAGTCGTGGTGTAGGGAGATATGGTATACACTGTTTGCAGCATGGAAGCATGGACTTTCGGGC
ATTTGGCTCGCATTCGAGTGCTTATACCAGATTACTTATTTCTTCCTGGTGATTTACCTATTCTCCCG
TTTGGCTGTTGAGGCTGACCCACGGGCGCAAACGGCCACCGTCATTGTTTCGACCACAGTTGCGCT
TATTAAGTGCGGTTACTTTAGCTTCAGAGCTAAAGACATTAGGGCCTTCTATTTCGTCCTCTACACG
TTCGTTTACTTCTTCTGCATGATTCCGGCACGAATAACTGCGATGATGACCCTGTGGGACATAGGTT
GGGGAACCAGGGGAGGAAATGAGAAGCCTTCCGTAGGCACCAGAGTTGCTTTGTGGGCCAAGCAA
TATTTGATTGCTTACATGTGGTGGGCCGCTGTCGTCGGTGCTGGCGTGTACTCCATCGTTCATAATT
GGATGTTTGACTGGAATTCACTTTCCTACAGGTTCGCACTGGTAGGCATCTGTTCGTATATAGTTTT
CATTGTAATAGTGCTGGTAGTCTACTTTACAGGAAAGATCACGACCTGGAACTTCACGAAGCTACA
GAAAGAATTGATCGAGGACCGCGTACTGTACGACGCAACGACCAATGCCCAGTCGGTATAA

SEQ ID NO: 8 is a reencoded nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a Galactofuranosyltransferase from Mycobacterium tuberculosis (HCOS3-Vir)

ATGGGTAAGAACATCATTATTATGGTTTCTTGGTACACTATTATTACAT
CCAATCTCATCGCAGTGGGTGGCGCCTCACTCATACTAGCCCCAGCTAT
TACGGGCTATGTCCTTCACTGGAACATTGCCCTTTCAACAATTTGGGGA
GTGTCGGCCTACGGAATTTTCGTGTTTGGTTTCTTTCTTGCCCAGGTAT
TATTTAGTGAACTCAACCGGAAAAGGCTCCGGAAGTGGATTTCCCTCCG
ACCCAAAGGGTGGAATGATGTTAGGTTGGCTGTAATTATTGCCGGCTAC
CGTGAGGACCCGTATATGTTCCAAAAGTGTCTTGAAAGTGTGCGTGACT
CAGACTATGGGAATGTAGCTAGACTAATATGCGTTATTGACGGCGATGA
AGACGACGACATGAGGATGGCTGCAGTGTACAAGGCTATCTATAACGAC
AACATCAAGAAACCTGAGTTTGTCCTCTGTGAGAGTGACGATAAGGAGG
GTGAGAGAATAGATAGCGATTTCAGCCGTGATATCTGCGTGCTGCAACC
GCATCGCGGAAAGCGTGAATGTTTGTACACAGGGTTCCAATTGGCAAAG
ATGGACCCCTCAGTTAATGCCGTCGTCCTAATCGACAGTGACACTGTGT
TAGAAAAGGACGCGATTCTCGAaGTAGTATACCCGCTGGCATGCGATCC
AGAAATACAGGCTGTAGCAGGCGAATGCAAAATATGGAATACTGACACA
CTGTTGAGTTTGCTGGTAGCCTGGCGATATTACAGCGCATTTTGCGTAG
AGCGTAGCGCCCAATCATTCTTCAGGACAGTACAATGCGTCGGAGGACC
TCTCGGCGCCTACAAGATTGATATAATTAAGGAAATCAAGGACCCATGG
ATCAGCCAACGTTTCCTTGGCCAAAAGTGCACATACGGCGACGATAGAC
GACTCACTAATGAAATACTAATGAGGGGTAAGAAAGTAGTTTTCACCCC
ATTCGCTGTTGGCTGGCACATGGCATGGTCCGACAAGGACGACGCAATC
GACCGTTACATTGTACAGCAAACACGGTGGTCCAAGTCGTGGTGTAGGG
AGATATGGTATACACTGTTTGCAGCATGGAAGCATGGACTTTCGGGCAT
TTGGCTCGCATTCGAGTGCTTATACCAGATTACTTATTTCTTCCTGGTG
ATTTACCTATTCTCCCGTTTGGCTGTTGAGGCTGACCCACGGGCGCAAA
CGGCCACCGTCATTGTTTCGACCACAGTTGCGCTTATTAAGTGCGGTTA
CTTTAGCTTCAGAGCTAAAGACATTAGGGCCTTCTATTTCGTCCTCTAC
ACGTTCGTTTACTTCTTCTGCATGATTCCGGCACGAATAACTGCGATGA
TGACCCTGTGGGACATAGGTTGGGGAACCAGGGGAGGAAATGAGAAGCC
TTCCGTAGGCACCAGAGTTGCTTTGTGGGCCAAGCAATATTTGATTGCT
TACATGTGGTGGGCCGCTGTCGTCGGTGCTGGCGTGTACTCCATCGTTC
ATAATTGGATGTTTGACTGGAATTCACTTTCCTACAGGTTCGCACTGGT
AGGCATCTGTTCGTATATAGTTTTCATTGTAATAGTGCTGGTAGTCTAC
TTTACAGGAAAGATCACGACCTGGAACTTCACGAAGCTACAGAAAGAAT
TGATCGAGGACCGCGTACTGTACGACGCAACGACCAATGCCCAGTCGGT
ATAA

SEQ ID NO: 9 is a reencoded nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin synthase from Escherichia coli (HCOS4-Vir)

ATGGGTAAGAACATCATTATTATGGTTTCTTGGTACACTATTATTACAT
CCAATCTCATCGCAGTGGGTGGCGCCTCACTCATACTAGCCCCAGCTAT
TACGGGCTATGTCCTTCACTGGAACATTGCCCTTTCAACAATTTGGGGA
GTGTCGGCCTACGGAATTTTCGTGTTTGGTTTCTTTCTTGCCCAGGTAT
TATTTAGTGAACTCAACCGGAAAAGGCTCCGGAAGTGGATTTCCCTCCG
ACCCAAAGGGTGGAATGATGTTAGGTTGGCTGTAATTATTGCCGGCTAC
CGTGAGGACCCGTATATGTTCCAAAAGTGTCTTGAAAGTGTGCGTGACT
CAGACTATGGGAATGTAGCTAGACTAATATGCGTTATTGACGGCGATGA
AGACGACGACATGAGGATGGCTGCAGTGTACAAGGCTATCTATAACGAC
AACATCAAGAAACCTGAGTTTGTCCTCTGTGAGAGTGACGATAAGGAGG
GTGAGAGAATAGATAGCGATTTCAGCCGTGATATCTGCGTGCTGCAACC
GCATCGCGGAAAGCGTGAATGTTTGTACACAGGGTTCCAATTGGCAAAG
ATGGACCCCTCAGTTAATGCCGTCGTCCTAATCGACAGTGACACTGTGT
TAGAAAAGGACGCGATTCTCGAaGTAGTATACCCGCTGGCATGCGATCC
AGAAATACAGGCTGTAGCAGGCGAATGCAAAATATGGAATACTGACACA
CTGTTGAGTTTGCTGGTAGCCTGGCGATATTACAGCGCATTTTGCGTAG
AGCGTAGCGCCCAATCATTCTTCAGGACAGTACAATGCGTCGGAGGACC
TCTCGGCGCCTACAAGATTGATATAATTAAGGAAATCAAGGACCCATGG
ATCAGCCAACGTTTCCTTGGCCAAAAGTGCACATACGGCGACGATAGAC
GACTCACTAATGAAATACTAATGAGGGGTAAGAAAGTAGTTTTCACCCC
ATTCGCTGTTGGCTGGTTCGACGAGGAGTTCACACACTGGGGTGGCCGT
TACATTGTACAGCAAACACGGTGGTCCAAGTCGTGGTGTAGGGAGATAT
GGTATACACTGTTTGCAGCATGGAAGCATGGACTTTCGGGCATTTGGCT
CGCATTCGAGTGCTTATACCAGATTACTTATTTCTTCCTGGTGATTTAC
CTATTCTCCCGTTTGGCTGTTGAGGCTGACCCACGGGCGCAAACGGCCA
CCGTCATTGTTTCGACCACAGTTGCGCTTATTAAGTGCGGTTACTTTAG
CTTCAGAGCTAAAGACATTAGGGCCTTCTATTTCGTCCTCTACACGTTC
GTTTACTTCTTCTGCATGATTCCGGCACGAATAACTGCGATGATGACCC
TGTGGGACATAGGTTGGGGAACCAGGGGAGGAAATGAGAAGCCTTCCGT
AGGCACCAGAGTTGCTTTGTGGGCCAAGCAATATTTGATTGCTTACATG
TGGTGGGCCGCTGTCGTCGGTGCTGGCGTGTACTCCATCGTTCATAATT
GGATGTTTGACTGGAATTCACTTTCCTACAGGTTCGCACTGGTAGGCAT
CTGTTCGTATATAGTTTTCATTGTAATAGTGCTGGTAGTCTACTTTACA
GGAAAGATCACGACCTGGAACTTCACGAAGCTACAGAAAGAATTGATCG
AGGACCGCGTACTGTACGACGCAACGACCAATGCCCAGTCGGTATAA

SEQ ID NO: 10 is a nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin synthase from Penicillium oxalicum (HCOS5-Vir)

ATGGGTAAGAACATCATTATTATGGTTTCTTGGTACACTATTATTACAT
CCAATCTCATCGCAGTGGGTGGCGCCTCACTCATACTAGCCCCAGCTAT
TACGGGCTATGTCCTTCACTGGAACATTGCCCTTTCAACAATTTGGGGA
GTGTCGGCCTACGGAATTTTCGTGTTTGGTTTCTTTCTTGCCCAGGTAT
TATTTAGTGAACTCAACCGGAAAAGGCTCCGGAAGTGGATTTCCCTCCG
ACCCAAATCCATTCACCATCCAATTTCGAAAACGACTCACCCCGACACA
GAAAATGCAGGTGACTCAACATTGTTGTGCCCGCCCATGGATGGTATGG
ACGACATATTAGTCGTTATGAAGACAGGTGTCACAGAGGCTAAGGAAAA
GGTGCCAGTACATTTTCAGACAACGCTGAGGTGTGTGCCGCACTACGTT
ATCTATAGCGACTTCGAAGAAGAGATAGAAGGTGTAAAGATTTACGACG
TCTTGCAAGGAATGGACCCCGATGTTATGACGCAGGTTCCGGACTTCGA
TCTTTACAATCGTATTAAGAAGTTAGGGCGTCAGGGCCTTGCGGAGAAC
GACTTCGCGGACGAAGCGAACTCTGCGGTGGGTAAGCCTAATAACCCTG
GTTGGAAGTTAGATAAATGGAAGTTCTTACCTATGATCATTGAGGCTCT
GAGATACAAACCAAAGGCTAAGTGGTATGTTTTCATGGAAGCAGACACT
TATATATCTTGGCCGACTTTATTAGCCTGGCTAAAACACTTCAACCACA
AGAAGCCGTGGTATCTTGGCACTGAGACACAGATAGCCGATGTGATTTT
CGCTCATGGTGGTTCAGGATTCATGATTAGTCATGCCGCGCTTAAGCTT
GCTGCTGAAGAATATACCGCACGTCGTACTGAGTTGCACCACTACACAG
ACGGTCACTGGGCAGGAGACTGCGTATTAGGTAAAGTTCTTGCTGACGC
AGGTGTGAACTTGCATTTCTCCTGGCCAATTTTACAGAATTCCAATCTG
GGCGAACTGGATGAGTTCAACGTTGACTTATACAGGCAACCTTGGTGCT
TTCCAGCTGCCGCTTTCCACCACTTGAACGCTCAGAAGATCCAGGACTT
ATGGTCTTACGAGCAGAAAAGGTGGCAGGACAAGGACAAGAGGGTTCTG
TTACATAGCGACGTCTTCAGGGACCTATTATATCCGGATATAGCATTAA
AACCCGCGAGGATCGGTTGGGACAACCTGAGTGCAGACGAACAGTCTAC
GGCCTCTAGTTTCGAGGAATGCAGGCAAATTTGTGATCAATTAGACGAT
TGTACCCAGTTTAGTTTCAGGGACAAATCCTGTTTTACATCAAGAACTC
CACGTTTGGGCGCAGCCAATCCAAATACAGTTAGCGGATGGATGGTTGA
TCGTATCCGTGCGAATATGGAGAAGTCTGGACTGTTTGCAGCATGGAAG
CATGGACTTTCGGGCATTTGGCTCGCATTCGAGTGCTTATACCAGATTA
CTTATTTCTTCCTGGTGATTTACCTATTCTCCCGTTTGGCTGTTGAGGC
TGACCCACGGGCGCAAACGGCCACCGTCATTGTTTCGACCACAGTTGCG
CTTATTAAGTGCGGTTACTTTAGCTTCAGAGCTAAAGACATTAGGGCCT
TCTATTTCGTCCTCTACACGTTCGTTTACTTCTTCTGCATGATTCCGGC
ACGAATAACTGCGATGATGACCCTGTGGGACATAGGTTGGGGAACCAGG
GGAGGAAATGAGAAGCCTTCCGTAGGCACCAGAGTTGCTTTGTGGGCCA
AGCAATATTTGATTGCTTACATGTGGTGGGCCGCTGTCGTCGGTGCTGG
CGTGTACTCCATCGTTCATAATTGGATGTTTGACTGGAATTCACTTTCC
TACAGGTTCGCACTGGTAGGCATCTGTTCGTATATAGTTTTCATTGTAA
TAGTGCTGGTAGTCTACTTTACAGGAAAGATCACGACCTGGAACTTCAC
GAAGCTACAGAAAGAATTGATCGAGGACCGCGTACTGTACGACGCAACG
ACCAATGCCCAGTCGGTAtaa

SEQ ID NO: 11: is a nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin synthase from Pasteurella multocida (HCOS6-Vir)

ATGGGTAAGAACATCATTATTATGGTTTCTTGGTACACTATTATTACAT
CCAATCTCATCGCAGTGGGTGGCGCCTCACTCATACTAGCCCCAGCTAT
TACGGGCTATGTCCTTCACTGGAACATTGCCCTTTCAACAATTTGGGGA
GTGTCGGCCTACGGAATTTTCGTGTTTGGTTTCTTTCTTGCCCAGGTAT
TATTTAGTGAACTCAACCGGAAAAGGCTCCGGAAGTGGATTTCCCTCCG
ACCCAAATTAGACATTGCAACACAGTTATTACTATCGAATGTTAAGAAA
TTGACCTTAAGCGAGTCCGAAAAGAACAGCTTAAAGAATAAGTGGAAGA
GTATAACAGGCAAGAAGTCCGAGAATGCTGAAATTAGGAAGGTTGAGTT
AGTCCCAAAGGATTTTCCCAAAGACTTAGTTTTAGCGCCACTACCAGAC
CATGTGAACGATTTTACCTGGTACAAGAATAGAAAGAAGAGCCTGGGCA
TAAAGCCTGTGAACAAGAACATCGGGTTGTCAATCATAATCCCAACTTT
CAATCGTAGTAGGATATTAGACATTACCCTGGCGTGTCTAGTTAATCAG
AAAACTAACTATCCGTTTGAGGTGGTTGTTGCAGATGATGGGTCGAAGG
AAAATCTATTGACTATAGTTCAGAAATACGAGCAGAAACTAGACATTAA
GTATGTGAGACAGAAGGACTATGGGTATCAGCTGTGCGCCGTTAGAAAC
CTTGGTCTAAGGACAGCGAAGTATGACTTTGTCTCTATCTTGGATTGCG
ACATGGCCCCGCAGCAGCTATGGGTTCACTCTTACCTTACAGAGTTACT
GGAAGACAACGACATCGTCTTGATCGGGCCAAGGAAATACGTTGATACC
CACAACATCACGGCTGAGCAGTTTCTGAACGACCCATATTTAATTGAGA
GTTTACCGGAAACCGCCACAAACAATAATCCATCGATTACCAGTAAGGG
TAACATCTCATTAGATTGGAGATTAGAACATTTTAAGAAGACAGACAAC
CTAAGACTATGCGACTCCCCGTTCAGGTATTTCTCATGCGGAAATGTGG
CCTTCTCCAAAGAGTGGCTGAACAAAGTTGGCTGGTTCGACGAAGAATT
TAATCACTGGGGCGGCGAAGACGTCGAATTCGGCTACAGGTTATTTGCA
AAAGGGTGCTTCTTTAGGGTTATCGACGGTGGTATGGCCTACCATCAAG
AGCCACCAGGAAAGGAGAACGAAACGGATCGTGAGGCCGGTAAGTCTAT
CACTCTTAAAATAGTAAAGGAGAAAGTGCCCTACATCTACAGGAAGTTG
TTGCCGATTGAGGACTCCCACATACACCGTATACCGCTTGTGAGCATAT
ACATTCCAGCGTACAACTGCGCCAATTATATTCAAAGGTGTGTAGATAG
CGCGTTGAATCAAACAGTGGTAGATTTGGAAGTTTGTATATGTAACGAC
GGAAGTACTGACAATACTTTAGAGGTGATTAACAAGCTGTATGGCAACA
ACCCGCGTGTCAGGATAATGAGCAAGCCCAATGGTGGAATTGCTTCCGC
GTCTAACGCTGCCGTTTCATTCGCGAAAGGCTACTACATTGGCCAGTTG
GACTCGGATGATTACTTGGAACCTGACGCAGTGGAGCTTTGTTTAAAGG
AGTTCTTGAAGGACAAAACACTAGCCTGTGTATATACAACGAACAGAAA
TGTTAATCCTGACGGTTCCCTAATCGCTAACGGATACAATTGGCCTGAG
TTCAGCAGGGAGAAGCTGACCACTGCTATGATCGCCCACCATTTCAGGA
TGTTCACAATCAGGGCTTGGCATTTAACAGATGGTTTTAATGAGAAAAT
AGAGAACGCAGTAGACTACGATATGTTTCTGAAGTTATCAGAGGTCGGG
AAATTCAAACACTTGAACAAAATTTGTTACAATAGGGTATTACACGGTG
ACAACACCAGTATTAAGAATCTTGACACCCAAAAGAAGAACCACTTCGT
AGTAGTGAACCAGTCTCTGAACAGACAAAGGGTGTCGAATTATAACTAC
GATGAGTTTGATAACCTGGACGAGAGCCGTAAGTACATATTCAACAAGC
TGTTTGCAGCATGGAAGCATGGACTTTCGGGCATTTGGCTCGCATTCGA
GTGCTTATACCAGATTACTTATTTCTTCCTGGTGATTTACCTATTCTCC
CGTTTGGCTGTTGAGGCTGACCCACGGGCGCAAACGGCCACCGTCATTG
TTTCGACCACAGTTGCGCTTATTAAGTGCGGTTACTTTAGCTTCAGAGC
TAAAGACATTAGGGCCTTCTATTTCGTCCTCTACACGTTCGTTTACTTC
TTCTGCATGATTCCGGCACGAATAACTGCGATGATGACCCTGTGGGACA
TAGGTTGGGGAACCAGGGGAGGAAATGAGAAGCCTTCCGTAGGCACCAG
AGTTGCTTTGTGGGCCAAGCAATATTTGATTGCTTACATGTGGTGGGCC
GCTGTCGTCGGTGCTGGCGTGTACTCCATCGTTCATAATTGGATGTTTG
ACTGGAATTCACTTTCCTACAGGTTCGCACTGGTAGGCATCTGTTCGTA
TATAGTTTTCATTGTAATAGTGCTGGTAGTCTACTTTACAGGAAAGATC
ACGACCTGGAACTTCACGAAGCTACAGAAAGAATTGATCGAGGACCGCG
TACTGTACGACGCAACGACCAATGCCCAGTCGGTAtaa

SEQ ID NO: 12: is a reencoded nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin synthase from Pasteurella multocida (HCOS7-Vir)

ATGGGTAAGAACATCATTATTATGGTTTCTTGGTACACTATTATTACAT
CCAATCTCATCGCAGTGGGTGGCGCCTCACTCATACTAGCCCCAGCTAT
TACGGGCTATGTCCTTCACTGGAACATTGCCCTTTCAACAATTTGGGGA
GTGTCGGCCTACGGAATTTTCGTGTTTGGTTTCTTTCTTGCCCAGGTAT
TATTTAGTGAACTCAACCGGAAAAGGCTCCGGAAGTGGATTTCCCTCCG
ACCCAAAATCCCAACTTTCAATCGTAGTAGGATATTAGACATTACCCTG
GCGTGTCTAGTTAATCAGAAAACTAACTATCCGTTTGAGGTGGTTGTTG
CAGATGATGGGTCGAAGGAAAATCTATTGACTATAGTTCAGAAATACGA
GCAGAAACTAGACATTAAGTATGTGAGACAGAAGGACTATGGGTATCAG
CTGTGCGCCGTTAGAAACCTTGGTCTAAGGACAGCGAAGTATGACTTTG
TCTCTATCTTGGATTGCGACATGGCCCCGCAGCAGCTATGGGTTCACTC
TTACCTTACAGAGTTACTGGAAGACAACGACATCGTCTTGATCGGGCCA
AGGAAATACGTTGATACCCACAACATCACGGCTGAGCAGTTTCTGAACG
ACCCATATTTAATTGAGAGTTTACCGGAAACCGCCACAAACAATAATCC
ATCGATTACCAGTAAGGGTAACATCTCATTAGATTGGAGATTAGAACAT
TTTAAGAAGACAGACAACCTAAGACTATGCGACTCCCCGTTCAGGTATT
TCTCATGCGGAAATGTGGCCTTCTCCAAAGAGTGGCTGAACAAAGTTGG
CTGGTTCGACGAAGAATTTAATCACTGGGGCGGCGAAGACGTCGAATTC
GGCTACAGGTTATTTGCAAAAGGGTGCTTCTTTAGGGTTATCGACGGTG
GTATGGCCTACCATCAAGAGCCACCAGGAAAGGAGAACGAAACGGATCG
TGAGGCCGGTAAGTCTATCACTCTTAAAATAGTAAAGGAGAAAGTGCCC
TACATCTACAGGAAGTTGTTGCCGATTGAGGACTCCCACATACACCGTA
TACCGCTTGTGAGCATATACATTCCAGCGTACAACTGCGCCAATTATAT
TCAAAGGTGTGTAGATAGCGCGTTGAATCAAACAGTGGTAGATTTGGAA
GTTTGTATATGTAACGACGGAAGTACTGACAATACTTTAGAGGTGATTA
ACAAGCTGTATGGCAACAACCCGCGTGTCAGGATAATGAGCAAGCCCAA
TGGTGGAATTGCTTCCGCGTCTAACGCTGCCGTTTCATTCGCGAAAGGC
TACTACATTGGCCAGTTGGACTCGGATGATTACTTGGAACCTGACGCAG
TGGAGCTTTGTTTAAAGGAGTTCTTGAAGGACAAAACACTAGCCTGTGT
ATATACAACGAACAGAAATGTTAATCCTGACGGTTCCCTAATCGCTAAC
GGATACAATTGGCCTCTGTTTGCAGCATGGAAGCATGGACTTTCGGGCA
TTTGGCTCGCATTCGAGTGCTTATACCAGATTACTTATTTCTTCCTGGT
GATTTACCTATTCTCCCGTTTGGCTGTTGAGGCTGACCCACGGGCGCAA
ACGGCCACCGTCATTGTTTCGACCACAGTTGCGCTTATTAAGTGCGGTT
ACTTTAGCTTCAGAGCTAAAGACATTAGGGCCTTCTATTTCGTCCTCTA
CACGTTCGTTTACTTCTTCTGCATGATTCCGGCACGAATAACTGCGATG
ATGACCCTGTGGGACATAGGTTGGGGAACCAGGGGAGGAAATGAGAAGC
CTTCCGTAGGCACCAGAGTTGCTTTGTGGGCCAAGCAATATTTGATTGC
TTACATGTGGTGGGCCGCTGTCGTCGGTGCTGGCGTGTACTCCATCGTT
CATAATTGGATGTTTGACTGGAATTCACTTTCCTACAGGTTCGCACTGG
TAGGCATCTGTTCGTATATAGTTTTCATTGTAATAGTGCTGGTAGTCTA
CTTTACAGGAAAGATCACGACCTGGAACTTCACGAAGCTACAGAAAGAA
TTGATCGAGGACCGCGTACTGTACGACGCAACGACCAATGCCCAGTCGG
TAtaa

SEQ ID NO: 13: is a reencoded nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin synthase from Pasteurella multocida (HCOS8-Vir)

ATGGGTAAGAACATCATTATTATGGTTTCTTGGTACACTATTATTACAT
CCAATCTCATCGCAGTGGGTGGCGCCTCACTCATACTAGCCCCAGCTAT
TACGGGCTATGTCCTTCACTGGAACATTGCCCTTTCAACAATTTGGGGA
GTGTCGGCCTACGGAATTTTCGTGTTTGGTTTCTTTCTTGCCCAGGTAT
TATTTAGTGAACTCAACCGGAAAAGGCTCCGGAAGTGGATTTCCCTCCG
ACCCAAAGGGTGGAATGATGTTAGGTTGGCTGTAATTATTGCCGGCTAC
CGTGAGGACCCGTATATGTTCCAAAAGTGTCTTGAAAGTGTGCGTGACT
CAGACTATGGGAATGTAGCTAGACTAATATGCGTTATTGACGGCGATGA
AGACGACGACATGAGGATGGCTGCAGTGTACAAGGCTATCTATAACGAC
AACATCAAGAAACCTGAGTTTGTCCTCTGTGAGAGTGACGATAAGGAGG
GTGAGAGAATAGATAGCGATTTCAGCCGTGATATCTGCGTGCTGCAACC
GCATCGCGGAAAGCGTGAATGTTTGTACACAGGGTTCCAATTGGCAAAG
ATGGACCCCTCAGTTAATGCCGTCGTCCTAATCGACAGTGACACTGTGT
TAGAAAAGGACGCGATTCTCGAaGTAGTATACCCGCTGGCATGCGATCC
AGAAATACAGGCTGTAGCAGGCGAATGCAAAATATGGAATACTGACACA
CTGTTGAGTTTGCTGGTAGCCTGGCGATATTACAGCGCATTTTGCGTAG
AGCGTAGCGCCCAATCATTCTTCAGGACAGTACAATGCGTCAGACTATG
CGACTCCCCGTTCAGGTATTTCTCATGCGGAAATGTGGCCTTCTCCAAA
GAGTGGCTGAACAAAGTTGGCTGGTTCGACGAAGAATTTAATCACTGGG
GCGGCGAAGACGTCGAATTCGGCTACAGGTTAAAGAAAGTAGTTTTCAC
CCCATTCGCTGTTGGCTGGTCCGACAGCCCGACGAACGTCTTCCGTTAC
ATTGTACAGCAAACACGGTGGTCCAAGTCGTGGTGTAGGGAGATATGGT
ATACACTGTTTGCAGCATGGAAGCATGGACTTTCGGGCATTTGGCTCGC
ATTCGAGTGCTTATACCAGATTACTTATTTCTTCCTGGTGATTTACCTA
TTCTCCCGTTTGGCTGTTGAGGCTGACCCACGGGCGCAAACGGCCACCG
TCATTGTTTCGACCACAGTTGCGCTTATTAAGTGCGGTTACTTTAGCTT
CAGAGCTAAAGACATTAGGGCCTTCTATTTCGTCCTCTACACGTTCGTT
TACTTCTTCTGCATGATTCCGGCACGAATAACTGCGATGATGACCCTGT
GGGACATAGGTTGGGGAACCAGGGGAGGAAATGAGAAGCCTTCCGTAGG
CACCAGAGTTGCTTTGTGGGCCAAGCAATATTTGATTGCTTACATGTGG
TGGGCCGCTGTCGTCGGTGCTGGCGTGTACTCCATCGTTCATAATTGGA
TGTTTGACTGGAATTCACTTTCCTACAGGTTCGCACTGGTAGGCATCTG
TTCGTATATAGTTTTCATTGTAATAGTGCTGGTAGTCTACTTTACAGGA
AAGATCACGACCTGGAACTTCACGAAGCTACAGAAAGAATTGATCGAGG
ACCGCGTACTGTACGACGCAACGACCAATGCCCAGTCGGTAtaa

SEQ ID NO: 14: is a nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin sulfate synthase from Homo sapiens (HCOS9-Vir)

ATGGGTAAGAACATCATTATTATGGTTTCTTGGTACACTATTATTACAT
CCAATCTCATCGCAGTGGGTGGCGCCTCACTCATACTAGCCCCAGCTAT
TACGGGCTATGTCCTTCACTGGAACATTGCCCTTTCAACAATTTGGGGA
GTGTCGGCCTACGGAATTTTCGTGTTTGGTTTCTTTCTTGCCCAGGTAT
TATTTAGTGAACTCAACCGGAAAAGGCTCCGGAAGTGGATTTCCCTCCG
ACCCAAACACATTGGTAAGTGTCTAAGGGAAATGTATACGACTCATGAG
GACGTGGAAGTAGGAAGATGCGTTAGGAGATTCGCTGGAGTACAGTGTG
TTTGGTCTTACGAGATGCAACAGTTATTCTACGAGAACTACGAGCAGAA
TAAGAAGGGATACATCCGTGATCTGCACAACTCCAAAATTCACCAAGCA
ATAACGCTGCATCCTAACAAGAATCCGCCCTATCAGTATAGGTTGCACT
CTTACATGTTGAGTAGAAAGATATCTGAGCTACGTCATAGAACTATTCA
GTTGCACAGGGAGATAGTACTGATGTCTAAATACTCAAACACGGAGATC
CATAAGGAAGACCTACAGTTGGGTATACCACCTTCGTTCATGAGATTCC
AGCCAAGACAGAGGGAAGAAATTTTGGAGTGGGAGTTCTTGACTGGTAA
ATACTTGTATTCTGCTGTTGACGGTCAACCACCTAGGAGAGGGATGGAC
TCTGCACAGCGTGAGGCTTTAGACGATATAGTGATGCAAGTAATGGAAA
TGATCAACGCTAATGCAAAGACACGTGGTCGTATCATAGATTTCAAAGA
GATCCAGTATGGATATCGTCGTGTGAACCCAATGTACGGCGCGGAGTAT
ATTCTTGACTTATTGTTGCTATACAAGAAACATAAGGGTAAGAAGATGA
CTGTCCCGGTTCGTAGGCACGCGTATTTGCAACAGACTTTCTCGAAAAT
ACAGTTTGTAGAGCATGAGGAACTTGACGCGCAAGAGTTAGCGAAACGT
ATAAACCAAGAGTCGGGGAGTTTATCCTTCTTAAGTAATTCCCTTAAGA
AGTTGGTTCCTTTCCAGCTGCCTGGAAGTAAAAGTGAGCACAAGGAGCC
AAAGGATAAGAAAATCAACATTCTTATACCTCTTTCCGGCAGATTCGAC
ATGTTCGTTAGGTTCATGGGTAACTTCGAGAAGACCTGCTTAATTCCTA
ACCAGAATGTTAAGTTGGTAGTTTTGTTATTCAATTCTGACAGTAACCC
CGACAAAGCAAAGCAGGTTGAACTGATGAGAGACTATAGGATTAAGTAC
CCAAAAGCAGATATGCAGATACTACCTGTTTCCGGAGAATTCTCAAGAG
CCTTGGCTCTGGAAGTAGGCAGCTCTCAGTTTAATAATGAGTCTCTTCT
GTTCTTCTGCGACGTTGACTTAGTTTTCACGACAGAGTTTCTGCAGAGA
TGTCGTGCAAACACTGTACTAGGTCAGCAGATATATTTCCCCATTATTT
TCAGTCAGTACGATCCAAAGATTGTTTATAGTGGAAAAGTTCCATCTGA
CAACCATTTTGCGTTTACTCAAAAGACAGGGTTTTGGCGTAACTACGGG
TTCGGCATAACATGTATCTATAAAGGGGACCTGGTAAGAGTTGGCGGAT
TCGACGTATCTATACAGGGCTGGGGTTTAGAAGACGTCGATCTATTTAA
TAAAGTAGTTCAGGCAGGTTTGAAGACATTCCGTAGTCAAGAGGTAGGC
GTTGTACACGTGCACCATCCAGTATTTTGCGACCCAAACTTAGACCCGA
AGCAATATAAAATGTGCTTGGGTTCCAAGGCATCGACTTACGGTTCCAC
GCAGCAACTACTGTTTGCAGCATGGAAGCATGGACTTTCGGGCATTTGG
CTCGCATTCGAGTGCTTATACCAGATTACTTATTTCTTCCTGGTGATTT
ACCTATTCTCCCGTTTGGCTGTTGAGGCTGACCCACGGGCGCAAACGGC
CACCGTCATTGTTTCGACCACAGTTGCGCTTATTAAGTGCGGTTACTTT
AGCTTCAGAGCTAAAGACATTAGGGCCTTCTATTTCGTCCTCTACACGT
TCGTTTACTTCTTCTGCATGATTCCGGCACGAATAACTGCGATGATGAC
CCTGTGGGACATAGGTTGGGGAACCAGGGGAGGAAATGAGAAGCCTTCC
GTAGGCACCAGAGTTGCTTTGTGGGCCAAGCAATATTTGATTGCTTACA
TGTGGTGGGCCGCTGTCGTCGGTGCTGGCGTGTACTCCATCGTTCATAA
TTGGATGTTTGACTGGAATTCACTTTCCTACAGGTTCGCACTGGTAGGC
ATCTGTTCGTATATAGTTTTCATTGTAATAGTGCTGGTAGTCTACTTTA
CAGGAAAGATCACGACCTGGAACTTCACGAAGCTACAGAAAGAATTGAT
CGAGGACCGCGTACTGTACGACGCAACGACCAATGCCCAGTCGGTATAA

SEQ ID NO: 15: is a reencoded nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin sulfate synthase from Homo sapiens (HCOS10-Vir)

ATGGGTAAGAACATCATTATTATGGTTTCTTGGTACACTATTATTACAT
CCAATCTCATCGCAGTGGGTGGCGCCTCACTCATACTAGCCCCAGCTAT
TACGGGCTATGTCCTTCACTGGAACATTGCCCTTTCAACAATTTGGGGA
GTGTCGGCCTACGGAATTTTCGTGTTTGGTTTCTTTCTTGCCCAGGTAT
TATTTAGTGAACTCAACCGGAAAAGGCTCCGGAAGTGGATTTCCCTCCG
ACCCAAAAGAGATAGAAACTTCCTATTCGTTGGTGTTATGACAGCTCAG
AAGTACTTACAGACCAGGGCGGTTGCAGCATATAGAACCTGGTCAAAGA
CAATTCCAGGTAAGGTACAGTTCTTCAGCTCCGAGGGTTCTGACACCAG
CGTCCCTATCCCAGTAGTGCCTTTGCGTGGAGTGGATGATTCATACCCA
CCTCAGAAGAAGTCCTTCATGATGCTTAAGTATATGCACGACCACTATC
TGGACAAATACGAGTGGTTCATGAGAGCAGACGACGACGTTTACATAAA
GGGTGATAGACTGGAGAATTTCTTGAGGTCACTAAACAGTTCAGAACCA
CTTTTCCTAGGACAGACGGGCCTTGGTACGACAGAGGAGATGGGTAAGT
TGGCATTGGAGCCAGGGGAGAACTTCTGCATGGGTGGCCCTGGTGTGAT
TATGAGTAGGGAAGTTTTAAGAAGAATGGTTCCGCACATTGGTAAGTGT
CTAAGGGAAATGTATACGACTCATGAGGACGTGGAAGTAGGAAGATGCG
TTAGGAGATTCGCTGGAGTACAGTGTGTTTGGTCTTACGAGATGCAACA
GTTATTCTACGAGAACTACGAGCAGAATAAGAAGGGATACATCCGTGAT
CTGCACAACTCCAAAATTCACCAAGCAATAACGCTGCATCCTAACAAGA
ATCCGCCCTATCAGTATAGGTTGCACTCTTACATGTTGAGTAGAAAGAT
ATCTGAGCTACGTCATAGAACTATTCAGTTGCACAGGGAGATAGTACTG
ATGTCTAAATACTCAAACACGGAGATCCATAAGGAAGACCTACAGTTGG
GTATACCACCTTCGTTCATGAGATTCCAGCCAAGACAGAGGGAAGAAAT
TTTGGAGTGGGAGTTCTTGACTGGTAAATACTTGTATTCTGCTGTTGAC
GGTCAACCACCTAGGAGAGGGATGGACTCTGCACAGCGTGAGGCTTTAG
ACGATATAGTGATGCAAGTAATGGAAATGATCAACGCTAATGCAAAGAC
ACGTGGTCGTATCATAGATTTCAAAGAGATCCAGTATGGATATCGTCGT
GTGAACCCAATGTACGGCGCGGAGTATATTCTTGACTTATTGTTGCTAT
ACAAGAAACATAAGGGTAAGAAGATGACTGTCCCGGTTCGTAGGCACGC
GTATTTGCAACAGACTTTCTCGAAAATACAGTTTGTAGAGCATGAGGAA
CTTGACGCGCAAGAGTTAGCGAAACGTATAAACCAAGAGTCGGGGAGTT
TATCCTTCTTAAGTAATTCCCTTAAGAAGTTGGTTCCTTTCCAGCTGCC
TGGAAGTAAAAGTGAGCACAAGGAGCCAAAGGATAAGAAAATCAACATT
CTTATACCTCTTTCCGGCAGATTCGACATGTTCGTTAGGTTCATGGGTA
ACTTCGAGAAGACCTGCTTAATTCCTAACCAGAATGTTAAGTTGGTAGT
TTTGTTATTCAATTCTGACAGTAACCCCGACAAAGCAAAGCAGGTTGAA
CTGATGAGAGACTATAGGATTAAGTACCCAAAAGCAGATATGCAGATAC
TACCTGTTTCCGGAGAATTCTCAAGAGCCTTGGCTCTGGAAGTAGGCAG
CTCTCAGTTTAATAATGAGTCTCTTCTGTTCTTCTGCGACGTTGACTTA
GTTTTCACGACAGAGTTTCTGCAGAGATGTCGTGCAAACACTGTACTAG
GTCAGCAGATATATTTCCCCATTATTTTCAGTCAGTACGATCCAAAGAT
TGTTTATAGTGGAAAAGTTCCATCTGACAACCATTTTGCGTTTACTCAA
AAGACAGGGTTTTGGCGTAACTACGGGTTCGGCATAACATGTATCTATA
AAGGGGACCTGGTAAGAGTTGGCGGATTCGACGTATCTATACAGGGCTG
GGGTTTAGAAGACGTCGATCTATTTAATAAAGTAGTTCAGGCAGGTTTG
AAGACATTCCGTAGTCAAGAGGTAGGCGTTGTACACGTGCACCATCCAG
TATTTTGCGACCCAAACTTAGACCCGAAGCAATATAAAATGTGCTTGGG
TTCCAAGGCATCGACTTACGGTTCCACGCAGCAACTAGCAGAGATGTGG
TTGGAGAAGAACGACCCATCTTATAGCCTGTTTGCAGCATGGAAGCATG
GACTTTCGGGCATTTGGCTCGCATTCGAGTGCTTATACCAGATTACTTA
TTTCTTCCTGGTGATTTACCTATTCTCCCGTTTGGCTGTTGAGGCTGAC
CCACGGGCGCAAACGGCCACCGTCATTGTTTCGACCACAGTTGCGCTTA
TTAAGTGCGGTTACTTTAGCTTCAGAGCTAAAGACATTAGGGCCTTCTA
TTTCGTCCTCTACACGTTCGTTTACTTCTTCTGCATGATTCCGGCACGA
ATAACTGCGATGATGACCCTGTGGGACATAGGTTGGGGAACCAGGGGAG
GAAATGAGAAGCCTTCCGTAGGCACCAGAGTTGCTTTGTGGGCCAAGCA
ATATTTGATTGCTTACATGTGGTGGGCCGCTGTCGTCGGTGCTGGCGTG
TACTCCATCGTTCATAATTGGATGTTTGACTGGAATTCACTTTCCTACA
GGTTCGCACTGGTAGGCATCTGTTCGTATATAGTTTTCATTGTAATAGT
GCTGGTAGTCTACTTTACAGGAAAGATCACGACCTGGAACTTCACGAAG
CTACAGAAAGAATTGATCGAGGACCGCGTACTGTACGACGCAACGACCA
ATGCCCAGTCGGTATAA

SEQ ID NO: 16: is a reencoded nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin sulfate synthase from Homo sapiens (HCOS11-Vir)

ATGGGTAAGAACATCATTATTATGGTTTCTTGGTACACTATTATTACAT
CCAATCTCATCGCAGTGGGTGGCGCCTCACTCATACTAGCCCCAGCTAT
TACGGGCTATGTCCTTCACTGGAACATTGCCCTTTCAACAATTTGGGGA
GTGTCGGCCTACGGAATTTTCGTGTTTGGTTTCTTTCTTGCCCAGGTAT
TATTTAGTGAACTCAACCGGAAAAGGCTCCGGAAGTGGATTTCCCTCCG
ACCCAAAAGAGATAGAAACTTCCTATTCGTTGGTGTTATGACAGCTCAG
AAGTACTTACAGACCAGGGCGGTTGCAGCATATAGAACCTGGTCAAAGA
CAATTCCAGGTAAGGTACAGTTCTTCAGCTCCGAGGGTTCTGACACCAG
CGTCCCTATCCCAGTAGTGCCTTTGCGTGGAGTGGATGATTCATACCCA
CCTCAGAAGAAGTCCTTCATGATGCTTAAGTATATGCACGACCACTATC
TGGACAAATACGAGTGGTTCATGAGAGCAGACGACGACGTTTACATAAA
GGGTGATAGACTGGAGAATTTCTTGAGGTCACTAAACAGTTCAGAACCA
CTTTTCCTAGGACAGACGGGCCTTGGTACGACAGAGGAGATGGGTAAGT
TGGCATTGGAGCCAGGGGAGAACTTCTGCATGGGTGGCCCTGGTGTGAT
TATGAGTAGGGAAGTTTTAAGAAGAATGGTTCCGCACATTGGTAAGTGT
CTAAGGGAAATGTATACGACTCATGAGGACGTGGAAGTAGGAAGATGCG
TTAGGAGATTCGCTGGAGTACAGTGTGTTTGGTCTTACGAGATGCAACA
GTTATTCTACGAGAACTACGAGCAGAATAAGAAGGGATACATCCGTGAT
CTGCACAACTCCAAAATTCACCAAGCAATAACGCTGCATCCTAACAAGA
ATCCGCCCTATCAGTATAGGTTGCACTCTTACATGTTGCTGTTTGCAGC
ATGGAAGCATGGACTTTCGGGCATTTGGCTCGCATTCGAGTGCTTATAC
CAGATTACTTATTTCTTCCTGGTGATTTACCTATTCTCCCGTTTGGCTG
TTGAGGCTGACCCACGGGCGCAAACGGCCACCGTCATTGTTTCGACCAC
AGTTGCGCTTATTAAGTGCGGTTACTTTAGCTTCAGAGCTAAAGACATT
AGGGCCTTCTATTTCGTCCTCTACACGTTCGTTTACTTCTTCTGCATGA
TTCCGGCACGAATAACTGCGATGATGACCCTGTGGGACATAGGTTGGGG
AACCAGGGGAGGAAATGAGAAGCCTTCCGTAGGCACCAGAGTTGCTTTG
TGGGCCAAGCAATATTTGATTGCTTACATGTGGTGGGCCGCTGTCGTCG
GTGCTGGCGTGTACTCCATCGTTCATAATTGGATGTTTGACTGGAATTC
ACTTTCCTACAGGTTCGCACTGGTAGGCATCTGTTCGTATATAGTTTTC
ATTGTAATAGTGCTGGTAGTCTACTTTACAGGAAAGATCACGACCTGGA
ACTTCACGAAGCTACAGAAAGAATTGATCGAGGACCGCGTACTGTACGA
CGCAACGACCAATGCCCAGTCGGTATAA

SEQ ID NO: 17: is a reencoded nucleic acid sequence of a polypeptide having chondroitin synthase activity:chimeric nucleic acid comprising a fragment from a nucleic acid encoding a hyaluronan synthase originating from Chlorella virus PBCV-1 and a fragment from a nucleic acid encoding a chondroitin sulfate synthase from Homo sapiens (HCOS12-Vir)

ATGGGTAAGAACATCATTATTATGGTTTCTTGGTACACTATTATTACAT
CCAATCTCATCGCAGTGGGTGGCGCCTCACTCATACTAGCCCCAGCTAT
TACGGGCTATGTCCTTCACTGGAACATTGCCCTTTCAACAATTTGGGGA
GTGTCGGCCTACGGAATTTTCGTGTTTGGTTTCTTTCTTGCCCAGGTAT
TATTTAGTGAACTCAACCGGAAAAGGCTCCGGAAGTGGATTTCCCTCCG
ACCCAAAAAGGATAAGAAAATCAACATTCTTATACCTCTTTCCGGCAGA
TTCGACATGTTCGTTAGGTTCATGGGTAACTTCGAGAAGACCTGCTTAA
TTCCTAACCAGAATGTTAAGTTGGTAGTTTTGTTATTCAATTCTGACAG
TAACCCCGACAAAGCAAAGCAGGTTGAACTGATGAGAGACTATAGGATT
AAGTACCCAAAAGCAGATATGCAGATACTACCTGTTTCCGGAGAATTCT
CAAGAGCCTTGGCTCTGGAAGTAGGCAGCTCTCAGTTTAATAATGAGTC
TCTTCTGTTCTTCTGCGACGTTGACTTAGTTTTCACGACAGAGTTTCTG
CAGAGATGTCGTGCAAACACTGTACTAGGTCAGCAGATATATTTCCCCA
TTATTTTCAGTCAGTACGATCCAAAGATTGTTTATAGTGGAAAAGTTCC
ATCTGACAACCATTTTGCGTTTACTCAAAAGACAGGGTTTTGGCGTAAC
TACGGGTTCGGCATAACATGTATCTATAAAGGGGACCTGGTAAGAGTTG
GCGGATTCGACGTATCTATACAGGGCTGGGGTTTAGAAGACGTCGATCT
ATTTAATAAAGTAGTTCAGGCAGGTTTGAAGACATTCCGTAGTCAAGAG
GTAGGCGTTGTACACGTGCACCATCCAGTATTTTGCGACCCAAACTTAG
ACCCGAAGCAATATAAAATGTGCTTGGGTTCCAAGGCATCGACTTACGG
TTCCACGCAGCAACTAGCAGAGATGTGGTTGGAGAAGAACGACCCATCT
TATAGCCTGTTTGCAGCATGGAAGCATGGACTTTCGGGCATTTGGCTCG
CATTCGAGTGCTTATACCAGATTACTTATTTCTTCCTGGTGATTTACCT
ATTCTCCCGTTTGGCTGTTGAGGCTGACCCACGGGCGCAAACGGCCACC
GTCATTGTTTCGACCACAGTTGCGCTTATTAAGTGCGGTTACTTTAGCT
TCAGAGCTAAAGACATTAGGGCCTTCTATTTCGTCCTCTACACGTTCGT
TTACTTCTTCTGCATGATTCCGGCACGAATAACTGCGATGATGACCCTG
TGGGACATAGGTTGGGGAACCAGGGGAGGAAATGAGAAGCCTTCCGTAG
GCACCAGAGTTGCTTTGTGGGCCAAGCAATATTTGATTGCTTACATGTG
GTGGGCCGCTGTCGTCGGTGCTGGCGTGTACTCCATCGTTCATAATTGG
ATGTTTGACTGGAATTCACTTTCCTACAGGTTCGCACTGGTAGGCATCT
GTTCGTATATAGTTTTCATTGTAATAGTGCTGGTAGTCTACTTTACAGG
AAAGATCACGACCTGGAACTTCACGAAGCTACAGAAAGAATTGATCGAG
GACCGCGTACTGTACGACGCAACGACCAATGCCCAGTCGGTATAA

SEQ ID NO: 18 is an amino acid sequence of a polypeptide having a chondroitin synthase activity:chimeric amino acid comprising a fragment from an amino acid encoding a hyaluronan synthase originating from Pasteurella multocida and a fragment from an amino acid encoding a chondroitin synthase from Pasteurella multocida (HCOS1-1)

MNTLSQAIKAYNSNDYQLALKLFEKSAEIYGRKIVEFQITKCKEKLSAH
PSVNSAHLSVNKEEKVNVCDSPLDIATQLLLSNVKKLVLSDSEKNTLKN
KWKLLTEKKSENAEVRAVALVPKDFPKDLVLAPLPDHVNDFTWYKKRKK
RLGIKPEHQHVGLSIIVTTFNRPAILSITLACLVNQKTHYPFEVIVTDD
GSQEDLSPIIRQYENKLDIRYVRQKDYGYQLCAVRNLGLRTAKYDFVSI
LDCDMAPQQLWVHSYLTELLEDDDLTIIGPRKYIDTQHIDPKDFLNNAS
LLESLPEVKTNNSVAAKGEGTVSLDWRLEQFEKTENLRLSDSPFRFFAA
GNVAFAKKWINKSGFFDEEFNHWGGEDVEFGYRLFRYGSFFKTIDGIMA
YHQEPPGKENETDREAGKNITLDIMREKVPYIYRKLLPIEDSHINRVPL
VSIYIPAYNCANYIQRCVDSALNQTVVDLEVCICNDGSTDNTLEVINKL
YGNNPRVRIMSKPNGGIASASNAAVSFAKGYYIGQLDSDDYLEPDAVEL
CLKEFLKDKTLACVYTTNRNVNPDGSLIANGYNWPEFSREKLTTAMIAH
HFRMFTIRAWHLTDGFNEKIENAVDYDMFLKLSEVGKFKHLNKICYNRV
LHGDNTSIKKLGIQKKNHFVVVNQSLNRQGITYYNYDEFDDLDESRKYI
FNKTAEYQEEIDILKDIKIIQNKDAKIAVSIFYPNTLNGLVKKLNNIIE
YNKNIFVIVLHVDKNHLTPDIKKEILAFYHKHQVNILLNNDISYYTSNR
LIKTEAHLSNINKLSQLNLNCEYIIFDNHDSLFVKNDSYAYMKKYDVGM
NFSALTHDWIEKINAHPPFKKLIKTYFNDNDLKSMNVKGASQGMFMTYA
LAHELLTIIKEVITSCQSIDSVPEYNTEDIWFQFALLILEKKTGHVFNK
TSTLTYMPWERKLQWTNEQIESAKRGENIPVNKFIINSITL

SEQ ID NO: 19 is an amino acid sequence of a polypeptide having a chondroitin synthase activity:chimeric amino acid comprising a fragment from an amino acid encoding a hyaluronan synthase originating from Pasteurella multocida and a fragment from an amino acid encoding a chondroitin synthase from Pasteurella multocida (HCOS1-2)

MNTLSQAIKAYNSNDYQLALKLFEKSAEIYGRKIVEFQITKCKEKLSAH
PSVNSAHLSVNKEEKVNVCDSPLDIATQLLLSNVKKLVLSDSEKNTLKN
KWKLLTEKKSENAEVRAVALVPKDFPKDLVLAPLPDHVNDFTWYKKRKK
RLGIKPVNKNIGLSIIIPTFNRSRILDITLACLVNQKTHYPFEVIVTDD
GSQEDLSPIIRQYENKLDIRYVRQKDYGYQLCAVRNLGLRTAKYDFVSI
LDCDMAPQQLWVHSYLTELLEDDDLTIIGPRKYIDTQHIDPKDFLNNAS
LLESLPEVKTNNSVAAKGEGTVSLDWRLEQFEKTENLRLSDSPFRFFSC
GNVAFSKEWLNKVGWFDEEFNHWGGEDVEFGYRLFRYGSFFKTIDGIMA
YHQEPPGKENETDREAGKNITLDIMREKVPYIYRKLLPIEDSHINRVPL
VSIYIPAYNCANYIQRCVDSALNQTVVDLEVCICNDGSTDNTLEVINKL
YGNNPRVRIMSKPNGGIASASNAAVSFAKGYYIGQLDSDDYLEPDAVEL
CLKEFLKDKTLACVYTTNRNVNPDGSLIANGYNWPEFSREKLTTAMIAH
HFRMFTIRAWHLTDGFNEKIENAVDYDMFLKLSEVGKFKHLNKICYNRV
LHGDNTSIKKLGIQKKNHFVVVNQSLNRQGITYYNYDEFDDLDESRKYI
FNKTAEYQEEIDILKDIKIIQNKDAKIAVSIFYPNTLNGLVKKLNNIIE
YNKNIFVIVLHVDKNHLTPDIKKEILAFYHKHQVNILLNNDISYYTSNR
LIKTEAHLSNINKLSQLNLNCEYIIFDNHDSLFVKNDSYAYMKKYDVGM
NFSALTHDWIEKINAHPPFKKLIKTYFNDNDLKSMNVKGASQGMFMTYA
LAHELLTIIKEVITSCQSIDSVPEYNTEDIWFQFALLILEKKTGHVFNK
TSTLTYMPWERKLQWTNEQIESAKRGENIPVNKFIINSITL

SEQ ID NO: 20 is an amino acid sequence of a polypeptide having a chondroitin synthase activity:chimeric amino acid comprising a fragment from an amino acid encoding a hyaluronan synthase originating from Pasteurella multocida and a fragment from an amino acid encoding a chondroitin synthase from Pasteurella multocida (HCOS1-3)

MNTLSQAIKAYNSNDYQLALKLFEKSAEIYGRKIVEFQITKCKEKLSAH
PSVNSAHLSVNKEEKVNVCDSPLDIATQLLLSNVKKLVLSDSEKNTLKN
KWKLLTEKKSENAEVRAVALVPKDFPKDLVLAPLPDHVNDFTWYKNRKK
SLGIKPVNKNIGLSIIIPTFNRSRILDITLACLVNQKTNYPFEVVVADD
GSKENLLTIVQKYEQKLDIKYVRQKDYGYQLCAVRNLGLRTAKYDFVSI
LDCDMAPQQLWVHSYLTELLEDNDIVLIGPRKYVDTHNITAEQFLNDPY
LIESLPETATNNNPSITSKGNISLDWRLEHFKKTDNLRLCDSPFRYFSC
GNVAFSKEWLNKVGWFDEEFNHWGGEDVEFGYRLFAKGCFFRVIDGGMA
YHQEPPGKENETDREAGKSITLKIVKEKVPYIYRKLLPIEDSHINRVPL
VSIYIPAYNCANYIQRCVDSALNQTVVDLEVCICNDGSTDNTLEVINKL
YGNNPRVRIMSKPNGGIASASNAAVSFAKGYYIGQLDSDDYLEPDAVEL
CLKEFLKDKTLACVYTTNRNVNPDGSLIANGYNWPEFSREKLTTAMIAH
HFRMFTIRAWHLTDGFNEKIENAVDYDMFLKLSEVGKFKHLNKICYNRV
LHGDNTSIKKLGIQKKNHFVVVNQSLNRQGITYYNYDEFDDLDESRKYI
FNKTAEYQEEIDILKDIKIIQNKDAKIAVSIFYPNTLNGLVKKLNNIIE
YNKNIFVIVLHVDKNHLTPDIKKEILAFYHKHQVNILLNNDISYYTSNR
LIKTEAHLSNINKLSQLNLNCEYIIFDNHDSLFVKNDSYAYMKKYDVGM
NFSALTHDWIEKINAHPPFKKLIKTYFNDNDLKSMNVKGASQGMFMTYA
LAHELLTIIKEVITSCQSIDSVPEYNTEDIWFQFALLILEKKTGHVFNK
TSTLTYMPWERKLQWTNEQIESAKRGENIPVNKFIINSITL

SEQ ID NO: 21: is an amino acid sequence of a polypeptide having chondroitin synthase activity:chimeric amino acid comprising a fragment from an amino acid encoding a hyaluronan synthase originating from Pasteurella multocida, a fragment from an amino acid encoding a chondroitin synthase from Pasteurella multocida and a fragment from an amino acid encoding a chitin synthase 2 from Saccharomyces cerevisiae (HCOS.Sc)

MNTLSQAIKAYNSNDYQLALKLFEKSAEIYGRKIVEFQITKCKEKLSAH
PSVNSAHLSVNKEEKVNVCDSPLDIATQLLLSNVKKLVLSDSEKNTLKN
KWKLLTEKKSENAEVRAVALVPKDFPKDLVLAPLPDHVNDFTWYKNRKK
SLGIKPVNKNIGLSIIIPTFNRSRILDITLACLVNQKTNYPFEVVVADD
GSKENLLTIVQKYEQKLDIKYVRQKDYGYQLCAVRNLGLRTAKYDFVSI
LDCDMAPQQLWVHSYLTELLEDNDIVLIGPRKYVDTHNITAEQFLNDPY
LIESLPETATNNNPSITSKGNISLDWRLEHFKKTDNLRLCDSPFRYFSC
GNVAFSKEWLNKVGWFDEEFNHWGGEDVEFGYRLFAKGCFFRVIDGGMA
YHQEPPGKENETDREAGKSITLKIVKEKVPYIYRKLLPIEDSHINRVPL
VSIYIPAYNCANYIQRCVDSALNQTVVDLEVCICNDGSTDNTLEVINKL
YGNNPRVRIMSKPNGGIASASNAAVSFAKGYYIGOLDSDDYLEPDAVEL
CLKEFLKDKTLACVYTTNRNVNPDGSLIANGYNWPEFSREKLTTAMIAH
HFRMFTIRAWHLTDGFNEKIENAVDYDMFLKLSEVGKFKHLNKICYNRV
LHGDNTSIKKLGIQKKNHFVVVNQSLNRQGITYYNYDEFDDLDESRKYI
FNKTAEYQEEIDILKDIFIQMLFSWFSIANFVLTFYYLAGSMNLVIKHG
EALFIFFKYLIFCDLASLFIISMGNRPQGAKHLFITSMVILSICATYSL
ICGFVFAFKSLASGTESHKIFVDIVISLLSTYGLYFFSSLMYLDPWHMF
TSSIQYFLTLPAFTCTLQIFAFCNTHDVSWGTKGSTQESKQLSKAIVVQ
GPDGKQIVETDWPQEVDKKFLEIKSRLKEPEFEESSGNEKQSKNDYYRD
IRTRIVMIWMLSNLILIMSIIQVFTPQDTDNGYLIFILWSVAALAAFRV
VGSMAFLFMKYLRIIVSYRNKVEGSGSWEVSKLDLPNVFHKKG

SEQ ID NO: 22: is an amino acid sequence of a polypeptide having chondroitin synthase activity:chimeric amino acid comprising a fragment from an amino acid encoding a hyaluronan synthase originating from Pasteurella multocida and a fragment from an amino acid encoding a chitin synthase 2 from Saccharomyces cerevisiae (HHASA.Sc)

MNTLSQAIKAYNSNDYQLALKLFEKSAEIYGRKIVEFQITKCKEKLSAH
PSVNSAHLSVNKEEKVNVCDSPLDIATQLLLSNVKKLVLSDSEKNTLKN
KWKLLTEKKSENAEVRAVALVPKDFPKDLVLAPLPDHVNDFTWYKKRKK
RLGIKPEHQHVGLSIIVTTFNRPAILSITLACLVNQKTHYPFEVIVTDD
GSQEDLSPIIRQYENKLDIRYVRQKDNGFQASAARNMGLRLAKYDFIGL
LDCDMAPNPLWVHSYVAELLEDDDLTIIGPRKYIDTQHIDPKDFLNNAS
LLESLPEVKTNNSVAAKGEGTVSLDWRLEQFEKTENLRLSDSPFRFFAA
GNVAFAKKWLNKSGFFDEEFNHWGGEDVEFGYRLFRYGSFFKTIDGIMA
YHQEPPGKENETDREAGKNITLDIMREKVPYIYRKLLPIEDSHINRVPL
VSIYIPAYNCANYIQRCVDSALNQTVVDLEVCICNDGSTDNTLEVINKL
YGNNPRVRIMSKPNGGIASASNAAVSFAKGYYIGQLDSDDYLEPDAVEL
CLKEFLKDKTLACVYTTNRNVNPDGSLIANGYNWPEFSREKLTTAMIAH
HFRMFTIRAWHLTDGFNEKIENAVDYDMFLKLSEVGKFKHLNKICYNRV
LHGDNTSIKKLGIQKKNHFVVVNQSLNRQGITYYNYDEFDDLDESRKYI
FNKTAEYQEEIDILKDIFIQMLFSWFSIANFVLTFYYLAGSMNLVIKHG
EALFIFFKYLIFCDLASLFIISMGNRPQGAKHLFITSMVILSICATYSL
ICGFVFAFKSLASGTESHKIFVDIVISLLSTYGLYFFSSLMYLDPWHMF
TSSIQYFLTLPAFTCTLQIFAFCNTHDVSWGTKGSTQESKQLSKAIVVQ
GPDGKQIVETDWPQEVDKKFLEIKSRLKEPEFEESSGNEKQSKNDYYRD
IRTRIVMIWMLSNLILIMSIIQVFTPQDTDNGYLIFILWSVAALAAFRV
VGSMAFLFMKYLRIIVSYRNKVEGSGSWEVSKLDLPNVFHKKG

SEQ ID NO: 23: is an amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin synthase from Escherichia coli (HCOS1-Vir)

MGKNIIIMVSWYTIITSNLIAVGGASLILAPAITGYVLHWNIALSTIWG
VSAYGIFVFGFFLAQVLFSELNRKRLRKWISLRPKGWNDVRLAVIIAGY
REDPYMFQKCLESVRDSDYGNVARLICVIDGDEDDDMRMAAVYKAIYND
NIKKPEFVLCESDDKEGERIDSDFSRDICVLQPHRGKRECLYTGFQLAK
MDPSVNAVVLIDSDTVLEKDAILEVVYPLACDPEIQAVAGECKIWNTDT
LLSLLVAWRYYSAFCVERSAQSFFRTVQCVGGPLGAYKIDIIKEIKDPW
ISQRFLGQKCTYGDDRRLTNEILMRGKKVVFTPFAVGWGWFDEEFTHWG
GRYIVQQTRWSKSWCREIWYTLFAAWKHGLSGIWLAFECLYQITYFFLV
IYLFSRLAVEADPRAQTATVIVSTTVALIKCGYFSFRAKDIRAFYFVLY
TFVYFFCMIPARITAMMTLWDIGWGTRGGNEKPSVGTRVALWAKQYLIA
YMWWAAVVGAGVYSIVHNWMFDWNSLSYRFALVGICSYIVFIVIVLVVY
FTGKITTWNFTKLQKELIEDRVLYDATTNAQSV

SEQ ID NO: 24: is a reencoded amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin synthase from Escherichia coli (HCOS2-Vir)

MGKNIIIMVSWYTIITSNLIAVGGASLILAPAITGYVLHWNIALSTIWG
VSAYGIFVFGFFLAQVLFSELNRKRLRKWISLRPKGWNDVRLAVIIAGY
REDPYMFQKCLESVRDSDYGNVARLICVIDGDEDDDMRMAAVYKAIYND
NIKKPEFVLCESDDKEGERIDSDFSRDICVLQPHRGKRECLYTGFQLAK
MDPSVNAVVLIDSDTVLEKDAILEVVYPLACDPEIQAVAGECKIWNTDT
LLSLLVAWRYYSAFCVERSAQSFFRTVQCVGGPLGAYKIDIIKEIKDPW
ISQRFLGQKCTYGDDRRLTNEILMRGKKVVFTPFAVGWLHGENTSIKKL
RYIVQQTRWSKSWCREIWYTLFAAWKHGLSGIWLAFECLYQITYFFLVI
YLFSRLAVEADPRAQTATVIVSTTVALIKCGYFSFRAKDIRAFYFVLYT
FVYFFCMIPARITAMMTLWDIGWGTRGGNEKPSVGTRVALWAKQYLIAY
MWWAAVVGAGVYSIVHNWMFDWNSLSYRFALVGICSYIVFIVIVLVVYF
TGKITTWNFTKLQKELIEDRVLYDATTNAQSV

SEQ ID NO: 25: is a reencoded amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a Galactofuranosyltransferase from Mycobacterium tuberculosis (HCOS3-Vir)

MGKNIIIMVSWYTIITSNLIAVGGASLILAPAITGYVLHWNIALSTIWG
VSAYGIFVFGFFLAQVLFSELNRKRLRKWISLRPKGWNDVRLAVIIAGY
REDPYMFQKCLESVRDSDYGNVARLICVIDGDEDDDMRMAAVYKAIYND
NIKKPEFVLCESDDKEGERIDSDFSRDICVLQPHRGKRECLYTGFQLAK
MDPSVNAVVLIDSDTVLEKDAILEVVYPLACDPEIQAVAGECKIWNTDT
LLSLLVAWRYYSAFCVERSAQSFFRTVQCVGGPLGAYKIDIIKEIKDPW
ISQRFLGQKCTYGDDRRLTNEILMRGKKVVFTPFAVGWHMAWSDKDDAI
DRYIVQQTRWSKSWCREIWYTLFAAWKHGLSGIWLAFECLYQITYFFLV
IYLFSRLAVEADPRAQTATVIVSTTVALIKCGYFSFRAKDIRAFYFVLY
TFVYFFCMIPARITAMMTLWDIGWGTRGGNEKPSVGTRVALWAKQYLIA
YMWWAAVVGAGVYSIVHNWMFDWNSLSYRFALVGICSYIVFIVIVLVVY
FTGKITTWNFTKLQKELIEDRVLYDATTNAQSV

SEQ ID NO: 26: is a reencoded amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin synthase from Escherichia coli (HCOS4-Vir)

MGKNIIIMVSWYTIITSNLIAVGGASLILAPAITGYVLHWNIALSTIWG
VSAYGIFVFGFFLAQVLFSELNRKRLRKWISLRPKGWNDVRLAVIIAGY
REDPYMFQKCLESVRDSDYGNVARLICVIDGDEDDDMRMAAVYKAIYND
NIKKPEFVLCESDDKEGERIDSDFSRDICVLQPHRGKRECLYTGFQLAK
MDPSVNAVVLIDSDTVLEKDAILEVVYPLACDPEIQAVAGECKIWNTDT
LLSLLVAWRYYSAFCVERSAQSFFRTVQCVGGPLGAYKIDIIKEIKDPW
ISQRFLGQKCTYGDDRRLTNEILMRGKKVVFTPFAVGWFDEEFTHWGGR
YIVQQTRWSKSWCREIWYTLFAAWKHGLSGIWLAFECLYQITYFFLVIY
LFSRLAVEADPRAQTATVIVSTTVALIKCGYFSFRAKDIRAFYFVLYTF
VYFFCMIPARITAMMTLWDIGWGTRGGNEKPSVGTRVALWAKQYLIAYM
WWAAVVGAGVYSIVHNWMFDWNSLSYRFALVGICSYIVFIVIVLVVYFT
GKITTWNFTKLQKELIEDRVLYDATTNAQSV

SEQ ID NO: 27: is an amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin synthase from Penicillium oxalicum (HCOS5-Vir)

MGKNIIIMVSWYTIITSNLIAVGGASLILAPAITGYVLHWNIALSTIWG
VSAYGIFVFGFFLAQVLFSELNRKRLRKWISLRPKSIHHPISKTTHPDT
ENAGDSTLLCPPMDGMDDILVVMKTGVTEAKEKVPVHFQTTLRCVPHYV
IYSDFEEEIEGVKIYDVLQGMDPDVMTQVPDFDLYNRIKKLGRQGLAEN
DFADEANSAVGKPNNPGWKLDKWKFLPMIIEALRYKPKAKWYVFMEADT
YISWPTLLAWLKHFNHKKPWYLGTETQIADVIFAHGGSGFMISHAALKL
AAEEYTARRTELHHYTDGHWAGDCVLGKVLADAGVNLHFSWPILQNSNL
GELDEFNVDLYRQPWCFPAAAFHHLNAQKIQDLWSYEQKRWQDKDKRVL
LHSDVFRDLLYPDIALKPARIGWDNLSADEQSTASSFEECRQICDQLDD
CTQFSFRDKSCFTSRTPRLGAANPNTVSGWMVDRIRANMEKSGLFAAWK
HGLSGIWLAFECLYQITYFFLVIYLFSRLAVEADPRAQTATVIVSTTVA
LIKCGYFSFRAKDIRAFYFVLYTFVYFFCMIPARITAMMTLWDIGWGTR
GGNEKPSVGTRVALWAKQYLIAYMWWAAVVGAGVYSIVHNWMFDWNSLS
YRFALVGICSYIVFIVIVLVVYFTGKITTWNFTKLQKELIEDRVLYDAT
TNAQSV

SEQ ID NO: 28: is an amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin synthase from Pasteurella multocida (HCOS6-Vir)

MGKNIIIMVSWYTIITSNLIAVGGASLILAPAITGYVLHWNIALSTIWG
VSAYGIFVFGFFLAQVLFSELNRKRLRKWISLRPKLDIATQLLLSNVKK
LTLSESEKNSLKNKWKSITGKKSENAEIRKVELVPKDFPKDLVLAPLPD
HVNDFTWYKNRKKSLGIKPVNKNIGLSIIIPTFNRSRILDITLACLVNQ
KTNYPFEVVVADDGSKENLLTIVQKYEQKLDIKYVRQKDYGYQLCAVRN
LGLRTAKYDFVSILDCDMAPQQLWVHSYLTELLEDNDIVLIGPRKYVDT
HNITAEQFLNDPYLIESLPETATNNNPSITSKGNISLDWRLEHFKKTDN
LRLCDSPFRYFSCGNVAFSKEWLNKVGWFDEEFNHWGGEDVEFGYRLFA
KGCFFRVIDGGMAYHQEPPGKENETDREAGKSITLKIVKEKVPYIYRKL
LPIEDSHIHRIPLVSIYIPAYNCANYIQRCVDSALNQTVVDLEVCICND
GSTDNTLEVINKLYGNNPRVRIMSKPNGGIASASNAAVSFAKGYYIGQL
DSDDYLEPDAVELCLKEFLKDKTLACVYTTNRNVNPDGSLIANGYNWPE
FSREKLTTAMIAHHFRMFTIRAWHLTDGFNEKIENAVDYDMFLKLSEVG
KFKHLNKICYNRVLHGDNTSIKNLDTQKKNHFVVVNQSLNRQRVSNYNY
DEFDNLDESRKYIFNKLFAAWKHGLSGIWLAFECLYQITYFFLVIYLFS
RLAVEADPRAQTATVIVSTTVALIKCGYFSFRAKDIRAFYFVLYTFVYF
FCMIPARITAMMTLWDIGWGTRGGNEKPSVGTRVALWAKQYLIAYMWWA
AVVGAGVYSIVHNWMFDWNSLSYRFALVGICSYIVFIVIVLVVYFTGKI
TTWNFTKLQKELIEDRVLYDATTNAQSV

SEQ ID NO: 29: is a reencoded amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin synthase from Pasteurella multocida (HCOS7-Vir)

MGKNIIIMVSWYTIITSNLIAVGGASLILAPAITGYVLHWNIALSTIWG
VSAYGIFVFGFFLAQVLFSELNRKRLRKWISLRPKIPTFNRSRILDITL
ACLVNQKTNYPFEVVVADDGSKENLLTIVQKYEQKLDIKYVRQKDYGYQ
LCAVRNLGLRTAKYDFVSILDCDMAPQQLWVHSYLTELLEDNDIVLIGP
RKYVDTHNITAEQFLNDPYLIESLPETATNNNPSITSKGNISLDWRLEH
FKKTDNLRLCDSPFRYFSCGNVAFSKEWLNKVGWFDEEFNHWGGEDVEF
GYRLFAKGCFFRVIDGGMAYHQEPPGKENETDREAGKSITLKIVKEKVP
YIYRKLLPIEDSHIHRIPLVSIYIPAYNCANYIQRCVDSALNQTVVDLE
VCICNDGSTDNTLEVINKLYGNNPRVRIMSKPNGGIASASNAAVSFAKG
YYIGQLDSDDYLEPDAVELCLKEFLKDKTLACVYTTNRNVNPDGSLIAN
GYNWPLFAAWKHGLSGIWLAFECLYQITYFFLVIYLFSRLAVEADPRAQ
TATVIVSTTVALIKCGYFSFRAKDIRAFYFVLYTFVYFFCMIPARITAM
MTLWDIGWGTRGGNEKPSVGTRVALWAKQYLIAYMWWAAVVGAGVYSIV
HNWMFDWNSLSYRFALVGICSYIVFIVIVLVVYFTGKITTWNFTKLQKE
LIEDRVLYDATTNAQSV

SEQ ID NO: 30: is a reencoded amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin synthase from Pasteurella multocida (HCOS8-Vir)

MGKNIIIMVSWYTIITSNLIAVGGASLILAPAITGYVLHWNIALSTIWG
VSAYGIFVFGFFLAQVLFSELNRKRLRKWISLRPKGWNDVRLAVIIAGY
REDPYMFQKCLESVRDSDYGNVARLICVIDGDEDDDMRMAAVYKAIYND
NIKKPEFVLCESDDKEGERIDSDFSRDICVLQPHRGKRECLYTGFQLAK
MDPSVNAVVLIDSDTVLEKDAILEVVYPLACDPEIQAVAGECKIWNTDT
LLSLLVAWRYYSAFCVERSAQSFFRTVQCVRLCDSPFRYFSCGNVAFSK
EWLNKVGWFDEEFNHWGGEDVEFGYRLKKVVFTPFAVGWSDSPTNVFRY
IVQQTRWSKSWCREIWYTLFAAWKHGLSGIWLAFECLYQITYFFLVIYL
FSRLAVEADPRAQTATVIVSTTVALIKCGYFSFRAKDIRAFYFVLYTFV
YFFCMIPARITAMMTLWDIGWGTRGGNEKPSVGTRVALWAKQYLIAYMW
WAAVVGAGVYSIVHNWMFDWNSLSYRFALVGICSYIVFIVIVLVVYFTG
KITTWNFTKLQKELIEDRVLYDATTNAQSV

SEQ ID NO: 31: is an amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin sulfate synthase from Homo sapiens (HCOS9-Vir)

MGKNIIIMVSWYTIITSNLIAVGGASLILAPAITGYVLHWNIALSTIWG
VSAYGIFVFGFFLAQVLFSELNRKRLRKWISLRPKHIGKCLREMYTTHE
DVEVGRCVRRFAGVQCVWSYEMQQLFYENYEQNKKGYIRDLHNSKIHQA
ITLHPNKNPPYQYRLHSYMLSRKISELRHRTIQLHREIVLMSKYSNTEI
HKEDLQLGIPPSFMRFQPRQREEILEWEFLTGKYLYSAVDGQPPRRGMD
SAQREALDDIVMQVMEMINANAKTRGRIIDFKEIQYGYRRVNPMYGAEY
ILDLLLLYKKHKGKKMTVPVRRHAYLQQTFSKIQFVEHEELDAQELAKR
INQESGSLSFLSNSLKKLVPFQLPGSKSEHKEPKDKKINILIPLSGRFD
MFVRFMGNFEKTCLIPNQNVKLVVLLFNSDSNPDKAKQVELMRDYRIKY
PKADMQILPVSGEFSRALALEVGSSQFNNESLLFFCDVDLVFTTEFLQR
CRANTVLGQQIYFPIIFSQYDPKIVYSGKVPSDNHFAFTQKTGFWRNYG
FGITCIYKGDLVRVGGFDVSIQGWGLEDVDLFNKVVQAGLKTFRSQEVG
VVHVHHPVFCDPNLDPKQYKMCLGSKASTYGSTQQLLFAAWKHGLSGIW
LAFECLYQITYFFLVIYLFSRLAVEADPRAQTATVIVSTTVALIKCGYF
SFRAKDIRAFYFVLYTFVYFFCMIPARITAMMTLWDIGWGTRGGNEKPS
VGTRVALWAKQYLIAYMWWAAVVGAGVYSIVHNWMFDWNSLSYRFALVG
ICSYIVFIVIVLVVYFTGKITTWNFTKLQKELIEDRVLYDATTNAQSV

SEQ ID NO: 32: is a reencoded amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin sulfate synthase from Homo sapiens (HCOS10-Vir)

MGKNIIIMVSWYTIITSNLIAVGGASLILAPAITGYVLHWNIALSTIWG
VSAYGIFVFGFFLAQVLFSELNRKRLRKWISLRPKRDRNFLFVGVMTAQ
KYLQTRAVAAYRTWSKTIPGKVQFFSSEGSDTSVPIPVVPLRGVDDSYP
PQKKSFMMLKYMHDHYLDKYEWFMRADDDVYIKGDRLENFLRSLNSSEP
LFLGQTGLGTTEEMGKLALEPGENFCMGGPGVIMSREVLRRMVPHIGKC
LREMYTTHEDVEVGRCVRRFAGVQCVWSYEMQQLFYENYEQNKKGYIRD
LHNSKIHQAITLHPNKNPPYQYRLHSYMLSRKISELRHRTIQLHREIVL
MSKYSNTEIHKEDLQLGIPPSFMRFQPRQREEILEWEFLTGKYLYSAVD
GQPPRRGMDSAQREALDDIVMQVMEMINANAKTRGRIIDFKEIQYGYRR
VNPMYGAEYILDLLLLYKKHKGKKMTVPVRRHAYLQQTFSKIQFVEHEE
LDAQELAKRINQESGSLSFLSNSLKKLVPFQLPGSKSEHKEPKDKKINI
LIPLSGRFDMFVRFMGNFEKTCLIPNQNVKLVVLLFNSDSNPDKAKQVE
LMRDYRIKYPKADMQILPVSGEFSRALALEVGSSQFNNESLLFFCDVDL
VFTTEFLQRCRANTVLGQQIYFPIIFSQYDPKIVYSGKVPSDNHFAFTQ
KTGFWRNYGFGITCIYKGDLVRVGGFDVSIQGWGLEDVDLFNKVVQAGL
KTFRSQEVGVVHVHHPVFCDPNLDPKQYKMCLGSKASTYGSTQQLAEMW
LEKNDPSYSLFAAWKHGLSGIWLAFECLYQITYFFLVIYLFSRLAVEAD
PRAQTATVIVSTTVALIKCGYFSFRAKDIRAFYFVLYTFVYFFCMIPAR
ITAMMTLWDIGWGTRGGNEKPSVGTRVALWAKQYLIAYMWWAAVVGAGV
YSIVHNWMFDWNSLSYRFALVGICSYIVFIVIVLVVYFTGKITTWNFTK
LQKELIEDRVLYDATTNAQSV

SEQ ID NO: 33: is a reencoded amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin sulfate synthase from Homo sapiens (HCOS11-Vir)

MGKNIIIMVSWYTIITSNLIAVGGASLILAPAITGYVLHWNIALSTIWG
VSAYGIFVFGFFLAQVLFSELNRKRLRKWISLRPKRDRNFLFVGVMTAQ
KYLQTRAVAAYRTWSKTIPGKVQFFSSEGSDTSVPIPVVPLRGVDDSYP
PQKKSFMMLKYMHDHYLDKYEWFMRADDDVYIKGDRLENFLRSLNSSEP
LFLGQTGLGTTEEMGKLALEPGENFCMGGPGVIMSREVLRRMVPHIGK
CLREMYTTHEDVEVGRCVRRFAGVQCVWSYEMQQLFYENYEQNKKGYIR
DLHNSKIHQAITLHPNKNPPYQYRLHSYMLLFAAWKHGLSGIWLAFECL
YQITYFFLVIYLFSRLAVEADPRAQTATVIVSTTVALIKCGYFSFRAKD
IRAFYFVLYTFVYFFCMIPARITAMMTLWDIGWGTRGGNEKPSVGTRVA
LWAKQYLIAYMWWAAVVGAGVYSIVHNWMFDWNSLSYRFALVGICSYIV
FIVIVLVVYFTGKITTWNFTKLQKELIEDRVLYDATTNAQSV

SEQ ID NO: 34: is a reencoded amino acid sequence of a polypeptide having chondroitin synthase activity:chimeric amino acid comprising a fragment from an amino acid encoding a hyaluronan synthase originating from Chlorella virus PBCV-1 and a fragment from an amino acid encoding a chondroitin sulfate synthase from Homo sapiens (HCOS12-Vir)

MGKNIIIMVSWYTIITSNLIAVGGASLILAPAITGYVLHWNIALSTIWG
VSAYGIFVFGFFLAQVLFSELNRKRLRKWISLRPKKDKKINILIPLSGR
FDMFVRFMGNFEKTCLIPNQNVKLVVLLFNSDSNPDKAKQVELMRDYRI
KYPKADMQILPVSGEFSRALALEVGSSQFNNESLLFFCDVDLVFTTEFL
QRCRANTVLGQQIYFPIIFSQYDPKIVYSGKVPSDNHFAFTQKTGFWRN
YGFGITCIYKGDLVRVGGFDVSIQGWGLEDVDLFNKVVQAGLKTFRSQE
VGVVHVHHPVFCDPNLDPKQYKMCLGSKASTYGSTQQLAEMWLEKNDPS
YSLFAAWKHGLSGIWLAFECLYQITYFFLVIYLFSRLAVEADPRAQTAT
VIVSTTVALIKCGYFSFRAKDIRAFYFVLYTFVYFFCMIPARITAMMTL
WDIGWGTRGGNEKPSVGTRVALWAKQYLIAYMWWAAVVGAGVYSIVHNW
MFDWNSLSYRFALVGICSYIVFIVIVLVVYFTGKITTWNFTKLQKELIE
DRVLYDATTNAQSV

SEQ ID NO: 35 is a nucleic acid sequence of UDP-Glucose dehydrogenase (HASB) originating from Arabidopsis thaliana

ATGGTCAAAATATGCTGTATCGGGGCTGGATATGTCGGTGGGCCTACAA
TGGCCGTTATGGCCCTAAAGTGTCCAGAGATTGAAGTCGTGGTAGTTGA
TATATCGGAGCCACGAATTAACGCATGGAACTCAGACCGTCTACCAATT
TATGAGCCAGGGTTAGAGGATGTCGTCAAACAGTGTAGAGGGAAGAATT
TGTTCTTCTCTACAGATGTAGAGAAGCATGTATTTGAGTCAGATATAGT
GTTTGTTTCGGTAAACACTCCTACGAAAACGCAGGGTCTGGGTGCAGGT
AAAGCAGCAGATTTGACATATTGGGAGTCCGCTGCTCGCATGATAGCTG
ATGTGAGCAAATCGTCGAAAATCGTCGTTGAAAAGAGTACAGTACCAGT
TCGTACAGCCGAGGCTATAGAAAAGATTTTAACGCACAATTCGAAGGGT
ATCGAATTCCAGATCTTATCAAATCCAGAATTCTTGGCCGAAGGGACGG
CGATTAAAGACTTATATAACCCTGATAGAGTTCTAATCGGTGGCAGGGA
CACCGCTGCGGGACAAAAGGCCATTAAGGCGTTGCGTGACGTGTATGCC
CACTGGGTTCCTGTTGAGCAAATAATCTGTACTAATTTATGGAGTGCCG
AGCTATCAAAGTTGGCTGCGAACGCATTTCTAGCTCAAAGGATAAGTTC
AGTAAACGCAATGTCAGCGCTTTGTGAGGCAACTGGTGCTGACGTAACC
CAAGTCGCTCACGCCGTTGGAACCGACACTAGAATTGGACCGAAGTTTC
TTAACGCATCCGTAGGCTTCGGCGGATCTTGCTTTCAGAAAGACATCCT
GAATCTTATTTACATCTGCGAATGCAATGGTCTTCCAGAAGCAGCCAAT
TATTGGAAACAGGTAGTCAAGGTAAATGACTACCAAAAGATTAGGTTTG
CTAATCGAGTCGTATCTTCTATGTTCAACACCGTCTCCGGTAAGAAAAT
TGCTATTTTGGGATTTGCGTTCAAGAAGGACACCGGCGACACGCGTGAA
ACTCCTGCCATAGATGTGTGTAATCGCCTCGTGGCTGATAAAGCAAAGC
TGTCGATCTATGATCCGCAAGTATTAGAAGAGCAGATCCGCCGTGATCT
GTCCATGGCCCGATTCGATTGGGACCATCCAGTCCCACTCCAGCAGATC
AAAGCTGAAGGTATCTCCGAACAGGTTAACGTTGTGTCCGACGCCTACG
AGGCTACGAAGGATGCTCATGGTTTATGTGTTTTAACCGAATGGGACGA
ATTCAAGTCACTTGATTTTAAGAAGATCTTTGATAATATGCAGAAACCC
GCTTTCGTTTTCGACGGAAGAAACGTGGTCGACGCTGTGAAATTGAGAG
AAATTGGATTCATAGTATATTCCATAGGTAAACCTCTGGATAGTTGGCT
CAAGGATATGCCGGCTGTTGCATAA

SEQ ID NO: 36 is a reencoded nucleic acid sequence of UDP-Glucose dehydrogenase (HASB) originating from Chlorella virus PBCV-1

ATGAGTAGAATTGCTGTCGTTGGATGCGGTTACGTGGGTACGGCCTGCG
CCGTACTTTTGGCGCAGAAGAACGAAGTTATCGTTTTGGATATCTCGGA
GGACCGGGTACAACTGATTAAGAATAAGAAGTCACCTATAGAAGATAAG
GAAATCGAAGAATTCCTGGAGACGAAAGATTTGAATCTAACAGCGACGA
CGGATAAGGTGCTCGCCTATGAGAATGCTGAATTCGTTATAATAGCTAC
ACCGACCGATTACGATGTCGTCACTAGATATTTCAACACTAAGTCTGTT
GAAAATGTGATAGGCGATGTCATTAAGAACACTCAGACACACCCTACTA
TAGTGATCAAGAGTACTATTCCAATCGGTTTCGTGGATAAAGTTAGAGA
ACAGTTCGATTATCAAAATATTATCTTCTCGCCGGAATTCTTGAGAGAA
GGACGAGCATTATATGATAATCTTTACCCCTCCCGTATCATCGTCGGTG
ATGACTCCCCAATTGCCTTAAAATTCGCGAATCTCTTGGTCGAGGGTAG
CAAAACTCCACTAGCTCCCGTATTAACTATGGGTACGCGAGAGGCCGAA
GCTGTAAAACTATTTTCAAATACATATTTGGCTATGAGAGTAGCATACT
TCAATGAGCTAGACACATTTGCAATGTCCCATGGTATGAACGCAAAAGA
GATTATCGACGGAGTCACACTAGAACCAAGGATAGGGCAGGGTTATTCG
AATCCTTCATTTGGGTATGGAGCCTATTGTTTCCCAAAAGATACAAAGC
AATTGTTGGCTAATTTTGAAGGGGTTCCACAAGATATTATTGGCGCAAT
CGTAGAGTCTAACGAAACTAGAAAGGAGGTTATTGTTTCTGAGGTCGAG
AACAGATTTCCGACCACAGTTGGCGTATATAAGTTGGCTGCTAAAGCTG
GTTCGGATAATTTCAGGTCTAGTGCGATAGTCGATATCATGGAAAGGTT
GGCTAATAAGGGCTACCACATCAAAATATTTGAACCTACCGTTGAACAA
TTTGAGAATTTTGAGGTAGATAATAATCTCACAACTTTTGCAACCGAGT
CTGATGTAATAATTGCGAACAGAGTTCCAGTCGAACATCGCATTCTGTT
TGGGAAGAAGTTAATAACACGGGATGTCTATGGGGATAA

SEQ ID NO: 37 is a reencoded nucleic acid sequence of UDP-Glucose dehydrogenase (HASB-A) originating from Chlorella virus PBCV-1

ATGTCACGAATCGCAGTAGTTGGCTGTGGGTACGTGGGAACCGCATGCG
CTGTACTGCTGGCGCAAAAGAATGAGGTAATCGTTCTTGATATTAGTGA
GGATAGGGTCCAACTAATCAAGAACAAGAAATCCCCGATCGAAGATAAG
GAGATTGAAGAATTCTTGGAAACAAAAGACCTAAACTTAACTGCAACAA
CTGACAAAGTTTTAGCCTACGAAAACGCTGAGTTTGTGATTATAGCGAC
ACCCACAGATTATGACGTAGTTACCAGATACTTTAACACGAAGTCCGTA
GAGAACGTCATTGGAGATGTTATAAAGAATACTCAGACTCATCCTACGA
TAGTAATAAAGTCAACCATTCCCATAGGTTTCGTAGATAAGGTTAGGGA
GCAATTCGATTACCAGAACATTATATTTTCGCCAGAATTTCTGAGAGAG
GGTCGCGCCCTGTATGATAATCTATATCCATCACGGATTATAGTGGGCG
ATGACTCTCCGATTGCACTTAAGTTTGCTAATCTTTTAGTTGAGGGCTC
CAAAACTCCGCTCGCCCCAGTACTTACGATGGGTACACGTGAGGCTGAA
GCTGTCAAGCTGTTTTCAAACACATACCTTGCTATGCGAGTCGCATACT
TTAACGAACTAGATACCTTTGCTATGTCGCACGGTATGAATGCTAAAGA
AATCATAGATGGCGTAACGTTGGAGCCTCGGATAGGTCAAGGATATTCC
AATCCATCTTTTGGCTACGGTGCGTATTGTTTCCCAAAGGACACGAAGC
AATTATTAGCTAACTTTGAGGGTGTTCCGCAAGATATAATTGGGGCGAT
AGTAGAAAGCAATGAAACACGGAAGGAGGTAATCGTGAGTGAAGTGGAA
AACCGATTCCCCACTACGGTCGGCGTTTACAAATTAGCCGCCAAGGCTG
GTTCCGACAATTTCCGATCCAGCGCAATAGTAGATATTATGGAAAGATT
AGCTAATAAGGGATACCACATTAAAATCTTTGAACCTACTGTCGAACAG
TTCGAGAACTTCGAGGTTGATAATAACTTGACGACTTTCGCAACGGAGA
GCGATGTAATTATTGCAAACCGCGTACCTGTGGAACATCGAATTTTGTT
CGGAAAGAAGCTGATTACACGCGATGTATATGGCGATAACTAA

SEQ ID NO: 38 is a nucleic acid sequence of UDP-Glucose dehydrogenase (HASB) originating from Streptococcus zooepidemicus

ATGAAGATATCGGTAGCGGGTTCGGGGTACGTGGGGTTATCCTTGTCAAT
CTTGCTTGCCCAACATAACGATGTGACTGTTGTAGATATAATCGACGAAA
AGGTACGGCTAATTAACCAGGGCATATCTCCGATTAAGGATGCGGACATT
GAGGAATATCTGAAGAATGCACCGTTGAATCTTACGGCTACACTAGACGG
AGCTTCGGCTTATAGTAATGCTGATCTGATTATAATCGCAACGCCAACTA
ATTACGATTCAGAACGCAATTATTTCGACACCAGACACGTTGAAGAAGTA
ATTGAGCAAGTATTGGATTTAAATGCCTCCGCTACTATAATCATCAAGAG
TACCATACCCTTGGGTTTTATTAAACACGTAAGAGAGAAATACCAAACAG
ACAGAATCATCTTTTCTCCAGAGTTCTTAAGAGAGTCAAAGGCATTGTAC
GATAACTTATACCCCTCTCGTATAATAGTCAGTTATGAGAAGGATGACTC
TCCAAGAGTTATACAAGCAGCTAAGGCGTTCGCGGGTTTATTAAAAGAGG
GGGCAAAGAGCAAGGATACCCCAGTTCTGTTTATGGGCTCTCAAGAAGCT
GAAGCTGTCAAGCTGTTTGCTAATACTTTTCTCGCCATGAGGGTCAGTTA
CTTCAACGAGCTTGACACTTATAGCGAATCAAAAGGACTAGACGCCCAAA
GAGTTATAGAAGGCGTCTGCCATGATCAAAGGATAGGTAATCATTACAAT
AATCCATCCTTCGGATATGGCGGTTATTGTTTACCCAAAGACTCAAAGCA
ACTTTTGGCTAATTATAGAGGCATACCTCAGTCTCTAATGTCTGCCATCG
TTGAATCGAACAAGATCCGTAAGTCGTATTTAGCTGAACAAATATTAGAT
AGGGCTTCTTCACAAAAGCAGGCTGGTGTACCTTTAACCATAGGATTTTA
CCGTTTGATTATGAAGTCCAACTCCGATAACTTTAGAGAATCAGCCATTA
AAGATATTATTGACATCATTAATGACTACGGTGTCAATATTGTCATTTAT
GAACCTATGTTGGGAGAAGACATTGGTTATAGAGTCGTTAAAGATTTGGA
ACAGTTTAAGAACGAAAGTACAATTATTGTTAGTAACAGGTTTGAAGATG
ATTTAGGTGATGTTATTGATAAAGTTTATACACGTGACGTCTTTGGTAG

SEQ ID NO: 39 is an amino acid sequence of UDP-Glucose dehydrogenase (HASB) originating from Arabidopsis thaliana

MVKICCIGAGYVGGPTMAVMALKCPEIEVVVVDISEPRINAWNSDRLPIY
EPGLEDVVKQCRGKNLFFSTDVEKHVFESDIVFVSVNTPTKTQGLGAGKA
ADLTYWESAARMIADVSKSSKIVVEKSTVPVRTAEAIEKILTHNSKGIEF
QILSNPEFLAEGTAIKDLYNPDRVLIGGRDTAAGQKAIKALRDVYAHWVP
VEQIICTNLWSAELSKLAANAFLAQRISSVNAMSALCEATGADVTQVAHA
VGTDTRIGPKFLNASVGFGGSCFQKDILNLIYICECNGLPEAANYWKQVV
KVNDYQKIRFANRVVSSMFNTVSGKKIAILGFAFKKDTGDTRETPAIDVC
NRLVADKAKLSIYDPQVLEEQIRRDLSMARFDWDHPVPLQQIKAEGISEQ
VNVVSDAYEATKDAHGLCVLTEWDEFKSLDFKKIFDNMQKPAFVFDGRNV
VDAVKLREIGFIVYSIGKPLDSWLKDMPAVA

SEQ ID NO: 40 is an amino acid sequence of UDP-Glucose dehydrogenase (HASB) originating from Chlorella virus PBCV1

MSRIAVVGCGYVGTACAVLLAQKNEVIVLDISEDRVQLIKNKKSPIEDKE
IEEFLETKDLNLTATTDKVLAYENAEFVIIATPTDYDVVTRYFNTKSVEN
VIGDVIKNTQTHPTIVIKSTIPIGFVDKVREQFDYQNIIFSPEFLREGRA
LYDNLYPSRIIVGDDSPIALKFANLLVEGSKTPLAPVLTMGTREAEAVKL
FSNTYLAMRVAYFNELDTFAMSHGMNAKEIIDGVTLEPRIGQGYSNPSFG
YGAYCFPKDTKQLLANFEGVPQDIIGAIVESNETRKEVIVSEVENRFPTT
VGVYKLAAKAGSDNFRSSAIVDIMERLANKGYHIKIFEPTVEQFENFEVD
NNLTTFATESDVIIANRVPVEHRILFGKKLITRDVYGDN

SEQ ID NO: 41 is an amino acid sequence of UDP-Glucose dehydrogenase (HASB) originating from Streptococcus zooepidemicus

MKISVAGSGYVGLSLSILLAQHNDVTVVDIIDEKVRLINQGISPIKDADI
EEYLKNAPLNLTATLDGASAYSNADLIIIATPTNYDSERNYFDTRHVEEV
IEQVLDLNASATIIIKSTIPLGFIKHVREKYQTDRIIFSPEFLRESKALY
DNLYPSRIIVSYEKDDSPRVIQAAKAFAGLLKEGAKSKDTPVLFMGSQEA
EAVKLFANTFLAMRVSYFNELDTYSESKGLDAQRVIEGVCHDQRIGNHYN
NPSFGYGGYCLPKDSKQLLANYRGIPQSLMSAIVESNKIRKSYLAEQILD
RASSQKQAGVPLTIGFYRLIMKSNSDNFRESAIKDIIDIINDYGVNIVIY
EPMLGEDIGYRVVKDLEQFKNESTIIVSNRFEDDLGDVIDKVYTRDVFGR
D

SEQ ID NO: 42 is a nucleic acid sequence of UDP-glucose-4-epimerase (GNE1) originating from Pseudomonas aeruginosa

ATGATGTCCCGTTACGAGGAACTTCGTAAAGAGCTGCCTGCACAGCCGAA
GGTATGGTTGATCACTGGTGTCGCGGGCTTCATCGGGTCAAATCTGCTAG
AGACCTTGCTTAAATTGGATCAGAAGGTTGTTGGCTTGGACAACTTTGCA
ACTGGCCACCAGAGGAATCTGGACGAGGTGAGGTCATTGGTTTCTGAGAA
ACAGTGGAGCAACTTTAAGTTTATTCAAGGGGACATTAGAAATCTGGACG
ACTGCAACAACGCATGTGCAGGCGTCGATTATGTCTTACACCAGGCAGCA
TTGGGCAGTGTGCCGAGGTCTATAAACGACCCTATAACATCGAATGCAAC
TAACATAGACGGTTTCTTGAACATGCTGATTGCAGCGAGGGATGCAAAAG
TTCAGTCTTTTACCTATGCCGCTTCATCCTCTACATACGGGGATCACCCC
GGTTTGCCAAAGGTGGAAGACACTATCGGCAAGCCTCTATCTCCATACGC
TGTGACGAAGTATGTTAATGAGTTGTACGCAGACGTGTTTTCACGTTGTT
ATGGATTTTCAACCATCGGTCTGAGATACTTTAACGTCTTCGGCCGTAGA
CAAGATCCGAATGGGGCATACGCCGCCGTAATCCCTAAGTGGACATCCTC
AATGATACAGGGTGACGACGTCTACATTAACGGAGATGGAGAGACATCAC
GTGACTTCTGCTACATAGAAAACACAGTTCAAGCAAACTTATTAGCCGCA
ACGGCCGGCCTAGACGCCAGAAACCAGGTGTATAATATCGCGGTGGGTGG
TAGAACGTCACTAAATCAGCTTTTCTTCGCCTTGAGAGACGGTCTAGCTG
AGAACGGCGTTAGCTACCATAGGGAACCAGTTTATAGGGATTTTAGAGAA
GGAGATGTGAGACACAGCTTGGCAGATATTTCCAAGGCGGCTAAACTTCT
TGGGTATGCTCCTAAATATGACGTATCAGCAGGGGTGGCGCTTGCAATGC
CATGGTATATAATGTTTCTTAAGTAA

SEQ ID NO: 43 is a nucleic acid sequence of UDP-glucose-4-epimerase (KFOA) originating from Pasteurella multocida

ATGAACATTCTAGTGACCGGCGGTGCTGGCTATATCGGCTCCCATACCAG
TTTATGCTTACTGAATAAAGGATATAACGTGGTCATCATTGACAACCTGA
TCAATAGTTCATGCGAGTCGATCCGTAGGATAGAACTGATAGCTAAGAAG
AAGGTCACCTTCTACGAGCTGAACATTAATAATGAAAAGGAAGTGAACCA
GATCCTTAAGAAACACAAATTCGATTGCATAATGCACTTCGCAGGTGCCA
AGTCGGTGGCCGAGTCACTAATTAAGCCAATTTTCTACTATGACAACAAC
GTCTCTGGAACTTTGCAGCTGATTAATTGTGCTATAAAGAACGACGTCGC
AAATTTCATCTTTTCTTCATCAGCAACTGTTTATGGTGAGTCAAAGATTA
TGCCAGTGACAGAGGACTGCCACATCGGCGGCACACTAAATCCATACGGA
ACATCAAAATACATTTCAGAGTTGATGATAAGGGACATCGCCAAGAAGTA
CTCAGACACTAATTTCTTATGCTTGCGTTATTTCAATCCTACCGGGGCCC
ACGAGTCCGGCATGATCGGCGAGTCTCCAGCTGATATACCATCCAATCTG
GTGCCTTACATTTTACAGGTGGCCATGGGCAAGTTGGAGAAGTTAATGGT
ATTCGGCGGTGACTACCCAACAAAGGACGGCACGGGTGTGAGAGACTATA
TTCACGTGATGGACCTAGCGGAAGGTCATGTGGCAGCCCTAAGCTACCTT
TTCAGAGATAATAACACTAATTACCATGTATTCAATCTGGGCACGGGTAA
GGGCTACAGTGTCCTTGAATTAGTAAGCACATTCGAGAAAATCTCGGGTG
TCAGGATTCCATACGAGATCGTTTCTCGTCGTGATGGGGACATCGCAGAG
AGTTGGTCATCACCGGAGAAAGCGAACAAGTACTTAAATTGGAAGGCCAA
GAGGGAGCTTGAGACAATGCTAGAAGACGCATGGCGTTGGCAGATGAAGA
ACCCGAATGGTTACATATAA

SEQ ID NO: 44 is a nucleic acid sequence of UDP-glucose-4-epimerase (KFOA) originating from Escherichia coli

AATGAACATTCTAGTGACCGGCGGTGCTGGCTATATCGGCTCCCATACCA
GTTTATGCTTACTGAATAAAGGATATAACGTGGTCATCATTGACAACCTG
ATCAATAGTTCATGCGAGTCGATCCGTAGGATAGAACTGATAGCTAAGAA
GAAGGTCACCTTCTACGAGCTGAACATTAATAATGAAAAGGAAGTGAACC
AGATCCTTAAGAAACACAAATTCGATTGCATAATGCACTTCGCAGGTGCC
AAGTCGGTGGCCGAGTCACTAATTAAGCCAATTTTCTACTATGACAACAA
CGTCTCTGGAACTTTGCAGCTGATTAATTGTGCTATAAAGAACGACGTCG
CAAATTTCATCTTTTCTTCATCAGCAACTGTTTATGGTGAGTCAAAGATT
ATGCCAGTGACAGAGGACTGCCACATCGGCGGCACACTAAATCCATACGG
AACATCAAAATACATTTCAGAGTTGATGATAAGGGACATCGCCAAGAAGT
ACTCAGACACTAATTTCTTATGCTTGCGTTATTTCAATCCTACCGGGGCC
CACGAGTCCGGCATGATCGGCGAGTCTCCAGCTGATATACCATCCAATCT
GGTGCCTTACATTTTACAGGTGGCCATGGGCAAGTTGGAGAAGTTAATGG
TATTCGGCGGTGACTACCCAACAAAGGACGGCACGGGTGTGAGAGACTAT
ATTCACGTGATGGACCTAGCGGAAGGTCATGTGGCAGCCCTAAGCTACCT
TTTCAGAGATAATAACACTAATTACCATGTATTCAATCTGGGCACGGGTA
AGGGCTACAGTGTCCTTGAATTAGTAAGCACATTCGAGAAAATCTCGGGT
GTCAGGATTCCATACGAGATCGTTTCTCGTCGTGATGGGGACATCGCAGA
GAGTTGGTCATCACCGGAGAAAGCGAACAAGTACTTAAATTGGAAGGCCA
AGAGGGAGCTTGAGACAATGCTAGAAGACGCATGGCGTTGGCAGATGAAG
AACCCGAATGGTTACATATAA

SEQ ID NO: 45 is an amino acid sequence of UDP-glucose-4-epimerase (GNE1) originating from Pseudomonas aeruginosa

MMSRYEELRKELPAQPKVWLITGVAGFIGSNLLETLLKLDQKVVGLDNFA
TGHQRNLDEVRSLVSEKQWSNFKFIQGDIRNLDDCNNACAGVDYVLHQAA
LGSVPRSINDPITSNATNIDGFLNMLIAARDAKVQSFTYAASSSTYGDHP
GLPKVEDTIGKPLSPYAVTKYVNELYADVFSRCYGFSTIGLRYFNVFGRR
QDPNGAYAAVIPKWTSSMIQGDDVYINGDGETSRDFCYIENTVQANLLAA
TAGLDARNQVYNIAVGGRTSLNQLFFALRDGLAENGVSYHREPVYRDFRE
GDVRHSLADISKAAKLLGYAPKYDVSAGVALAMPWYIMFLK

SEQ ID NO: 46 is an amino acid sequence of UDP-glucose-4-epimerase (KFOA) originating from Pasteurella multocida

MAILVTGGAGYIGSHTVVELLNANKDVVVLDNLCNSSPKSLERVAQITGK
QVKFYQGDILDTALLQKIFAENQIQSVIHFAGLKAVGESVQKPAEYYMNN
VTGSLVLIQEMKKAGVWNFVFSSSATVYGDPEIIPITESCKVGGTTNPYG
TSKFMVEQILKDIAKATPEFSITILRYFNPVGAHESGLIGEDPNGIPNNL
LPYISQVAIGKLPQLSVFGSDYETHDGTGVRDYIHVVDLAIGHLKALDRH
EGDAGLHIYNLGTGSGYSVLDMVKAFEKVNDIKIPYKLVDRRPGDIATCY
SDPSLAKTELNWTAARGLEQMMKDTWHWQKKNPKGYRD

SEQ ID NO: 47 is an amino acid sequence of UDP-glucose-4-epimerase (KFOA) originating from Escherichia coli

MNILVTGGAGYIGSHTSLCLLNKGYNVVIIDNLINSSCESIRRIELIAKK
KVTFYELNINNEKEVNQILKKHKFDCIMHFAGAKSVAESLIKPIFYYDNN
VSGTLQLINCAIKNDVANFIFSSSATVYGESKIMPVTEDCHIGGTLNPYG
TSKYISELMIRDIAKKYSDTNFLCLRYFNPTGAHESGMIGESPADIPSNL
VPYILQVAMGKLEKLMVFGGDYPTKDGTGVRDYIHVMDLAEGHVAALSYL
FRDNNTNYHVFNLGTGKGYSVLELVSTFEKISGVRIPYEIVSRRDGDIAE
SWSSPEKANKYLNWKAKRELETMLEDAWRWQMKNPNGYI

SEQ ID NO: 48 is a reencoded nucleic acid sequence of hyaluronidase (HYAL) originating from Cupiennius salei with a N-terminal secretion signal

ATGCAATTCAGCACTGTTGCATCAGTTGCATTTGTCGCCCTGGCGAATTT
TGTAGCCGCGTTCAAGATTTACTGGAACGTCCCAACTTTTCAGTGCACGC
ATAACTACAAAATCGATTATGTCAAATTGTTGTCCACTTACGGGATACAG
GTCAATGATGGCGGTAAGTTTCAAGGAAACCAAGTGACTATCTTTTATGA
AACCCAGTTGGGTTTGTATCCACGAATCCTAAAATCTGGTAAAATGGAAA
ACGGCGGAATCCCTCAACGCGGTAACTTTGAGAAACACCTAGAAAAGGCA
AGCACGGACCTCCAGAAAGTGATCCCTTGGAAAGAGTTTAGCGGATTAGG
TGTGATAGATTGGGAGGCTTGGAGACCCACATGGGAATTTAACTGGGAAC
CGTTGAGGATATATCAAACCGAATCAATTAAGAGAGCTAAAGAACTACAC
CCTACCGCAAACGATTCCGCAGTAAAAGAAATTGCAGAGCGGCAATGGGA
AGATTCAGCCAAGTTATACATGTTAGAAACACTGCGGCTGGCAAAGAAAC
TTCGACCTCAAGCGCCTTGGTGTTACTACTTATTTCCTGATTGCTATAAT
TACGTCGGAAAGAAACCAAAAGATTTCCAATGTAGTGCCTCGATACGTAA
AGGTAACGATAAGCTAAGCTGGTTGTGGAAAGATTCTACGGCATTGTGTC
CATCGATATACGTATATGAATCACAATTAGACAGGTATTCTTTTGAACAA
AGGACATGGCGCGACAATGAGAAACTTCGGGAAGCGTTGCGTGTAGCCAC
GAGAACCTCTAAAATATACCCATACGTTAACTATTTCGATAAGGAGCTTA
TACCGGAGCAAGAAGTATGGAGAATGCTTGCGCAGGCAGCTGCTGTCGGT
GGCAGTGGTGCGGTAATTTGGGGCTCATCTGCTGCAGTTGCATCTGAAGA
GTTATGTAAATCTTTAAAACAGTATATTATTGAAACGCTTGGGCCGGCGG
CAGAGAAGGTGGCTTGGCGTAGTGACTTATGCAGCAAAGAAATTTGTAAT
AATCAGGGTCGCTGCACATTCCCGGACGATGATTATGCAAACGCATGGAA
ATTATTTACAGATGATACTGTTAAGTTTTATGCTGGTAATATTACATGTA
GGTGCTCCGAGAATTATTCTGGTCGTTTCTGCGAAAAGAAGAATTAA

SEQ ID NO: 49 is a reencoded nucleic acid sequence of hyaluronidase (HYAL) originating from Tityus serrulatus with a N-terminal secretion signal

ATGCAATTCTCTACTGTCGCTTCCGTTGCTTTCGTCGCTTTGGCTAACTT
TGTTGCCGCTGCTGATTTTAAAGTTTACTGGGAAGTGCCTTCCTTCCTTT
GTTCTAAACGTTTTAAAATTAATGTAACGGAAGTTTTAACAAGTCACGAG
ATTCTTGTCAATCAGGGTGAGAGTTTCAACGGTGACAAGATAGTAATCTT
TTACGAAAACCAATTGGGGAAGTACCCGCATATTGACTCAAACAATGTGG
AGATCAATGGAGGAATACTTCAAGTAGCCGATTTGGCGAAGCATTTGAAA
GTAGCCAAGGATAATATCACTAAATTCGTCCCGAATCCTAATTTCAACGG
TGTCGGAGTGATCGACTGGGAAGCTTGGCGGCCATCATGGGAATTTAACT
GGGGTAAGTTAAAAGTATATAAAGAAAAGAGCATTGACTTGGTCAAGTCG
AAACATCCGGAGTGGCCCTCCGACAGGGTTGAAAAGGTTGCTAAAGAGGA
GTGGGAGGAGAGTGCCAAAGAATGGATGGTGAAGACCCTGAAGTTAGCAC
AGGAAATGCGACCGAACGCAGTTTGGTGCTATTATCTATTCCCTGACTGC
TACAATTATTTCGGTAAGGATCAACCCTCTCAATTCAGCTGCTCGTCTCG
AATTCAGAAGGAAAATTCTCGTCTTTCTTGGCTCTGGAATCAATCAACAG
CCATTTGCCTAAGCATTTATATCCAGGAATCCCATGTTACCAAATATAAT
ATGTCCCAGCGGACATGGTGGATCGATGCGAGATTAAGAGAAGCAATTCG
AGTCAGCGAACACAGACCAAACATACCCATCTACCCTTACATTAATTATA
TTCTACCTGGAACTAATCAAACTGTACCAGCAATGGACTTTAAAAGGACA
CTGGGTCAAATAGCTAGCCTCGGCCTAGATGGTGCTTTGTTATGGGGATC
TAGCTATCATGTTTTAACAGAATCTCAATGCAAAATCACTTCTGATTATG
TGAAATCAGTGATTGCTCCTACCGTGGCTACTGTCGTTCTCAATACAAAC
AGATGCTCACAGATAATTTGTAAGGGTCGCGGCAACTGTGTTTGGCCTGA
AGAACCATTTAGTTCTTGGAAATACTTAGTTGACCCCAAAATGCCAGTGT
TCAAGCCAACCAACATCCACTGTAAATGTAAAGGTTACCTAGGTAGATAC
TGTGAGATCCCAAAGTAA

SEQ ID NO: 50 is a reencoded nucleic acid sequence of hyaluronidase (HYAL) originating from Bos Taurus with a N-terminal secretion signal

ATGCAATTTTCTACTGTTGCTTCTGTCGCTTTCGTAGCACTGGCCAATTT
TGTAGCTGCTGGAAGCCGTGACCCCGTTGTCCCAAACCAGCCTTTCACAA
CAATATGGAACGCAAATACGGAGTGGTGCATGAAGAAGCATGGAGTGGAC
GTCGATATTTCAATCTTCGACGTGGTCACTAATCCAGGACAGACATTTAG
AGGTCCAAATATGACAATATTTTACAGTTCACAATTGGGGACCTACCCTT
ACTACACATCCGCGGGTGAGCCAGTATTCGGTGGTCTACCCCAGAACGCA
AGTTTGAATGCCCACTTGGCTAGGACATTTCAAGATATTCTGGCAGCCAT
GCCAGAGCCCAGGTTCTCAGGTTTGGCTGTCATAGACTGGGAAGCGTGGA
GACCTCGTTGGGCATTTAATTGGGATACGAAAGACATATACAGGCAGAGG
TCCAGAGCATTGGTTCAGAAACAGCACCCGGATTGGCTGGCGCCACGTGT
TGAGGCCGCAGCGCAAGACCAGTTCGAGGGCGCTGCTGAGGAGTGGATGG
CCGGCACACTTAAATTAGGGCAGGCGCTTCGTCCTCAAGGCTTGTGGGGT
TTCTATAACTTCCCGGAGTGTTACAATTATGACTTCAAATCTCCGAACTA
CACAGGAAGGTGCCCTTTAAACATCTGTGCGCAGAACGACCAGTTAGGTT
GGCTATGGGGTCAAAGTCGTGCACTATATCCATCAATCTACTTGCCGGCG
GCTTTGGAAGGCACGAAGAAGACTCAGATGTTCGTTCAGCACCGTGTAGC
AGAGGCATTTAGGGTGGCGGCAGGCGCTGGTGACCCCAAGCTACCCGTCT
TACCCTATATGCAGCTATTTTACGATATGACAAACCACTTCTTGCCCGCG
GAAGAACTGGAACATAGCTTAGGTGAGTCAGCAGCGCAGGGTGCAGCCGG
GGTTGTTCTGTGGGTTTCCTGGTTATCAACTTCGACAAAGGAAAGCTGTC
AAGCAATCAAGGAATACGTAGATACTACGTTGGGCCCTTCAATCTTGAAC
GTCACATCTGGTGCCAGGTTGTGTTCTCAGGTATTGTGCAGTGGCCACGG
GAGATGCGCGAGAAGACCTTCATATCCTAAAGCCAGGCTGATTCTAAATT
CAACGTCGTTCTCCATCAAGCCAACACCCGGTGGTGGTCCATTGACACTT
CAAGGCGCTTTGAGTTTGGAAGACAGATTACGTATGGCAGTTGAGTTTGA
ATGCAGATGTTACAGAGGATGGAGAGGGACACGTTGTGAGCAGTGGGGCA
TGTGGTAA

SEQ ID NO: 51 is a reencoded nucleic acid sequence of hyaluronidase (HYAL) originating from Apis mellifera with a N-terminal secretion signal

ATGCAGTTTTCCACAGTTGCTTCCGTAGCTTTCGTAGCTTTAGCAAATTT
TGTCGCAGCTTTGTTGTTGGGCTTCGTTCAGTCTACTCCAGATAATAATA
AGACTGTGCGTGAATTTAACGTGTATTGGAACGTTCCTACATTCATGTGC
CATAAGTACGGGTTGAGATTCGAAGAGGTGAGCGAGAAGTATGGCATTTT
GCAAAATTGGATGGACAAATTCAGAGGAGAGGAAATTGCGATCTTATACG
ACCCCGGCATGTTCCCCGCACTGTTGAAAGACCCCAATGGGAATGTAGTA
GCTAGGAATGGCGGAGTGCCCCAGCTTGGCAATTTAACTAAACATTTACA
GGTGTTCAGGGACCACCTAATCAATCAGATTCCAGATAAAAGCTTTCCCG
GAGTTGGTGTAATAGATTTCGAGTCATGGAGACCTATTTTCAGACAGAAC
TGGGCCTCTTTGCAACCGTACAAGAAGCTATCAGTCGAAGTCGTACGTCG
TGAGCACCCATTCTGGGATGATCAGAGAGTGGAGCAAGAGGCGAAAAGAA
GATTTGAAAAGTACGGCCAGCTTTTCATGGAAGAGACCTTGAAAGCCGCA
AAACGTATGCGTCCTGCAGCCAATTGGGGCTACTACGCTTACCCTTATTG
CTACAACCTGACCCCAAACCAGCCCTCAGCTCAATGCGAGGCTACAACAA
TGCAGGAGAACGACAAGATGTCATGGTTATTCGAGTCCGAGGACGTCTTA
CTGCCCAGCGTCTATTTGAGGTGGAACCTGACTAGCGGAGAGAGGGTTGG
TTTAGTTGGTGGTCGTGTCAAGGAAGCCCTGAGAATAGCTCGTCAGATGA
CCACCTCTAGGAAGAAGGTCCTACCATACTACTGGTACAAATACCAGGAT
AGGAGAGACACGGACCTAAGTAGGGCTGATCTTGAGGCGACTCTACGTAA
AATCACGGACTTAGGAGCCGACGGTTTCATAATCTGGGGATCTTCCGATG
ACATCAACACCAAGGCAAAATGTTTGCAGTTCAGAGAATATTTGAATAAC
GAGTTGGGCCCGGCAGTGAAGAGAATAGCCTTAAACAATAATGCGAATGA
CAGGCTAACAGTGGATGTGTCTGTTGACCAGGTTTAA

SEQ ID NO: 52 is a reencoded nucleic acid sequence of hyaluronidase (HYAL) originating from Macaca mulatta with a N-terminal secretion signal

ATGCAATTCAGTACTGTCGCTAGCGTCGCCTTTGTGGCTCTTGCAAATTT
CGTCGCTGCTTCACGTGGGCCTTTATTGCCGAACAGACCATTTGCAACGG
TATGGAATGCTAACACCCAATGGTGTTTAGAGAGGCACTCAGTAGACGTC
GATGTCTCCATATTTGATGTTGTAGCAAACCCCGGCCAGACTTTTCGTGG
GCCGGACATGACTATTTTCTATTCCAGTCAGCTAGGTACTTACCCTTATT
ATACCCCGACGGGTGAACCTGTCTTTGGTGGCCTTCCACAGAACGCATCA
CTTATAGCGCACTTGGCTAGGACTTTCCAGGATATATTGGCAGCTATACC
AGCCCCCGACTTTTCTGGGCTAGCCGTAATTGACTGGGAAGCATGGAGAC
CGAGATGGGCATTTAACTGGGACACGAAAGACATTTACAGGCAAAGGTCC
CGTGCCTTAGTCCAAGCACAGCATCCCGACTGGCCAGTTACTCAAGTTGA
GGCTGTGGCACAAGACCAGTTCCAGGGTGCTGCGAGGGCATGGATGGCAG
GAACCTTGCAATTAGGCCGTGCTTTAAGACCAAGGGGACTATGGGGCTTC
TACGGTTTTCCCGATTGCTACAATTACGATTTCCTATCTCCTAATTACAC
AGGGCAATGCCCATCAGGGATCAGAGCACAAAATGACCAGTTAGGATGGT
TGTGGGGTCAGAGTCGTGCATTATATCCCAGTATCTACATGCCGGCTGTT
TTGGAAGGAACAGGCAAATCCCAAATGTACGTGCAGCACAGGGTCGCGGA
AGCCTTTAGGGTTGCTGTTGCAGCAGGCGACCCAAACTTACCCGTCCTAC
CGTACGTCCAGATTTTCTACGATATGACCAACCACTTTCTTCCATTGGAC
GAGTTAGAGCATAGTCTTGGAGAATCAGCGGCTCAGGGCGCAGCAGGAGT
CGTGTTATGGGTGTCATGGGAGAATACTAGAACAAAAGAATCTTGCCAGG
CAATTAAAGAATATATGGACACTACCTTGGGGCCTTTTATTCTGAACGTC
ACTTCGGGTGCGTTGTTATGCTCGCAAGCCTTATGTTCAAGCCATGGTCG
TTGTGTCCGTCGTCCCTCACATCCAAAAGCCTTGCTAATCCTTAATCCCG
CAAGCTTTAGTATTCAGTTGACACCAGATGGTGGTCCCTTGTCGTTAAGG
GGAGCATTGTCACTTGAGGACCAGGCACAAATGGCTGTTGAATTCAAATG
CAGGTGCTACCCCGGGTGGCAAGGCCCTTGGTGTGAGCAGAAGAGTATGT
GGTAA

SEQ ID NO: 53 is a reencoded nucleic acid sequence of hyaluronidase (HYAL) originating from Vespa magnifica with a N-terminal secretion signal

ATGCAATTTTCTACAGTGGCAAGTGTTGCATTCGTTGCACTAGCCAACT
TTGTGGCGGCAGATAGCTGTGGGTCAAACTGCGAAAAGAGTGAGAGACC
GAAAAGGGTCTTCAACATTTACTGGAACGTACCTACATTCATGTGTCAC
CAGTACGGACTATACTTTGACGAGGTCACGAATTTTAATATAAAGCACA
ACAGCAAAGACAATTTTCAAGGGGACAAGATCGCGATCTTTTATGACCC
CGGGGAGTTTCCCGCTCTGCTGCCACTAAACTATGGTAAGTACAAGATC
AGGAATGGTGGTGTTCCACAAGAGGGTAACATCACCATCCATCTGCAGA
GATTCATAGAGTACCTAGATAAGACCTATCCGAACCGTAACTTTTCAGG
CATCGGTGTGATCGATTTCGAGAGGTGGAGACCAATTTTCAGACAGAAT
TGGGGTAATATGAAGATTTACAAGAACTTCTCCATCGATCTTGTGCGTA
AAGAGCATCCTTTCTGGAATAAGAAAATGATCGAGTTGGAAGCTTCTAA
AAGATTCGAGAAATACGCCCGTCTGTTCATGGAAGAAACATTAAAGTTG
GCTAAGAAAACTAGAAAACAGGCCGATTGGGGCTACTACGGTTACCCCT
ATTGCTTCAACATGTCTCCTACTAATTTCGTTCCTGACTGCGATGTCAC
AGCTAGGGATGAGAACAACGAGATGTCTTGGTTGTTTAACAACCAGAAT
GTCCTATTACCAAGTGTATACATTAGGAGAGAGCTAACTCCTGACCAGA
GGATTGGGCTTGTACAGGGGAGAGTGAAGGAAGCTGTGAGAATTTCAAA
TAAACTGAAGCACTCACCTAAAGTCTTCAGCTATTGGTGGTATGTTTAC
CAAGACGAGACCAACACCTTCTTAACGGAGACCGACGTCAAGAAGACGT
TTCAGGAGATTGTGATCAACGGTGGAGATGGGATTATAATCTGGGGTTC
GTCCTCTGATGTAAACAGCTTGTCCAAGTGTACGAGGTTAAGGGAGTAC
CTATTGACAGTCTTGGGACCAATTGCTGTTAACGTGACTGAAGCAGTAA
ACTAA

SEQ ID NO: 54 is a reencoded nucleic acid sequence of hyaluronidase (HYAL) originating from Cupiennius salei with a N-terminal secretion signal and a C-terminal anchoring signal

ATGCAATTCAGCACTGTTGCATCAGTTGCATTTGTCGCCCTGGCGAATT
TTGTAGCCGCGTTCAAGATTTACTGGAACGTCCCAACTTTTCAGTGCAC
GCATAACTACAAAATCGATTATGTCAAATTGTTGTCCACTTACGGGATA
CAGGTCAATGATGGCGGTAAGTTTCAAGGAAACCAAGTGACTATCTTTT
ATGAAACCCAGTTGGGTTTGTATCCACGAATCCTAAAATCTGGTAAAAT
GGAAAACGGCGGAATCCCTCAACGCGGTAACTTTGAGAAACACCTAGAA
AAGGCAAGCACGGACCTCCAGAAAGTGATCCCTTGGAAAGAGTTTAGCG
GATTAGGTGTGATAGATTGGGAGGCTTGGAGACCCACATGGGAATTTAA
CTGGGAACCGTTGAGGATATATCAAACCGAATCAATTAAGAGAGCTAAA
GAACTACACCCTACCGCAAACGATTCCGCAGTAAAAGAAATTGCAGAGC
GGCAATGGGAAGATTCAGCCAAGTTATACATGTTAGAAACACTGCGGCT
GGCAAAGAAACTTCGACCTCAAGCGCCTTGGTGTTACTACTTATTTCCT
GATTGCTATAATTACGTCGGAAAGAAACCAAAAGATTTCCAATGTAGTG
CCTCGATACGTAAAGGTAACGATAAGCTAAGCTGGTTGTGGAAAGATTC
TACGGCATTGTGTCCATCGATATACGTATATGAATCACAATTAGACAGG
TATTCTTTTGAACAAAGGACATGGCGCGACAATGAGAAACTTCGGGAAG
CGTTGCGTGTAGCCACGAGAACCTCTAAAATATACCCATACGTTAACTA
TTTCGATAAGGAGCTTATACCGGAGCAAGAAGTATGGAGAATGCTTGCG
CAGGCAGCTGCTGTCGGTGGCAGTGGTGCGGTAATTTGGGGCTCATCTG
CTGCAGTTGCATCTGAAGAGTTATGTAAATCTTTAAAACAGTATATTAT
TGAAACGCTTGGGCCGGCGGCAGAGAAGGTGGCTTGGCGTAGTGACTTA
TGCAGCAAAGAAATTTGTAATAATCAGGGTCGCTGCACATTCCCGGACG
ATGATTATGCAAACGCATGGAAATTATTTACAGATGATACTGTTAAGTT
TTATGCTGGTAATATTACATGTAGGTGCTCCGAGAATTATTCTGGTCGT
TTCTGCGAAAAGAAGAATGGATCCGCCATTTCTCAAATCACTGACGGTC
AAATCCAAGCTACTACCACTGCTACCACCGAAGCTACCACCACTGCTGC
CCCATCTTCCACCGTTGAAACTGTTTCTCCATCCAGCACCGAAACTATC
TCTCAACAAACTGAAAATGGTGCTGCTAAGGCCGCTGTCGGTATGGGTG
CCGGTGCTCTAGCTGCTGCTGCTATGTTGTTATAA

SEQ ID NO: 55 is a reencoded nucleic acid sequence of hyaluronidase (HYAL) originating from Tityus serrulatus with a N-terminal secretion signal and a C-terminal anchoring signal

ATGCAATTCTCTACTGTCGCTTCCGTTGCTTTCGTCGCTTTGGCTAACT
TTGTTGCCGCTGCTGATTTTAAAGTTTACTGGGAAGTGCCTTCCTTCCT
TTGTTCTAAACGTTTTAAAATTAATGTAACGGAAGTTTTAACAAGTCAC
GAGATTCTTGTCAATCAGGGTGAGAGTTTCAACGGTGACAAGATAGTAA
TCTTTTACGAAAACCAATTGGGGAAGTACCCGCATATTGACTCAAACAA
TGTGGAGATCAATGGAGGAATACTTCAAGTAGCCGATTTGGCGAAGCAT
TTGAAAGTAGCCAAGGATAATATCACTAAATTCGTCCCGAATCCTAATT
TCAACGGTGTCGGAGTGATCGACTGGGAAGCTTGGCGGCCATCATGGGA
ATTTAACTGGGGTAAGTTAAAAGTATATAAAGAAAAGAGCATTGACTTG
GTCAAGTCGAAACATCCGGAGTGGCCCTCCGACAGGGTTGAAAAGGTTG
CTAAAGAGGAGTGGGAGGAGAGTGCCAAAGAATGGATGGTGAAGACCCT
GAAGTTAGCACAGGAAATGCGACCGAACGCAGTTTGGTGCTATTATCTA
TTCCCTGACTGCTACAATTATTTCGGTAAGGATCAACCCTCTCAATTCA
GCTGCTCGTCTCGAATTCAGAAGGAAAATTCTCGTCTTTCTTGGCTCTG
GAATCAATCAACAGCCATTTGCCTAAGCATTTATATCCAGGAATCCCAT
GTTACCAAATATAATATGTCCCAGCGGACATGGTGGATCGATGCGAGAT
TAAGAGAAGCAATTCGAGTCAGCGAACACAGACCAAACATACCCATCTA
CCCTTACATTAATTATATTCTACCTGGAACTAATCAAACTGTACCAGCA
ATGGACTTTAAAAGGACACTGGGTCAAATAGCTAGCCTCGGCCTAGATG
GTGCTTTGTTATGGGGATCTAGCTATCATGTTTTAACAGAATCTCAATG
CAAAATCACTTCTGATTATGTGAAATCAGTGATTGCTCCTACCGTGGCT
ACTGTCGTTCTCAATACAAACAGATGCTCACAGATAATTTGTAAGGGTC
GCGGCAACTGTGTTTGGCCTGAAGAACCATTTAGTTCTTGGAAATACTT
AGTTGACCCCAAAATGCCAGTGTTCAAGCCAACCAACATCCACTGTAAA
TGTAAAGGTTACCTAGGTAGATACTGTGAGATCCCAAAGGGATCCGCCA
TTTCTCAAATCACTGACGGTCAAATCCAAGCTACTACCACTGCTACCAC
CGAAGCTACCACCACTGCTGCCCCATCTTCCACCGTTGAAACTGTTTCT
CCATCCAGCACCGAAACTATCTCTCAACAAACTGAAAATGGTGCTGCTA
AGGCCGCTGTCGGTATGGGTGCCGGTGCTCTAGCTGCTGCTGCTATGTT
GTTATAA

SEQ ID NO: 56 is a reencoded nucleic acid sequence of hyaluronidase (HYAL) originating from Bos taurus with a N-terminal secretion signal and a C-terminal anchoring signal

ATGCAATTTTCTACTGTTGCTTCTGTCGCTTTCGTAGCACTGGCCAATT
TTGTAGCTGCTGGAAGCCGTGACCCCGTTGTCCCAAACCAGCCTTTCAC
AACAATATGGAACGCAAATACGGAGTGGTGCATGAAGAAGCATGGAGTG
GACGTCGATATTTCAATCTTCGACGTGGTCACTAATCCAGGACAGACAT
TTAGAGGTCCAAATATGACAATATTTTACAGTTCACAATTGGGGACCTA
CCCTTACTACACATCCGCGGGTGAGCCAGTATTCGGTGGTCTACCCCAG
AACGCAAGTTTGAATGCCCACTTGGCTAGGACATTTCAAGATATTCTGG
CAGCCATGCCAGAGCCCAGGTTCTCAGGTTTGGCTGTCATAGACTGGGA
AGCGTGGAGACCTCGTTGGGCATTTAATTGGGATACGAAAGACATATAC
AGGCAGAGGTCCAGAGCATTGGTTCAGAAACAGCACCCGGATTGGCTGG
CGCCACGTGTTGAGGCCGCAGCGCAAGACCAGTTCGAGGGCGCTGCTGA
GGAGTGGATGGCCGGCACACTTAAATTAGGGCAGGCGCTTCGTCCTCAA
GGCTTGTGGGGTTTCTATAACTTCCCGGAGTGTTACAATTATGACTTCA
AATCTCCGAACTACACAGGAAGGTGCCCTTTAAACATCTGTGCGCAGAA
CGACCAGTTAGGTTGGCTATGGGGTCAAAGTCGTGCACTATATCCATCA
ATCTACTTGCCGGCGGCTTTGGAAGGCACGAAGAAGACTCAGATGTTCG
TTCAGCACCGTGTAGCAGAGGCATTTAGGGTGGCGGCAGGCGCTGGTGA
CCCCAAGCTACCCGTCTTACCCTATATGCAGCTATTTTACGATATGACA
AACCACTTCTTGCCCGCGGAAGAACTGGAACATAGCTTAGGTGAGTCAG
CAGCGCAGGGTGCAGCCGGGGTTGTTCTGTGGGTTTCCTGGTTATCAAC
TTCGACAAAGGAAAGCTGTCAAGCAATCAAGGAATACGTAGATACTACG
TTGGGCCCTTCAATCTTGAACGTCACATCTGGTGCCAGGTTGTGTTCTC
AGGTATTGTGCAGTGGCCACGGGAGATGCGCGAGAAGACCTTCATATCC
TAAAGCCAGGCTGATTCTAAATTCAACGTCGTTCTCCATCAAGCCAACA
CCCGGTGGTGGTCCATTGACACTTCAAGGCGCTTTGAGTTTGGAAGACA
GATTACGTATGGCAGTTGAGTTTGAATGCAGATGTTACAGAGGATGGAG
AGGGACACGTTGTGAGCAGTGGGGCATGTGGGGATCCGCCATTTCTCAA
ATCACTGACGGTCAAATCCAAGCTACTACCACTGCTACCACCGAAGCTA
CCACCACTGCTGCCCCATCTTCCACCGTTGAAACTGTTTCTCCATCCAG
CACCGAAACTATCTCTCAACAAACTGAAAATGGTGCTGCTAAGGCCGCT
GTCGGTATGGGTGCCGGTGCTCTAGCTGCTGCTGCTATGTTGTTATAA

SEQ ID NO: 57 is a reencoded nucleic acid sequence of hyaluronidase (HYAL) originating from Apis mellifera with a N-terminal secretion signal and a C-terminal anchoring signal

ATGCAGTTTTCCACAGTTGCTTCCGTAGCTTTCGTAGCTTTAGCAAATT
TTGTCGCAGCTTTGTTGTTGGGCTTCGTTCAGTCTACTCCAGATAATAA
TAAGACTGTGCGTGAATTTAACGTGTATTGGAACGTTCCTACATTCATG
TGCCATAAGTACGGGTTGAGATTCGAAGAGGTGAGCGAGAAGTATGGCA
TTTTGCAAAATTGGATGGACAAATTCAGAGGAGAGGAAATTGCGATCTT
ATACGACCCCGGCATGTTCCCCGCACTGTTGAAAGACCCCAATGGGAAT
GTAGTAGCTAGGAATGGCGGAGTGCCCCAGCTTGGCAATTTAACTAAAC
ATTTACAGGTGTTCAGGGACCACCTAATCAATCAGATTCCAGATAAAAG
CTTTCCCGGAGTTGGTGTAATAGATTTCGAGTCATGGAGACCTATTTTC
AGACAGAACTGGGCCTCTTTGCAACCGTACAAGAAGCTATCAGTCGAAG
TCGTACGTCGTGAGCACCCATTCTGGGATGATCAGAGAGTGGAGCAAGA
GGCGAAAAGAAGATTTGAAAAGTACGGCCAGCTTTTCATGGAAGAGACC
TTGAAAGCCGCAAAACGTATGCGTCCTGCAGCCAATTGGGGCTACTACG
CTTACCCTTATTGCTACAACCTGACCCCAAACCAGCCCTCAGCTCAATG
CGAGGCTACAACAATGCAGGAGAACGACAAGATGTCATGGTTATTCGAG
TCCGAGGACGTCTTACTGCCCAGCGTCTATTTGAGGTGGAACCTGACTA
GCGGAGAGAGGGTTGGTTTAGTTGGTGGTCGTGTCAAGGAAGCCCTGAG
AATAGCTCGTCAGATGACCACCTCTAGGAAGAAGGTCCTACCATACTAC
TGGTACAAATACCAGGATAGGAGAGACACGGACCTAAGTAGGGCTGATC
TTGAGGCGACTCTACGTAAAATCACGGACTTAGGAGCCGACGGTTTCAT
AATCTGGGGATCTTCCGATGACATCAACACCAAGGCAAAATGTTTGCAG
TTCAGAGAATATTTGAATAACGAGTTGGGCCCGGCAGTGAAGAGAATAG
CCTTAAACAATAATGCGAATGACAGGCTAACAGTGGATGTGTCTGTTGA
CCAGGTTGGATCCGCCATTTCTCAAATCACTGACGGTCAAATCCAAGCT
ACTACCACTGCTACCACCGAAGCTACCACCACTGCTGCCCCATCTTCCA
CCGTTGAAACTGTTTCTCCATCCAGCACCGAAACTATCTCTCAACAAAC
TGAAAATGGTGCTGCTAAGGCCGCTGTCGGTATGGGTGCCGGTGCTCTA
GCTGCTGCTGCTATGTTGTTATAA

SEQ ID NO: 58 is a reencoded nucleic acid sequence of hyaluronidase (HYAL) originating from Macaca mulatta with a N-terminal secretion signal and a C-terminal anchoring signal

ATGCAATTCAGTACTGTCGCTAGCGTCGCCTTTGTGGCTCTTGCAAATT
TCGTCGCTGCTTCACGTGGGCCTTTATTGCCGAACAGACCATTTGCAAC
GGTATGGAATGCTAACACCCAATGGTGTTTAGAGAGGCACTCAGTAGAC
GTCGATGTCTCCATATTTGATGTTGTAGCAAACCCCGGCCAGACTTTTC
GTGGGCCGGACATGACTATTTTCTATTCCAGTCAGCTAGGTACTTACCC
TTATTATACCCCGACGGGTGAACCTGTCTTTGGTGGCCTTCCACAGAAC
GCATCACTTATAGCGCACTTGGCTAGGACTTTCCAGGATATATTGGCAG
CTATACCAGCCCCCGACTTTTCTGGGCTAGCCGTAATTGACTGGGAAGC
ATGGAGACCGAGATGGGCATTTAACTGGGACACGAAAGACATTTACAGG
CAAAGGTCCCGTGCCTTAGTCCAAGCACAGCATCCCGACTGGCCAGTTA
CTCAAGTTGAGGCTGTGGCACAAGACCAGTTCCAGGGTGCTGCGAGGGC
ATGGATGGCAGGAACCTTGCAATTAGGCCGTGCTTTAAGACCAAGGGGA
CTATGGGGCTTCTACGGTTTTCCCGATTGCTACAATTACGATTTCCTAT
CTCCTAATTACACAGGGCAATGCCCATCAGGGATCAGAGCACAAAATGA
CCAGTTAGGATGGTTGTGGGGTCAGAGTCGTGCATTATATCCCAGTATC
TACATGCCGGCTGTTTTGGAAGGAACAGGCAAATCCCAAATGTACGTGC
AGCACAGGGTCGCGGAAGCCTTTAGGGTTGCTGTTGCAGCAGGCGACCC
AAACTTACCCGTCCTACCGTACGTCCAGATTTTCTACGATATGACCAAC
CACTTTCTTCCATTGGACGAGTTAGAGCATAGTCTTGGAGAATCAGCGG
CTCAGGGCGCAGCAGGAGTCGTGTTATGGGTGTCATGGGAGAATACTAG
AACAAAAGAATCTTGCCAGGCAATTAAAGAATATATGGACACTACCTTG
GGGCCTTTTATTCTGAACGTCACTTCGGGTGCGTTGTTATGCTCGCAAG
CCTTATGTTCAAGCCATGGTCGTTGTGTCCGTCGTCCCTCACATCCAAA
AGCCTTGCTAATCCTTAATCCCGCAAGCTTTAGTATTCAGTTGACACCA
GATGGTGGTCCCTTGTCGTTAAGGGGAGCATTGTCACTTGAGGACCAGG
CACAAATGGCTGTTGAATTCAAATGCAGGTGCTACCCCGGGTGGCAAGG
CCCTTGGTGTGAGCAGAAGAGTATGTGGGGATCCGCCATTTCTCAAATC
ACTGACGGTCAAATCCAAGCTACTACCACTGCTACCACCGAAGCTACCA
CCACTGCTGCCCCATCTTCCACCGTTGAAACTGTTTCTCCATCCAGCAC
CGAAACTATCTCTCAACAAACTGAAAATGGTGCTGCTAAGGCCGCTGTC
GGTATGGGTGCCGGTGCTCTAGCTGCTGCTGCTATGTTGTTATAA

SEQ ID NO: 59 is a reencoded nucleic acid sequence of hyaluronidase (HYAL) originating from Vespa magnifica with a N-terminal secretion signal and a C-terminal anchoring signal

ATGCAATTTTCTACAGTGGCAAGTGTTGCATTCGTTGCACTAGCCAACT
TTGTGGCGGCAGATAGCTGTGGGTCAAACTGCGAAAAGAGTGAGAGACC
GAAAAGGGTCTTCAACATTTACTGGAACGTACCTACATTCATGTGTCAC
CAGTACGGACTATACTTTGACGAGGTCACGAATTTTAATATAAAGCACA
ACAGCAAAGACAATTTTCAAGGGGACAAGATCGCGATCTTTTATGACCC
CGGGGAGTTTCCCGCTCTGCTGCCACTAAACTATGGTAAGTACAAGATC
AGGAATGGTGGTGTTCCACAAGAGGGTAACATCACCATCCATCTGCAGA
GATTCATAGAGTACCTAGATAAGACCTATCCGAACCGTAACTTTTCAGG
CATCGGTGTGATCGATTTCGAGAGGTGGAGACCAATTTTCAGACAGAAT
TGGGGTAATATGAAGATTTACAAGAACTTCTCCATCGATCTTGTGCGTA
AAGAGCATCCTTTCTGGAATAAGAAAATGATCGAGTTGGAAGCTTCTAA
AAGATTCGAGAAATACGCCCGTCTGTTCATGGAAGAAACATTAAAGTTG
GCTAAGAAAACTAGAAAACAGGCCGATTGGGGCTACTACGGTTACCCCT
ATTGCTTCAACATGTCTCCTACTAATTTCGTTCCTGACTGCGATGTCAC
AGCTAGGGATGAGAACAACGAGATGTCTTGGTTGTTTAACAACCAGAAT
GTCCTATTACCAAGTGTATACATTAGGAGAGAGCTAACTCCTGACCAGA
GGATTGGGCTTGTACAGGGGAGAGTGAAGGAAGCTGTGAGAATTTCAAA
TAAACTGAAGCACTCACCTAAAGTCTTCAGCTATTGGTGGTATGTTTAC
CAAGACGAGACCAACACCTTCTTAACGGAGACCGACGTCAAGAAGACGT
TTCAGGAGATTGTGATCAACGGTGGAGATGGGATTATAATCTGGGGTTC
GTCCTCTGATGTAAACAGCTTGTCCAAGTGTACGAGGTTAAGGGAGTAC
CTATTGACAGTCTTGGGACCAATTGCTGTTAACGTGACTGAAGCAGTAA
ACGGATCCGCCATTTCTCAAATCACTGACGGTCAAATCCAAGCTACTAC
CACTGCTACCACCGAAGCTACCACCACTGCTGCCCCATCTTCCACCGTT
GAAACTGTTTCTCCATCCAGCACCGAAACTATCTCTCAACAAACTGAAA
ATGGTGCTGCTAAGGCCGCTGTCGGTATGGGTGCCGGTGCTCTAGCTGC
TGCTGCTATGTTGTTATAA

SEQ ID NO: 60 is an amino acid sequence of hyaluronidase (HYAL) originating from Cupiennius salei with a N-terminal secretion signal

MQFSTVASVAFVALANFVAAFKIYWNVPTFQCTHNYKIDYVKLLSTYGI
QVNDGGKFQGNQVTIFYETQLGLYPRILKSGKMENGGIPQRGNFEKHLE
KASTDLQKVIPWKEFSGLGVIDWEAWRPTWEFNWEPLRIYQTESIKRAK
ELHPTANDSAVKEIAERQWEDSAKLYMLETLRLAKKLRPQAPWCYYLFP
DCYNYVGKKPKDFQCSASIRKGNDKLSWLWKDSTALCPSIYVYESQLDR
YSFEQRTWRDNEKLREALRVATRTSKIYPYVNYFDKELIPEQEVWRMLA
QAAAVGGSGAVIWGSSAAVASEELCKSLKQYIIETLGPAAEKVAWRSDL
CSKEICNNQGRCTFPDDDYANAWKLFTDDTVKFYAGNITCRCSENYSGR
FCEKKN

SEQ ID NO: 61 is an amino acid sequence of hyaluronidase (HYAL) originating from Tityus serrulatus with a N-terminal secretion signal

MQFSTVASVAFVALANFVAAADFKVYWEVPSFLCSKRFKINVTEVLTSH
EILVNQGESENGDKIVIFYENQLGKYPHIDSNNVEINGGILQVADLAKH
LKVAKDNITKFVPNPNFNGVGVIDWEAWRPSWEFNWGKLKVYKEKSIDL
VKSKHPEWPSDRVEKVAKEEWEESAKEWMVKTLKLAQEMRPNAVWCYYL
FPDCYNYFGKDQPSQFSCSSRIQKENSRLSWLWNQSTAICLSIYIQESH
VTKYNMSQRTWWIDARLREAIRVSEHRPNIPIYPYINYILPGTNQTVPA
MDFKRTLGQIASLGLDGALLWGSSYHVLTESQCKITSDYVKSVIAPTVA
TVVLNTNRCSQIICKGRGNCVWPEEPFSSWKYLVDPKMPVFKPTNIHCK
CKGYLGRYCEIPK

SEQ ID NO: 62 is an amino acid sequence of hyaluronidase (HYAL) originating from Bos taurus with a N-terminal secretion signal

MQFSTVASVAFVALANFVAAGSRDPVVPNQPFTTIWNANTEWCMKKHGV
DVDISIFDVVTNPGQTFRGPNMTIFYSSQLGTYPYYTSAGEPVFGGLPQ
NASLNAHLARTFQDILAAMPEPRESGLAVIDWEAWRPRWAFNWDTKDIY
RQRSRALVQKQHPDWLAPRVEAAAQDQFEGAAEEWMAGTLKLGQALRPQ
GLWGFYNFPECYNYDFKSPNYTGRCPLNICAQNDQLGWLWGQSRALYPS
IYLPAALEGTKKTQMFVQHRVAEAFRVAAGAGDPKLPVLPYMQLFYDMT
NHFLPAEELEHSLGESAAQGAAGVVLWVSWLSTSTKESCQAIKEYVDTT
LGPSILNVTSGARLCSQVLCSGHGRCARRPSYPKARLILNSTSFSIKPT
PGGGPLTLQGALSLEDRLRMAVEFECRCYRGWRGTRCEQWGMW

SEQ ID NO: 63 is an amino acid sequence of hyaluronidase (HYAL) originating from Apis mellifera with a N-terminal secretion signal

MQFSTVASVAFVALANFVAALLLGFVQSTPDNNKTVREFNVYWNVPTFM
CHKYGLRFEEVSEKYGILQNWMDKFRGEEIAILYDPGMFPALLKDPNGN
VVARNGGVPQLGNLTKHLQVFRDHLINQIPDKSFPGVGVIDFESWRPIF
RQNWASLQPYKKLSVEVVRREHPFWDDQRVEQEAKRRFEKYGQLFMEET
LKAAKRMRPAANWGYYAYPYCYNLTPNQPSAQCEATTMQENDKMSWLFE
SEDVLLPSVYLRWNLTSGERVGLVGGRVKEALRIARQMTTSRKKVLPYY
WYKYQDRRDTDLSRADLEATLRKITDLGADGFIIWGSSDDINTKAKCLQ
FREYLNNELGPAVKRIALNNNANDRLTVDVSVDQV

SEQ ID NO: 64 is an amino acid sequence of hyaluronidase (HYAL) originating from Macaca mulatta with a N-terminal secretion signal

MQFSTVASVAFVALANFVAASRGPLLPNRPFATVWNANTQWCLERHSVD
VDVSIFDVVANPGQTFRGPDMTIFYSSQLGTYPYYTPTGEPVFGGLPQN
ASLIAHLARTFQDILAAIPAPDFSGLAVIDWEAWRPRWAFNWDTKDIYR
QRSRALVQAQHPDWPVTQVEAVAQDQFQGAARAWMAGTLQLGRALRPRG
LWGFYGFPDCYNYDFLSPNYTGQCPSGIRAQNDQLGWLWGQSRALYPSI
YMPAVLEGTGKSQMYVQHRVAEAFRVAVAAGDPNLPVLPYVQIFYDMTN
HFLPLDELEHSLGESAAQGAAGVVLWVSWENTRTKESCQAIKEYMDTTL
GPFILNVTSGALLCSQALCSSHGRCVRRPSHPKALLILNPASFSIQLTP
DGGPLSLRGALSLEDQAQMAVEFKCRCYPGWQGPWCEQKSMW

SEQ ID NO: 65 is an amino acid sequence of hyaluronidase (HYAL) originating from Vespa magnifica with a N-terminal secretion signal

MQFSTVASVAFVALANFVAADSCGSNCEKSERPKRVFNIYWNVPTFMCH
QYGLYFDEVTNFNIKHNSKDNFQGDKIAIFYDPGEFPALLPLNYGKYKI
RNGGVPQEGNITIHLQRFIEYLDKTYPNRNFSGIGVIDFERWRPIFRQN
WGNMKIYKNFSIDLVRKEHPFWNKKMIELEASKRFEKYARLFMEETLKL
AKKTRKQADWGYYGYPYCFNMSPTNFVPDCDVTARDENNEMSWLFNNQN
VLLPSVYIRRELTPDQRIGLVQGRVKEAVRISNKLKHSPKVFSYWWYVY
QDETNTFLTETDVKKTFQEIVINGGDGIIIWGSSSDVNSLSKCTRLREY
LLTVLGPIAVNVTEAVN

SEQ ID NO: 66 is an amino acid sequence of hyaluronidase (HYAL) originating from Cupiennius salei with a N-terminal secretion signal and a C-terminal anchoring signal

MQFSTVASVAFVALANFVAAFKIYWNVPTFQCTHNYKIDYVKLLSTYGI
QVNDGGKFQGNQVTIFYETQLGLYPRILKSGKMENGGIPQRGNFEKHLE
KASTDLQKVIPWKEFSGLGVIDWEAWRPTWEFNWEPLRIYQTESIKRAK
ELHPTANDSAVKEIAERQWEDSAKLYMLETLRLAKKLRPQAPWCYYLFP
DCYNYVGKKPKDFQCSASIRKGNDKLSWLWKDSTALCPSIYVYESQLDR
YSFEQRTWRDNEKLREALRVATRTSKIYPYVNYFDKELIPEQEVWRMLA
QAAAVGGSGAVIWGSSAAVASEELCKSLKQYIIETLGPAAEKVAWRSDL
CSKEICNNQGRCTFPDDDYANAWKLFTDDTVKFYAGNITCRCSENYSGR
FCEKKNGSAISQITDGQIQATTTATTEATTTAAPSSTVETVSPSSTETI
SQQTENGAAKAAVGMGAGALAAAAMLL

SEQ ID NO: 67 is an amino acid sequence of hyaluronidase (HYAL) originating from Tityus serrulatus with a N-terminal secretion signal and a C-terminal anchoring signal

MQFSTVASVAFVALANFVAAADFKVYWEVPSFLCSKRFKINVTEVLTSH
EILVNQGESENGDKIVIFYENQLGKYPHIDSNNVEINGGILQVADLAKH
LKVAKDNITKFVPNPNFNGVGVIDWEAWRPSWEFNWGKLKVYKEKSIDL
VKSKHPEWPSDRVEKVAKEEWEESAKEWMVKTLKLAQEMRPNAVWCYYL
FPDCYNYFGKDQPSQFSCSSRIQKENSRLSWLWNQSTAICLSIYIQESH
VTKYNMSQRTWWIDARLREAIRVSEHRPNIPIYPYINYILPGTNQTVPA
MDFKRTLGQIASLGLDGALLWGSSYHVLTESQCKITSDYVKSVIAPTVA
TVVLNTNRCSQIICKGRGNCVWPEEPFSSWKYLVDPKMPVFKPTNIHCK
CKGYLGRYCEIPKGSAISQITDGQIQATTTATTEATTTAAPSSTVETVS
PSSTETISQQTENGAAKAAVGMGAGALAAAAMLL

SEQ ID NO: 68 is an amino acid sequence of hyaluronidase (HYAL) originating from Bos taurus with a N-terminal secretion signal and a C-terminal anchoring signal

MQFSTVASVAFVALANFVAAGSRDPVVPNQPFTTIWNANTEWCMKKHGV
DVDISIFDVVTNPGQTFRGPNMTIFYSSQLGTYPYYTSAGEPVFGGLPQ
NASLNAHLARTFQDILAAMPEPRESGLAVIDWEAWRPRWAFNWDTKDIY
RQRSRALVQKQHPDWLAPRVEAAAQDQFEGAAEEWMAGTLKLGQALRPQ
GLWGFYNFPECYNYDFKSPNYTGRCPLNICAQNDQLGWLWGQSRALYPS
IYLPAALEGTKKTQMFVQHRVAEAFRVAAGAGDPKLPVLPYMQLFYDMT
NHFLPAEELEHSLGESAAQGAAGVVLWVSWLSTSTKESCQAIKEYVDTT
LGPSILNVTSGARLCSQVLCSGHGRCARRPSYPKARLILNSTSFSIKPT
PGGGPLTLQGALSLEDRLRMAVEFECRCYRGWRGTRCEQWGMWGSAISQ
ITDGQIQATTTATTEATTTAAPSSTVETVSPSSTETISQQTENGAAKAA
VGMGAGALAAAAMLL

SEQ ID NO: 69 is an amino acid sequence of hyaluronidase (HYAL) originating from Apis mellifera with a N-terminal secretion signal and a C-terminal anchoring signal

MQFSTVASVAFVALANFVAALLLGFVQSTPDNNKTVREFNVYWNVPTFM
CHKYGLRFEEVSEKYGILQNWMDKFRGEEIAILYDPGMFPALLKDPNGN
VVARNGGVPQLGNLTKHLQVFRDHLINQIPDKSFPGVGVIDFESWRPIF
RQNWASLQPYKKLSVEVVRREHPFWDDQRVEQEAKRRFEKYGQLFMEET
LKAAKRMRPAANWGYYAYPYCYNLTPNQPSAQCEATTMQENDKMSWLFE
SEDVLLPSVYLRWNLTSGERVGLVGGRVKEALRIARQMTTSRKKVLPYY
WYKYQDRRDTDLSRADLEATLRKITDLGADGFIIWGSSDDINTKAKCLQ
FREYLNNELGPAVKRIALNNNANDRLTVDVSVDQVGSAISQITDGQIQA
TTTATTEATTTAAPSSTVETVSPSSTETISQQTENGAAKAAVGMGAGAL
AAAAMLL

SEQ ID NO: 70 is an amino acid sequence of hyaluronidase (HYAL) originating from Macaca mulatta with a N-terminal secretion signal and a C-terminal anchoring signal

MQFSTVASVAFVALANFVAASRGPLLPNRPFATVWNANTQWCLERHSVD
VDVSIFDVVANPGQTFRGPDMTIFYSSQLGTYPYYTPTGEPVFGGLPQN
ASLIAHLARTFQDILAAIPAPDFSGLAVIDWEAWRPRWAFNWDTKDIYR
QRSRALVQAQHPDWPVTQVEAVAQDQFQGAARAWMAGTLQLGRALRPRG
LWGFYGFPDCYNYDFLSPNYTGQCPSGIRAQNDQLGWLWGQSRALYPSI
YMPAVLEGTGKSQMYVQHRVAEAFRVAVAAGDPNLPVLPYVQIFYDMTN
HFLPLDELEHSLGESAAQGAAGVVLWVSWENTRTKESCQAIKEYMDTTL
GPFILNVTSGALLCSQALCSSHGRCVRRPSHPKALLILNPASFSIQLTP
DGGPLSLRGALSLEDQAQMAVEFKCRCYPGWQGPWCEQKSMWGSAISQI
TDGQIQATTTATTEATTTAAPSSTVETVSPSSTETISQQTENGAAKAAV
GMGAGALAAAAMLL

SEQ ID NO: 71 is an amino acid sequence of hyaluronidase (HYAL) originating from Vespa magnifica with a N-terminal secretion signal and a C-terminal anchoring signal

MQFSTVASVAFVALANFVAADSCGSNCEKSERPKRVFNIYWNVPTFMCH
QYGLYFDEVTNFNIKHNSKDNFQGDKIAIFYDPGEFPALLPLNYGKYKI
RNGGVPQEGNITIHLQRFIEYLDKTYPNRNFSGIGVIDFERWRPIFRQN
WGNMKIYKNFSIDLVRKEHPFWNKKMIELEASKRFEKYARLFMEETLKL
AKKTRKQADWGYYGYPYCFNMSPTNFVPDCDVTARDENNEMSWLFNNQN
VLLPSVYIRRELTPDQRIGLVQGRVKEAVRISNKLKHSPKVFSYWWYVY
QDETNTFLTETDVKKTFQEIVINGGDGIIIWGSSSDVNSLSKCTRLREY
LLTVLGPIAVNVTEAVNAISQITDGQIQATTTATTEATTTAAPSSTVET
VSPSSTETISQQTENGAAKAAVGMGAGALAAAAMLL

SEQ ID NO: 72 is a nucleic acid sequence of glutamine-fructose-6-phosphate amidotransferase (GFA1) originating from Saccharomyces cerevisiae

ATGTGTGGTATCTTTGGTTACTGCAATTATCTAGTGGAAAGATCCAGAG
GAGAAATTATCGACACCTTAGTGGATGGTTTACAAAGATTAGAATATAG
AGGCTATGATTCCACCGGTATTGCTATCGATGGTGACGAAGCTGATTCT
ACTTTCATCTATAAGCAAATCGGTAAAGTGAGTGCTTTGAAAGAGGAGA
TTACTAAGCAAAATCCGAACAGAGACGTTACTTTTGTCTCTCATTGTGG
TATTGCGCATACTAGATGGGCTACTCACGGTCGACCAGAACAAGTTAAC
TGTCACCCTCAAAGATCTGACCCAGAAGACCAATTTGTGGTCGTTCATA
ATGGTATCATCACAAATTTTAGAGAACTGAAGACTCTTTTAATTAACAA
AGGTTATAAATTCGAAAGTGATACCGATACCGAGTGTATTGCTAAACTA
TATTTGCATTTATACAATACAAATTTACAAAATGGGCATGACTTAGATT
TCCACGAATTAACCAAGCTAGTTCTTTTAGAACTAGAAGGTTCATACGG
GTTATTATGTAAATCTTGTCACTATCCTAATGAGGTTATCGCCACTAGA
AAAGGGTCCCCTTTACTGATTGGTGTCAAATCTGAAAAAAAACTAAAAG
TCGACTTCGTGGATGTGGAATTTCCCGAAGAAAACGCTGGTCAACCGGA
AATTCCATTGAAATCTAACAACAAATCATTTGGCTTGGGCCCAAAGAAA
GCTCGTGAATTTGAAGCTGGTTCCCAAAATGCCAATTTACTACCAATTG
CCGCCAATGAATTTAACTTGAGACATTCTCAATCCAGGGCTTTCCTATC
AGAAGATGGATCTCCAACACCGGTGGAATTTTTTGTTTCTTCGGATGCG
GCATCTGTTGTTAAACATACCAAGAAGGTGCTATTTTTAGAAGATGACG
ATTTGGCTCATATTTACGATGGTGAGTTACATATTCATAGATCTAGAAG
AGAAGTAGGCGCATCAATGACAAGGTCCATTCAAACTTTAGAGATGGAG
TTAGCTCAGATCATGAAGGGCCCTTACGACCATTTTATGCAAAAGGAAA
TCTATGAGCAACCAGAATCTACTTTCAATACTATGAGAGGTAGAATCGA
CTATGAAAATAATAAAGTGATATTGGGTGGTTTAAAGGCATGGTTACCA
GTTGTCAGAAGAGCACGGAGACTGATCATGATCGCATGCGGTACTTCTT
ATCATTCATGTTTGGCTACTCGTGCTATCTTCGAAGAATTATCAGATAT
CCCAGTTAGTGTGGAATTAGCGTCTGACTTTCTGGACAGAAAATGCCCT
GTCTTCAGAGACGATGTATGCGTGTTTGTTTCACAAAGTGGTGAAACTG
CGGATACCATGCTGGCTCTAAATTATTGTTTAGAAAGAGGAGCCTTAAC
TGTCGGAATTGTTAACAGTGTTGGTTCTTCTATCTCTCGTGTCACCCAC
TGTGGTGTTCATATTAACGCTGGTCCTGAAATTGGTGTTGCCTCTACAA
AAGCTTATACTTCCCAGTATATTGCCTTAGTGATGTTTGCTCTATCGCT
GTCAGATGACCGTGTATCGAAAATAGACAGAAGAATTGAAATCATTCAA
GGCTTGAAGTTAATCCCGGGCCAAATTAAGCAGGTATTAAAGCTGGAAC
CAAGAATAAAAAAGCTCTGTGCGACTGAATTAAAGGATCAAAAATCTCT
ATTGTTATTGGGTAGAGGTTACCAATTTGCTGCTGCTCTGGAAGGTGCT
TTGAAGATCAAAGAAATTTCTTATATGCATTCTGAAGGTGTTTTGGCAG
GTGAGTTGAAGCACGGTGTCTTGGCCTTGGTGGACGAAAACTTGCCAAT
CATTGCTTTTGGTACCAGAGACTCTCTATTCCCTAAAGTAGTTTCCTCT
ATTGAGCAAGTTACTGTAAGAAAGGGCCATCCAATTATTATTTGTAACG
AAAATGATGAAGTGTGGGCGCAAAAATCTAAATCAATCGACCTGCAAAC
CTTAGAAGTTCCACAAACTGTTGATTGTTTACAAGGTCTAATTAATATT
ATTCCATTACAACTAATGTCATATTGGTTGGCTGTTAATAAAGGGATTG
ATGTTGATTTTCCAAGAAACTTGGCTAAATCTGTTACCGTCGAATAA

SEQ ID NO: 73 is a reencoded nucleic acid sequence of glutamine-fructose-6-phosphate amidotransferase (GFA1) originating from Chlorella virus 1 (PBCV-1)

ATGTGCGGGATCTTCGGTGCTGTGTCGAACAATAATAGCATAGAAGTTT
CCATCAAGGGTATACAGAAGCTAGAGTACCGCGGGTACGATTCGTGTGG
AATAGCCTATACAGACGGAGGCGCCATTGAACGGATCAGGTCAATAGAT
GGGATCGACGACTTAAGGAAGAAAACAATAACAGAGTCTTCCCCGGTAG
CTATCGCTCATTCAAGGTGGAGTACCACTGGGATTCCAAGTGTTGTGAA
CGCCCACCCGCACATCTCTCGGGGCACGTCTGGATGCGAGTCGCGCATT
GCAGTCGTACACAATGGCATTATTGAAAATTACCAGCAGATCCGCAAGT
ACTTGATAAATCTTGGGTATACTTTCGATTCACAGACCGACACGGAAGT
CATTGCTCATCTAATAGACTCGCAGTATAACGGGAACATCCTGCACACC
GTCCAAATGGCAGTTAAGCACCTCAAAGGAAGCTACGCCATAGCAGTCA
TGTGCCATAAGGAGTCCGGGAAGATCGTGGTGGCAAAACAGAAGAGTCC
GTTGGTATTGGGTATAGGAAGCGACGGAGCTTATTATATCGCATCGGAC
GTATTGGCTCTTCCCACTAACAAAGTGGTCTACATAAGTGACGGATTCT
CTGCTGAGTTAAGTCCTGGATCGATGACTATCTATGATCTGGATGGCAA
TAAGGTAGAATACGAGGTGGAAGACGTTGAAATGGAGCAGACGTCGATG
TCTTTAGATAACTTTGATCACTACATGATCAAAGAGATCAATGAACAGC
CGATATCAATTCTCAATACTATTAAGAACAAAGGATTCTATGCAGAGAT
ATTTGGTGATCTAGCACATGAAATATTTCAGAAGATCGACAATATATTG
ATTCTAGCCTGCGGAACTTCGTATCATGCAGGTCTCGTCGGGAAGCAAT
GGATAGAGACCATCTCACGCATCCCGGTGGATGTTCATATTGCAAGCGA
GTATGAGCCGACAATTCCACGAGCCAATACACTTGTGATAACGATTAGT
CAAAGCGGCGAGACTGCGGATACAATCGCAGCCCTTCAACGAGCGCAAA
ACGCAGGAATGATATACACTTTGTGTATCTGCAACAGCCCCAAGTCAAC
TCTAGTGCGCGAGTCGATAATGAAATACATAACCAAATGCGGTTCAGAA
GTTTCTGTTGCCAGTACTAAAGCATTTACGTCGCAACTTGTGGTGTTGT
ATATGCTTGCGAACGTTCTTGCGAACAAAACTGATGATCTGCTAGGGGA
TCTTCCTCAAGCTATCGAGAGAGTAATCTGTCTTACTAATGACGAAATG
AAGCGTTGGGCGGATGAAATTTGTACTGCCAAAAGTGCCATCTTCTTAG
GGAGAGGACTGAACGCACCTGTAGCATTTGAGGGCGCGCTAAAGTTGAA
GGAAATCTCATATATCCATGCCGAGGGTTTCCTCGGTGGAGAGTTGAAG
CATGGGCCGCTGGCTTTGTTGGACGATAAGATCCCTGTAATCGTGACTG
TTGCTGACCACGCTTATTTGGATCACATTAAGGCTAACATCGACGAGGT
ATTGGCACGAAACGTTACCGTATACGCGATCGTCGATCAGTATGTTAAT
ATTGAACCACAGGAGCGACTGCATGTAGTAAAAGTGCCGTTTGTGAGTA
AAGAGTTTTCTCCCATCATCCACACAATTCCGATGCAATTGTTATCGTA
TTATGTCGCGATCAAGCTGGGCAAGAACGTTGACAAGCCACGTAACCTG
GCGAAAAGTGTTACAACATTCtaa

SEQ ID NO: 74 is a reencoded nucleic acid sequence of glutamine-fructose-6-phosphate amidotransferase (GFA1) originating from Chlorella virus 1 (PBCV-1)

ATGTGTGGCATCTTTGGAGCAGTGTCAAACAACAACTCTATCGAGGTGT
CAATCAAGGGTATTCAGAAGCTAGAATATCGTGGGTATGATTCGTGCGG
TATTGCGTATACAGATGGGGGTGCGATTGAGCGTATACGTTCTATTGAC
GGCATTGACGATCTGCGTAAGAAAACAATCACAGAATCATCACCAGTGG
CCATTGCTCACTCGCGGTGGAGCACCACTGGAATTCCATCAGTGGTGAA
CGCACATCCTCATATTTCTCGCGGAACCAGTGGGTGTGAGTCTCGTATC
GCGGTAGTCCACAACGGTATCATTGAAAACTATCAGCAGATCCGAAAAT
ATCTCATCAATCTTGGTTATACGTTTGATAGTCAAACGGACACAGAGGT
CATTGCGCATTTGATTGATTCTCAGTACAATGGGAATATCTTGCACACC
GTCCAAATGGCTGTCAAGCACCTGAAGGGCTCTTATGCCATTGCAGTTA
TGTGTCATAAAGAGTCTGGTAAAATAGTCGTGGCGAAACAGAAGTCACC
CCTCGTACTTGGAATCGGCTCAGATGGTGCTTACTACATCGCTTCGGAC
GTGCTGGCGCTGCCGACAAATAAAGTTGTTTATATTTCAGACGGTTTCT
CCGCAGAACTATCTCCAGGGAGTATGACCATTTACGATCTTGATGGAAA
TAAAGTAGAATATGAAGTAGAGGACGTTGAAATGGAACAAACTAGTATG
TCTCTCGATAACTTTGATCATTACATGATTAAGGAAATTAATGAGCAAC
CAATCAGTATTCTAAACACTATAAAAAATAAAGGGTTCTATGCAGAAAT
ATTCGGTGATTTGGCTCATGAAATCTTCCAAAAAATAGACAACATCCTG
ATACTGGCTTGTGGTACAAGTTATCACGCCGGTCTTGTAGGAAAACAGT
GGATAGAGACCATCTCtAGAATCCCCGTGGATGTTCACATCGCGAGTGA
ATACGAACCTACTATTCCGAGAGCGAACACATTGGTAATCACTATTTCA
CAGTCGGGTGAAACTGCGGACACGATAGCGGCTTTGCAACGGGCCCAAA
ACGCCGGGATGATTTATACATTGTGTATTTGCAATTCACCAAAGAGCAC
TCTTGTTCGTGAGAGCATTATGAAGTACATCACGAAATGTGGTTCTGAG
GTGTCAGTGGCATCAACGAAGGCGTTTACTTCTCAGCTCGTAGTACTGT
ACATGCTGGCAAACGTATTGGCAAATAAAACCGATGATTTGCTGGGAGA
CCTCCCACAGGCAATAGAACGGGTAATTTGTTTGACAAATGACGAAATG
AAACGATGGGCCGACGAAATTTGCACTGCGAAATCCGCGATCTTCCTGG
GAAGAGGACTAAACGCACCAGTTGCCTTTGAGGGAGCGTTGAAGCTCAA
AGAAATCTCTTACATTCATGCAGAGGGCTTCCTGGGAGGTGAGTTGAAA
CACGGCCCCCTCGCACTCCTTGATGACAAGATTCCTGTTATCGTAACCG
TAGCAGATCATGCTTATTTGGACCATATCAAAGCAAATATCGACGAAGT
GCTTGCGAGGAACGTTACGGTATACGCCATAGTAGACCAGTATGTGAAC
ATCGAGCCCCAGGAACGCCTTCACGTCGTCAAGGTTCCGTTTGTATCCA
AAGAATTTTCTCCGATAATTCACACTATCCCGATGCAACTGCTTTCGTA
TTACGTGGCAATTAAGCTTGGAAAGAACGTTGACAAACCAAGGAATCTT
GCAAAATCCGTGACTACCTTTTAA

SEQ ID NO: 75 is an amino acid sequence of glutamine-fructose-6-phosphate amidotransferase (GFA1) originating from Saccharomyces cerevisiae

MCGIFGYCNYLVERSRGEIIDTLVDGLQRLEYRGYDSTGIAIDGDEADS
TFIYKQIGKVSALKEEITKQNPNRDVTFVSHCGIAHTRWATHGRPEQVN
CHPQRSDPEDQFVVVHNGIITNFRELKTLLINKGYKFESDTDTECIAKL
YLHLYNTNLQNGHDLDFHELTKLVLLELEGSYGLLCKSCHYPNEVIATR
KGSPLLIGVKSEKKLKVDFVDVEFPEENAGQPEIPLKSNNKSFGLGPKK
AREFEAGSQNANLLPIAANEFNLRHSQSRAFLSEDGSPTPVEFFVSSDA
ASVVKHTKKVLFLEDDDLAHIYDGELHIHRSRREVGASMTRSIQTLEME
LAQIMKGPYDHFMQKEIYEQPESTENTMRGRIDYENNKVILGGLKAWLP
VVRRARRLIMIACGTSYHSCLATRAIFEELSDIPVSVELASDFLDRKCP
VFRDDVCVFVSQSGETADTMLALNYCLERGALTVGIVNSVGSSISRVTH
CGVHINAGPEIGVASTKAYTSQYIALVMFALSLSDDRVSKIDRRIEIIQ
GLKLIPGQIKQVLKLEPRIKKLCATELKDQKSLLLLGRGYQFAAALEGA
LKIKEISYMHSEGVLAGELKHGVLALVDENLPIIAFGTRDSLFPKVVSS
IEQVTARKGHPIIICNENDEVWAQKSKSIDLQTLEVPQTVDCLQGLINI
IPLQLMSYWLAVNKGIDVDFPRNLAKSVTVE

SEQ ID NO: 76 is an amino acid sequence of glutamine-fructose-6-phosphate amidotransferase (GFA1) originating from Chlorella virus 1 (PBCV-1)

MCGIFGAVSNNNSIEVSIKGIQKLEYRGYDSCGIAYTDGGAIERIRSID
GIDDLRKKTITESSPVAIAHSRWSTTGIPSVVNAHPHISRGTSGCESRI
AVVHNGIIENYQQIRKYLINLGYTFDSQTDTEVIAHLIDSQYNGNILHT
VQMAVKHLKGSYAIAVMCHKESGKIVVAKQKSPLVLGIGSDGAYYIASD
VLALPTNKVVYISDGFSAELSPGSMTIYDLDGNKVEYEVEDVEMEQTSM
SLDNFDHYMIKEINEQPISILNTIKNKGFYAEIFGDLAHEIFQKIDNIL
ILACGTSYHAGLVGKQWIETISRIPVDVHIASEYEPTIPRANTLVITIS
QSGETADTIAALQRAQNAGMIYTLCICNSPKSTLVRESIMKYITKCGSE
VSVASTKAFTSQLVVLYMLANVLANKTDDLLGDLPQAIERVICLTNDEM
KRWADEICTAKSAIFLGRGLNAPVAFEGALKLKEISYIHAEGFLGGELK
HGPLALLDDKIPVIVTVADHAYLDHIKANIDEVLARNVTVYAIVDQYVN
IEPQERLHVVKVPFVSKEFSPIIHTIPMQLLSYYVAIKLGKNVDKPRNL
AKSVTTF

SEQ ID NO: 77 is a nucleic acid sequence of UDP-N-acetylglucosamine pyrophosphorylase (QRI1) originating from Saccharomyces cerevisiae

ATGACTGACACAAAACAGCTATTCATTGAAGCCGGACAAAGTCAACTTT
TCCACAATTGGGAAAGCTTGTCTCGCAAAGACCAAGAAGAATTGCTTTC
AAACCTGGAGCAAATATCTTCCAAGAGGTCCCCTGCAAAACTACTGGAA
GACTGTCAAAATGCTATTAAATTCTCACTAGCTAACTCTTCTAAGGATA
CTGGCGTCGAAATTTCACCATTGCCCCCTACTTCGTACGAGTCGCTTAT
TGGCAACAGTAAGAAAGAAAATGAATACTGGCGTTTAGGCCTTGAAGCT
ATTGGCAAGGGTGAAGTCGCAGTGATTTTAATGGCTGGCGGACAAGGTA
CGCGGTTAGGATCCTCTCAACCAAAGGGCTGTTACGACATTGGATTGCC
TTCTAAGAAATCTCTTTTTCAAATTCAAGCTGAAAAGTTGATCAGGTTG
CAAGATATGGTAAAGGACAAAAAGGTAGAAATTCCTTGGTATATTATGA
CATCAGGCCCCACTAGAGCGGCTACTGAGGCATACTTTCAAGAACACAA
TTATTTTGGCTTGAATAAAGAACAAATTACGTTCTTCAACCAGGGAACC
CTGCCTGCCTTTGATTTAACCGGGAAGCATTTCCTAATGAAAGACCCAG
TAAACCTATCTCAATCACCAGATGGAAATGGTGGACTCTACCGTGCCAT
CAAGGAAAACAAGTTGAACGAAGACTTTGATAGGAGAGGAATCAAGCAT
GTTTACATGTACTGTGTCGATAATGTCCTATCTAAAATCGCAGACCCTG
TATTTATTGGTTTTGCCATCAAGCATGGCTTCGAACTGGCCACCAAAGC
CGTTAGAAAGAGAGATGCGCATGAATCAGTTGGGTTAATTGCTACTAAA
AACGAGAAACCATGTGTCATAGAATATTCTGAAATTTCCAATGAATTGG
CTGAAGCAAAGGATAAAGATGGCTTATTAAAACTACGCGCAGGCAACAT
TGTAAATCATTATTACCTAGTGGATTTACTAAAACGTGATTTGGATCAG
TGGTGTGAGAATATGCCATATCACATTGCGAAGAAGAAAATTCCAGCTT
ATGATAGTGTTACCGGCAAGTACACTAAGCCTACCGAACCAAACGGTAT
AAAATTAGAGCAATTCATATTTGATGTCTTTGACACTGTACCACTGAAC
AAGTTTGGGTGCTTAGAAGTAGATAGATGCAAAGAATTTTCACCTTTAA
AAAACGGTCCTGGTTCTAAGAACGATAATCCTGAGACCAGCAGACTAGC
ATATTTGAAACTAGGAACCTCGTGGTTGGAAGATGCAGGCGCTATTGTA
AAAGATGGGGTACTAGTCGAAGTTTCCAGCAAATTGAGTTATGCAGGTG
AAAATCTATCCCAGTTCAAAGGTAAAGTCTTTGACAGAAGTGGTATAGT
ATTAGAAAAATAA

SEQ ID NO: 78 is an amino acid sequence of UDP-N-acetylglucosamine pyrophosphorylase (QRI1) originating from Saccharomyces cerevisiae

MTDTKQLFIEAGQSQLFHNWESLSRKDQEELLSNLEQISSKRSPAKLLE
DCQNAIKFSLANSSKDTGVEISPLPPTSYESLIGNSKKENEYWRLGLEA
IGKGEVAVILMAGGQGTRLGSSQPKGCYDIGLPSKKSLFQIQAEKLIRL
QDMVKDKKVEIPWYIMTSGPTRAATEAYFQEHNYFGLNKEQITFFNQGT
LPAFDLTGKHFLMKDPVNLSQSPDGNGGLYRAIKENKLNEDFDRRGIKH
VYMYCVDNVLSKIADPVFIGFAIKHGFELATKAVRKRDAHESVGLIATK
NEKPCVIEYSEISNELAEAKDKDGLLKLRAGNIVNHYYLVDLLKRDLDQ
WCENMPYHIAKKKIPAYDSVTGKYTKPTEPNGIKLEQFIFDVFDTVPLN
KFGCLEVDRCKEFSPLKNGPGSKNDNPETSRLAYLKLGTSWLEDAGAIV
KDGVLVEVSSKLSYAGENLSQFKGKVFDRSGIVLEK

SEQ ID NO: 79 is a nucleic acid sequence of Phosphoglucomutase-1 (PGM1) originating from Saccharomyces cerevisiae

ATGTCACTTCTAATAGATTCTGTACCAACAGTTGCTTATAAGGACCAAA
AACCGGGTACTTCAGGTTTACGTAAGAAGACCAAGGTTTTCATGGATGA
GCCTCATTATACTGAGAACTTCATTCAAGCAACAATGCAATCTATCCCT
AATGGCTCAGAGGGAACCACTTTAGTTGTTGGAGGAGATGGTCGTTTCT
ACAACGATGTTATCATGAACAAGATTGCCGCAGTAGGTGCTGCAAACGG
TGTCAGAAAGTTAGTCATTGGTCAAGGCGGTTTACTTTCAACACCAGCT
GCTTCTCATATAATTAGAACATACGAGGAAAAGTGTACCGGTGGTGGTA
TCATATTAACTGCCTCACACAACCCAGGCGGTCCAGAGAATGATTTAGG
TATCAAGTATAATTTACCTAATGGTGGGCCAGCTCCAGAGAGTGTCACT
AACGCTATCTGGGAAGCGTCTAAAAAATTAACTCACTATAAAATTATAA
AGAACTTCCCCAAGTTGAATTTGAACAAGCTTGGTAAAAACCAAAAATA
TGGCCCATTGTTAGTGGACATAATTGATCCTGCCAAAGCATACGTTCAA
TTTCTGAAGGAAATTTTTGATTTTGACTTAATTAAAAGCTTCTTAGCGA
AACAGCGCAAAGACAAAGGGTGGAAGTTGTTGTTTGACTCCTTAAATGG
TATTACAGGACCATATGGTAAGGCTATATTTGTTGATGAATTTGGTTTA
CCGGCAGAGGAAGTTCTTCAAAATTGGCACCCTTTACCTGATTTCGGCG
GTTTACATCCCGATCCGAATCTAACCTATGCACGAACTCTTGTTGACAG
GGTTGACCGCGAAAAAATTGCCTTTGGAGCAGCCTCCGATGGTGATGGT
GATAGGAATATGATTTACGGTTATGGCCCTGCTTTCGTTTCGCCAGGTG
ATTCTGTTGCCATTATTGCCGAATATGCACCCGAAATTCCATACTTCGC
CAAACAAGGTATTTATGGCTTGGCACGTTCATTTCCTACATCCTCAGCC
ATTGATCGTGTTGCAGCAAAAAAGGGATTAAGATGTTACGAAGTTCCAA
CCGGCTGGAAATTCTTCTGTGCCTTATTTGATGCTAAAAAGCTATCAAT
CTGTGGTGAAGAATCCTTCGGTACAGGTTCCAATCATATCAGAGAAAAG
GACGGTCTATGGGCCATTATTGCTTGGTTAAATATCTTGGCTATCTACC
ATAGGCGTAACCCTGAAAAGGAAGCTTCGATCAAAACTATTCAGGACGA
ATTTTGGAACGAGTATGGCCGTACTTTCTTCACAAGATACGATTACGAA
CATATCGAATGCGAGCAGGCCGAAAAAGTTGTAGCTCTTTTGAGTGAAT
TTGTATCAAGGCCAAACGTTTGTGGCTCCCACTTCCCAGCTGATGAGTC
TTTAACCGTTATCGATTGTGGTGATTTTTCGTATAGAGATCTAGATGGC
TCCATCTCTGAAAATCAAGGCCTTTTCGTAAAGTTTTCGAATGGGACTA
AATTTGTTTTGAGGTTATCCGGCACAGGCAGTTCTGGTGCAACAATAAG
ATTATACGTAGAAAAGTATACTGATAAAAAGGAGAACTATGGCCAAACA
GCTGACGTCTTCTTGAAACCCGTCATCAACTCCATTGTAAAATTCTTAA
GATTTAAAGAAATTTTAGGAACAGACGAACCAACAGTCCGCACATAG

SEQ ID NO: 80 is an amino acid sequence Phosphoglucomutase-1 (PGM1) originating from Saccharomyces cerevisiae

MSLLIDSVPTVAYKDQKPGTSGLRKKTKVFMDEPHYTENFIQATMQSIP
NGSEGTTLVVGGDGRFYNDVIMNKIAAVGAANGVRKLVIGQGGLLSTPA
ASHIIRTYEEKCTGGGIILTASHNPGGPENDLGIKYNLPNGGPAPESVT
NAIWEASKKLTHYKIIKNFPKLNLNKLGKNQKYGPLLVDIIDPAKAYVQ
FLKEIFDFDLIKSFLAKQRKDKGWKLLFDSLNGITGPYGKAIFVDEFGL
PAEEVLQNWHPLPDFGGLHPDPNLTYARTLVDRVDREKIAFGAASDGDG
DRNMIYGYGPAFVSPGDSVAIIAEYAPEIPYFAKQGIYGLARSFPTSSA
IDRVAAKKGLRCYEVPTGWKFFCALFDAKKLSICGEESFGTGSNHIREK
DGLWAIIAWLNILAIYHRRNPEKEASIKTIQDEFWNEYGRTFFTRYDYE
HIECEQAEKVVALLSEFVSRPNVCGSHFPADESLTVIDCGDFSYRDLDG
SISENQGLFVKFSNGTKFVLRLSGTGSSGATIRLYVEKYTDKKENYGQT
ADVFLKPVINSIVKFLRFKEILGTDEPTVRT

SEQ ID NO: 81 is a nucleic acid sequence of UTP-glucose-1-phosphate uridylyltransferase (UGP1) originating from Saccharomyces cerevisiae

ATGTCCACTAAGAAGCACACCAAAACACATTCCACTTATGCATTCGAGA
GCAACACAAACAGCGTTGCTGCCTCACAAATGAGAAACGCCTTAAACAA
GTTGGCGGACTCTAGTAAACTTGACGATGCTGCTCGCGCTAAGTTTGAG
AACGAACTGGATTCGTTTTTCACGCTTTTCAGGAGATATTTGGTAGAGA
AGTCTTCTAGAACCACCTTGGAATGGGACAAGATCAAGTCTCCCAACCC
GGATGAAGTGGTTAAGTATGAAATTATTTCTCAGCAGCCCGAGAATGTC
TCAAACCTTTCCAAATTGGCTGTTTTGAAGTTGAACGGTGGGCTGGGTA
CCTCCATGGGCTGCGTTGGCCCTAAATCTGTTATTGAAGTGAGAGAGGG
AAACACCTTTTTGGATTTGTCTGTTCGTCAAATTGAATACTTGAACAGA
CAGTACGATAGCGACGTGCCATTGTTATTGATGAATTCTTTCAACACTG
ACAAGGATACGGAACACTTGATTAAGAAGTATTCCGCTAACAGAATCAG
AATCAGATCTTTCAATCAATCCAGGTTCCCAAGAGTCTACAAGGATTCT
TTATTGCCTGTCCCCACCGAATACGATTCTCCACTGGATGCTTGGTATC
CACCAGGTCACGGTGATTTGTTTGAATCTTTACACGTATCTGGTGAACT
GGATGCCTTAATTGCCCAAGGAAGAGAAATATTATTTGTTTCTAACGGT
GACAACTTGGGTGCTACCGTCGACTTAAAAATTTTAAACCACATGATCG
AGACTGGTGCCGAATATATAATGGAATTGACTGATAAGACCAGAGCCGA
TGTTAAAGGTGGTACTTTGATTTCTTACGATGGTCAAGTCCGTTTATTG
GAAGTCGCCCAAGTTCCAAAAGAACACATTGACGAATTCAAAAATATCA
GAAAGTTTACCAACTTCAACACGAATAACTTATGGATCAATCTGAAAGC
AGTAAAGAGGTTGATCGAATCGAGCAATTTGGAGATGGAAATCATTCCA
AACCAAAAAACTATAACAAGAGACGGTCATGAAATTAATGTCTTACAAT
TAGAAACCGCTTGTGGTGCTGCTATCAGGCATTTTGATGGTGCTCACGG
TGTTGTCGTTCCAAGATCAAGATTCTTGCCTGTCAAGACCTGTTCCGAT
TTGTTGCTGGTTAAATCAGATCTATTCCGTCTGGAACACGGTTCTTTGA
AGTTAGACCCATCCCGTTTTGGTCCAAACCCATTAATCAAGTTGGGCTC
GCATTTCAAAAAGGTTTCTGGTTTTAACGCAAGAATCCCTCACATCCCA
AAAATCGTCGAGCTAGATCATTTGACCATCACTGGTAACGTCTTTTTAG
GTAAAGATGTCACTTTGAGGGGTACTGTCATCATCGTTTGCTCCGACGG
TCATAAAATCGATATTCCAAACGGCTCCATATTGGAAAATGTTGTCGTT
ACTGGTAATTTGCAAATCTTGGAACATTGA

SEQ ID NO: 82 is an amino acid sequence of UTP-glucose-1-phosphate uridylyltransferase (UGP1) originating from Saccharomyces cerevisiae

MSTKKHTKTHSTYAFESNTNSVAASQMRNALNKLADSSKLDDAARAKFE
NELDSFFTLFRRYLVEKSSRTTLEWDKIKSPNPDEVVKYEIISQQPENV
SNLSKLAVLKLNGGLGTSMGCVGPKSVIEVREGNTFLDLSVRQIEYLNR
QYDSDVPLLLMNSFNTDKDTEHLIKKYSANRIRIRSFNQSRFPRVYKDS
LLPVPTEYDSPLDAWYPPGHGDLFESLHVSGELDALIAQGREILFVSNG
DNLGATVDLKILNHMIETGAEYIMELTDKTRADVKGGTLISYDGQVRLL
EVAQVPKEHIDEFKNIRKFTNFNTNNLWINLKAVKRLIESSNLEMEIIP
NQKTITRDGHEINVLQLETACGAAIRHFDGAHGVVVPRSRFLPVKTCSD
LLLVKSDLFRLEHGSLKLDPSRFGPNPLIKLGSHFKKVSGFNARIPHIP
KIVELDHLTITGNVFLGKDVTLRGTVIIVCSDGHKIDIPNGSILENVVV
TGNLQILEH

SEQ ID NO: 83 is a nucleic acid sequence of Glucosamine 6-phosphate N-acetyltransferase (GNA1) originating from Saccharomyces cerevisiae

ATGAGCTTACCCGATGGATTTTATATAAGGCGAATGGAAGAGGGG
GATTTGGAACAGGTCACTGAGACGCTAAAGGTTTTGACCACCGTG
GGCACTATTACCCCCGAATCCTTCAGCAAACTCATAAAATACTGG
AATGAAGCCACAGTATGGAATGATAACGAAGATAAAAAAATAATG
CAATATAACCCCATGGTGATTGTGGACAAGCGCACCGAGACGGTT
GCCGCTACGGGGAATATCATCATCGAAAGAAAGATCATTCATGAA
CTGGGGCTATGTGGCCACATCGAGGACATTGCAGTAAACTCCAAG
TATCAGGGCCAAGGTTTGGGCAAGCTCTTGATTGATCAATTGGTA
ACTATCGGCTTTGACTACGGTTGTTATAAGATTATTTTAGATTGC
GATGAGAAAAATGTCAAATTCTATGAAAAATGTGGGTTTAGCAAC
GCAGGCGTGGAAATGCAAATTAGAAAATAG

SEQ ID NO: 84 is an amino acid sequence of Glucosamine 6-phosphate N-acetyltransferase (GNA1) originating from Saccharomyces cerevisiae

MSLPDGFYIRRMEEGDLEQVTETLKVLTTVGTITPESFSKLIKYW
NEATVWNDNEDKKIMQYNPMVIVDKRTETVAATGNIIIERKIIHE
LGLCGHIEDIAVNSKYQGQGLGKLLIDQLVTIGFDYGCYKIILDC
DEKNVKFYEKCGFSNAGVEMQIRK

SEQ ID NO: 85 is a nucleic acid sequence of phosphoacetylglucosamine mutase (PCM1) originating from Saccharomyces cerevisiae

ATGAAGGTTGATTACGAGCAATTGTGCAAACTCTACGATGACACG
TGCCGCACAAAGAATGTGCAGTTCAGTTACGGTACGGCCGGATTC
AGAACGCTGGCCAAGAATTTGGATACGGTGATGTTCAGTACTGGT
ATACTGGCGGTTCTCAGGTCGCTGAAGCTTCAGGGTCAGTATGTG
GGGGTGATGATCACGGCGTCGCACAACCCATACCAGGACAACGGG
GTCAAGATCGTGGAACCAGACGGATCGATGCTTTTGGCCACATGG
GAGCCATATGCCATGCAGTTGGCCAATGCGGCCTCTTTTGCCACT
AATTTTGAAGAATTTCGTGTTGAGTTGGCCAAGCTGATTGAACAC
GAAAAGATTGATTTGAATACAACCGTCGTGCCTCACATCGTGGTT
GGGAGAGACTCTAGGGAAAGTAGTCCATACTTGCTGCGCTGCTTG
ACTTCCTCCATGGCCAGCGTCTTCCACGCGCAAGTTTTGGACCTA
GGCTGTGTCACTACGCCTCAATTGCATTACATTACTGATTTGTCC
AACAGGCGGAAACTGGAAGGAGACACAGCGCCAGTTGCCACAGAA
CAGGACTACTATTCGTTCTTTATAGGAGCCTTCAACGAGCTCTTC
GCCACGTATCAGCTGGAGAAGAGGCTGTCTGTCCCAAAATTGTTC
ATAGACACAGCCAATGGTATCGGTGGTCCACAGTTGAAAAAACTA
CTGGCCTCCGAAGATTGGGACGTGCCAGCGGAGCAAGTTGAGGTA
ATCAACGACAGGTCCGATGTTCCAGAACTGTTGAATTTTGAATGC
GGTGCGGATTATGTGAAGACTAACCAGAGATTACCCAAGGGTCTT
TCTCCATCCTCGTTTGATTCGCTATATTGCTCCTTTGATGGTGAC
GCAGACAGGGTTGTGTTCTACTATGTCGACTCAGGATCAAAATTT
CATTTGTTGGATGGTGACAAAATTTCCACTTTGTTTGCAAAGTTC
TTGTCTAAACAACTAGAATTGGCACACCTAGAACATTCTTTGAAG
ATTGGTGTTGTGCAAACTGCCTATGCAAACGGCAGTTCCACCGCT
TACATAAAAAATACGTTGCACTGTCCCGTGTCTTGCACTAAGACA
GGTGTTAAACACTTGCATCATGAAGCTGCCACTCAGTACGATATT
GGCATTTATTTCGAAGCAAATGGACATGGTACGATTATATTCAGC
GAAAAATTTCATCGAACTATCAAATCTGAATTATCCAAGTCCAAG
TTAAATGGTGATACGTTAGCTTTGAGAACTTTGAAGTGTTTCTCT
GAATTGATTAATCAGACCGTGGGAGATGCTATTTCAGACATGCTT
GCTGTCCTTGCTACTTTGGCGATTTTGAAAATGTCGCCAATGGAT
TGGGATGAAGAGTATACTGATTTGCCCAACAAGCTGGTTAAGTGC
ATCGTTCCTGATAGGTCAATTTTCCAAACCACGGACCAGGAAAGA
AAATTGCTCAATCCAGTGGGGTTGCAAGACAAGATAGATCTTGTG
GTAGCCAAGTATCCCATGGGAAGAAGCTTTGTCAGAGCCAGTGGT
ACGGAGGATGCGGTGAGGGTTTATGCGGAATGTAAGGACTCCTCT
AAGTTAGGTCAATTTTGTGACGAAGTGGTGGAGCACGTTAAGGCA
TCTGCTTGA

SEQ ID NO: 86 is an amino acid sequence of phosphoacetylglucosamine mutase (PCM1) originating from Saccharomyces cerevisiae

MKVDYEQLCKLYDDTCRTKNVQFSYGTAGFRTLAKNLDTVMESTG
ILAVLRSLKLQGQYVGVMITASHNPYQDNGVKIVEPDGSMLLATW
EPYAMQLANAASFATNFEEFRVELAKLIEHEKIDLNTTVVPHIVV
GRDSRESSPYLLRCLTSSMASVFHAQVLDLGCVTTPQLHYITDLS
NRRKLEGDTAPVATEQDYYSFFIGAFNELFATYQLEKRLSVPKLF
IDTANGIGGPQLKKLLASEDWDVPAEQVEVINDRSDVPELLNFEC
GADYVKTNQRLPKGLSPSSFDSLYCSFDGDADRVVFYYVDSGSKF
HLLDGDKISTLFAKFLSKQLELAHLEHSLKIGVVQTAYANGSSTA
YIKNTLHCPVSCTKTGVKHLHHEAATQYDIGIYFEANGHGTIIFS
EKFHRTIKSELSKSKLNGDTLALRTLKCFSELINQTVGDAISDML
AVLATLAILKMSPMDWDEEYTDLPNKLVKCIVPDRSIFQTTDQER
KLLNPVGLQDKIDLVVAKYPMGRSFVRASGTEDAVRVYAECKDSS
KLGQFCDEVVEHVKASA

SEQ ID NO: 87 is the nucleic acid sequence of promoter pTDH3

CTGCTGTAACCCGTACATGCCCAAAATAGGGGGCGGGTTACACAG
AATATATAACATCGTAGGTGTCTGGGTGAACAGTTTATTCCTGGC
ATCCACTAAATATAATGGAGCCCGCTTTTTAAGCTGGCATCCAGA
AAAAAAAAGAATCCCAGCACCAAAATATTGTTTTCTTCACCAACC
ATCAGTTCATAGGTCCATTCTCTTAGCGCAACTACAGAGAACAGG
GGCACAAACAGGCAAAAAACGGGCACAACCTCAATGGAGTGATGC
AACCTGCCTGGAGTAAATGATGACACAAGGCAATTGACCCACGCA
TGTATCTATCTCATTTTCTTACACCTTCTATTACCTTCTGCTCTC
TCTGATTTGGAAAAAGCTGAAAAAAAAGGTTGAAACCAGTTCCCT
GAAATTATTCCCCTACTTGACTAATAAGTATATAAAGACGGTAGG
TATTGATTGTAATTCTGTAAATCTATTTCTTAAACTTCTTAAATT
CTACTTTTATAGTTAGTCTTTTTTTTAGTTTTAAAACACCAAGAA
CTTAGTTTCGAATAAACACACATAAACAAACAAA

SEQ ID NO: 88 is the nucleic acid sequence of promoter pTDH3.Sk

CAATTCATCGGCCCTTTTAGCGGCTACCCGCGCCATCTAAATGAT
AGGGGGGTGACACTATGGTAAATCCCATAATTAGGTGTCTGGGTG
AGTGGTTCTGATGCCGGCATCCACTAAATATATTGGAGCCCATTT
TTTACGCGGGCTTCCAGAAAAAAAGAGAATCCCAGCACCAAAAGG
TGGTTCTCTTCACCAACCATCAGATCATAGGTCCACAACCACACA
TAACAGGGGCACAAAAAGGCAAAAAACGGACATAACCTCAATGGA
GTGATGCAAATTGACTGGAGCAAAAGCTGACACAAGGCATTGATT
GACCTACGCATGTATCTGTATTCTTTTCTTACACCTTCTATTACC
TTCTAACTCTTTGGGTTGGAAAAAACTGAAAAAAAAGGTTGGGAC
CTGGTTCCCCCAAGTTGTCCCCCTACTTGGTTATTAAATATATAA
AGACAGCAAGTGTTGATTATAATCTTGTAAATCTATAGTTCTTAA
TCTATACTTCTATTTATATTTTAAATTAGTCTTTTTATTTCCAAG
TCCCCAAGAACTTAGTTTCGAATAAACACACACAAATAAACACA

SEQ ID NO: 89 is the nucleic acid sequence of promoter pTDH3-1.sba

GCAGCGCTTCTTCCGCTCTAGTTTTTATAGTTATTATTACTACCA
CCTTAAAAATACGTAAATACTCAAAATAGTAGTGATATTCCCAAC
CTTATTCATCCAAGGCACATCATCATCATCAGCCATTCATCTTTC
ACCTGCCATTAGTAACCCGTCTTCTCATTGAGCGGGTTACGGCAG
CCACAGGCCACATTCCGAATGTCTGGGTGAGCGGTCCCTTTTCCA
GCATCCACTAAATATCTCCGATCCCGCTTTTTAATCTGGCTTCCT
GAAAAAAAGAGAATCCCAGCACCAAAAAATGGCTCTCTTCACCAA
CCATCAGATCATAGGTCCCATTCTCTTACCGCAACCGTACAGAAC
AGGGGAAAACGGGTACAACCTCAATGGAGTGATGCAAACTGACTG
GAGCAAAAAGCTGACACAAGGCAATCGACCTACGTGTCTGTCTAT
TTTCTCACACCTTCTATTACCTTCTAACTCTCTGGGTTGGAAAAA
ACTGAAAAAAAGGTTGAGACCAGTTTCCACAAATCATCCCCCTGT
TTGATTAATAAATATATAAAGACGACAACTATCGATCATAAACTC
ATAAAACTATAACTCCTTTACACTTCTTATTTTATAGTTATTCTA
TTTTAATTCTTATTGATTTTAAAACCCCAAGAACTTAGTTTCGAA
AACACACACACACAAACAATTAAAA

SEQ ID NO: 90 is the nucleic acid sequence of promoter pTDH3.Sar

GAGCTAAATATCAGCCCTTCGGGTCCTGCCTGCTACCCGGTCCTG
TTCGAATAAAAACGCGGGTAACACGACCCAGTAACACCTGTCGTT
GGGTGTCTGGGTCAGAAGTTCTGATACCGGCTTCCACTAAATAGA
TTGGGTTCCGCTCTTTACGCTGGCTTCCTGAAAAAAGAGATTCCG
GGCACCAAAAAATTGGTCTCTTTGCCAACCATCAGATCATAGGTC
CATTCTCTTACCATAACCACACAGGATAGGGGCACCACAGGCGAA
AATGGGCACAAAATCTCAATGGAGTGATGCAAATTAGCTGGAACA
AAAGCTGACACAAGGCAATTAACCTGCGCATGTATCCATCTCCTT
TTCTTACACCTTCTCTTACGTTCTAACTGTTTGGGTTGGAAAAAT
TAAAAAAAAAAGGTTGAGACCAGTTTCCCCAAATCGTCCCCCTAC
TTGATTCATAAATATATAAAGACGACAACTATTGATTATAATCTT
GTAAATCTATAACTCTTTACTTTCTCCTATTTATAATTTAACTTA
ATCTTTTTAGATTTAAAACCCCAAGAACTTAGTTTCGAACAAACA
CACACAAATAAACAAAA

SEQ ID NO: 91 is the nucleic acid sequence of promoter pENO2

CGCTCAGCATCTGCTTCTTCCCAAAGATGAACGCGGCGTTATGTC
ACTAACGACGTGCACCAACTTGCGGAAAGTGGAATCCCGTTCCAA
AACTGGCATCCACTAATTGATACATCTACACACCGCACGCCTTTT
TTCTGAAGCCCACTTTCGTGGACTTTGCCATATGCAAAATTCATG
AAGTGTGATACCAAGTCAGCATACACCTCACTAGGGTAGTTTCTT
TGGTTGTATTGATCATTTGGTTCATCGTGGTTCATTAATTTTTTT
TCTCCATTGCTTTCTGGCTTTGATCTTACTATCATTTGGATTTTT
GTCGAAGGTTGTAGAATTGTATGTGACAAGTGGCACCAAGCATAT
ATAAAAAAAAAAAGCATTATCTTCCTACCAGAGTTGATTGTTAAA
AACGTATTTATAGCAAACGCAATTGTAATTAATTCTTATTTTGTA
TCTTTTCTTCCCTTGTCTCAATCTTTTATTTTTATTTTATTTTTC
TTTTCTTAGTTTCTTTCATAACACCAAGCAACTAATACTATAACA
TACAATAATA

SEQ ID NO: 92 is the nucleic acid sequence of promoter pTEF3

GGCTGATAATAGCGTATAAACAATGCATACTTTGTACGTTCAAAA
TACAATGCAGTAGATATATTTATGCATATTACATATAATACATAT
CACATAGGAAGCAACAGGCGCGTTGGACTTTTAATTTTCGAGGAC
CGCGAATCCTTACATCACACCCAATCCCCCACAAGTGATCCCCCA
CACACCATAGCTTCAAAATGTTTCTACTCCTTTTTTACTCTTCCA
GATTTTCTCGGACTCCGCGCATCGCCGTACCACTTCAAAACACCC
AAGCACAGCATACTAAATTTCCCCTCTTTCTTCCTCTAGGGTGTC
GTTAATTACCCGTACTAAAGGTTTGGAAAAGAAAAAAGAGACCGC
CTCGTTTCTTTTTCTTCGTCGAAAAAGGCAATAAAAATTTTTATC
ACGTTTCTTTTTCTTGAAAATTTTTTTTTTTGATTTTTTTCTCTT
TCGATGACCTCCCATTGATATTTAAGTTAATAAACGGTCTTCAAT
TTCTCAAGTTTCAGTTTCATTTTTCTTGTTCTATTACAACTTTTT
TTACTTCTTGCTCATTAGAAAGAAAGCATAGCAATCTAATCTAAG
TTTTAATTACAAA

SEQ ID NO: 93 is the nucleic acid sequence of promoter pTEF1

GTTTAGCTTGCCTCGTCCCCGCCGGGTCACCCGGCCAGCGACATG
GAGGCCCAGAATACCCTCCTTGACAGTCTTGACGTGCGCAGCTCA
GGGGCATGATGTGACTGTCGCCCGTACATTTAGCCCATACATCCC
CATGTATAATCATTTGCATCCATACATTTTGATGGCCGCACGGCG
CGAAGCAAAAATTACGGCTCCTCGCTGCAGACCTGCGAGCAGGGA
AACGCTCCCCTCACAGACGCGTTGAATTGTCCCCACGCCGCGCCC
CTGTAGAGAAATATAAAAGGTTAGGATTTGCCACTGAGGTTCTTC
TTTCATATACTTCCTTTTAAAATCTTGCTACGATACAGTTCTCAC
ATCACATCCGAACATAAACAACC

SEQ ID NO: 94 is the nucleic acid sequence of promoter pTEF1.ago

GTTTAGCTTGCCTCGTCCCCGCCGGGTCACCCGGCCAGCGACATG
GAGGCCCAGAATACCCTCCTTGACAGTCTTGACGTGCGCAGCTCA
GGGGCATGATGTGACTGTCGCCCGTACATTTAGCCCATACATCCC
CATGTATAATCATTTGCATCCATACATTTTGATGGCCGCACGGCG
CGAAGCAAAAATTACGGCTCCTCGCTGCAGACCTGCGAGCAGGGA
AACGCTCCCCTCACAGACGCGTTGAATTGTCCCCACGCCGCGCCC
CTGTAGAGAAATATAAAAGGTTAGGATTTGCCACTGAGGTTCTTC
TTTCATATACTTCCTTTTAAAATCTTGCTACGATACAGTTCTCAC
ATCACATCCGAACATAAACAACC

SEQ ID NO: 95 is the nucleic acid sequence of promoter pTEF1.Sba

CGCCAACAAACCTTCGAACACTTTAATTTTCGAGGACCGCAGATC
CTCACATCACACCCACACCCAAGACTGCTTCCCCCACACACCCTG
CATCTGTACACTTTCTTCTGCTCTGTTTTTCTCTCCGGCGTTCTC
TCGGGTCGCCCGCATCGCCGCGCCGGCTGGAACCGCCCACGCACC
GCATATTGCAAATCGCCTGCCCCCTCTTGCTCCTTTTTGAGGGCG
CGCCGTTACCCGCGCCCAGGGTCCGGAAAAAGAAACAAGGCTCTA
CCGCGTTTCTTTTTCCTTGTCGAAAAAGGCAAAAATGAAAATTTT
TATCACGTTTCTTTTTTTTTGAAAAATTTTTTTTTTGGTTTTTTT
TCTTTCGATGGCCTCCCATTGATATTTAAGTTAATAAATGGTTTT
CAGTTTTCAAGTTTCAGTTTGTGTTCTTCTTTGCTAACTTTCACT
TACACCTCGAAAGAAAGTATAGCAATCTAATCTTAGTTTTAATTA
CAA

SEQ ID NO: 96 is the nucleic acid sequence of promoter pPDC1 TTATTTACCTATCTCTAAACTTCAACACCTTATATCATAACTAATATTTCTTGAGATAAGCACACTG CACCCATACCTTCCTTAAAAACGTAGCTTCCAGTTTTTGGTGGTTCCGGCTTCCTTCCCGATTCCGCC CGCTAAACGCATATTTTTGTTGCCTGGTGGCATTTGCAAAATGCATAACCTATGCATTTAAAAGATT ATGTATGCTCTTCTGACTTTTCGTGTGATGAGGCTCGTGGAAAAAATGAATAATTTATGAATTTGAG AACAATTTTGTGTTGTTACGGTATTTTACTATGGAATAATCAATCAATTGAGGATTTTATGCAAATA TCGTTTGAATATTTTTCCGACCCTTTGAGTACTTTTCTTCATAATTGCATAATATTGTCCGCTGCCCC TTTTTCTGTTAGACGGTGTCTTGATCTACTTGCTATCGTTCAACACCACCTTATTTTCTAACTATTTTT TTTTTAGCTCATTTGAATCAGCTTATGGTGATGGCACATTTTTGCATAAACCTAGCTGTCCTCGTTG AACATAGGAAAAAAAAATATATAAACAAGGCTCTTTCACTCTCCTTGCAATCAGATTTGGGTTTGT TCCCTTTATTTTCATATTTCTTGTCATATTCCTTTCTCAATTATTATTTTCTACTCATAACCTCACGCA AAATAACACAGTCAAATCAATCAAA
SEQ ID NO: 97 is the nucleic acid sequence of promoter pCCW12

aaccagggcaaagcaaaataaaagAAACTTAATACGTTATGCCGT
AATGAAGGGCTACCAAAAACGATAATCTCAACTGTAAACAGGTAC
AATGCGGACCCTTTTGCCACAAAACATACATCATTCATTGCCGGA
AAAAGAAAGAAGTGAAGACAGCAGTGCAGCCAGCCATGTTGCGCC
AATCTAATTATAGATGCTGGTGCCCTGAGGATGTATCTGGAGCCA
GCCATGGCATCATGCGCTACCGCCGGATGTAAAATCCGACACGCA
AAAGAAAACCTTCGAGGTTGCGCACTTCGCCCACCCATGAACCAC
ACGGTTAGTCCAAAAGGGGCAGTTCAGATTCCAGATGCGGGAATT
AGCTTGCTGCCACCCTCACCTCACTAACGCTGCGGTGTGCGGATA
CTTCATGCTATTTATAGACGCGCGTGTCGGAATCAGCACGCGCAA
GAACCAAATGGGAAAATCGGAATGGGTCCAGAACTGCTTTGAGTG
CTGGCTATTGGCGTCTGATTTCCGTTTTGGGAATCCTTTGCCGCG
CGCCCCTCTCAAAACTCCGCACAAGTCCCAGAAAGCGGGAAAGAA
ATAAAACGCCACCAAAAAAAAAAATAAAAGCCAATCCTCGAAGCG
TGGGTGGTAGGCCCTGGATTATCCCGTACAAGTATTTCTCAGGAG
TAAAAAAACCGTTTGTTTTGGAATTCCCCATTTCGCGGCCACCTA
CGCCGCTATCTTTGCAACAACTATCTGCGATAACTCAGCAAATTT
TGCATATTCGTGTTGCAGTATTGCGATAATGGGAGTCTTACTTCC
AACATAACGGCAGAAAGAAATGTGAGAAAATTTTGCATCCTTTGC
CTCCGTTCAAGTATATAAAGTCGGCATGCTTGATAATCTTTCTTT
CCATCCTACATTGTTCTAATTATTCTTATTCTCCTTTATTCTTTC
CTAACATACCAAGAAATTAATCTTctgtcattcgcttaaacacta
tatcaata

SEQ ID NO: 98 is the nucleic acid sequence of promoter pCCW12.Sm

CGTTGTGCTGTAGTGAAGGAAGACTAAAAAGGATAATCACAGTTG
TAAAGAGGTATAATGCGGACCCTTTTGCCACAAAACACACATCTT
TCGTTTCCAAAATAAGAAAGAAGAAAGCAAAAAGATTAGCAGCCA
CGTTGCTGCGATCTAATTATAGACGGTGGCGTCATCATTCTCACC
CAAGATTGTGTCTTGAACCTGCCACGGGTCCTGCGTTATCGCCGG
ATGTAAAACTAGACATGCAAAAAAAGGACCTTCCAGGTAGCGTGC
TCCACACCACCCATGACCACCACAGTTAGTCCAAAAGAGGCAGCA
CCACTTCCCGATGGGGGAATTAGATTGCTGCCACCCTCACCTCAC
TAATGCTGCGGTGTGCGGATATGCCCTGCTATATATAGCTCCGCG
TTTTTGAACCAGCACAGCGCGAGCACCAAAAAGGAAAATCGCATA
GGCCCAGAACTGATTTCAGCACGGGCTATTGGCGTTGGGTTTCCG
TTCTGGGAAACCTTCGCCGCGTCCCCCTCACAAACCTCCGCACAA
GTTCGAGCAAGCGGGAAAAAACGAAAAACGCCATTAATACTAAAT
AAAGCAAATCCTCGAAGCGTGGGTGGCAAGCCCCTGGATTTTTCC
GCACAAGTACTCTTCTCAGGAGTAAAAAAACCCGTTTGTTTTGGA
ATTCCCCATTTCGCGGCCATCTACGCCGGTATCTTCGCAATATCT
ATCAGCGATAACTCAGCAATTTTAATATTCGTGTTGCAGTGCTGC
GATAGCGGGAGTCTTGTTTGTAACATAACGGCAGAAAGGAATGAG
AGAAAATTTTCCATTCTTTGGCCTCCGTTCAAGTATATAAAGCCG
GCATGCTTGGTAATCTTTCTCTCTCTTCTGTATTGTTTCTATAAT
ACTTTTATCTTCTAATTATTTTCTGAAAAAACCAAGAAATTAATC
TTCTGTCATTCGCTTAAACACTATATCAATA

SEQ ID NO: 99 is the nucleic acid sequence of promoter pCCW12.Sk

TGTAAAATCCTACACGCAAAAAACCTCTTGGGTTGCGCGTTTTCC
ACCACCCACGACCCGCACAATCAATCCAAAAGGGGCAACGCCAGT
TCCCAATGCGGGAATTAGCTCCTCACCCCCCTCACCCGCTAACGC
TGCGGTGTGCGGACACGCAGCAGTATTTATAGATCCTCGTGTTGG
AACCAGCCCGCGTGAGCACCAAATTGGAAAGTCGCAATGGGCCCA
GAACCGCTTTCAGTACTGGGCCATTGGCGTCTAGTTTCCGTTTTG
AGCGTCCTTCGCCGCGTCTCTCTGTGAAATCCCCGCACAAGTCTG
AGCAGGCAAAAAAAAAAAACGCCACCAAAAATAAATAAAGCCAAT
CCTCGAAGGATGAGTAGGAAAGGAAGCCCCTGGTTTTTTCCCGCA
CGAATATTTTTCAGGAGTGAAAAAATCCGTTTGTTTTGGAATTCC
CCATTTCGCGCTCACCTACGCCGGTATCTTTGGAACAACTATCAG
CGATAACTCAGCAAAATTTGCATATCCGTGCTGCAATAGTGCGAT
AGTGGGATTGGGAGTCTTGTTGCATCATAACGGCAGAAAGGAATG
AATAAAAATTTTCCGTTCTTTGTCTCCGTTCAAGTATATAAAGTC
GGCATGCTTGATTATCTTTTCTTCTCTTCTACTACATTTCTATTT
CACTTTCTACTCTATTCTTCCCTGAAAAACCCAAGAAAATAATCT
TCTGTCATTCGCTTAAACAATATATCAAAA

SEQ ID NO: 100 is the nucleic acid sequence of promoter pCCW12.Sba

ACCATGCCACGGTGCTGGCCCCACTTCCACCCACGACCCGCACGG
TTGGCCCGAAAGGGGCAACACCAGTTCCCAATACGGAAATTAGTC
CGCCACCACCCTCACCTCGCTAGAGCTGCGGTGTGCGGGCGTGCA
TCGCTATTTATAGACATGCCTGCTGGCGTCACCGCGCGCGAGCAC
CAAACAGGAAAATCGCACTGGGCCCAGAACCACGCTATGCGCTGG
GCCGATGGCGTCCGGTTTCCCTTTGGGAGCCCCCTGCCACGTTCG
CCTAACAAATCCCCGCACCGGCTTGAGAAAAAAGCGAAAAGCGAA
AAAAAAAAATCAACGCCACCAAAATTAAAAAAAAAGAGCCATCCT
CGAAGGGTGAATAGTAGCCCCTGACTTTTCCCGCACAGACAGACA
CCTTTCAGGAGTGAACAAAAAAGCAGTTTGTTTTGGAATTCCCCC
ATTTCGCGGTGGCCTGCGCAGGTATCTCTGCGTCAACTATCAGCG
ATAACTCAGCAAATTTTGCATATTCGTGTTGCGATACTACGATAA
TGGGAGTCTGTCGCCTAATAACGGCAACAAGGAATGAGAGAGAAA
AATTTTCTTCATTCTCCAGCTCCCGTTCAAGTATATAAGGTCGGC
ATGGTCGATTGTCTTTCCTTCTCTTCAGTTACGTCTCTCTATTTA
CATTATTCTTATTTTTATTTAATAAAAACCCCAAGAAATTAATCT
TCTGTCATTCGCTTAAACACTATATCAAAA

SEQ ID NO: 101 is the nucleic acid sequence of promoter pCCW12.Sar

AAAAAACAACCTTCTGCCAACCTGCGTGCTTCTCACCACCCATGA
CCCACACAATTGACCCGAATGGGGCAACTCCAGTTCCCAATACGG
GAATTAACTCGCCACCATATTTACCGCGTTGAAGCTGTGGTGTGC
GGACACTCCGTACTATTTATAGACCCACGCGGTGGAACCAGCACG
CGCGCGCACTAAACAGGAAAATCGCATTGAGTCCAGAACCGCCAC
CAGCACTTGGCCATTGGCGTCTAATTTCCGTTTTCGGCGCCCCTC
ACCGCGTCCTTCTAACAAAGCGCGCACAAGCTTGAGCAAGTGAAA
AGAAAATTAAAAATAAAAAACCGCCACCAAAACAAATAAAGCAAT
CTTCGAAGTGTGGGTTGGTGGGAAGCCCCTGGCTTTTCCCGCACC
AGTCGTTTTCAGGAGTAAAAAAATACCCGTTTGTTTTGGAATTCC
CCATTTCGCGGCGACCTGCGCCGGTATCTTTGCAACAACTATTTG
CGATAACTCAGCAAAATTTGCATATTCGTGTTGGGATATTGCGAT
AGTGGGAGTCTTGTTGCATAATAACGGTAAAAAGAAGTGAAGGAA
AAAAATTTGCATCCTTTAGTCTCAGTTCAAGTATATAAAGTCGGG
ATATTCAATTATCTTTCTTTCTCTTGCTCAAAGGTTTCTATATTT
TTTTTATAGTATTTCTTTTGTTATAAAATACCAAGAAATTAATCT
TCTGTCATTCGCTTAAATACTACATCAATA

SEQ ID NO: 102 is the nucleic acid sequence of promoter pNUP57

TCATCTGCGCAATGACTATCAAGACCTTCTGCAAGAATTTCAAAT
CTCACTGAAAATCTTGACCGAAAAGTGTCTTGAAAACCCATCAAG
CCTGCAAAACCTATCTTTGACATTAGTCTCCATTATAAAAACGGC
ATAGTTGGGAGAAAACTTTCCATACTTCAATTGTGGACTGATATA
AGTATTTTAGTTTTGCCCGCATGATCATCCCACATGGCTACAGCA
GTTCTCTCATAGGAAAATAGTACAATAGCTACGTGATATAATCTA
AATAATTGTTGCCAATGTGTAATTATATCATTTTGAACGTACGCG
AAATGGATTATTTTCAAAAATTTTGTTTCTTGAAATGAGTAAAAG
CAAAAGTCCAACTCTCCAAGTCGATGTAAACAACTTTTTGCCAAA
GGGACTGAAAGACTAAATCGAGGATTATCCCGTTCAAACTATTCC
AGAAACGCTCGTTAGTAACAAAAGACATACCTTGTTGACCAATTG
ATCAC

SEQ ID NO: 103 is the nucleic acid sequence of promoter pCCW10.ago

GGTACCACGGCAACCTCGTTCGCTGTTCATCCCCTTCGTCACACA
GGACGTTGGATGCCGTAAGCAGCGTTGCTTTTGATCCTCagGATC
GGCCGGGTAACCCGCGGCtGCTTCTATTTTAGTATTCATATCTCA
AGCACATCCATTCCGGCCGTTTGGGGGCGCCGCCGCACTCGTGTC
CATTCCTACCGTGGCACTTAGGGCTATCCTGTCGGAGCGCCCCGC
CGACCGCCTTATCGGCACCAAAAGTAGAAGCCCCGGCCCCGCGTG
GCTCAGACTCACCATCGGTGCTATTTACTTTTCGATCAGATCGCG
GCGCGCGGTGGCCGGCATTTCCGGAAGCGGCCACGGAGCAGAGGT
GGCGCATTCGAATCGCATACGTCTTCGCCACGCCGGAAAAAAAAT
TTTCGGCTATATAAGGAGAGGCGGCCGTCTTGCTGCAGGCAGTTT
CACTTTCTCTAAAACCAAAGAACATCGATTTCTTTAGTCACTCGC
TTCCTTACACCGAACTCGAGGCGGCCGC

SEQ ID NO: 104 is the nucleic acid sequence of promoter pCWP2

CTAGCCTCCCCTTTTTATTTTGTGCGGTCACCGCAAGGGACAAAG
CTTTTCTTAGAAAACCGTCTGAGAAGCATAACGTACGCCATCCCC
TAGACATATTAATAATGCTACAGATACTATGCTGCTCGTCTTTTT
TTGACGACCCTTTTATTGCAATGTGCAACTAATGGCAAACAACCA
CATAGTATCACAGTATTACATTGCCTCCACCGATGCGGATGTTAG
GGCGCCAAGTCTGTCATGAAGCATGTTCCTGTCATAATCTTGTAT
GCAAAATACCGCGTTCTGCGCCACTGATATGCTAGGCAGCAGCAA
CCTATGCAGAAGATTGCTTTTCCCACGCCTGTTTTACGTCTCCAG
GGCACTTGAAACAATGCAGCGATCGCCGCCACAACACGCCAAAGA
GAAGCGAAAGTGGGCCTGGGCGGCCTCAGTTTCGGCAGAGGTAAA
CAACACGAACTGAACTGCCTTAGCTCCGAAGGGCAATTCCACAGG
CACTCCGCGGGGCCCGGCCAAGGCCCAAAAGGCGTGGAATATGCG
CGTTTTGGGGCCATAACACCCAGTACCACGGCCGGAACGGGCCAT
ATAATAAGTTTTTCACTCTCAAGAATGGTAAACGTAAATAGGAAC
ATCCCACTACCCTAGAAATTGCGGAAATTTCGCGCTTATCATTAG
AAAATCTGGAACCGTCCTTTTTCCTCTTTCTTGCATTTCCCTTTC
CGTATTATTGCCATTCTTTAACTGCATTTGGGGAACCGTAGACCA
AAAGCCAAACAGAGAAATGTAACGTTCTAAAAAAAAAACAACGAA
AAAATTGAAAAATAAGATACAATAATCGTATATAAATCAGGCTTC
TTGTTCATCATTTTCAATTCTCTTCTTGCCATCCCTTTTCCTATC
TTTGTTCTTTTCTTCTCATAATCAAGAATAAATAACTTCATCA

SEQ ID NO: 105 is the nucleic acid sequence of promoter pFBA1

ACGCAAGCCCTAAGAAATGAATAACAATACTGACAGTACTAAATA
ATTGCCTACTTGGCTTCACATACGTTGCATACGTCGATATAGATA
ATAATGATAATGACAGCAGGATTATCGTAATACGTAATAGTTGAA
AATCTCAAAAATGTGTGGGTCATTACGTAAATAATGATAGGAATG
GGATTCTTCTATTTTTCCTTTTTCCATTCTAGCAGCCGTCGGGAA
AACGTGGCATCCTCTCTTTCGGGCTCAATTGGAGTCACGCTGCCG
TGAGCATCCTCTCTTTCCATATCTAACAACTGAGCACGTAACCAA
TGGAAAAGCATGAGCTTAGCGTTGCTCCAAAAAAGTATTGGATGG
TTAATACCATTTGTCTGTTCTCTTCTGACTTTGACTCCTCAAAAA
AAAAAAATCTACAATCAACAGATCGCTTCAATTACGCCCTCACAA
AAACTTTTTTCCTTCTTCTTCGCCCACGTTAAATTTTATCCCTCA
TGTTGTCTAACGGATTTCTGCACTTGATTTATTATAAAAAGACAA
AGACATAATACTTCTCTATCAATTTCAGTTATTGTTCTTCCTTGC
GTTATTCTTCTGTTCTTCTTTTTCTTTTGTCATATATAACCATAA
CCAAGTAATACATATTCAAA

SEQ ID NO: 106 is the nucleic acid sequence of promoter pCCW120.Sm

CGTTGTGCTGTAGTGAAGGAAGACTAAAAAGGATAATCACAGTTG
TAAAGAGGTATAATGCGGACCCTTTTGCCACAAAACACACATCTT
TCGTTTCCAAAATAAGAAAGAAGAAAGCAAAAAGATTAGCAGCCA
CGTTGCTGCGATCTAATTATAGACGGTGGCGTCATCATTCTCACC
CAAGATTGTGTCTTGAACCTGCCACGGGTCCTGCGTTATCGCCGG
ATGTAAAACTAGACATGCAAAAAAAGGACCTTCCAGGTAGCGTGC
TCCACACCACCCATGACCACCACAGTTAGTCCAAAAGAGGCAGCA
CCACTTCCCGATGGGGGAATTAGATTGCTGCCACCCTCACCTCAC
TAATGCTGCGGTGTGCGGATATGCCCTGCTATATATAGCTCCGCG
TTTTTGAACCAGCACAGCGCGAGCACCAAAAAGGAAAATCGCATA
GGCCCAGAACTGATTTCAGCACGGGCTATTGGCGTTGGGTTTCCG
TTCTGGGAAACCTTCGCCGCGTCCCCCTCACAAACCTCCGCACAA
GTTCGAGCAAGCGGGAAAAAACGAAAAACGCCATTAATACTAAAT
AAAGCAAATCCTCGAAGCGTGGGTGGCAAGCCCCTGGATTTTTCC
GCACAAGTACTCTTCTCAGGAGTAAAAAAACCCGTTTGTTTTGGA
ATTCCCCATTTCGCGGCCATCTACGCCGGTATCTTCGCAATATCT
ATCAGCGATAACTCAGCAATTTTAATATTCGTGTTGCAGTGCTGC
GATAGCGGGAGTCTTGTTTGTAACATAACGGCAGAAAGGAATGAG
AGAAAATTTTCCATTCTTTGGCCTCCGTTCAAGTATATAAAGCCG
GCATGCTTGGTAATCTTTCTCTCTCTTCTGTATTGTTTCTATAAT
ACTTTTATCTTCTAATTATTTTCTGAAAAAACCAAGAAATTAATC
TTCTGTCATTCGCTTAAACACTATATCAATACTCGAAATCCAAGG
TGGTGT

SEQ ID NO: 107 is the nucleic acid sequence of promoter pCUP1

CGGCAAACTTCAACGATTTCTATGATGCATTTTATAATTAGTAAG
CCGATCCCATTACCGACATTTGGGCGCTATACGTGCATATGTTCA
TGTATGTATCTGTATTTAAAACACTTTTGTATTATTTTTCCTCAT
ATATGTGTATAGGTTTATACGGATGATTTAATTATTACTTCACCA
CCCTTTATTTCAGGCTGATATCTTAGCCTTGTTACTAGTTAGAAA
AAGACATTTTTGCTGTCAGTCACTGTCAAGAGATTCTTTTGCTGG
CATTTCTTCTAGAAGCAAAAAGAGCGATGCGTCTTTTCCGCTGAA
CCGTTCCAGCAAAAAAGACTACCAACGCAATATGGATTGTCAGAA
TCATATAAAAGAGAAGCAAATAACTCCTTGTCTTGTATCAATTGC
ATTATAATATCTTCTTGTTAGTGCAATATCATATAGAAGTCATCG
AAATAGATATTAAGAAAAACAAACTGTACAATCAATCAATCAATC
ATCACATAAA

SEQ ID NO: 108 is the nucleic acid sequence of promoter pMET6

CCACAGGAAATATTTCACGTGACTTACAAACAGAGTCGTACGTCA
GGACCGGAGTCAGGTGAAAAAATGTGGGCCGGTAAAGGGAAAAAA
CCAGAAACGGGACTACTATCGAACTCGTTTAGTCGCGAACGTGCA
AAAGGCCAATATTTTTCGCTAGAGTCATCGCAGTCATGGCAGCTC
TTTCGCTCTATCTCCCGGTCGCAAAACTGTGGTAGTCATAGCTCG
TTCTGCTCAATTGAGAACTGTGAATGTGAATATGGAACAAATGCG
ATAGATGCACTAATTTAAGGGAAGCTAGCTAGTTTTCCCAACTGC
GAAAGAAAAAAAGGAAAGAAAAAAAAATTCTATATAAGTGATAGA
TATTTCCATCTTTACTAGCATTAGTTTCTCTTTTACGTATTCAAT
ATTTTTGTTAAACTCTTCCTTTATCATAAAAAAGCAAGCATCTAA
GAGCATTGACAACACTCTAAGAAACAAAATACCAATATAATTTCA
AAGTACATATCAAAA

SEQ ID NO: 109 is the nucleic acid sequence of promoter pMET25

TTACATTATCAATCCTTGCGTTTCAGCTTCCACTAATTTAGATGA
CTATTTCTCATCATTTGCGTCATCTTCTAACACCGTATATGATAA
TATACTAGTAACGTAAATACTAGTTAGTAGATGATAGTTGATTTT
TATTCCAACACTAAGAAATAATTTCGCCATTTCTTGAATGTATTT
AAAGATATTTAATGCTATAATAGACATTTAAATCCAATTCTTCCA
ACATACAATGGGAGTTTGGCCGAGTGGTTTAAGGCGTCAGATTTA
GGTGGATTTAACCTCTAAAATCTCTGATATCTTCGGATGCAAGGG
TTCGAATCCCTTAGCTCTCATTATTTTTTGCTTTTTCTCTTGAGG
TCACATGATCGCAAAATGGCAAATGGCACGTGAAGCTGTCGATAT
TGGGGAACTGTGGTGGTTGGCAAATGACTAATTAAGTTAGTCAAG
GCGCCATCCTCATGAAAACTGTGTAACATAATAACCGAAGTGTCG
AAAAGGTGGCACCTTGTCCAATTGAACACGCTCGATGAAAAAAAT
AAGATATATATAAGGTTAAGTAAAGCGTCTGTTAGAAAGGAAGTT
TTTCCTTTTTCTTGCTCTCTTGTCTTTTCATCTACTATTTCCTTC
GTGTAATACAGGGTCGTCAGATACATAGATACAATTCTATTACCC
CCATCCATACA

SEQ ID NO: 110 is the nucleic acid sequence of promoter pSAM1

GAAACGGACGTAAGACGGAAATAGAATTTGAAGATAAAGTTATAT
ATCACTACACACGAATACTTTCTTTTTTTTTTTTCACAGGAAAAC
TGTGGTGGCGCCCTTGCCTACTAGTGCATTTCTTTTTTCGGGTTC
TTGTCTCGAcGAAATTTTAGCCTCATCGTAGTTTTTCACTCTGGT
ATCGATGAAAAAGGGAAGAGTAAAAAGTTTTCCGTTTAGTACTTA
ATGGGATTGGTTTGGGACGTATATATCGACTGGTGTTGTCTGTTA
TTCATCGTTGTTTTTCGGTTAGCTTCGAAAAAAAAATAGAGTAAA
AACCAGGAATTTACCCTAAAAACAAGAAAAAATAAGATAAACGAA
AAT

SEQ ID NO: 111 is the nucleic acid sequence of terminator tTPI1

GATTAATATAATTATATAAAAATATTATCTTCTTTTCTTTATATC
TAGTGTTATGTAAAATAAATTGATGACTACGGAAAGCTTTTTTAT
ATTGTTTCTTTTTCATTCTGAGCCACTTAAATTTCGTGAATGTTC
TTGTAAGGGACGGTAGATTTACAAGTGATACAACAAAAAGCAAGG
CGCTTTTTCTAATAAAAAGAAGAAAAGCATTTAACAATTGAACAC
CTCTATATCAACGAAGAATATTACTTTGTCTCTAAATCCTTGTAA
AATGTGTACGATCTCTATATGGGTTACTCA

SEQ ID NO: 112 is the nucleic acid sequence of terminator tMET25

GTGTGCGTAATGAGTTGTAAAATTATGTATAAACCTACTTTCTCT
CACAAGTACTATACTTTTATAAAACGAACTTTATTGAAATGAATA
TCCTTTTTTTCCCTTGTTACATGTCGTGACTCGTACTTTGAACCT
AAATTGTTCTAACATCAAAGAACAGTGTTAATTCGCAGTCGAGAA
GAAAAATATGGTGAACAAGACTCATCTACTTCATGAGACTACTTT
ACGCCTCCTATAAAGCTGTCACACTGGATAAATTTATTGTAGGAC
CAAGTTACAAAAGAGGATGATGGAGGTTT

SEQ ID NO: 113 is the nucleic acid sequence of terminator tDIT1

TAAAGTAAGAGCGCTACATTGGTCTACCTTTTTGTTCTTTTACTT
AAACATTAGTTAGTTCGTTTTCTTTTTCTCATTTTTTTATGTTTC
CCCCCCAAAGTTCTGATTTTATAATATTTTATTTCACACAATTCC
ATTTAACAGAGGGGGAATAGATTCTTTAGCTTAGAAAATTAGTGA
TCAATATATATTTGCCTTTCTTTTCATCTTTTCAGTGATATTAAT
GGTTTCGAGACACTGCAATGGCCCTAGTTGTCTAAGAGGATAGAT
GTTACTGTCAAAGATGATATTTTGAATTTC

SEQ ID NO: 114 is the nucleic acid sequence of terminator tRPL3

GAAGTTTTGTTAGAAAATAAATCATTTTTTAATTGAGCATTCTTATTCC
TATGTATTGTTAGCTACATACAACAGTTTAAATCAAATTTTCTTTTTCC
CTATTTTATTTAAATAGTTTAAGTCCAAAATGGAGGTTTATTTTGATGA
CCCGCATGCGATTATGTTTTGAAAGTATAAGACTACATACATGTACATA
TATTTAAACATGTAAACCCGTCCATTATATTGCTTACTTTCTTCTTTTT
TGCCGTTTTGACTTGGACCTCTGGTTTGCTATTTCCTTACAATCTTTGC
TACAAT

SEQ ID NO: 115 is the nucleic acid sequence of terminator tRPL3.sm

GAAGTTTTTAAAGCATTTTTTAGACACTTCTCATTTTTCTAAGTTTTTT
TAAAATAGTTTTATGTATTTACTACGTATCACAATTTGAAATAATTCAT
CTTCCCAAAAAACTAAGATTTTTATCCTTGTCACGATCCGTAACCAGTT
TATAATATTTTAGAGCTTATACACGTACGTATACACACGTGTCGGTACA
TGAGAATTACGTTCAAAATTATTCACTTTTTTTTTCTCTGCCGTTTTAC
TTTTGAACTCTGTCTCGCTATTTCCTTACAATCTTCGCTACAATACCAC
TTGCCCTTGG

SEQ ID NO: 116 is the nucleic acid sequence of terminator tRPL3.sba

GAAGTTTTCTGACAAAAACATAACGTTTTTTCCAATCATTTCTTATTTT
TCCGGTTTATTTAAATAGTTTTTATGTACTATTATACGTATGACTATTT
AACTTAAATTCTTCCTCCCAAGAAATCTCCCAAGTTTTTCATTATCATG
GCATACACCACTATCAGTTACAAAATGGTAGCTCAACCATATATATATC
TCTATATACACATATAAATGCAAACAGGTCCAAGTCACCGCTCACTGCA
GTTTCTTTTGCCGTTTTGACTTCGATCTCTGCTTGGCTATTTTCTCACA
ATCCT

SEQ ID NO: 117 is the nucleic acid sequence of terminator tRPL41B

GCGGATTGAGAGCAAATCGTTAAGTTCAGGTCAAGTAAAAATTGATTTC
GAAAACTAATTTCTCTTATACAATCCTTTGATTGGACCGTCATCCTTTC
GAATATAAGATTTTGTTAAGAATATTTTAGACAGAGATCTACTTTATAT
TTAATATCTAGATATTACATAATTTCCTCTCTAATAAAATATCATTAAT
AAAATAAAAATGAAGCGATTTGATTTTGTGTTGTCAACTTAGTTTGCCG
CTATGCCTCTTGGGTAATGCTATTATTGAATCGAAGGGCTTTATTATAT
TACCCT

SEQ ID NO: 118 is the nucleic acid sequence of terminator tRPL41B.Sba

GCGGATTGCGAGTGATTTTTTGAGTACAGACCAAGTAAAATTGATCTAT
GCATAATCTTTTCTCATCCACAATCCCTAGAATGGACCGTCATCCTTTC
TAAAATGTTTTATTAAGAATATTTAAAGTGGAAGTCAGGTTTATATCTA
TCAATTAAATATTACATAATTTCCCCTTAATAAAATACTGTTAATAAAA
TAACGAAACGTTCATAGTTTTTCATAAATAGTCCTGATGCTTACTGCAC
CGGAACGTACTGTTTTAGTGTATTGCTCCCTTCATTGCCATTTTTCCTA
GTTATTG

SEQ ID NO: 119 is the nucleic acid sequence of terminator tRPL15A

GCTGGTTGATGGAAAATATAATTTTATTGGGCAAACTTTTGTTTATCTG
ATGTGTTTTATACTATTATCTTTTTAATTAATGATTCTATATACAAACC
TGTATATTTTTTCTTTAACCAATTTTTTTTTTTATAGACCTAGAGCTGT
ACTTTTATTCTGCTATCAAGCAAACCCCTACCCCCTCTTCTCAATCCTC
CCCTCAGGCAGAACTTATCTACCTGTATCAAGGAGCGGACGAGGGAGTC
CTAATTGTTCTACGTATACCAATGCTAGCAGCTTACATAGGTGGTGGCA
CTACCA

SEQ ID NO: 120 is the nucleic acid sequence of terminator tRPL15A.Sm

GCTAATTAGAACCATATATACATCGATAAAAACACTTGAATGTATTTTT
CTTTTTCTTCTTATTTTTTCATTTTCCTTCGCTTTCTATTAGCGTGCGA
ATCTAAAGTGTGCAGCGCTATCTTTGACTCTTTTTTTCCTTTAGTGGGA
ACGAACAATATTAATCATGCCAAGTTAAGTCATTGAAGAAGTCAGTAGT
ATGTTATTCGGAGAGAAGACCCTGCTATGTATCTCAGCCTACCCAAAAA
ATGAATTGACTCCTCTCATAACTTGTTAATATTCGGATGAGCACATCAC
GTTCG

SEQ ID NO: 121 is the nucleic acid sequence of terminator tRPL15A.sba

GCTGGCTGATGAAAAATATAGTTCTGTTGGGCAAGCATTTGTTTACCTA
GCATCTCTTTTATACTATTATTATCTTTATATTTGATGATTTTATATAC
AAGTTGTATACCTTTTCTTTAACCAATTTTTTTTTTTCTAATGGTGCAC
CTAGAAGTACATTTTTTCTCACCAATAGATAGTCAAGATACTCCCAGCC
TCTATGGCGTTACCACGGAGCCGACAAGGGAAAGCTGCTTATCTTACAT
ATGCAGATGCCAAGGCCCGTTACAGGCCGCCTTATTGATGTTTAGAAAT
AGCTTC

SEQ ID NO: 122 is the nucleic acid sequence of terminator tIDP1

TCGAATTTACGTAGCCCAATCTACCACTTTTTTTTTTCATTTTTTAAAG
TGTTATACTTAGTTATGCTCTAGGATAATGAACTACTTTTTTTTTTTTT
TTTTTACTGTTATCATAAATATATATACCTTATTGTTGTTTGCAACCGT
CGGTTAATTCCTTATCAAGGTTCCCCAAGTTCGGATCATTACCATCAAT
TTCCAACATTTTCATGAGTTCTTCTTCTTCATTACCGTGTTTTAGGGGG
CTGTTCGCACTTCTAATAGGGCTATCACCAAGCTGTTCTAATTCGTCCA
AAAGTT

SEQ ID NO: 123 is the nucleic acid sequence of terminator tIDP1.Sba

TCTGATCTTTGGAATGCTCCATTTACTTTTTTATTTCATATTTAGGTAC
GATATATAGTTACGCTTGAGAACTATGAACTAATAACTTCCTTTTTCTC
TAATACATACTTACTTCAGCTGATGGTTGCAAACAACAATAAAATTCCT
TGTCGAGGTTCCCTAAATTAGGATCATTGCCATCGACTTCTAACATTTT
CATGAGATCCTCCTCTTCGTTCCCATTCTTTAAAAGACTGTTTCCACTT
CTAATAGGGTTATCGCCCAATTGTTCTAGTTCGTCTAGCAATTCG

SEQ ID NO: 124 is the nucleic acid sequence of terminator tTEF1.sba

GGAGATTGATAGGACTTTTCTAGTTGCATATCTTTTATTTTTAAATCTT
ATCTATTAGTTAATTTTTTGTAATTTATCCTTATATATATAGTTTGGTT
ATTCTAAAACATCATTTCAGTATCTAAAACCTCTCTTATTCATTACCTT
TTTATTTAATGGTTTTTGCTACAGGCAAAAATTTAATGGTTTTTGCTAC
AGGCAAAAATCCCGCCGTGGACTTATTCCACGTTAACTCGGTTACAGGG
TCATGAACCATTTTGTCAATTATCGAAATAACTTCTTCAAAAGTCCCTC
TTACTT

SEQ ID NO: 125 is a nucleic acid sequence of the secretion sequence added

5′
ATGCAATTTAGCACAGTCGCATCAGTAGCCTTCGTTGCCTTGGCCAACT
TCGTGGCAGCA

SEQ ID NO: 126 is an amino acid sequence of the secretion sequence added in N-ter

MQFSTVASVAFVALANFVAA

SEQ ID NO: 127 is a nucleic acid sequence of the anchoring sequence added in 3′

3′
GGATCCGCCATTTCTCAAATCACTGACGGTCAAATCCAAGCTACTACCA
CTGCTACCACCGAAGCTACCACCACTGCTGCCCCATCTTCCACCGTTGA
AACTGTTTCTCCATCCAGCACCGAAACTATCTCTCAACAAACTGAAAAT
GGTGCTGCTAAGGCCGCTGTCGGTATGGGTGCCGGTGCTCTAGCTGCTG
CTGCTATGTTGTTATAA

SEQ ID NO: 128 is an amino acid sequence of the anchoring sequence added in C-ter

AISQITDGQIQATTTATTEATTTAAPSSTVETVSPSSTETISQQTENGA
AKAAVGMGAGALAAAAMLL

1. A recombinant host cell (i) or a recombinant yeast cell ii) producing chondroitin wherein the recombinant host cell (i) or the recombinant yeast cell ii) comprises:
(a) one or more recombinant nucleic acids encoding a polypeptide having a chondroitin synthase (HCOS) activity;
(b) one or more recombinant nucleic acids encoding a polypeptide having UDP-Glucose dehydrogenase (UDP-GlcDH or HASB) activity; and
(c) one or more recombinant nucleic acids encoding a polypeptide having a UDP-glucose-4-epimerase (kfoA or GNE1) activity,
wherein the recombinant host cell (i) further comprises (d) one or more recombinant nucleic acids encoding a polypeptide having a chondroitinase activity wherein the polypeptide having chondroitinase activity comprises a secretion signal so that chondroitin is produced by the recombinant host cell (i), and
wherein the recombinant yeast cell (ii) optionally further comprises (d) one or more recombinant nucleic acids encoding a polypeptide having a chondroitinase activity wherein the polypeptide having chondroitinase activity comprises a secretion signal.

2. The recombinant host cell (i) or the recombinant yeast cell (ii) according to claim 1, wherein the polypeptide having chondroitinase activity comprises a secretion signal, and an anchoring signal.

3. The recombinant host cell (i) according to claim 1 wherein the chondroitin is of a desired molecular weight.

4. The recombinant host cell (i) or the recombinant yeast cell (ii) according to claim 1, wherein the recombinant nucleic acid encoding a polypeptide having a chondroitinase activity is obtained or derived from at least one of Cupiennius salei, Tityus serrulatus, Bos taurus, Vespa magmfica, Macaca mulatta or Apis mellifera.

5. The recombinant host cell (i) or the recombinant yeast cell (ii) according to claim 1, wherein the molecular weight of the chondroitin is less than 50 kDa, or is in a range of 20 kDa to 50 kDa.

6. The recombinant host cell (i) or the recombinant yeast cell (ii) according to claim 1, wherein the molecular weight of the chondroitin is greater than 50 kDa, or is in a range of 50 kDa to 250 kDa or is greater than 100 kDa or is in a range of 100 kDa to 1500 kDa.

7. (canceled)

8. The recombinant host cell (i) or the recombinant yeast cell (ii) according to claim 1, wherein the nucleic acid encoding a polypeptide having a UDP-Glucose dehydrogenase (UDP-GlcDH or HASB) activity is obtained or derived from at least one of Arabidopsis thaliana, Chlorella virus PBCV1 or Streptococcus zooepidemicus.

9. The recombinant host cell (i) or the recombinant yeast cell (ii) according to claim 1, wherein the nucleic acid encoding a polypeptide having a chondroitin synthase (HCOS) activity is
(i) a nucleic acid encoding a chondroitin synthase; or
(ii) a nucleic acid encoding a chimeric polypeptide having a chondroitin synthase activity.

10. The recombinant host cell (i) or the recombinant yeast cell (ii) according to claim 9, wherein the nucleic acid is obtained or derived from at least one of Pasteurella multocida, Chlorella virus PBCV1, Mycobacterium tuberculosis, Homo Sapiens, Escherichia coli, Saccharomyces cerevisiae or Penicillium oxalicum.

11. The recombinant host cell (i) or the recombinant yeast cell (ii) according to claim 1, wherein the nucleic acid encoding a polypeptide having a UDP-glucose-4-epimerase (kfoA or GNE1) activity is obtained or derived from a bacteria.

12. The recombinant host cell (i) or the recombinant yeast cell (ii) according to claim 1, wherein the recombinant cell further comprises at least one recombinant nucleic acid encoding one or more of:
(i) a polypeptide having glutamine-fructose-6-phosphate amidotransferase (GFA1) activity; and/or
(ii) a polypeptide having UDP-N-acetylglucosamine pyrophosphorylase (QRI1) activity; and/or
(iii) a polypeptide having phosphoglucomutase-1 (PGM1) activity; and/or
(iv) a polypeptide having UTP-glucose-1-phosphate uridylyltransferase (UGP1) activity;
(v) a polypeptide having glucosamine-6-phosphate N-acetyltransferase (GNA1) activity; and/or
(vi) a polypeptide having phosphoacetylglucosamine mutase (PCM1) activity.

13. (canceled)

14. The recombinant host cell (i) or the recombinant yeast cell (ii) according to claim 12, wherein:
the nucleic acid encoding the polypeptide having glutamine-fructose-6-phosphate amidotransferase (GFA1) activity;
the nucleic acid encoding the polypeptide having UDP-N-acetylglucosamine pyrophosphorylase (QRI1) activity;
the nucleic acid encoding the polypeptide having Phosphoglucomutase-1 (PGM1) activity;
the nucleic acid encoding the polypeptide having UTP-glucose-1-phosphate uridylyltransferase (UGP1) activity;
the nucleic acid encoding the polypeptide having Glucosamine-6-phosphate N-acetyltransferase (GNA1) activity; and/or
the nucleic acid encoding the polypeptide having phosphoacetylglucosamine mutase (PCM1);
are obtained or derived from Saccharomyces cerevisiae.

15. (canceled)

16. The recombinant yeast cell ii) according to claim 1, wherein the yeast belongs to a genus selected from the group consisting of Saccharomyces, Candida, Kluyveromyces, Ogataea, Yarrowia, Debaryomyces and Ashbya.

17. The recombinant yeast cell ii) according to claim 16, wherein the yeast is selected from the group consisting of Saccharomyces cerevisiae, Saccharomyces boulardii, Saccharomyces bayanus, Saccharomyces paradoxus, Saccharomyces mikatae, Saccharomyces castelli, Candida albicans, Candida glabrata, Candida tropicalis, Kluyveromyces lactis, Kluyveromyces marxianus, Kluyveromyces polysporus, Kluyveromyces thermotolerans Ogataea polymorpha, Yarrowia lypolytica, Debaryomyces hansenii, and Ashbya gossypii.

18. A method of producing chondroitin of a desired molecular weight comprising:
(a) cultivating the recombinant host cell (i) or the recombinant yeast cell (ii) as defined in claim 1 in a cultivation medium for a time sufficient to produce chondroitin of the desired molecular weight; and
(b) optionally isolating or recovering the chondroitin from the recombinant host cell (i) or the recombinant yeast cell (ii) and/or from the cultivation medium.

19. The method of claim 18, wherein the chondroitin has a molecular weight of from 20 kDa to 50 kDa, or from 50 kDa to 150 kDa or from 150 kDa to 1500 kDa.

20. (canceled)

21. (canceled)

22. (canceled)

23. The method according to claim 18, wherein the time sufficient to produce chondroitin of a desired molecular weight is a period of from 35 hours to 50 hours, preferably or from 40 hours to 50 hours, or is 48 hours.

24. The method according to claim 18, wherein the molecular weight of the chondroitin is controlled by regulating the pH of the cultivation medium during step (a).

25. The method according to claim 18, wherein the method is carried out on an industrial scale, where the cultivation medium is at least 100 L, or is in the range of 1,000 L to 3,000 L, or is about 10,000 L or is 100,000 L, or is about 250,000 L.

26. Chondroitin obtainable from the recombinant host cell (i) or the recombinant yeast cell (ii) according to claim 1.

27. A cultivation medium comprising the chondroitin according to claim 26.

28. A composition comprising the chondroitin according to claim 26.

29. An industrial product or a consumer product or a consumable comprising (i) the chondroitin of claim 5, (ii) a cultivation medium comprising the chondroitin or (iii) a composition comprising the chondroitin.

30. The industrial product or the consumer product or the consumable of claim 29, wherein the industrial product or the consumer product or the consumable is a cosmetic product, a flavour product, a fragrance product, a food product, a food, a beverage, a texturant, a pharmaceutical composition, a dietary supplement, a nutraceutical, a cleaning product, a dental and/or an oral hygiene composition.

31. (canceled)

32. A method for producing chondroitin, comprising the steps of:
(a) culturing the recombinant yeast cell ii) according to claim 1 in a cultivation medium; and
(b) recovering the chondroitin from said cultivation medium,
wherein the chondroitin recovered in step (b) has a molecular weight controlled through the selection of:
the nature and origin of the recombinant nucleic acid encoding a polypeptide having a chondroitinase activity of the recombinant yeast ii),
the nature and origin of the promoter controlling expression of a recombinant nucleic acid encoding a polypeptide having a chondroitinase activity of the recombinant yeast,
the presence of an anchoring and/or of a secretion signal associated with the polypeptide having a chondroitinase activity of the recombinant yeast,
the pH of the cultivation medium during the step of culturing the recombinant yeast, and/or
the duration of the step of culturing of the recombinant yeast.


SEQUENCE LISTING (128 sequences)

SEQ ID NO: 1 - DNA, 2919 bp, Artificial Sequence
  Synthetic reencoded nucleic acid sequence of a polypeptide having a chondroitin synthase activity (HCOS-1)
atgaacaccctttcccaggcaattaaggcttataatagtaatgattatcaactagccctcaagctgttcgaaaagtcggcagaaatctacggcagaaagatagttgagttccaaattacgaagtgcaaggagaaactttctgcgcacccatccgttaacagtgcgcatctctcggttaacaaagaagagaaggtgaatgtttgcgacagtccattagacatagccactcaattattgttatcgaatgtgaaaaaactcgtccttagtgatagcgagaaaaatacactaaagaataaatggaagttgttgactgaaaagaagtctgagaacgcagaggtccgggccgtagctttagttcccaaggactttccgaaagatttagtgcttgcgccgttaccagaccatgtgaacgattttacgtggtataagaagcggaagaaacgcctaggaatcaaaccagaacaccaacacgttggcctctccattattgtgacaacgttcaaccgtcctgccatcctcagcatcaccctggcttgcctcgtcaatcaaaagacccattatccttttgaggtgatcgtgaccgacgatggttctcaagaggatttatcgcctataatccgtcaatatgagaacaagctagacatccgttatgttcggcaaaaggactatgggtatcagctgtgcgccgttagaaaccttggtctaaggacagcgaagtatgactttgtctctatcttggattgcgacatggccccgcagcagctatgggttcactcttaccttacagaactattagaggacgatgatttgaccataataggccccaggaagtacattgacacgcaacacatagatcccaaggatttccttaataacgcctctctgttagagtcgttgccagaggttaagaccaataattccgtcgcggctaagggcgaggggaccgtatctttagactggcgtttggaacaatttgagaagaccgagaacttgaggctatccgatagccctttccgattcttcgcagctgggaatgtggctttcgccaagaagtggcttaacaagtcaggattcttcgacgaggaattcaatcactggggaggtgaagatgtagagttcggttatcgtctgtttcggtacggttcgttcttcaaaactatagacggcatcatggcctatcatcaggaaccgccaggtaaagaaaacgaaactgacagagaagcgggcaagaacattaccctcgatataatgagggagaaggtgccttacatctaccgtaaactcctgcctatagaagacagtcatatcaaccgagtaccattggtatcaatttacatcccggcctacaactgtgctaactatattcaaagatgtgttgattcagctttgaatcaaacggtagttgatctcgaagtgtgcatttgcaacgacggtagtactgacaacacgctggaagttattaacaagctgtatggtaataatccgcgtgtgcgtataatgtctaaacccaatggcggcattgcgagtgcatccaacgcagcggtcagcttcgcaaagggttattacataggacagttggacagcgacgattacttagaacccgacgcagtggagttatgtctcaaggaatttcttaaggataagacccttgcgtgcgtttacaccactaatcgtaacgtcaacccagatggctctttaatagccaatggctataactggccagagttcagtcgtgagaagttgactacggccatgattgctcatcacttccggatgtttaccattcgtgcttggcatctgacggatgggttcaatgagaagattgagaacgctgttgactacgacatgtttctcaagctcagtgaagttggtaaatttaagcatctgaacaaaatatgttataatcgggtgttacacggcgataacacctcaatcaagaagcttggcatacaaaagaagaatcatttcgtagttgtcaatcagtctctaaaccgccaaggtataacttattataactacgatgaatttgatgatctcgatgagagtcggaaatacattttcaacaagactgcagagtatcaagaagagatagatattcttaaagatattaagatcattcagaacaaagacgccaaaatagctgtctccattttctatccgaacactctaaacgggttggtgaagaagctaaataatattatagagtataataagaatatcttcgttattgtacttcatgtcgataagaatcacttaaccccagacatcaagaaggagatattagccttctaccataagcatcaggtgaacatcttattgaacaatgacatctcctactatacatcaaatcgtctgattaagacagaagcccatttgagtaacattaacaagctaagtcaattaaatcttaactgcgaatacattatattcgacaatcacgattccttatttgtgaagaacgattcctatgcatacatgaagaagtatgatgttggaatgaacttctctgcattgactcatgattggattgaaaagataaacgctcacccgccatttaagaagctgatcaaaacttacttcaatgacaatgatcttaagtcgatgaatgtaaagggtgcctcccagggaatgtttatgacatacgcattagcccacgagttattgacgatcatcaaggaggtgataacctcttgtcaatccattgactccgtccccgaatacaacacagaagatatttggtttcagtttgcacttttaattctggaaaagaagaccggccacgtattcaacaagacaagcactctcacgtatatgccatgggaacgtaaactgcagtggacgaatgaacaaatagagtccgcaaagaggggcgaaaacattccggtaaacaagttcatcattaacagcattaccctttaa

SEQ ID NO: 2 - DNA, 2919 bp, Artificial Sequence
  Synthetic reencoded nucleic acid sequence of a polypeptide having a chondroitin synthase activity (HCOS1-2)
atgaacaccctttcccaggcaattaaggcttataatagtaatgattatcaactagccctcaagctgttcgaaaagtcggcagaaatctacggcagaaagatagttgagttccaaattacgaagtgcaaggagaaactttctgcgcacccatccgttaacagtgcgcatctctcggttaacaaagaagagaaggtgaatgtttgcgacagtccattagacatagccactcaattattgttatcgaatgtgaaaaaactcgtccttagtgatagcgagaaaaatacactaaagaataaatggaagttgttgactgaaaagaagtctgagaacgcagaggtccgggccgtagctttagttcccaaggactttccgaaagatttagtgcttgcgccgttaccagaccatgtgaacgattttacgtggtataagaagcggaagaaacgcctaggaatcaaaccagtgaacaagaacatcgggttgtcaatcataatcccaactttcaatcgtagtaggatattagacatcaccctggcttgcctcgtcaatcaaaagacccattatccttttgaggtgatcgtgaccgacgatggttctcaagaggatttatcgcctataatccgtcaatatgagaacaagctagacatccgttatgttcggcaaaaggactatgggtatcagctgtgcgccgttagaaaccttggtctaaggacagcgaagtatgactttgtctctatcttggattgcgacatggccccgcagcagctatgggttcactcttaccttacagaactattagaggacgatgatttgaccataataggccccaggaagtacattgacacgcaacacatagatcccaaggatttccttaataacgcctctctgttagagtcgttgccagaggttaagaccaataattccgtcgcggctaagggcgaggggaccgtatctttagactggcgtttggaacaatttgagaagaccgagaacttgaggctatccgatagccctttccgattcttctcatgcggaaatgtggccttctccaaagagtggctgaacaaagttggctggttcgacgaggaattcaatcactggggaggtgaagatgtagagttcggttatcgtctgtttcggtacggttcgttcttcaaaactatagacggcatcatggcctatcatcaggaaccgccaggtaaagaaaacgaaactgacagagaagcgggcaagaacattaccctcgatataatgagggagaaggtgccttacatctaccgtaaactcctgcctatagaagacagtcatatcaaccgagtaccattggtatcaatttacatcccggcctacaactgtgctaactatattcaaagatgtgttgattcagctttgaatcaaacggtagttgatctcgaagtgtgcatttgcaacgacggtagtactgacaacacgctggaagttattaacaagctgtatggtaataatccgcgtgtgcgtataatgtctaaacccaatggcggcattgcgagtgcatccaacgcagcggtcagcttcgcaaagggttattacataggacagttggacagcgacgattacttagaacccgacgcagtggagttatgtctcaaggaatttcttaaggataagacccttgcgtgcgtttacaccactaatcgtaacgtcaacccagatggctctttaatagccaatggctataactggccagagttcagtcgtgagaagttgactacggccatgattgctcatcacttccggatgtttaccattcgtgcttggcatctgacggatgggttcaatgagaagattgagaacgctgttgactacgacatgtttctcaagctcagtgaagttggtaaatttaagcatctgaacaaaatatgttataatcgggtgttacacggcgataacacctcaatcaagaagcttggcatacaaaagaagaatcatttcgtagttgtcaatcagtctctaaaccgccaaggtataacttattataactacgatgaatttgatgatctcgatgagagtcggaaatacattttcaacaagactgcagagtatcaagaagagatagatattcttaaagatattaagatcattcagaacaaagacgccaaaatagctgtctccattttctatccgaacactctaaacgggttggtgaagaagctaaataatattatagagtataataagaatatcttcgttattgtacttcatgtcgataagaatcacttaaccccagacatcaagaaggagatattagccttctaccataagcatcaggtgaacatcttattgaacaatgacatctcctactatacatcaaatcgtctgattaagacagaagcccatttgagtaacattaacaagctaagtcaattaaatcttaactgcgaatacattatattcgacaatcacgattccttatttgtgaagaacgattcctatgcatacatgaagaagtatgatgttggaatgaacttctctgcattgactcatgattggattgaaaagataaacgctcacccgccatttaagaagctgatcaaaacttacttcaatgacaatgatcttaagtcgatgaatgtaaagggtgcctcccagggaatgtttatgacatacgcattagcccacgagttattgacgatcatcaaggaggtgataacctcttgtcaatccattgactccgtccccgaatacaacacagaagatatttggtttcagtttgcacttttaattctggaaaagaagaccggccacgtattcaacaagacaagcactctcacgtatatgccatgggaacgtaaactgcagtggacgaatgaacaaatagagtccgcaaagaggggcgaaaacattccggtaaacaagttcatcattaacagcattaccctttaa

SEQ ID NO: 3 - DNA, 2919 bp, Artificial Sequence
  Synthetic reencoded nucleic acid sequence of a polypeptide having a chondroitin synthase activity (HCOS1-3)
atgaacaccctttcccaggcaattaaggcttataatagtaatgattatcaactagccctcaagctgttcgaaaagtcggcagaaatctacggcagaaagatagttgagttccaaattacgaagtgcaaggagaaactttctgcgcacccatccgttaacagtgcgcatctctcggttaacaaagaagagaaggtgaatgtttgcgacagtccattagacatagccactcaattattgttatcgaatgtgaaaaaactcgtccttagtgatagcgagaaaaatacactaaagaataaatggaagttgttgactgaaaagaagtctgagaacgcagaggtccgggccgtagctttagtcccaaaggattttcccaaagacttagttttagcgccactaccagaccatgtgaacgattttacctggtacaagaatagaaagaagagcctgggcataaagcctgtgaacaagaacatcgggttgtcaatcataatcccaactttcaatcgtagtaggatattagacattaccctggcgtgtctagttaatcagaaaactaactatccgtttgaggtggttgttgcagatgatgggtcgaaggaaaatctattgactatagttcagaaatacgagcagaaactagacattaagtatgtgagacagaaggactatgggtatcagctgtgcgccgttagaaaccttggtctaaggacagcgaagtatgactttgtctctatcttggattgcgacatggccccgcagcagctatgggttcactcttaccttacagagttactggaagacaacgacatcgtcttgatcgggccaaggaaatacgttgatacccacaacatcacggctgagcagtttctgaacgacccatatttaattgagagtttaccggaaaccgccacaaacaataatccatcgattaccagtaagggtaacatctcattagattggagattagaacattttaagaagacagacaacctaagactatgcgactccccgttcaggtatttctcatgcggaaatgtggccttctccaaagagtggctgaacaaagttggctggttcgacgaagaatttaatcactggggcggcgaagacgtcgaattcggctacaggttatttgcaaaagggtgcttctttagggttatcgacggtggtatggcctaccatcaagagccaccaggaaaggagaacgaaacggatcgtgaggccggtaagtctatcactcttaaaatagtaaaggagaaagtgccctacatctacaggaagttgttgccgattgaggactcccacataaaccgagtaccattggtatcaatttacatcccggcctacaactgtgctaactatattcaaagatgtgttgattcagctttgaatcaaacggtagttgatctcgaagtgtgcatttgcaacgacggtagtactgacaacacgctggaagttattaacaagctgtatggtaataatccgcgtgtgcgtataatgtctaaacccaatggcggcattgcgagtgcatccaacgcagcggtcagcttcgcaaagggttattacataggacagttggacagcgacgattacttagaacccgacgcagtggagttatgtctcaaggaatttcttaaggataagacccttgcgtgcgtttacaccactaatcgtaacgtcaacccagatggctctttaatagccaatggctataactggccagagttcagtcgtgagaagttgactacggccatgattgctcatcacttccggatgtttaccattcgtgcttggcatctgacggatgggttcaatgagaagattgagaacgctgttgactacgacatgtttctcaagctcagtgaagttggtaaatttaagcatctgaacaaaatatgttataatcgggtgttacacggcgataacacctcaatcaagaagcttggcatacaaaagaagaatcatttcgtagttgtcaatcagtctctaaaccgccaaggtataacttattataactacgatgaatttgatgatctcgatgagagtcggaaatacattttcaacaagactgcagagtatcaagaagagatagatattcttaaagatattaagatcattcagaacaaagacgccaaaatagctgtctccattttctatccgaacactctaaacgggttggtgaagaagctaaataatattatagagtataataagaatatcttcgttattgtacttcatgtcgataagaatcacttaaccccagacatcaagaaggagatattagccttctaccataagcatcaggtgaacatcttattgaacaatgacatctcctactatacatcaaatcgtctgattaagacagaagcccatttgagtaacattaacaagctaagtcaattaaatcttaactgcgaatacattatattcgacaatcacgattccttatttgtgaagaacgattcctatgcatacatgaagaagtatgatgttggaatgaacttctctgcattgactcatgattggattgaaaagataaacgctcacccgccatttaagaagctgatcaaaacttacttcaatgacaatgatcttaagtcgatgaatgtaaagggtgcctcccagggaatgtttatgacatacgcattagcccacgagttattgacgatcatcaaggaggtgataacctcttgtcaatccattgactccgtccccgaatacaacacagaagatatttggtttcagtttgcacttttaattctggaaaagaagaccggccacgtattcaacaagacaagcactctcacgtatatgccatgggaacgtaaactgcagtggacgaatgaacaaatagagtccgcaaagaggggcgaaaacattccggtaaacaagttcatcattaacagcattaccctttaa

SEQ ID NO: 4 - DNA, 3072 bp, Artificial Sequence
  Synthetic nucleic acid sequence of a polypeptide having chondroitin synthase activity (HCOS.Sc)
atgaacaccctttcccaggcaattaaggcttataatagtaatgattatcaactagccctcaagctgttcgaaaagtcggcagaaatctacggcagaaagatagttgagttccaaattacgaagtgcaaggagaaactttctgcgcacccatccgttaacagtgcgcatctctcggttaacaaagaagagaaggtgaatgtttgcgacagtccattagacatagccactcaattattgttatcgaatgtgaaaaaactcgtccttagtgatagcgagaaaaatacactaaagaataaatggaagttgttgactgaaaagaagtctgagaacgcagaggtccgggccgtagctttagtcccaaaggattttcccaaagacttagttttagcgccactaccagaccatgtgaacgattttacctggtacaagaatagaaagaagagcctgggcataaagcctgtgaacaagaacatcgggttgtcaatcataatcccaactttcaatcgtagtaggatattagacattaccctggcgtgtctagttaatcagaaaactaactatccgtttgaggtggttgttgcagatgatgggtcgaaggaaaatctattgactatagttcagaaatacgagcagaaactagacattaagtatgtgagacagaaggactatgggtatcagctgtgcgccgttagaaaccttggtctaaggacagcgaagtatgactttgtctctatcttggattgcgacatggccccgcagcagctatgggttcactcttaccttacagagttactggaagacaacgacatcgtcttgatcgggccaaggaaatacgttgatacccacaacatcacggctgagcagtttctgaacgacccatatttaattgagagtttaccggaaaccgccacaaacaataatccatcgattaccagtaagggtaacatctcattagattggagattagaacattttaagaagacagacaacctaagactatgcgactccccgttcaggtatttctcatgcggaaatgtggccttctccaaagagtggctgaacaaagttggctggttcgacgaagaatttaatcactggggcggcgaagacgtcgaattcggctacaggttatttgcaaaagggtgcttctttagggttatcgacggtggtatggcctaccatcaagagccaccaggaaaggagaacgaaacggatcgtgaggccggtaagtctatcactcttaaaatagtaaaggagaaagtgccctacatctacaggaagttgttgccgattgaggactcccacataaaccgagtaccattggtatcaatttacatcccggcctacaactgtgctaactatattcaaagatgtgttgattcagctttgaatcaaacggtagttgatctcgaagtgtgcatttgcaacgacggtagtactgacaacacgctggaagttattaacaagctgtatggtaataatccgcgtgtgcgtataatgtctaaacccaatggcggcattgcgagtgcatccaacgcagcggtcagcttcgcaaagggttattacataggacagttggacagcgacgattacttagaacccgacgcagtggagttatgtctcaaggaatttcttaaggataagacccttgcgtgcgtttacaccactaatcgtaacgtcaacccagatggctctttaatagccaatggctataactggccagagttcagtcgtgagaagttgactacggccatgattgctcatcacttccggatgtttaccattcgtgcttggcatctgacggatgggttcaatgagaagattgagaacgctgttgactacgacatgtttctcaagctcagtgaagttggtaaatttaagcatctgaacaaaatatgttataatcgggtgttacacggcgataacacctcaatcaagaagcttggcatacaaaagaagaatcatttcgtagttgtcaatcagtctctaaaccgccaaggtataacttattataactacgatgaatttgatgatctcgatgagagtcggaaatacattttcaacaagactgcagagtatcaagaagagatagatattcttaaagatatttttattcagatgcttttttcctggttttctattgcaaatttcgttcttaccttttattatttagcaggatcaatgaatttagttattaaacatggtgaggccttattcattttttttaaatacctgatcttttgtgacttggcaagtttattcattatttccatgggtaatagaccccagggcgcgaaacatttattcattacctccatggttatactgtctatatgtgccacatattctctaatttgtgggtttgtttttgctttcaagtcgttagcttctggaacggaatcccacaaaatatttgtcgacatcgttatctcattgctctccacctatggcctatactttttctcatcactgatgtacctagatccttggcacatgtttacatcatccatacaatactttttgacacttcccgcctttacgtgtactttacagatttttgccttctgtaatacacacgacgtttcctggggtactaaaggttccacacaggagtccaagcaattgtccaaggccattgtcgttcaaggtccagatgggaaacagattgtggaaacagattggcctcaggaagttgataagaagtttttggaaataaaaagtcgtttgaaagaaccagaatttgaagaatcaagcggcaatgaaaaacaatccaagaatgattattatagagatataagaaccagaattgtgatgatttggatgctatcaaatctaatactgatcatgtctataattcaagtctttacaccacaagatactgacaatggttatttgatattcattttatggtctgtggccgctttagctgcctttagggtggttggttccatggcctttttgttcatgaaatacttgcgtataatagtgagttacagaaataaagttgaaggtagcggctcatgggaagtctctaaattagacttaccaaatgttttccacaaaaagggctaa

SEQ ID NO: 5 - DNA, 3072 bp, Artificial Sequence
  Synthetic nucleic acid sequence of a polypeptide having chondroitin synthase activity (HHASA.Sc)
atgaacaccctttcccaggcaattaaggcttataatagtaatgattatcaactagccctcaagctgttcgaaaagtcggcagaaatctacggcagaaagatagttgagttccaaattacgaagtgcaaggagaaactttctgcgcacccatccgttaacagtgcgcatctctcggttaacaaagaagagaaggtgaatgtttgcgacagtccattagacatagccactcaattattgttatcgaatgtgaaaaaactcgtccttagtgatagcgagaaaaatacactaaagaataaatggaagttgttgactgaaaagaagtctgagaacgcagaggtccgggccgtagctttagttcccaaggactttccgaaagatttagtgcttgcgccgttaccagaccatgtgaacgattttacgtggtataagaagcggaagaaacgcctaggaatcaaaccagaacaccaacacgttggcctctccattattgtgacaacgttcaaccgtcctgccatcctcagcatcaccctggcttgcctcgtcaatcaaaagacccattatccttttgaggtgatcgtgaccgacgatggttctcaagaggatttatcgcctataatccgtcaatatgagaacaagctagacatccgttatgttcggcaaaaggacaacggttttcaggcatcggccgcacgtaatatgggcctacgtctagccaaatatgacttcattgggctccttgattgtgatatggctcccaaccctttgtgggtacactcatacgttgcggaactattagaggacgatgatttgaccataataggccccaggaagtacattgacacgcaacacatagatcccaaggatttccttaataacgcctctctgttagagtcgttgccagaggttaagaccaataattccgtcgcggctaagggcgaggggaccgtatctttagactggcgtttggaacaatttgagaagaccgagaacttgaggctatccgatagccctttccgattcttcgcagctgggaatgtggctttcgccaagaagtggcttaacaagtcaggattcttcgacgaggaattcaatcactggggaggtgaagatgtagagttcggttatcgtctgtttcggtacggttcgttcttcaaaactatagacggcatcatggcctatcatcaggaaccgccaggtaaagaaaacgaaactgacagagaagcgggcaagaacattaccctcgatataatgagggagaaggtgccttacatctaccgtaaactcctgcctatagaagacagtcatatcaaccgagtaccattggtatcaatttacatcccggcctacaactgtgctaactatattcaaagatgtgttgattcagctttgaatcaaacggtagttgatctcgaagtgtgcatttgcaacgacggtagtactgacaacacgctggaagttattaacaagctgtatggtaataatccgcgtgtgcgtataatgtctaaacccaatggcggcattgcgagtgcatccaacgcagcggtcagcttcgcaaagggttattacataggacagttggacagcgacgattacttagaacccgacgcagtggagttatgtctcaaggaatttcttaaggataagacccttgcgtgcgtttacaccactaatcgtaacgtcaacccagatggctctttaatagccaatggctataactggccagagttcagtcgtgagaagttgactacggccatgattgctcatcacttccggatgtttaccattcgtgcttggcatctgacggatgggttcaatgagaagattgagaacgctgttgactacgacatgtttctcaagctcagtgaagttggtaaatttaagcatctgaacaaaatatgttataatcgggtgttacacggcgataacacctcaatcaagaagcttggcatacaaaagaagaatcatttcgtagttgtcaatcagtctctaaaccgccaaggtataacttattataactacgatgaatttgatgatctcgatgagagtcggaaatacattttcaacaagactgcagagtatcaagaagagatagatattcttaaagatatttttattcagatgcttttttcctggttttctattgcaaatttcgttcttaccttttattatttagcaggatcaatgaatttagttattaaacatggtgaggccttattcattttttttaaatacctgatcttttgtgacttggcaagtttattcattatttccatgggtaatagaccccagggcgcgaaacatttattcattacctccatggttatactgtctatatgtgccacatattctctaatttgtgggtttgtttttgctttcaagtcgttagcttctggaacggaatcccacaaaatatttgtcgacatcgttatctcattgctctccacctatggcctatactttttctcatcactgatgtacctagatccttggcacatgtttacatcatccatacaatactttttgacacttcccgcctttacgtgtactttacagatttttgccttctgtaatacacacgacgtttcctggggtactaaaggttccacacaggagtccaagcaattgtccaaggccattgtcgttcaaggtccagatgggaaacagattgtggaaacagattggcctcaggaagttgataagaagtttttggaaataaaaagtcgtttgaaagaaccagaatttgaagaatcaagcggcaatgaaaaacaatccaagaatgattattatagagatataagaaccagaattgtgatgatttggatgctatcaaatctaatactgatcatgtctataattcaagtctttacaccacaagatactgacaatggttatttgatattcattttatggtctgtggccgctttagctgcctttagggtggttggttccatggcctttttgttcatgaaatacttgcgtataatagtgagttacagaaataaagttgaaggtagcggctcatgggaagtctctaaattagacttaccaaatgttttccacaaaaagggctaa

SEQ ID NO: 6 - DNA, 1719 bp, Artificial Sequence
  Synthetic nucleic acid sequence of a polypeptide having chondroitin synthase activity (HHASA.Sc)
atgggtaagaacatcattattatggtttcttggtacactattattacatccaatctcatcgcagtgggtggcgcctcactcatactagccccagctattacgggctatgtccttcactggaacattgccctttcaacaatttggggagtgtcggcctacggaattttcgtgtttggtttctttcttgcccaggtattatttagtgaactcaaccggaaaaggctccggaagtggatttccctccgacccaaagggtggaatgatgttaggttggctgtaattattgccggctaccgtgaggacccgtatatgttccaaaagtgtcttgaaagtgtgcgtgactcagactatgggaatgtagctagactaatatgcgttattgacggcgatgaagacgacgacatgaggatggctgcagtgtacaaggctatctataacgacaacatcaagaaacctgagtttgtcctctgtgagagtgacgataaggagggtgagagaatagatagcgatttcagccgtgatatctgcgtgctgcaaccgcatcgcggaaagcgtgaatgtttgtacacagggttccaattggcaaagatggacccctcagttaatgccgtcgtcctaatcgacagtgacactgtgttagaaaaggacgcgattctcgaagtagtatacccgctggcatgcgatccagaaatacaggctgtagcaggcgaatgcaaaatatggaatactgacacactgttgagtttgctggtagcctggcgatattacagcgcattttgcgtagagcgtagcgcccaatcattcttcaggacagtacaatgcgtcggaggacctctcggcgcctacaagattgatataattaaggaaatcaaggacccatggatcagccaacgtttccttggccaaaagtgcacatacggcgacgatagacgactcactaatgaaatactaatgaggggtaagaaagtagttttcaccccattcgctgttggctggggatggttcgacgaggagttcacacactggggtggccgttacattgtacagcaaacacggtggtccaagtcgtggtgtagggagatatggtatacactgtttgcagcatggaagcatggactttcgggcatttggctcgcattcgagtgcttataccagattacttatttcttcctggtgatttacctattctcccgtttggctgttgaggctgacccacgggcgcaaacggccaccgtcattgtttcgaccacagttgcgcttattaagtgcggttactttagcttcagagctaaagacattagggccttctatttcgtcctctacacgttcgtttacttcttctgcatgattccggcacgaataactgcgatgatgaccctgtgggacataggttggggaaccaggggaggaaatgagaagccttccgtaggcaccagagttgctttgtgggccaagcaatatttgattgcttacatgtggtgggccgctgtcgtcggtgctggcgtgtactccatcgttcataattggatgtttgactggaattcactttcctacaggttcgcactggtaggcatctgttcgtatatagttttcattgtaatagtgctggtagtctactttacaggaaagatcacgacctggaacttcacgaagctacagaaagaattgatcgaggaccgcgtactgtacgacgcaacgaccaatgcccagtcggtataa

SEQ ID NO: 7 - DNA, 1716 bp, Artificial Sequence
  Synthetic reencoded nucleic acid sequence of a polypeptide having chondroitin synthase activity (HCOS2-Vir)
atgggtaagaacatcattattatggtttcttggtacactattattacatccaatctcatcgcagtgggtggcgcctcactcatactagccccagctattacgggctatgtccttcactggaacattgccctttcaacaatttggggagtgtcggcctacggaattttcgtgtttggtttctttcttgcccaggtattatttagtgaactcaaccggaaaaggctccggaagtggatttccctccgacccaaagggtggaatgatgttaggttggctgtaattattgccggctaccgtgaggacccgtatatgttccaaaagtgtcttgaaagtgtgcgtgactcagactatgggaatgtagctagactaatatgcgttattgacggcgatgaagacgacgacatgaggatggctgcagtgtacaaggctatctataacgacaacatcaagaaacctgagtttgtcctctgtgagagtgacgataaggagggtgagagaatagatagcgatttcagccgtgatatctgcgtgctgcaaccgcatcgcggaaagcgtgaatgtttgtacacagggttccaattggcaaagatggacccctcagttaatgccgtcgtcctaatcgacagtgacactgtgttagaaaaggacgcgattctcgaagtagtatacccgctggcatgcgatccagaaatacaggctgtagcaggcgaatgcaaaatatggaatactgacacactgttgagtttgctggtagcctggcgatattacagcgcattttgcgtagagcgtagcgcccaatcattcttcaggacagtacaatgcgtcggaggacctctcggcgcctacaagattgatataattaaggaaatcaaggacccatggatcagccaacgtttccttggccaaaagtgcacatacggcgacgatagacgactcactaatgaaatactaatgaggggtaagaaagtagttttcaccccattcgctgttggctggctacacggtgagaatacgtctatcaagaagttacgttacattgtacagcaaacacggtggtccaagtcgtggtgtagggagatatggtatacactgtttgcagcatggaagcatggactttcgggcatttggctcgcattcgagtgcttataccagattacttatttcttcctggtgatttacctattctcccgtttggctgttgaggctgacccacgggcgcaaacggccaccgtcattgtttcgaccacagttgcgcttattaagtgcggttactttagcttcagagctaaagacattagggccttctatttcgtcctctacacgttcgtttacttcttctgcatgattccggcacgaataactgcgatgatgaccctgtgggacataggttggggaaccaggggaggaaatgagaagccttccgtaggcaccagagttgctttgtgggccaagcaatatttgattgcttacatgtggtgggccgctgtcgtcggtgctggcgtgtactccatcgttcataattggatgtttgactggaattcactttcctacaggttcgcactggtaggcatctgttcgtatatagttttcattgtaatagtgctggtagtctactttacaggaaagatcacgacctggaacttcacgaagctacagaaagaattgatcgaggaccgcgtactgtacgacgcaacgaccaatgcccagtcggtataa

SEQ ID NO: 8 - DNA, 1719 bp, Artificial Sequence
  Synthetic reencoded nucleic acid sequence of a polypeptide having chondroitin synthase activity (HCOS3-Vir)
atgggtaagaacatcattattatggtttcttggtacactattattacatccaatctcatcgcagtgggtggcgcctcactcatactagccccagctattacgggctatgtccttcactggaacattgccctttcaacaatttggggagtgtcggcctacggaattttcgtgtttggtttctttcttgcccaggtattatttagtgaactcaaccggaaaaggctccggaagtggatttccctccgacccaaagggtggaatgatgttaggttggctgtaattattgccggctaccgtgaggacccgtatatgttccaaaagtgtcttgaaagtgtgcgtgactcagactatgggaatgtagctagactaatatgcgttattgacggcgatgaagacgacgacatgaggatggctgcagtgtacaaggctatctataacgacaacatcaagaaacctgagtttgtcctctgtgagagtgacgataaggagggtgagagaatagatagcgatttcagccgtgatatctgcgtgctgcaaccgcatcgcggaaagcgtgaatgtttgtacacagggttccaattggcaaagatggacccctcagttaatgccgtcgtcctaatcgacagtgacactgtgttagaaaaggacgcgattctcgaagtagtatacccgctggcatgcgatccagaaatacaggctgtagcaggcgaatgcaaaatatggaatactgacacactgttgagtttgctggtagcctggcgatattacagcgcattttgcgtagagcgtagcgcccaatcattcttcaggacagtacaatgcgtcggaggacctctcggcgcctacaagattgatataattaaggaaatcaaggacccatggatcagccaacgtttccttggccaaaagtgcacatacggcgacgatagacgactcactaatgaaatactaatgaggggtaagaaagtagttttcaccccattcgctgttggctggcacatggcatggtccgacaaggacgacgcaatcgaccgttacattgtacagcaaacacggtggtccaagtcgtggtgtagggagatatggtatacactgtttgcagcatggaagcatggactttcgggcatttggctcgcattcgagtgcttataccagattacttatttcttcctggtgatttacctattctcccgtttggctgttgaggctgacccacgggcgcaaacggccaccgtcattgtttcgaccacagttgcgcttattaagtgcggttactttagcttcagagctaaagacattagggccttctatttcgtcctctacacgttcgtttacttcttctgcatgattccggcacgaataactgcgatgatgaccctgtgggacataggttggggaaccaggggaggaaatgagaagccttccgtaggcaccagagttgctttgtgggccaagcaatatttgattgcttacatgtggtgggccgctgtcgtcggtgctggcgtgtactccatcgttcataattggatgtttgactggaattcactttcctacaggttcgcactggtaggcatctgttcgtatatagttttcattgtaatagtgctggtagtctactttacaggaaagatcacgacctggaacttcacgaagctacagaaagaattgatcgaggaccgcgtactgtacgacgcaacgaccaatgcccagtcggtataa

SEQ ID NO: 9 - DNA, 1713 bp, Artificial Sequence
  Synthetic reencoded nucleic acid sequence of a polypeptide having chondroitin synthase activity (HCOS4-Vir)
atgggtaagaacatcattattatggtttcttggtacactattattacatccaatctcatcgcagtgggtggcgcctcactcatactagccccagctattacgggctatgtccttcactggaacattgccctttcaacaatttggggagtgtcggcctacggaattttcgtgtttggtttctttcttgcccaggtattatttagtgaactcaaccggaaaaggctccggaagtggatttccctccgacccaaagggtggaatgatgttaggttggctgtaattattgccggctaccgtgaggacccgtatatgttccaaaagtgtcttgaaagtgtgcgtgactcagactatgggaatgtagctagactaatatgcgttattgacggcgatgaagacgacgacatgaggatggctgcagtgtacaaggctatctataacgacaacatcaagaaacctgagtttgtcctctgtgagagtgacgataaggagggtgagagaatagatagcgatttcagccgtgatatctgcgtgctgcaaccgcatcgcggaaagcgtgaatgtttgtacacagggttccaattggcaaagatggacccctcagttaatgccgtcgtcctaatcgacagtgacactgtgttagaaaaggacgcgattctcgaagtagtatacccgctggcatgcgatccagaaatacaggctgtagcaggcgaatgcaaaatatggaatactgacacactgttgagtttgctggtagcctggcgatattacagcgcattttgcgtagagcgtagcgcccaatcattcttcaggacagtacaatgcgtcggaggacctctcggcgcctacaagattgatataattaaggaaatcaaggacccatggatcagccaacgtttccttggccaaaagtgcacatacggcgacgatagacgactcactaatgaaatactaatgaggggtaagaaagtagttttcaccccattcgctgttggctggttcgacgaggagttcacacactggggtggccgttacattgtacagcaaacacggtggtccaagtcgtggtgtagggagatatggtatacactgtttgcagcatggaagcatggactttcgggcatttggctcgcattcgagtgcttataccagattacttatttcttcctggtgatttacctattctcccgtttggctgttgaggctgacccacgggcgcaaacggccaccgtcattgtttcgaccacagttgcgcttattaagtgcggttactttagcttcagagctaaagacattagggccttctatttcgtcctctacacgttcgtttacttcttctgcatgattccggcacgaataactgcgatgatgaccctgtgggacataggttggggaaccaggggaggaaatgagaagccttccgtaggcaccagagttgctttgtgggccaagcaatatttgattgcttacatgtggtgggccgctgtcgtcggtgctggcgtgtactccatcgttcataattggatgtttgactggaattcactttcctacaggttcgcactggtaggcatctgttcgtatatagttttcattgtaatagtgctggtagtctactttacaggaaagatcacgacctggaacttcacgaagctacagaaagaattgatcgaggaccgcgtactgtacgacgcaacgaccaatgcccagtcggtataa

SEQ ID NO: 10 - DNA, 2079 bp, Artificial Sequence
  Synthetic nucleic acid sequence of a polypeptide having chondroitin synthase activity (HCOS5-Vir)
atgggtaagaacatcattattatggtttcttggtacactattattacatccaatctcatcgcagtgggtggcgcctcactcatactagccccagctattacgggctatgtccttcactggaacattgccctttcaacaatttggggagtgtcggcctacggaattttcgtgtttggtttctttcttgcccaggtattatttagtgaactcaaccggaaaaggctccggaagtggatttccctccgacccaaatccattcaccatccaatttcgaaaacgactcaccccgacacagaaaatgcaggtgactcaacattgttgtgcccgcccatggatggtatggacgacatattagtcgttatgaagacaggtgtcacagaggctaaggaaaaggtgccagtacattttcagacaacgctgaggtgtgtgccgcactacgttatctatagcgacttcgaagaagagatagaaggtgtaaagatttacgacgtcttgcaaggaatggaccccgatgttatgacgcaggttccggacttcgatctttacaatcgtattaagaagttagggcgtcagggccttgcggagaacgacttcgcggacgaagcgaactctgcggtgggtaagcctaataaccctggttggaagttagataaatggaagttcttacctatgatcattgaggctctgagatacaaaccaaaggctaagtggtatgttttcatggaagcagacacttatatatcttggccgactttattagcctggctaaaacacttcaaccacaagaagccgtggtatcttggcactgagacacagatagccgatgtgattttcgctcatggtggttcaggattcatgattagtcatgccgcgcttaagcttgctgctgaagaatataccgcacgtcgtactgagttgcaccactacacagacggtcactgggcaggagactgcgtattaggtaaagttcttgctgacgcaggtgtgaacttgcatttctcctggccaattttacagaattccaatctgggcgaactggatgagttcaacgttgacttatacaggcaaccttggtgctttccagctgccgctttccaccacttgaacgctcagaagatccaggacttatggtcttacgagcagaaaaggtggcaggacaaggacaagagggttctgttacatagcgacgtcttcagggacctattatatccggatatagcattaaaacccgcgaggatcggttgggacaacctgagtgcagacgaacagtctacggcctctagtttcgaggaatgcaggcaaatttgtgatcaattagacgattgtacccagtttagtttcagggacaaatcctgttttacatcaagaactccacgtttgggcgcagccaatccaaatacagttagcggatggatggttgatcgtatccgtgcgaatatggagaagtctggactgtttgcagcatggaagcatggactttcgggcatttggctcgcattcgagtgcttataccagattacttatttcttcctggtgatttacctattctcccgtttggctgttgaggctgacccacgggcgcaaacggccaccgtcattgtttcgaccacagttgcgcttattaagtgcggttactttagcttcagagctaaagacattagggccttctatttcgtcctctacacgttcgtttacttcttctgcatgattccggcacgaataactgcgatgatgaccctgtgggacataggttggggaaccaggggaggaaatgagaagccttccgtaggcaccagagttgctttgtgggccaagcaatatttgattgcttacatgtggtgggccgctgtcgtcggtgctggcgtgtactccatcgttcataattggatgtttgactggaattcactttcctacaggttcgcactggtaggcatctgttcgtatatagttttcattgtaatagtgctggtagtctactttacaggaaagatcacgacctggaacttcacgaagctacagaaagaattgatcgaggaccgcgtactgtacgacgcaacgaccaatgcccagtcggtataa

SEQ ID NO: 11 - DNA, 2733 bp, Artificial Sequence
  Synthetic nucleic acid sequence of a polypeptide having chondroitin synthase activity (HCOS6-Vir)
atgggtaagaacatcattattatggtttcttggtacactattattacatccaatctcatcgcagtgggtggcgcctcactcatactagccccagctattacgggctatgtccttcactggaacattgccctttcaacaatttggggagtgtcggcctacggaattttcgtgtttggtttctttcttgcccaggtattatttagtgaactcaaccggaaaaggctccggaagtggatttccctccgacccaaattagacattgcaacacagttattactatcgaatgttaagaaattgaccttaagcgagtccgaaaagaacagcttaaagaataagtggaagagtataacaggcaagaagtccgagaatgctgaaattaggaaggttgagttagtcccaaaggattttcccaaagacttagttttagcgccactaccagaccatgtgaacgattttacctggtacaagaatagaaagaagagcctgggcataaagcctgtgaacaagaacatcgggttgtcaatcataatcccaactttcaatcgtagtaggatattagacattaccctggcgtgtctagttaatcagaaaactaactatccgtttgaggtggttgttgcagatgatgggtcgaaggaaaatctattgactatagttcagaaatacgagcagaaactagacattaagtatgtgagacagaaggactatgggtatcagctgtgcgccgttagaaaccttggtctaaggacagcgaagtatgactttgtctctatcttggattgcgacatggccccgcagcagctatgggttcactcttaccttacagagttactggaagacaacgacatcgtcttgatcgggccaaggaaatacgttgatacccacaacatcacggctgagcagtttctgaacgacccatatttaattgagagtttaccggaaaccgccacaaacaataatccatcgattaccagtaagggtaacatctcattagattggagattagaacattttaagaagacagacaacctaagactatgcgactccccgttcaggtatttctcatgcggaaatgtggccttctccaaagagtggctgaacaaagttggctggttcgacgaagaatttaatcactggggcggcgaagacgtcgaattcggctacaggttatttgcaaaagggtgcttctttagggttatcgacggtggtatggcctaccatcaagagccaccaggaaaggagaacgaaacggatcgtgaggccggtaagtctatcactcttaaaatagtaaaggagaaagtgccctacatctacaggaagttgttgccgattgaggactcccacatacaccgtataccgcttgtgagcatatacattccagcgtacaactgcgccaattatattcaaaggtgtgtagatagcgcgttgaatcaaacagtggtagatttggaagtttgtatatgtaacgacggaagtactgacaatactttagaggtgattaacaagctgtatggcaacaacccgcgtgtcaggataatgagcaagcccaatggtggaattgcttccgcgtctaacgctgccgtttcattcgcgaaaggctactacattggccagttggactcggatgattacttggaacctgacgcagtggagctttgtttaaaggagttcttgaaggacaaaacactagcctgtgtatatacaacgaacagaaatgttaatcctgacggttccctaatcgctaacggatacaattggcctgagttcagcagggagaagctgaccactgctatgatcgcccaccatttcaggatgttcacaatcagggcttggcatttaacagatggttttaatgagaaaatagagaacgcagtagactacgatatgtttctgaagttatcagaggtcgggaaattcaaacacttgaacaaaatttgttacaatagggtattacacggtgacaacaccagtattaagaatcttgacacccaaaagaagaaccacttcgtagtagtgaaccagtctctgaacagacaaagggtgtcgaattataactacgatgagtttgataacctggacgagagccgtaagtacatattcaacaagctgtttgcagcatggaagcatggactttcgggcatttggctcgcattcgagtgcttataccagattacttatttcttcctggtgatttacctattctcccgtttggctgttgaggctgacccacgggcgcaaacggccaccgtcattgtttcgaccacagttgcgcttattaagtgcggttactttagcttcagagctaaagacattagggccttctatttcgtcctctacacgttcgtttacttcttctgcatgattccggcacgaataactgcgatgatgaccctgtgggacataggttggggaaccaggggaggaaatgagaagccttccgtaggcaccagagttgctttgtgggccaagcaatatttgattgcttacatgtggtgggccgctgtcgtcggtgctggcgtgtactccatcgttcataattggatgtttgactggaattcactttcctacaggttcgcactggtaggcatctgttcgtatatagttttcattgtaatagtgctggtagtctactttacaggaaagatcacgacctggaacttcacgaagctacagaaagaattgatcgaggaccgcgtactgtacgacgcaacgaccaatgcccagtcggtataa

SEQ ID NO: 12 - DNA, 2112 bp, Artificial Sequence
  Synthetic reencoded nucleic acid sequence of a polypeptide having chondroitin synthase activity (HCOS7-Vir)
atgggtaagaacatcattattatggtttcttggtacactattattacatccaatctcatcgcagtgggtggcgcctcactcatactagccccagctattacgggctatgtccttcactggaacattgccctttcaacaatttggggagtgtcggcctacggaattttcgtgtttggtttctttcttgcccaggtattatttagtgaactcaaccggaaaaggctccggaagtggatttccctccgacccaaaatcccaactttcaatcgtagtaggatattagacattaccctggcgtgtctagttaatcagaaaactaactatccgtttgaggtggttgttgcagatgatgggtcgaaggaaaatctattgactatagttcagaaatacgagcagaaactagacattaagtatgtgagacagaaggactatgggtatcagctgtgcgccgttagaaaccttggtctaaggacagcgaagtatgactttgtctctatcttggattgcgacatggccccgcagcagctatgggttcactcttaccttacagagttactggaagacaacgacatcgtcttgatcgggccaaggaaatacgttgatacccacaacatcacggctgagcagtttctgaacgacccatatttaattgagagtttaccggaaaccgccacaaacaataatccatcgattaccagtaagggtaacatctcattagattggagattagaacattttaagaagacagacaacctaagactatgcgactccccgttcaggtatttctcatgcggaaatgtggccttctccaaagagtggctgaacaaagttggctggttcgacgaagaatttaatcactggggcggcgaagacgtcgaattcggctacaggttatttgcaaaagggtgcttctttagggttatcgacggtggtatggcctaccatcaagagccaccaggaaaggagaacgaaacggatcgtgaggccggtaagtctatcactcttaaaatagtaaaggagaaagtgccctacatctacaggaagttgttgccgattgaggactcccacatacaccgtataccgcttgtgagcatatacattccagcgtacaactgcgccaattatattcaaaggtgtgtagatagcgcgttgaatcaaacagtggtagatttggaagtttgtatatgtaacgacggaagtactgacaatactttagaggtgattaacaagctgtatggcaacaacccgcgtgtcaggataatgagcaagcccaatggtggaattgcttccgcgtctaacgctgccgtttcattcgcgaaaggctactacattggccagttggactcggatgattacttggaacctgacgcagtggagctttgtttaaaggagttcttgaaggacaaaacactagcctgtgtatatacaacgaacagaaatgttaatcctgacggttccctaatcgctaacggatacaattggcctctgtttgcagcatggaagcatggactttcgggcatttggctcgcattcgagtgcttataccagattacttatttcttcctggtgatttacctattctcccgtttggctgttgaggctgacccacgggcgcaaacggccaccgtcattgtttcgaccacagttgcgcttattaagtgcggttactttagcttcagagctaaagacattagggccttctatttcgtcctctacacgttcgtttacttcttctgcatgattccggcacgaataactgcgatgatgaccctgtgggacataggttggggaaccaggggaggaaatgagaagccttccgtaggcaccagagttgctttgtgggccaagcaatatttgattgcttacatgtggtgggccgctgtcgtcggtgctggcgtgtactccatcgttcataattggatgtttgactggaattcactttcctacaggttcgcactggtaggcatctgttcgtatatagttttcattgtaatagtgctggtagtctactttacaggaaagatcacgacctggaacttcacgaagctacagaaagaattgatcgaggaccgcgtactgtacgacgcaacgaccaatgcccagtcggtataa

SEQ ID NO: 13 - DNA, 1710 bp, Artificial Sequence
  Synthetic reencoded nucleic acid sequence of a polypeptide having chondroitin synthase activity (HCOS8-Vir)
atgggtaagaacatcattattatggtttcttggtacactattattacatccaatctcatcgcagtgggtggcgcctcactcatactagccccagctattacgggctatgtccttcactggaacattgccctttcaacaatttggggagtgtcggcctacggaattttcgtgtttggtttctttcttgcccaggtattatttagtgaactcaaccggaaaaggctccggaagtggatttccctccgacccaaagggtggaatgatgttaggttggctgtaattattgccggctaccgtgaggacccgtatatgttccaaaagtgtcttgaaagtgtgcgtgactcagactatgggaatgtagctagactaatatgcgttattgacggcgatgaagacgacgacatgaggatggctgcagtgtacaaggctatctataacgacaacatcaagaaacctgagtttgtcctctgtgagagtgacgataaggagggtgagagaatagatagcgatttcagccgtgatatctgcgtgctgcaaccgcatcgcggaaagcgtgaatgtttgtacacagggttccaattggcaaagatggacccctcagttaatgccgtcgtcctaatcgacagtgacactgtgttagaaaaggacgcgattctcgaagtagtatacccgctggcatgcgatccagaaatacaggctgtagcaggcgaatgcaaaatatggaatactgacacactgttgagtttgctggtagcctggcgatattacagcgcattttgcgtagagcgtagcgcccaatcattcttcaggacagtacaatgcgtcagactatgcgactccccgttcaggtatttctcatgcggaaatgtggccttctccaaagagtggctgaacaaagttggctggttcgacgaagaatttaatcactggggcggcgaagacgtcgaattcggctacaggttaaagaaagtagttttcaccccattcgctgttggctggtccgacagcccgacgaacgtcttccgttacattgtacagcaaacacggtggtccaagtcgtggtgtagggagatatggtatacactgtttgcagcatggaagcatggactttcgggcatttggctcgcattcgagtgcttataccagattacttatttcttcctggtgatttacctattctcccgtttggctgttgaggctgacccacgggcgcaaacggccaccgtcattgtttcgaccacagttgcgcttattaagtgcggttactttagcttcagagctaaagacattagggccttctatttcgtcctctacacgttcgtttacttcttctgcatgattccggcacgaataactgcgatgatgaccctgtgggacataggttggggaaccaggggaggaaatgagaagccttccgtaggcaccagagttgctttgtgggccaagcaatatttgattgcttacatgtggtgggccgctgtcgtcggtgctggcgtgtactccatcgttcataattggatgtttgactggaattcactttcctacaggttcgcactggtaggcatctgttcgtatatagttttcattgtaatagtgctggtagtctactttacaggaaagatcacgacctggaacttcacgaagctacagaaagaattgatcgaggaccgcgtactgtacgacgcaacgaccaatgcccagtcggtataa

SEQ ID NO: 14 - DNA, 2499 bp, Artificial Sequence
  Synthetic  nucleic acid sequence of a polypeptide having chondroitin synthase activity (HCOS9-Vir)
atgggtaagaacatcattattatggtttcttggtacactattattacatccaatctcatcgcagtgggtggcgcctcactcatactagccccagctattacgggctatgtccttcactggaacattgccctttcaacaatttggggagtgtcggcctacggaattttcgtgtttggtttctttcttgcccaggtattatttagtgaactcaaccggaaaaggctccggaagtggatttccctccgacccaaacacattggtaagtgtctaagggaaatgtatacgactcatgaggacgtggaagtaggaagatgcgttaggagattcgctggagtacagtgtgtttggtcttacgagatgcaacagttattctacgagaactacgagcagaataagaagggatacatccgtgatctgcacaactccaaaattcaccaagcaataacgctgcatcctaacaagaatccgccctatcagtataggttgcactcttacatgttgagtagaaagatatctgagctacgtcatagaactattcagttgcacagggagatagtactgatgtctaaatactcaaacacggagatccataaggaagacctacagttgggtataccaccttcgttcatgagattccagccaagacagagggaagaaattttggagtgggagttcttgactggtaaatacttgtattctgctgttgacggtcaaccacctaggagagggatggactctgcacagcgtgaggctttagacgatatagtgatgcaagtaatggaaatgatcaacgctaatgcaaagacacgtggtcgtatcatagatttcaaagagatccagtatggatatcgtcgtgtgaacccaatgtacggcgcggagtatattcttgacttattgttgctatacaagaaacataagggtaagaagatgactgtcccggttcgtaggcacgcgtatttgcaacagactttctcgaaaatacagtttgtagagcatgaggaacttgacgcgcaagagttagcgaaacgtataaaccaagagtcggggagtttatccttcttaagtaattcccttaagaagttggttcctttccagctgcctggaagtaaaagtgagcacaaggagccaaaggataagaaaatcaacattcttatacctctttccggcagattcgacatgttcgttaggttcatgggtaacttcgagaagacctgcttaattcctaaccagaatgttaagttggtagttttgttattcaattctgacagtaaccccgacaaagcaaagcaggttgaactgatgagagactataggattaagtacccaaaagcagatatgcagatactacctgtttccggagaattctcaagagccttggctctggaagtaggcagctctcagtttaataatgagtctcttctgttcttctgcgacgttgacttagttttcacgacagagtttctgcagagatgtcgtgcaaacactgtactaggtcagcagatatatttccccattattttcagtcagtacgatccaaagattgtttatagtggaaaagttccatctgacaaccattttgcgtttactcaaaagacagggttttggcgtaactacgggttcggcataacatgtatctataaaggggacctggtaagagttggcggattcgacgtatctatacagggctggggtttagaagacgtcgatctatttaataaagtagttcaggcaggtttgaagacattccgtagtcaagaggtaggcgttgtacacgtgcaccatccagtattttgcgacccaaacttagacccgaagcaatataaaatgtgcttgggttccaaggcatcgacttacggttccacgcagcaactactgtttgcagcatggaagcatggactttcgggcatttggctcgcattcgagtgcttataccagattacttatttcttcctggtgatttacctattctcccgtttggctgttgaggctgacccacgggcgcaaacggccaccgtcattgtttcgaccacagttgcgcttattaagtgcggttactttagcttcagagctaaagacattagggccttctatttcgtcctctacacgttcgtttacttcttctgcatgattccggcacgaataactgcgatgatgaccctgtgggacataggttggggaaccaggggaggaaatgagaagccttccgtaggcaccagagttgctttgtgggccaagcaatatttgattgcttacatgtggtgggccgctgtcgtcggtgctggcgtgtactccatcgttcataattggatgtttgactggaattcactttcctacaggttcgcactggtaggcatctgttcgtatatagttttcattgtaatagtgctggtagtctactttacaggaaagatcacgacctggaacttcacgaagctacagaaagaattgatcgaggaccgcgtactgtacgacgcaacgaccaatgcccagtcggtataa

SEQ ID NO: 15 - DNA, 3006 bp, Artificial Sequence
  Synthetic reencoded nucleic acid sequence of a polypeptide having chondroitin synthase activity (HCOS10-Vir)
atgggtaagaacatcattattatggtttcttggtacactattattacatccaatctcatcgcagtgggtggcgcctcactcatactagccccagctattacgggctatgtccttcactggaacattgccctttcaacaatttggggagtgtcggcctacggaattttcgtgtttggtttctttcttgcccaggtattatttagtgaactcaaccggaaaaggctccggaagtggatttccctccgacccaaaagagatagaaacttcctattcgttggtgttatgacagctcagaagtacttacagaccagggcggttgcagcatatagaacctggtcaaagacaattccaggtaaggtacagttcttcagctccgagggttctgacaccagcgtccctatcccagtagtgcctttgcgtggagtggatgattcatacccacctcagaagaagtccttcatgatgcttaagtatatgcacgaccactatctggacaaatacgagtggttcatgagagcagacgacgacgtttacataaagggtgatagactggagaatttcttgaggtcactaaacagttcagaaccacttttcctaggacagacgggccttggtacgacagaggagatgggtaagttggcattggagccaggggagaacttctgcatgggtggccctggtgtgattatgagtagggaagttttaagaagaatggttccgcacattggtaagtgtctaagggaaatgtatacgactcatgaggacgtggaagtaggaagatgcgttaggagattcgctggagtacagtgtgtttggtcttacgagatgcaacagttattctacgagaactacgagcagaataagaagggatacatccgtgatctgcacaactccaaaattcaccaagcaataacgctgcatcctaacaagaatccgccctatcagtataggttgcactcttacatgttgagtagaaagatatctgagctacgtcatagaactattcagttgcacagggagatagtactgatgtctaaatactcaaacacggagatccataaggaagacctacagttgggtataccaccttcgttcatgagattccagccaagacagagggaagaaattttggagtgggagttcttgactggtaaatacttgtattctgctgttgacggtcaaccacctaggagagggatggactctgcacagcgtgaggctttagacgatatagtgatgcaagtaatggaaatgatcaacgctaatgcaaagacacgtggtcgtatcatagatttcaaagagatccagtatggatatcgtcgtgtgaacccaatgtacggcgcggagtatattcttgacttattgttgctatacaagaaacataagggtaagaagatgactgtcccggttcgtaggcacgcgtatttgcaacagactttctcgaaaatacagtttgtagagcatgaggaacttgacgcgcaagagttagcgaaacgtataaaccaagagtcggggagtttatccttcttaagtaattcccttaagaagttggttcctttccagctgcctggaagtaaaagtgagcacaaggagccaaaggataagaaaatcaacattcttatacctctttccggcagattcgacatgttcgttaggttcatgggtaacttcgagaagacctgcttaattcctaaccagaatgttaagttggtagttttgttattcaattctgacagtaaccccgacaaagcaaagcaggttgaactgatgagagactataggattaagtacccaaaagcagatatgcagatactacctgtttccggagaattctcaagagccttggctctggaagtaggcagctctcagtttaataatgagtctcttctgttcttctgcgacgttgacttagttttcacgacagagtttctgcagagatgtcgtgcaaacactgtactaggtcagcagatatatttccccattattttcagtcagtacgatccaaagattgtttatagtggaaaagttccatctgacaaccattttgcgtttactcaaaagacagggttttggcgtaactacgggttcggcataacatgtatctataaaggggacctggtaagagttggcggattcgacgtatctatacagggctggggtttagaagacgtcgatctatttaataaagtagttcaggcaggtttgaagacattccgtagtcaagaggtaggcgttgtacacgtgcaccatccagtattttgcgacccaaacttagacccgaagcaatataaaatgtgcttgggttccaaggcatcgacttacggttccacgcagcaactagcagagatgtggttggagaagaacgacccatcttatagcctgtttgcagcatggaagcatggactttcgggcatttggctcgcattcgagtgcttataccagattacttatttcttcctggtgatttacctattctcccgtttggctgttgaggctgacccacgggcgcaaacggccaccgtcattgtttcgaccacagttgcgcttattaagtgcggttactttagcttcagagctaaagacattagggccttctatttcgtcctctacacgttcgtttacttcttctgcatgattccggcacgaataactgcgatgatgaccctgtgggacataggttggggaaccaggggaggaaatgagaagccttccgtaggcaccagagttgctttgtgggccaagcaatatttgattgcttacatgtggtgggccgctgtcgtcggtgctggcgtgtactccatcgttcataattggatgtttgactggaattcactttcctacaggttcgcactggtaggcatctgttcgtatatagttttcattgtaatagtgctggtagtctactttacaggaaagatcacgacctggaacttcacgaagctacagaaagaattgatcgaggaccgcgtactgtacgacgcaacgaccaatgcccagtcggtataa

SEQ ID NO: 16 - DNA, 1596 bp, Artificial Sequence
  Synthetic reencoded nucleic acid sequence of a polypeptide having chondroitin synthase activity (HCOS11-Vir)
atgggtaagaacatcattattatggtttcttggtacactattattacatccaatctcatcgcagtgggtggcgcctcactcatactagccccagctattacgggctatgtccttcactggaacattgccctttcaacaatttggggagtgtcggcctacggaattttcgtgtttggtttctttcttgcccaggtattatttagtgaactcaaccggaaaaggctccggaagtggatttccctccgacccaaaagagatagaaacttcctattcgttggtgttatgacagctcagaagtacttacagaccagggcggttgcagcatatagaacctggtcaaagacaattccaggtaaggtacagttcttcagctccgagggttctgacaccagcgtccctatcccagtagtgcctttgcgtggagtggatgattcatacccacctcagaagaagtccttcatgatgcttaagtatatgcacgaccactatctggacaaatacgagtggttcatgagagcagacgacgacgtttacataaagggtgatagactggagaatttcttgaggtcactaaacagttcagaaccacttttcctaggacagacgggccttggtacgacagaggagatgggtaagttggcattggagccaggggagaacttctgcatgggtggccctggtgtgattatgagtagggaagttttaagaagaatggttccgcacattggtaagtgtctaagggaaatgtatacgactcatgaggacgtggaagtaggaagatgcgttaggagattcgctggagtacagtgtgtttggtcttacgagatgcaacagttattctacgagaactacgagcagaataagaagggatacatccgtgatctgcacaactccaaaattcaccaagcaataacgctgcatcctaacaagaatccgccctatcagtataggttgcactcttacatgttgctgtttgcagcatggaagcatggactttcgggcatttggctcgcattcgagtgcttataccagattacttatttcttcctggtgatttacctattctcccgtttggctgttgaggctgacccacgggcgcaaacggccaccgtcattgtttcgaccacagttgcgcttattaagtgcggttactttagcttcagagctaaagacattagggccttctatttcgtcctctacacgttcgtttacttcttctgcatgattccggcacgaataactgcgatgatgaccctgtgggacataggttggggaaccaggggaggaaatgagaagccttccgtaggcaccagagttgctttgtgggccaagcaatatttgattgcttacatgtggtgggccgctgtcgtcggtgctggcgtgtactccatcgttcataattggatgtttgactggaattcactttcctacaggttcgcactggtaggcatctgttcgtatatagttttcattgtaatagtgctggtagtctactttacaggaaagatcacgacctggaacttcacgaagctacagaaagaattgatcgaggaccgcgtactgtacgacgcaacgaccaatgcccagtcggtataa

SEQ ID NO: 17 - DNA, 1662 bp, Artificial Sequence
  Synthetic reencoded nucleic acid sequence of a polypeptide having chondroitin synthase activity (HCOS12-Vir)
atgggtaagaacatcattattatggtttcttggtacactattattacatccaatctcatcgcagtgggtggcgcctcactcatactagccccagctattacgggctatgtccttcactggaacattgccctttcaacaatttggggagtgtcggcctacggaattttcgtgtttggtttctttcttgcccaggtattatttagtgaactcaaccggaaaaggctccggaagtggatttccctccgacccaaaaaggataagaaaatcaacattcttatacctctttccggcagattcgacatgttcgttaggttcatgggtaacttcgagaagacctgcttaattcctaaccagaatgttaagttggtagttttgttattcaattctgacagtaaccccgacaaagcaaagcaggttgaactgatgagagactataggattaagtacccaaaagcagatatgcagatactacctgtttccggagaattctcaagagccttggctctggaagtaggcagctctcagtttaataatgagtctcttctgttcttctgcgacgttgacttagttttcacgacagagtttctgcagagatgtcgtgcaaacactgtactaggtcagcagatatatttccccattattttcagtcagtacgatccaaagattgtttatagtggaaaagttccatctgacaaccattttgcgtttactcaaaagacagggttttggcgtaactacgggttcggcataacatgtatctataaaggggacctggtaagagttggcggattcgacgtatctatacagggctggggtttagaagacgtcgatctatttaataaagtagttcaggcaggtttgaagacattccgtagtcaagaggtaggcgttgtacacgtgcaccatccagtattttgcgacccaaacttagacccgaagcaatataaaatgtgcttgggttccaaggcatcgacttacggttccacgcagcaactagcagagatgtggttggagaagaacgacccatcttatagcctgtttgcagcatggaagcatggactttcgggcatttggctcgcattcgagtgcttataccagattacttatttcttcctggtgatttacctattctcccgtttggctgttgaggctgacccacgggcgcaaacggccaccgtcattgtttcgaccacagttgcgcttattaagtgcggttactttagcttcagagctaaagacattagggccttctatttcgtcctctacacgttcgtttacttcttctgcatgattccggcacgaataactgcgatgatgaccctgtgggacataggttggggaaccaggggaggaaatgagaagccttccgtaggcaccagagttgctttgtgggccaagcaatatttgattgcttacatgtggtgggccgctgtcgtcggtgctggcgtgtactccatcgttcataattggatgtttgactggaattcactttcctacaggttcgcactggtaggcatctgttcgtatatagttttcattgtaatagtgctggtagtctactttacaggaaagatcacgacctggaacttcacgaagctacagaaagaattgatcgaggaccgcgtactgtacgacgcaacgaccaatgcccagtcggtataa

SEQ ID NO: 18 - protein, 972 aa, Artificial Sequence
  Synthetic amino acid sequence of a polypeptide having a chondroitin synthase activity (HCOS-1)
MNTLSQAIKAYNSNDYQLALKLFEKSAEIYGRKIVEFQITKCKEKLSAHPSVNSAHLSVNKEEKVNVCDSPLDIATQLLLSNVKKLVLSDSEKNTLKNKWKLLTEKKSENAEVRAVALVPKDFPKDLVLAPLPDHVNDFTWYKKRKKRLGIKPEHQHVGLSIIVTTFNRPAILSITLACLVNQKTHYPFEVIVTDDGSQEDLSPIIRQYENKLDIRYVRQKDYGYQLCAVRNLGLRTAKYDFVSILDCDMAPQQLWVHSYLTELLEDDDLTIIGPRKYIDTQHIDPKDFLNNASLLESLPEVKTNNSVAAKGEGTVSLDWRLEQFEKTENLRLSDSPFRFFAAGNVAFAKKWLNKSGFFDEEFNHWGGEDVEFGYRLFRYGSFFKTIDGIMAYHQEPPGKENETDREAGKNITLDIMREKVPYIYRKLLPIEDSHINRVPLVSIYIPAYNCANYIQRCVDSALNQTVVDLEVCICNDGSTDNTLEVINKLYGNNPRVRIMSKPNGGIASASNAAVSFAKGYYIGQLDSDDYLEPDAVELCLKEFLKDKTLACVYTTNRNVNPDGSLIANGYNWPEFSREKLTTAMIAHHFRMFTIRAWHLTDGFNEKIENAVDYDMFLKLSEVGKFKHLNKICYNRVLHGDNTSIKKLGIQKKNHFVVVNQSLNRQGITYYNYDEFDDLDESRKYIFNKTAEYQEEIDILKDIKIIQNKDAKIAVSIFYPNTLNGLVKKLNNIIEYNKNIFVIVLHVDKNHLTPDIKKEILAFYHKHQVNILLNNDISYYTSNRLIKTEAHLSNINKLSQLNLNCEYIIFDNHDSLFVKNDSYAYMKKYDVGMNFSALTHDWIEKINAHPPFKKLIKTYFNDNDLKSMNVKGASQGMFMTYALAHELLTIIKEVITSCQSIDSVPEYNTEDIWFQFALLILEKKTGHVFNKTSTLTYMPWERKLQWTNEQIESAKRGENIPVNKFIINSITL

SEQ ID NO: 19 - protein, 972 aa, Artificial Sequence
  Synthetic amino acid sequence of a polypeptide having a chondroitin synthase activity (HCOS1-2)
MNTLSQAIKAYNSNDYQLALKLFEKSAEIYGRKIVEFQITKCKEKLSAHPSVNSAHLSVNKEEKVNVCDSPLDIATQLLLSNVKKLVLSDSEKNTLKNKWKLLTEKKSENAEVRAVALVPKDFPKDLVLAPLPDHVNDFTWYKKRKKRLGIKPVNKNIGLSIIIPTFNRSRILDITLACLVNQKTHYPFEVIVTDDGSQEDLSPIIRQYENKLDIRYVRQKDYGYQLCAVRNLGLRTAKYDFVSILDCDMAPQQLWVHSYLTELLEDDDLTIIGPRKYIDTQHIDPKDFLNNASLLESLPEVKTNNSVAAKGEGTVSLDWRLEQFEKTENLRLSDSPFRFFSCGNVAFSKEWLNKVGWFDEEFNHWGGEDVEFGYRLFRYGSFFKTIDGIMAYHQEPPGKENETDREAGKNITLDIMREKVPYIYRKLLPIEDSHINRVPLVSIYIPAYNCANYIQRCVDSALNQTVVDLEVCICNDGSTDNTLEVINKLYGNNPRVRIMSKPNGGIASASNAAVSFAKGYYIGQLDSDDYLEPDAVELCLKEFLKDKTLACVYTTNRNVNPDGSLIANGYNWPEFSREKLTTAMIAHHFRMFTIRAWHLTDGFNEKIENAVDYDMFLKLSEVGKFKHLNKICYNRVLHGDNTSIKKLGIQKKNHFVVVNQSLNRQGITYYNYDEFDDLDESRKYIFNKTAEYQEEIDILKDIKIIQNKDAKIAVSIFYPNTLNGLVKKLNNIIEYNKNIFVIVLHVDKNHLTPDIKKEILAFYHKHQVNILLNNDISYYTSNRLIKTEAHLSNINKLSQLNLNCEYIIFDNHDSLFVKNDSYAYMKKYDVGMNFSALTHDWIEKINAHPPFKKLIKTYFNDNDLKSMNVKGASQGMFMTYALAHELLTIIKEVITSCQSIDSVPEYNTEDIWFQFALLILEKKTGHVFNKTSTLTYMPWERKLQWTNEQIESAKRGENIPVNKFIINSITL

SEQ ID NO: 20 - protein, 972 aa, Artificial Sequence
  Synthetic amino acid sequence of a polypeptide having a chondroitin synthase activity (HCOS1-3)
MNTLSQAIKAYNSNDYQLALKLFEKSAEIYGRKIVEFQITKCKEKLSAHPSVNSAHLSVNKEEKVNVCDSPLDIATQLLLSNVKKLVLSDSEKNTLKNKWKLLTEKKSENAEVRAVALVPKDFPKDLVLAPLPDHVNDFTWYKNRKKSLGIKPVNKNIGLSIIIPTFNRSRILDITLACLVNQKTNYPFEVVVADDGSKENLLTIVQKYEQKLDIKYVRQKDYGYQLCAVRNLGLRTAKYDFVSILDCDMAPQQLWVHSYLTELLEDNDIVLIGPRKYVDTHNITAEQFLNDPYLIESLPETATNNNPSITSKGNISLDWRLEHFKKTDNLRLCDSPFRYFSCGNVAFSKEWLNKVGWFDEEFNHWGGEDVEFGYRLFAKGCFFRVIDGGMAYHQEPPGKENETDREAGKSITLKIVKEKVPYIYRKLLPIEDSHINRVPLVSIYIPAYNCANYIQRCVDSALNQTVVDLEVCICNDGSTDNTLEVINKLYGNNPRVRIMSKPNGGIASASNAAVSFAKGYYIGQLDSDDYLEPDAVELCLKEFLKDKTLACVYTTNRNVNPDGSLIANGYNWPEFSREKLTTAMIAHHFRMFTIRAWHLTDGFNEKIENAVDYDMFLKLSEVGKFKHLNKICYNRVLHGDNTSIKKLGIQKKNHFVVVNQSLNRQGITYYNYDEFDDLDESRKYIFNKTAEYQEEIDILKDIKIIQNKDAKIAVSIFYPNTLNGLVKKLNNIIEYNKNIFVIVLHVDKNHLTPDIKKEILAFYHKHQVNILLNNDISYYTSNRLIKTEAHLSNINKLSQLNLNCEYIIFDNHDSLFVKNDSYAYMKKYDVGMNFSALTHDWIEKINAHPPFKKLIKTYFNDNDLKSMNVKGASQGMFMTYALAHELLTIIKEVITSCQSIDSVPEYNTEDIWFQFALLILEKKTGHVFNKTSTLTYMPWERKLQWTNEQIESAKRGENIPVNKFIINSITL

SEQ ID NO: 21 - protein, 1023 aa, Artificial Sequence
  Synthetic amino acid sequence of a polypeptide having chondroitin synthase activity (HCOS.Sc)
MNTLSQAIKAYNSNDYQLALKLFEKSAEIYGRKIVEFQITKCKEKLSAHPSVNSAHLSVNKEEKVNVCDSPLDIATQLLLSNVKKLVLSDSEKNTLKNKWKLLTEKKSENAEVRAVALVPKDFPKDLVLAPLPDHVNDFTWYKNRKKSLGIKPVNKNIGLSIIIPTFNRSRILDITLACLVNQKTNYPFEVVVADDGSKENLLTIVQKYEQKLDIKYVRQKDYGYQLCAVRNLGLRTAKYDFVSILDCDMAPQQLWVHSYLTELLEDNDIVLIGPRKYVDTHNITAEQFLNDPYLIESLPETATNNNPSITSKGNISLDWRLEHFKKTDNLRLCDSPFRYFSCGNVAFSKEWLNKVGWFDEEFNHWGGEDVEFGYRLFAKGCFFRVIDGGMAYHQEPPGKENETDREAGKSITLKIVKEKVPYIYRKLLPIEDSHINRVPLVSIYIPAYNCANYIQRCVDSALNQTVVDLEVCICNDGSTDNTLEVINKLYGNNPRVRIMSKPNGGIASASNAAVSFAKGYYIGQLDSDDYLEPDAVELCLKEFLKDKTLACVYTTNRNVNPDGSLIANGYNWPEFSREKLTTAMIAHHFRMFTIRAWHLTDGFNEKIENAVDYDMFLKLSEVGKFKHLNKICYNRVLHGDNTSIKKLGIQKKNHFVVVNQSLNRQGITYYNYDEFDDLDESRKYIFNKTAEYQEEIDILKDIFIQMLFSWFSIANFVLTFYYLAGSMNLVIKHGEALFIFFKYLIFCDLASLFIISMGNRPQGAKHLFITSMVILSICATYSLICGFVFAFKSLASGTESHKIFVDIVISLLSTYGLYFFSSLMYLDPWHMFTSSIQYFLTLPAFTCTLQIFAFCNTHDVSWGTKGSTQESKQLSKAIVVQGPDGKQIVETDWPQEVDKKFLEIKSRLKEPEFEESSGNEKQSKNDYYRDIRTRIVMIWMLSNLILIMSIIQVFTPQDTDNGYLIFILWSVAALAAFRVVGSMAFLFMKYLRIIVSYRNKVEGSGSWEVSKLDLPNVFHKKG

SEQ ID NO: 22 - protein, 1023 aa, Artificial Sequence
  Synthetic amino acid sequence of a polypeptide having chondroitin synthase activity (HHASA.Sc)
MNTLSQAIKAYNSNDYQLALKLFEKSAEIYGRKIVEFQITKCKEKLSAHPSVNSAHLSVNKEEKVNVCDSPLDIATQLLLSNVKKLVLSDSEKNTLKNKWKLLTEKKSENAEVRAVALVPKDFPKDLVLAPLPDHVNDFTWYKKRKKRLGIKPEHQHVGLSIIVTTFNRPAILSITLACLVNQKTHYPFEVIVTDDGSQEDLSPIIRQYENKLDIRYVRQKDNGFQASAARNMGLRLAKYDFIGLLDCDMAPNPLWVHSYVAELLEDDDLTIIGPRKYIDTQHIDPKDFLNNASLLESLPEVKTNNSVAAKGEGTVSLDWRLEQFEKTENLRLSDSPFRFFAAGNVAFAKKWLNKSGFFDEEFNHWGGEDVEFGYRLFRYGSFFKTIDGIMAYHQEPPGKENETDREAGKNITLDIMREKVPYIYRKLLPIEDSHINRVPLVSIYIPAYNCANYIQRCVDSALNQTVVDLEVCICNDGSTDNTLEVINKLYGNNPRVRIMSKPNGGIASASNAAVSFAKGYYIGQLDSDDYLEPDAVELCLKEFLKDKTLACVYTTNRNVNPDGSLIANGYNWPEFSREKLTTAMIAHHFRMFTIRAWHLTDGFNEKIENAVDYDMFLKLSEVGKFKHLNKICYNRVLHGDNTSIKKLGIQKKNHFVVVNQSLNRQGITYYNYDEFDDLDESRKYIFNKTAEYQEEIDILKDIFIQMLFSWFSIANFVLTFYYLAGSMNLVIKHGEALFIFFKYLIFCDLASLFIISMGNRPQGAKHLFITSMVILSICATYSLICGFVFAFKSLASGTESHKIFVDIVISLLSTYGLYFFSSLMYLDPWHMFTSSIQYFLTLPAFTCTLQIFAFCNTHDVSWGTKGSTQESKQLSKAIVVQGPDGKQIVETDWPQEVDKKFLEIKSRLKEPEFEESSGNEKQSKNDYYRDIRTRIVMIWMLSNLILIMSIIQVFTPQDTDNGYLIFILWSVAALAAFRVVGSMAFLFMKYLRIIVSYRNKVEGSGSWEVSKLDLPNVFHKKG

SEQ ID NO: 23 - protein, 572 aa, Artificial Sequence
  Synthetic amino acid sequence of a polypeptide having chondroitin synthase activity (HCOS1-Vir)
MGKNIIIMVSWYTIITSNLIAVGGASLILAPAITGYVLHWNIALSTIWGVSAYGIFVFGFFLAQVLFSELNRKRLRKWISLRPKGWNDVRLAVIIAGYREDPYMFQKCLESVRDSDYGNVARLICVIDGDEDDDMRMAAVYKAIYNDNIKKPEFVLCESDDKEGERIDSDFSRDICVLQPHRGKRECLYTGFQLAKMDPSVNAVVLIDSDTVLEKDAILEVVYPLACDPEIQAVAGECKIWNTDTLLSLLVAWRYYSAFCVERSAQSFFRTVQCVGGPLGAYKIDIIKEIKDPWISQRFLGQKCTYGDDRRLTNEILMRGKKVVFTPFAVGWGWFDEEFTHWGGRYIVQQTRWSKSWCREIWYTLFAAWKHGLSGIWLAFECLYQITYFFLVIYLFSRLAVEADPRAQTATVIVSTTVALIKCGYFSFRAKDIRAFYFVLYTFVYFFCMIPARITAMMTLWDIGWGTRGGNEKPSVGTRVALWAKQYLIAYMWWAAVVGAGVYSIVHNWMFDWNSLSYRFALVGICSYIVFIVIVLVVYFTGKITTWNFTKLQKELIEDRVLYDATTNAQSV

SEQ ID NO: 24 - protein, 571 aa, Artificial Sequence
  Synthetic reencoded amino acid sequence of a polypeptide having chondroitin synthase activity (HCOS2-Vir)
MGKNIIIMVSWYTIITSNLIAVGGASLILAPAITGYVLHWNIALSTIWGVSAYGIFVFGFFLAQVLFSELNRKRLRKWISLRPKGWNDVRLAVIIAGYREDPYMFQKCLESVRDSDYGNVARLICVIDGDEDDDMRMAAVYKAIYNDNIKKPEFVLCESDDKEGERIDSDFSRDICVLQPHRGKRECLYTGFQLAKMDPSVNAVVLIDSDTVLEKDAILEVVYPLACDPEIQAVAGECKIWNTDTLLSLLVAWRYYSAFCVERSAQSFFRTVQCVGGPLGAYKIDIIKEIKDPWISQRFLGQKCTYGDDRRLTNEILMRGKKVVFTPFAVGWLHGENTSIKKLRYIVQQTRWSKSWCREIWYTLFAAWKHGLSGIWLAFECLYQITYFFLVIYLFSRLAVEADPRAQTATVIVSTTVALIKCGYFSFRAKDIRAFYFVLYTFVYFFCMIPARITAMMTLWDIGWGTRGGNEKPSVGTRVALWAKQYLIAYMWWAAVVGAGVYSIVHNWMFDWNSLSYRFALVGICSYIVFIVIVLVVYFTGKITTWNFTKLQKELIEDRVLYDATTNAQSV

SEQ ID NO: 25 - protein, 572 aa, Artificial Sequence
  Synthetic reencoded amino acid sequence of a polypeptide having chondroitin synthase activity (HCOS3-Vir)
MGKNIIIMVSWYTIITSNLIAVGGASLILAPAITGYVLHWNIALSTIWGVSAYGIFVFGFFLAQVLFSELNRKRLRKWISLRPKGWNDVRLAVIIAGYREDPYMFQKCLESVRDSDYGNVARLICVIDGDEDDDMRMAAVYKAIYNDNIKKPEFVLCESDDKEGERIDSDFSRDICVLQPHRGKRECLYTGFQLAKMDPSVNAVVLIDSDTVLEKDAILEVVYPLACDPEIQAVAGECKIWNTDTLLSLLVAWRYYSAFCVERSAQSFFRTVQCVGGPLGAYKIDIIKEIKDPWISQRFLGQKCTYGDDRRLTNEILMRGKKVVFTPFAVGWHMAWSDKDDAIDRYIVQQTRWSKSWCREIWYTLFAAWKHGLSGIWLAFECLYQITYFFLVIYLFSRLAVEADPRAQTATVIVSTTVALIKCGYFSFRAKDIRAFYFVLYTFVYFFCMIPARITAMMTLWDIGWGTRGGNEKPSVGTRVALWAKQYLIAYMWWAAVVGAGVYSIVHNWMFDWNSLSYRFALVGICSYIVFIVIVLVVYFTGKITTWNFTKLQKELIEDRVLYDATTNAQSV

SEQ ID NO: 26 - protein, 570 aa, Artificial Sequence
  Synthetic reencoded amino acid sequence of a polypeptide having chondroitin synthase activity (HCOS4-Vir)
MGKNIIIMVSWYTIITSNLIAVGGASLILAPAITGYVLHWNIALSTIWGVSAYGIFVFGFFLAQVLFSELNRKRLRKWISLRPKGWNDVRLAVIIAGYREDPYMFQKCLESVRDSDYGNVARLICVIDGDEDDDMRMAAVYKAIYNDNIKKPEFVLCESDDKEGERIDSDFSRDICVLQPHRGKRECLYTGFQLAKMDPSVNAVVLIDSDTVLEKDAILEVVYPLACDPEIQAVAGECKIWNTDTLLSLLVAWRYYSAFCVERSAQSFFRTVQCVGGPLGAYKIDIIKEIKDPWISQRFLGQKCTYGDDRRLTNEILMRGKKVVFTPFAVGWFDEEFTHWGGRYIVQQTRWSKSWCREIWYTLFAAWKHGLSGIWLAFECLYQITYFFLVIYLFSRLAVEADPRAQTATVIVSTTVALIKCGYFSFRAKDIRAFYFVLYTFVYFFCMIPARITAMMTLWDIGWGTRGGNEKPSVGTRVALWAKQYLIAYMWWAAVVGAGVYSIVHNWMFDWNSLSYRFALVGICSYIVFIVIVLVVYFTGKITTWNFTKLQKELIEDRVLYDATTNAQSV

SEQ ID NO: 27 - protein, 692 aa, Artificial Sequence
  Synthetic amino acid sequence of a polypeptide having chondroitin synthase activity (HCOS5-Vir)
MGKNIIIMVSWYTIITSNLIAVGGASLILAPAITGYVLHWNIALSTIWGVSAYGIFVFGFFLAQVLFSELNRKRLRKWISLRPKSIHHPISKTTHPDTENAGDSTLLCPPMDGMDDILVVMKTGVTEAKEKVPVHFQTTLRCVPHYVIYSDFEEEIEGVKIYDVLQGMDPDVMTQVPDFDLYNRIKKLGRQGLAENDFADEANSAVGKPNNPGWKLDKWKFLPMIIEALRYKPKAKWYVFMEADTYISWPTLLAWLKHFNHKKPWYLGTETQIADVIFAHGGSGFMISHAALKLAAEEYTARRTELHHYTDGHWAGDCVLGKVLADAGVNLHFSWPILQNSNLGELDEFNVDLYRQPWCFPAAAFHHLNAQKIQDLWSYEQKRWQDKDKRVLLHSDVFRDLLYPDIALKPARIGWDNLSADEQSTASSFEECRQICDQLDDCTQFSFRDKSCFTSRTPRLGAANPNTVSGWMVDRIRANMEKSGLFAAWKHGLSGIWLAFECLYQITYFFLVIYLFSRLAVEADPRAQTATVIVSTTVALIKCGYFSFRAKDIRAFYFVLYTFVYFFCMIPARITAMMTLWDIGWGTRGGNEKPSVGTRVALWAKQYLIAYMWWAAVVGAGVYSIVHNWMFDWNSLSYRFALVGICSYIVFIVIVLVVYFTGKITTWNFTKLQKELIEDRVLYDATTNAQSV

SEQ ID NO: 28 - protein, 910 aa, Artificial Sequence
  Synthetic amino acid sequence of a polypeptide having chondroitin synthase activity (HCOS6-Vir)
MGKNIIIMVSWYTIITSNLIAVGGASLILAPAITGYVLHWNIALSTIWGVSAYGIFVFGFFLAQVLFSELNRKRLRKWISLRPKLDIATQLLLSNVKKLTLSESEKNSLKNKWKSITGKKSENAEIRKVELVPKDFPKDLVLAPLPDHVNDFTWYKNRKKSLGIKPVNKNIGLSIIIPTFNRSRILDITLACLVNQKTNYPFEVVVADDGSKENLLTIVQKYEQKLDIKYVRQKDYGYQLCAVRNLGLRTAKYDFVSILDCDMAPQQLWVHSYLTELLEDNDIVLIGPRKYVDTHNITAEQFLNDPYLIESLPETATNNNPSITSKGNISLDWRLEHFKKTDNLRLCDSPFRYFSCGNVAFSKEWLNKVGWFDEEFNHWGGEDVEFGYRLFAKGCFFRVIDGGMAYHQEPPGKENETDREAGKSITLKIVKEKVPYIYRKLLPIEDSHIHRIPLVSIYIPAYNCANYIQRCVDSALNQTVVDLEVCICNDGSTDNTLEVINKLYGNNPRVRIMSKPNGGIASASNAAVSFAKGYYIGQLDSDDYLEPDAVELCLKEFLKDKTLACVYTTNRNVNPDGSLIANGYNWPEFSREKLTTAMIAHHFRMFTIRAWHLTDGFNEKIENAVDYDMFLKLSEVGKFKHLNKICYNRVLHGDNTSIKNLDTQKKNHFVVVNQSLNRQRVSNYNYDEFDNLDESRKYIFNKLFAAWKHGLSGIWLAFECLYQITYFFLVIYLFSRLAVEADPRAQTATVIVSTTVALIKCGYFSFRAKDIRAFYFVLYTFVYFFCMIPARITAMMTLWDIGWGTRGGNEKPSVGTRVALWAKQYLIAYMWWAAVVGAGVYSIVHNWMFDWNSLSYRFALVGICSYIVFIVIVLVVYFTGKITTWNFTKLQKELIEDRVLYDATTNAQSV

SEQ ID NO: 29 - protein, 703 aa, Artificial Sequence
  Synthetic reencoded amino acid sequence of a polypeptide having chondroitin synthase activity (HCOS7-Vir)
MGKNIIIMVSWYTIITSNLIAVGGASLILAPAITGYVLHWNIALSTIWGVSAYGIFVFGFFLAQVLFSELNRKRLRKWISLRPKIPTFNRSRILDITLACLVNQKTNYPFEVVVADDGSKENLLTIVQKYEQKLDIKYVRQKDYGYQLCAVRNLGLRTAKYDFVSILDCDMAPQQLWVHSYLTELLEDNDIVLIGPRKYVDTHNITAEQFLNDPYLIESLPETATNNNPSITSKGNISLDWRLEHFKKTDNLRLCDSPFRYFSCGNVAFSKEWLNKVGWFDEEFNHWGGEDVEFGYRLFAKGCFFRVIDGGMAYHQEPPGKENETDREAGKSITLKIVKEKVPYIYRKLLPIEDSHIHRIPLVSIYIPAYNCANYIQRCVDSALNQTVVDLEVCICNDGSTDNTLEVINKLYGNNPRVRIMSKPNGGIASASNAAVSFAKGYYIGQLDSDDYLEPDAVELCLKEFLKDKTLACVYTTNRNVNPDGSLIANGYNWPLFAAWKHGLSGIWLAFECLYQITYFFLVIYLFSRLAVEADPRAQTATVIVSTTVALIKCGYFSFRAKDIRAFYFVLYTFVYFFCMIPARITAMMTLWDIGWGTRGGNEKPSVGTRVALWAKQYLIAYMWWAAVVGAGVYSIVHNWMFDWNSLSYRFALVGICSYIVFIVIVLVVYFTGKITTWNFTKLQKELIEDRVLYDATTNAQSV

SEQ ID NO: 30 - protein, 569 aa, Artificial Sequence
  Synthetic reencoded amino acid sequence of a polypeptide having chondroitin synthase activity (HCOS8-Vir)
MGKNIIIMVSWYTIITSNLIAVGGASLILAPAITGYVLHWNIALSTIWGVSAYGIFVFGFFLAQVLFSELNRKRLRKWISLRPKGWNDVRLAVIIAGYREDPYMFQKCLESVRDSDYGNVARLICVIDGDEDDDMRMAAVYKAIYNDNIKKPEFVLCESDDKEGERIDSDFSRDICVLQPHRGKRECLYTGFQLAKMDPSVNAVVLIDSDTVLEKDAILEVVYPLACDPEIQAVAGECKIWNTDTLLSLLVAWRYYSAFCVERSAQSFFRTVQCVRLCDSPFRYFSCGNVAFSKEWLNKVGWFDEEFNHWGGEDVEFGYRLKKVVFTPFAVGWSDSPTNVFRYIVQQTRWSKSWCREIWYTLFAAWKHGLSGIWLAFECLYQITYFFLVIYLFSRLAVEADPRAQTATVIVSTTVALIKCGYFSFRAKDIRAFYFVLYTFVYFFCMIPARITAMMTLWDIGWGTRGGNEKPSVGTRVALWAKQYLIAYMWWAAVVGAGVYSIVHNWMFDWNSLSYRFALVGICSYIVFIVIVLVVYFTGKITTWNFTKLQKELIEDRVLYDATTNAQSV

SEQ ID NO: 31 - protein, 832 aa, Artificial Sequence
  Synthetic amino acid sequence of a polypeptide having chondroitin synthase activity (HCOS9-Vir)
MGKNIIIMVSWYTIITSNLIAVGGASLILAPAITGYVLHWNIALSTIWGVSAYGIFVFGFFLAQVLFSELNRKRLRKWISLRPKHIGKCLREMYTTHEDVEVGRCVRRFAGVQCVWSYEMQQLFYENYEQNKKGYIRDLHNSKIHQAITLHPNKNPPYQYRLHSYMLSRKISELRHRTIQLHREIVLMSKYSNTEIHKEDLQLGIPPSFMRFQPRQREEILEWEFLTGKYLYSAVDGQPPRRGMDSAQREALDDIVMQVMEMINANAKTRGRIIDFKEIQYGYRRVNPMYGAEYILDLLLLYKKHKGKKMTVPVRRHAYLQQTFSKIQFVEHEELDAQELAKRINQESGSLSFLSNSLKKLVPFQLPGSKSEHKEPKDKKINILIPLSGRFDMFVRFMGNFEKTCLIPNQNVKLVVLLFNSDSNPDKAKQVELMRDYRIKYPKADMQILPVSGEFSRALALEVGSSQFNNESLLFFCDVDLVFTTEFLQRCRANTVLGQQIYFPIIFSQYDPKIVYSGKVPSDNHFAFTQKTGFWRNYGFGITCIYKGDLVRVGGFDVSIQGWGLEDVDLFNKVVQAGLKTFRSQEVGVVHVHHPVFCDPNLDPKQYKMCLGSKASTYGSTQQLLFAAWKHGLSGIWLAFECLYQITYFFLVIYLFSRLAVEADPRAQTATVIVSTTVALIKCGYFSFRAKDIRAFYFVLYTFVYFFCMIPARITAMMTLWDIGWGTRGGNEKPSVGTRVALWAKQYLIAYMWWAAVVGAGVYSIVHNWMFDWNSLSYRFALVGICSYIVFIVIVLVVYFTGKITTWNFTKLQKELIEDRVLYDATTNAQSV

SEQ ID NO: 32 - protein, 1001 aa, Artificial Sequence
  Synthetic reencoded amino acid sequence of a polypeptide having chondroitin synthase activity (HCOS10-Vir)
MGKNIIIMVSWYTIITSNLIAVGGASLILAPAITGYVLHWNIALSTIWGVSAYGIFVFGFFLAQVLFSELNRKRLRKWISLRPKRDRNFLFVGVMTAQKYLQTRAVAAYRTWSKTIPGKVQFFSSEGSDTSVPIPVVPLRGVDDSYPPQKKSFMMLKYMHDHYLDKYEWFMRADDDVYIKGDRLENFLRSLNSSEPLFLGQTGLGTTEEMGKLALEPGENFCMGGPGVIMSREVLRRMVPHIGKCLREMYTTHEDVEVGRCVRRFAGVQCVWSYEMQQLFYENYEQNKKGYIRDLHNSKIHQAITLHPNKNPPYQYRLHSYMLSRKISELRHRTIQLHREIVLMSKYSNTEIHKEDLQLGIPPSFMRFQPRQREEILEWEFLTGKYLYSAVDGQPPRRGMDSAQREALDDIVMQVMEMINANAKTRGRIIDFKEIQYGYRRVNPMYGAEYILDLLLLYKKHKGKKMTVPVRRHAYLQQTFSKIQFVEHEELDAQELAKRINQESGSLSFLSNSLKKLVPFQLPGSKSEHKEPKDKKINILIPLSGRFDMFVRFMGNFEKTCLIPNQNVKLVVLLFNSDSNPDKAKQVELMRDYRIKYPKADMQILPVSGEFSRALALEVGSSQFNNESLLFFCDVDLVFTTEFLQRCRANTVLGQQIYFPIIFSQYDPKIVYSGKVPSDNHFAFTQKTGFWRNYGFGITCIYKGDLVRVGGFDVSIQGWGLEDVDLFNKVVQAGLKTFRSQEVGVVHVHHPVFCDPNLDPKQYKMCLGSKASTYGSTQQLAEMWLEKNDPSYSLFAAWKHGLSGIWLAFECLYQITYFFLVIYLFSRLAVEADPRAQTATVIVSTTVALIKCGYFSFRAKDIRAFYFVLYTFVYFFCMIPARITAMMTLWDIGWGTRGGNEKPSVGTRVALWAKQYLIAYMWWAAVVGAGVYSIVHNWMFDWNSLSYRFALVGICSYIVFIVIVLVVYFTGKITTWNFTKLQKELIEDRVLYDATTNAQSV

SEQ ID NO: 33 - protein, 531 aa, Artificial Sequence
  Synthetic reencoded amino acid sequence of a polypeptide having chondroitin synthase activity (HCOS11-Vir)
MGKNIIIMVSWYTIITSNLIAVGGASLILAPAITGYVLHWNIALSTIWGVSAYGIFVFGFFLAQVLFSELNRKRLRKWISLRPKRDRNFLFVGVMTAQKYLQTRAVAAYRTWSKTIPGKVQFFSSEGSDTSVPIPVVPLRGVDDSYPPQKKSFMMLKYMHDHYLDKYEWFMRADDDVYIKGDRLENFLRSLNSSEPLFLGQTGLGTTEEMGKLALEPGENFCMGGPGVIMSREVLRRMVPHIGKCLREMYTTHEDVEVGRCVRRFAGVQCVWSYEMQQLFYENYEQNKKGYIRDLHNSKIHQAITLHPNKNPPYQYRLHSYMLLFAAWKHGLSGIWLAFECLYQITYFFLVIYLFSRLAVEADPRAQTATVIVSTTVALIKCGYFSFRAKDIRAFYFVLYTFVYFFCMIPARITAMMTLWDIGWGTRGGNEKPSVGTRVALWAKQYLIAYMWWAAVVGAGVYSIVHNWMFDWNSLSYRFALVGICSYIVFIVIVLVVYFTGKITTWNFTKLQKELIEDRVLYDATTNAQSV

SEQ ID NO: 34 - protein, 553 aa, Artificial Sequence
  Synthetic reencoded amino acid sequence of a polypeptide having chondroitin synthase activity (HCOS12-Vir)
MGKNIIIMVSWYTIITSNLIAVGGASLILAPAITGYVLHWNIALSTIWGVSAYGIFVFGFFLAQVLFSELNRKRLRKWISLRPKKDKKINILIPLSGRFDMFVRFMGNFEKTCLIPNQNVKLVVLLFNSDSNPDKAKQVELMRDYRIKYPKADMQILPVSGEFSRALALEVGSSQFNNESLLFFCDVDLVFTTEFLQRCRANTVLGQQIYFPIIFSQYDPKIVYSGKVPSDNHFAFTQKTGFWRNYGFGITCIYKGDLVRVGGFDVSIQGWGLEDVDLFNKVVQAGLKTFRSQEVGVVHVHHPVFCDPNLDPKQYKMCLGSKASTYGSTQQLAEMWLEKNDPSYSLFAAWKHGLSGIWLAFECLYQITYFFLVIYLFSRLAVEADPRAQTATVIVSTTVALIKCGYFSFRAKDIRAFYFVLYTFVYFFCMIPARITAMMTLWDIGWGTRGGNEKPSVGTRVALWAKQYLIAYMWWAAVVGAGVYSIVHNWMFDWNSLSYRFALVGICSYIVFIVIVLVVYFTGKITTWNFTKLQKELIEDRVLYDATTNAQSV

SEQ ID NO: 35 - DNA, 1446 bp, Arabidopsis thaliana
atggtcaaaatatgctgtatcggggctggatatgtcggtgggcctacaatggccgttatggccctaaagtgtccagagattgaagtcgtggtagttgatatatcggagccacgaattaacgcatggaactcagaccgtctaccaatttatgagccagggttagaggatgtcgtcaaacagtgtagagggaagaatttgttcttctctacagatgtagagaagcatgtatttgagtcagatatagtgtttgtttcggtaaacactcctacgaaaacgcagggtctgggtgcaggtaaagcagcagatttgacatattgggagtccgctgctcgcatgatagctgatgtgagcaaatcgtcgaaaatcgtcgttgaaaagagtacagtaccagttcgtacagccgaggctatagaaaagattttaacgcacaattcgaagggtatcgaattccagatcttatcaaatccagaattcttggccgaagggacggcgattaaagacttatataaccctgatagagttctaatcggtggcagggacaccgctgcgggacaaaaggccattaaggcgttgcgtgacgtgtatgcccactgggttcctgttgagcaaataatctgtactaatttatggagtgccgagctatcaaagttggctgcgaacgcatttctagctcaaaggataagttcagtaaacgcaatgtcagcgctttgtgaggcaactggtgctgacgtaacccaagtcgctcacgccgttggaaccgacactagaattggaccgaagtttcttaacgcatccgtaggcttcggcggatcttgctttcagaaagacatcctgaatcttatttacatctgcgaatgcaatggtcttccagaagcagccaattattggaaacaggtagtcaaggtaaatgactaccaaaagattaggtttgctaatcgagtcgtatcttctatgttcaacaccgtctccggtaagaaaattgctattttgggatttgcgttcaagaaggacaccggcgacacgcgtgaaactcctgccatagatgtgtgtaatcgcctcgtggctgataaagcaaagctgtcgatctatgatccgcaagtattagaagagcagatccgccgtgatctgtccatggcccgattcgattgggaccatccagtcccactccagcagatcaaagctgaaggtatctccgaacaggttaacgttgtgtccgacgcctacgaggctacgaaggatgctcatggtttatgtgttttaaccgaatgggacgaattcaagtcacttgattttaagaagatctttgataatatgcagaaacccgctttcgttttcgacggaagaaacgtggtcgacgctgtgaaattgagagaaattggattcatagtatattccataggtaaacctctggatagttggctcaaggatatgccggctgttgcataa

SEQ ID NO: 36 - DNA, 1166 bp, Chlorella virus
atgagtagaattgctgtcgttggatgcggttacgtgggtacggcctgcgccgtacttttggcgcagaagaacgaagttatcgttttggatatctcggaggaccgggtacaactgattaagaataagaagtcacctatagaagataaggaaatcgaagaattcctggagacgaaagatttgaatctaacagcgacgacggataaggtgctcgcctatgagaatgctgaattcgttataatagctacaccgaccgattacgatgtcgtcactagatatttcaacactaagtctgttgaaaatgtgataggcgatgtcattaagaacactcagacacaccctactatagtgatcaagagtactattccaatcggtttcgtggataaagttagagaacagttcgattatcaaaatattatcttctcgccggaattcttgagagaaggacgagcattatatgataatctttacccctcccgtatcatcgtcggtgatgactccccaattgccttaaaattcgcgaatctcttggtcgagggtagcaaaactccactagctcccgtattaactatgggtacgcgagaggccgaagctgtaaaactattttcaaatacatatttggctatgagagtagcatacttcaatgagctagacacatttgcaatgtcccatggtatgaacgcaaaagagattatcgacggagtcacactagaaccaaggatagggcagggttattcgaatccttcatttgggtatggagcctattgtttcccaaaagatacaaagcaattgttggctaattttgaaggggttccacaagatattattggcgcaatcgtagagtctaacgaaactagaaaggaggttattgtttctgaggtcgagaacagatttccgaccacagttggcgtatataagttggctgctaaagctggttcggataatttcaggtctagtgcgatagtcgatatcatggaaaggttggctaataagggctaccacatcaaaatatttgaacctaccgttgaacaatttgagaattttgaggtagataataatctcacaacttttgcaaccgagtctgatgtaataattgcgaacagagttccagtcgaacatcgcattctgtttgggaagaagttaataacacgggatgtctatggggataa

SEQ ID NO: 37 - DNA, 1170 bp, Chlorella virus
atgtcacgaatcgcagtagttggctgtgggtacgtgggaaccgcatgcgctgtactgctggcgcaaaagaatgaggtaatcgttcttgatattagtgaggatagggtccaactaatcaagaacaagaaatccccgatcgaagataaggagattgaagaattcttggaaacaaaagacctaaacttaactgcaacaactgacaaagttttagcctacgaaaacgctgagtttgtgattatagcgacacccacagattatgacgtagttaccagatactttaacacgaagtccgtagagaacgtcattggagatgttataaagaatactcagactcatcctacgatagtaataaagtcaaccattcccataggtttcgtagataaggttagggagcaattcgattaccagaacattatattttcgccagaatttctgagagagggtcgcgccctgtatgataatctatatccatcacggattatagtgggcgatgactctccgattgcacttaagtttgctaatcttttagttgagggctccaaaactccgctcgccccagtacttacgatgggtacacgtgaggctgaagctgtcaagctgttttcaaacacataccttgctatgcgagtcgcatactttaacgaactagatacctttgctatgtcgcacggtatgaatgctaaagaaatcatagatggcgtaacgttggagcctcggataggtcaaggatattccaatccatcttttggctacggtgcgtattgtttcccaaaggacacgaagcaattattagctaactttgagggtgttccgcaagatataattggggcgatagtagaaagcaatgaaacacggaaggaggtaatcgtgagtgaagtggaaaaccgattccccactacggtcggcgtttacaaattagccgccaaggctggttccgacaatttccgatccagcgcaatagtagatattatggaaagattagctaataagggataccacattaaaatctttgaacctactgtcgaacagttcgagaacttcgaggttgataataacttgacgactttcgcaacggagagcgatgtaattattgcaaaccgcgtacctgtggaacatcgaattttgttcggaaagaagctgattacacgcgatgtatatggcgataactaa

SEQ ID NO: 38 - DNA, 1199 bp, Streptococcus
atgaagatatcggtagcgggttcggggtacgtggggttatccttgtcaatcttgcttgcccaacataacgatgtgactgttgtagatataatcgacgaaaaggtacggctaattaaccagggcatatctccgattaaggatgcggacattgaggaatatctgaagaatgcaccgttgaatcttacggctacactagacggagcttcggcttatagtaatgctgatctgattataatcgcaacgccaactaattacgattcagaacgcaattatttcgacaccagacacgttgaagaagtaattgagcaagtattggatttaaatgcctccgctactataatcatcaagagtaccatacccttgggttttattaaacacgtaagagagaaataccaaacagacagaatcatcttttctccagagttcttaagagagtcaaaggcattgtacgataacttatacccctctcgtataatagtcagttatgagaaggatgactctccaagagttatacaagcagctaaggcgttcgcgggtttattaaaagagggggcaaagagcaaggataccccagttctgtttatgggctctcaagaagctgaagctgtcaagctgtttgctaatacttttctcgccatgagggtcagttacttcaacgagcttgacacttatagcgaatcaaaaggactagacgcccaaagagttatagaaggcgtctgccatgatcaaaggataggtaatcattacaataatccatccttcggatatggcggttattgtttacccaaagactcaaagcaacttttggctaattatagaggcatacctcagtctctaatgtctgccatcgttgaatcgaacaagatccgtaagtcgtatttagctgaacaaatattagatagggcttcttcacaaaagcaggctggtgtacctttaaccataggattttaccgtttgattatgaagtccaactccgataactttagagaatcagccattaaagatattattgacatcattaatgactacggtgtcaatattgtcatttatgaacctatgttgggagaagacattggttatagagtcgttaaagatttggaacagtttaagaacgaaagtacaattattgttagtaacaggtttgaagatgatttaggtgatgttattgataaagtttatacacgtgacgtctttggtag

SEQ ID NO: 39 - protein, 481 aa, Arabidopsis thaliana
MVKICCIGAGYVGGPTMAVMALKCPEIEVVVVDISEPRINAWNSDRLPIYEPGLEDVVKQCRGKNLFFSTDVEKHVFESDIVFVSVNTPTKTQGLGAGKAADLTYWESAARMIADVSKSSKIVVEKSTVPVRTAEAIEKILTHNSKGIEFQILSNPEFLAEGTAIKDLYNPDRVLIGGRDTAAGQKAIKALRDVYAHWVPVEQIICTNLWSAELSKLAANAFLAQRISSVNAMSALCEATGADVTQVAHAVGTDTRIGPKFLNASVGFGGSCFQKDILNLIYICECNGLPEAANYWKQVVKVNDYQKIRFANRVVSSMFNTVSGKKIAILGFAFKKDTGDTRETPAIDVCNRLVADKAKLSIYDPQVLEEQIRRDLSMARFDWDHPVPLQQIKAEGISEQVNVVSDAYEATKDAHGLCVLTEWDEFKSLDFKKIFDNMQKPAFVFDGRNVVDAVKLREIGFIVYSIGKPLDSWLKDMPAVA

SEQ ID NO: 40 - protein, 389 aa, Chlorella virus
MSRIAVVGCGYVGTACAVLLAQKNEVIVLDISEDRVQLIKNKKSPIEDKEIEEFLETKDLNLTATTDKVLAYENAEFVIIATPTDYDVVTRYFNTKSVENVIGDVIKNTQTHPTIVIKSTIPIGFVDKVREQFDYQNIIFSPEFLREGRALYDNLYPSRIIVGDDSPIALKFANLLVEGSKTPLAPVLTMGTREAEAVKLFSNTYLAMRVAYFNELDTFAMSHGMNAKEIIDGVTLEPRIGQGYSNPSFGYGAYCFPKDTKQLLANFEGVPQDIIGAIVESNETRKEVIVSEVENRFPTTVGVYKLAAKAGSDNFRSSAIVDIMERLANKGYHIKIFEPTVEQFENFEVDNNLTTFATESDVIIANRVPVEHRILFGKKLITRDVYGDN

SEQ ID NO: 41 - protein, 401 aa, Streptococcus
MKISVAGSGYVGLSLSILLAQHNDVTVVDIIDEKVRLINQGISPIKDADIEEYLKNAPLNLTATLDGASAYSNADLIIIATPTNYDSERNYFDTRHVEEVIEQVLDLNASATIIIKSTIPLGFIKHVREKYQTDRIIFSPEFLRESKALYDNLYPSRIIVSYEKDDSPRVIQAAKAFAGLLKEGAKSKDTPVLFMGSQEAEAVKLFANTFLAMRVSYFNELDTYSESKGLDAQRVIEGVCHDQRIGNHYNNPSFGYGGYCLPKDSKQLLANYRGIPQSLMSAIVESNKIRKSYLAEQILDRASSQKQAGVPLTIGFYRLIMKSNSDNFRESAIKDIIDIINDYGVNIVIYEPMLGEDIGYRVVKDLEQFKNESTIIVSNRFEDDLGDVIDKVYTRDVFGRD

SEQ ID NO: 42 - DNA, 1026 bp, Pseudomonas aeruginosa
atgatgtcccgttacgaggaacttcgtaaagagctgcctgcacagccgaaggtatggttgatcactggtgtcgcgggcttcatcgggtcaaatctgctagagaccttgcttaaattggatcagaaggttgttggcttggacaactttgcaactggccaccagaggaatctggacgaggtgaggtcattggtttctgagaaacagtggagcaactttaagtttattcaaggggacattagaaatctggacgactgcaacaacgcatgtgcaggcgtcgattatgtcttacaccaggcagcattgggcagtgtgccgaggtctataaacgaccctataacatcgaatgcaactaacatagacggtttcttgaacatgctgattgcagcgagggatgcaaaagttcagtcttttacctatgccgcttcatcctctacatacggggatcaccccggtttgccaaaggtggaagacactatcggcaagcctctatctccatacgctgtgacgaagtatgttaatgagttgtacgcagacgtgttttcacgttgttatggattttcaaccatcggtctgagatactttaacgtcttcggccgtagacaagatccgaatggggcatacgccgccgtaatccctaagtggacatcctcaatgatacagggtgacgacgtctacattaacggagatggagagacatcacgtgacttctgctacatagaaaacacagttcaagcaaacttattagccgcaacggccggcctagacgccagaaaccaggtgtataatatcgcggtgggtggtagaacgtcactaaatcagcttttcttcgccttgagagacggtctagctgagaacggcgttagctaccatagggaaccagtttatagggattttagagaaggagatgtgagacacagcttggcagatatttccaaggcggctaaacttcttgggtatgctcctaaatatgacgtatcagcaggggtggcgcttgcaatgccatggtatataatgtttcttaagtaa

SEQ ID NO: 43 - DNA, 1020 bp, Pasteurella multocida
atgaacattctagtgaccggcggtgctggctatatcggctcccataccagtttatgcttactgaataaaggatataacgtggtcatcattgacaacctgatcaatagttcatgcgagtcgatccgtaggatagaactgatagctaagaagaaggtcaccttctacgagctgaacattaataatgaaaaggaagtgaaccagatccttaagaaacacaaattcgattgcataatgcacttcgcaggtgccaagtcggtggccgagtcactaattaagccaattttctactatgacaacaacgtctctggaactttgcagctgattaattgtgctataaagaacgacgtcgcaaatttcatcttttcttcatcagcaactgtttatggtgagtcaaagattatgccagtgacagaggactgccacatcggcggcacactaaatccatacggaacatcaaaatacatttcagagttgatgataagggacatcgccaagaagtactcagacactaatttcttatgcttgcgttatttcaatcctaccggggcccacgagtccggcatgatcggcgagtctccagctgatataccatccaatctggtgccttacattttacaggtggccatgggcaagttggagaagttaatggtattcggcggtgactacccaacaaaggacggcacgggtgtgagagactatattcacgtgatggacctagcggaaggtcatgtggcagccctaagctaccttttcagagataataacactaattaccatgtattcaatctgggcacgggtaagggctacagtgtccttgaattagtaagcacattcgagaaaatctcgggtgtcaggattccatacgagatcgtttctcgtcgtgatggggacatcgcagagagttggtcatcaccggagaaagcgaacaagtacttaaattggaaggccaagagggagcttgagacaatgctagaagacgcatggcgttggcagatgaagaacccgaatggttacatataa

SEQ ID NO: 44 - DNA, 1021 bp, Escherichia coli
aatgaacattctagtgaccggcggtgctggctatatcggctcccataccagtttatgcttactgaataaaggatataacgtggtcatcattgacaacctgatcaatagttcatgcgagtcgatccgtaggatagaactgatagctaagaagaaggtcaccttctacgagctgaacattaataatgaaaaggaagtgaaccagatccttaagaaacacaaattcgattgcataatgcacttcgcaggtgccaagtcggtggccgagtcactaattaagccaattttctactatgacaacaacgtctctggaactttgcagctgattaattgtgctataaagaacgacgtcgcaaatttcatcttttcttcatcagcaactgtttatggtgagtcaaagattatgccagtgacagaggactgccacatcggcggcacactaaatccatacggaacatcaaaatacatttcagagttgatgataagggacatcgccaagaagtactcagacactaatttcttatgcttgcgttatttcaatcctaccggggcccacgagtccggcatgatcggcgagtctccagctgatataccatccaatctggtgccttacattttacaggtggccatgggcaagttggagaagttaatggtattcggcggtgactacccaacaaaggacggcacgggtgtgagagactatattcacgtgatggacctagcggaaggtcatgtggcagccctaagctaccttttcagagataataacactaattaccatgtattcaatctgggcacgggtaagggctacagtgtccttgaattagtaagcacattcgagaaaatctcgggtgtcaggattccatacgagatcgtttctcgtcgtgatggggacatcgcagagagttggtcatcaccggagaaagcgaacaagtacttaaattggaaggccaagagggagcttgagacaatgctagaagacgcatggcgttggcagatgaagaacccgaatggttacatataa

SEQ ID NO: 45 - protein, 341 aa, Pseudomonas aeruginosa
MMSRYEELRKELPAQPKVWLITGVAGFIGSNLLETLLKLDQKVVGLDNFATGHQRNLDEVRSLVSEKQWSNFKFIQGDIRNLDDCNNACAGVDYVLHQAALGSVPRSINDPITSNATNIDGFLNMLIAARDAKVQSFTYAASSSTYGDHPGLPKVEDTIGKPLSPYAVTKYVNELYADVFSRCYGFSTIGLRYFNVFGRRQDPNGAYAAVIPKWTSSMIQGDDVYINGDGETSRDFCYIENTVQANLLAATAGLDARNQVYNIAVGGRTSLNQLFFALRDGLAENGVSYHREPVYRDFREGDVRHSLADISKAAKLLGYAPKYDVSAGVALAMPWYIMFLK

SEQ ID NO: 46 - protein, 338 aa, Pasteurella multocida
MAILVTGGAGYIGSHTVVELLNANKDVVVLDNLCNSSPKSLERVAQITGKQVKFYQGDILDTALLQKIFAENQIQSVIHFAGLKAVGESVQKPAEYYMNNVTGSLVLIQEMKKAGVWNFVFSSSATVYGDPEIIPITESCKVGGTTNPYGTSKFMVEQILKDIAKATPEFSITILRYFNPVGAHESGLIGEDPNGIPNNLLPYISQVAIGKLPQLSVFGSDYETHDGTGVRDYIHVVDLAIGHLKALDRHEGDAGLHIYNLGTGSGYSVLDMVKAFEKVNDIKIPYKLVDRRPGDIATCYSDPSLAKTELNWTAARGLEQMMKDTWHWQKKNPKGYRD

SEQ ID NO: 47 - protein, 339 aa, Escherichia coli
MNILVTGGAGYIGSHTSLCLLNKGYNVVIIDNLINSSCESIRRIELIAKKKVTFYELNINNEKEVNQILKKHKFDCIMHFAGAKSVAESLIKPIFYYDNNVSGTLQLINCAIKNDVANFIFSSSATVYGESKIMPVTEDCHIGGTLNPYGTSKYISELMIRDIAKKYSDTNFLCLRYFNPTGAHESGMIGESPADIPSNLVPYILQVAMGKLEKLMVFGGDYPTKDGTGVRDYIHVMDLAEGHVAALSYLFRDNNTNYHVFNLGTGKGYSVLELVSTFEKISGVRIPYEIVSRRDGDIAESWSSPEKANKYLNWKAKRELETMLEDAWRWQMKNPNGYI

SEQ ID NO: 48 - DNA, 1197 bp, Cupiennius salei
atgcaattcagcactgttgcatcagttgcatttgtcgccctggcgaattttgtagccgcgttcaagatttactggaacgtcccaacttttcagtgcacgcataactacaaaatcgattatgtcaaattgttgtccacttacgggatacaggtcaatgatggcggtaagtttcaaggaaaccaagtgactatcttttatgaaacccagttgggtttgtatccacgaatcctaaaatctggtaaaatggaaaacggcggaatccctcaacgcggtaactttgagaaacacctagaaaaggcaagcacggacctccagaaagtgatcccttggaaagagtttagcggattaggtgtgatagattgggaggcttggagacccacatgggaatttaactgggaaccgttgaggatatatcaaaccgaatcaattaagagagctaaagaactacaccctaccgcaaacgattccgcagtaaaagaaattgcagagcggcaatgggaagattcagccaagttatacatgttagaaacactgcggctggcaaagaaacttcgacctcaagcgccttggtgttactacttatttcctgattgctataattacgtcggaaagaaaccaaaagatttccaatgtagtgcctcgatacgtaaaggtaacgataagctaagctggttgtggaaagattctacggcattgtgtccatcgatatacgtatatgaatcacaattagacaggtattcttttgaacaaaggacatggcgcgacaatgagaaacttcgggaagcgttgcgtgtagccacgagaacctctaaaatatacccatacgttaactatttcgataaggagcttataccggagcaagaagtatggagaatgcttgcgcaggcagctgctgtcggtggcagtggtgcggtaatttggggctcatctgctgcagttgcatctgaagagttatgtaaatctttaaaacagtatattattgaaacgcttgggccggcggcagagaaggtggcttggcgtagtgacttatgcagcaaagaaatttgtaataatcagggtcgctgcacattcccggacgatgattatgcaaacgcatggaaattatttacagatgatactgttaagttttatgctggtaatattacatgtaggtgctccgagaattattctggtcgtttctgcgaaaagaagaattaa

SEQ ID NO: 49 - DNA, 1218 bp, Tityus serrulatus
atgcaattctctactgtcgcttccgttgctttcgtcgctttggctaactttgttgccgctgctgattttaaagtttactgggaagtgccttccttcctttgttctaaacgttttaaaattaatgtaacggaagttttaacaagtcacgagattcttgtcaatcagggtgagagtttcaacggtgacaagatagtaatcttttacgaaaaccaattggggaagtacccgcatattgactcaaacaatgtggagatcaatggaggaatacttcaagtagccgatttggcgaagcatttgaaagtagccaaggataatatcactaaattcgtcccgaatcctaatttcaacggtgtcggagtgatcgactgggaagcttggcggccatcatgggaatttaactggggtaagttaaaagtatataaagaaaagagcattgacttggtcaagtcgaaacatccggagtggccctccgacagggttgaaaaggttgctaaagaggagtgggaggagagtgccaaagaatggatggtgaagaccctgaagttagcacaggaaatgcgaccgaacgcagtttggtgctattatctattccctgactgctacaattatttcggtaaggatcaaccctctcaattcagctgctcgtctcgaattcagaaggaaaattctcgtctttcttggctctggaatcaatcaacagccatttgcctaagcatttatatccaggaatcccatgttaccaaatataatatgtcccagcggacatggtggatcgatgcgagattaagagaagcaattcgagtcagcgaacacagaccaaacatacccatctacccttacattaattatattctacctggaactaatcaaactgtaccagcaatggactttaaaaggacactgggtcaaatagctagcctcggcctagatggtgctttgttatggggatctagctatcatgttttaacagaatctcaatgcaaaatcacttctgattatgtgaaatcagtgattgctcctaccgtggctactgtcgttctcaatacaaacagatgctcacagataatttgtaagggtcgcggcaactgtgtttggcctgaagaaccatttagttcttggaaatacttagttgaccccaaaatgccagtgttcaagccaaccaacatccactgtaaatgtaaaggttacctaggtagatactgtgagatcccaaagtaa

SEQ ID NO: 50 - DNA, 1308 bp, Bos taurus
atgcaattttctactgttgcttctgtcgctttcgtagcactggccaattttgtagctgctggaagccgtgaccccgttgtcccaaaccagcctttcacaacaatatggaacgcaaatacggagtggtgcatgaagaagcatggagtggacgtcgatatttcaatcttcgacgtggtcactaatccaggacagacatttagaggtccaaatatgacaatattttacagttcacaattggggacctacccttactacacatccgcgggtgagccagtattcggtggtctaccccagaacgcaagtttgaatgcccacttggctaggacatttcaagatattctggcagccatgccagagcccaggttctcaggtttggctgtcatagactgggaagcgtggagacctcgttgggcatttaattgggatacgaaagacatatacaggcagaggtccagagcattggttcagaaacagcacccggattggctggcgccacgtgttgaggccgcagcgcaagaccagttcgagggcgctgctgaggagtggatggccggcacacttaaattagggcaggcgcttcgtcctcaaggcttgtggggtttctataacttcccggagtgttacaattatgacttcaaatctccgaactacacaggaaggtgccctttaaacatctgtgcgcagaacgaccagttaggttggctatggggtcaaagtcgtgcactatatccatcaatctacttgccggcggctttggaaggcacgaagaagactcagatgttcgttcagcaccgtgtagcagaggcatttagggtggcggcaggcgctggtgaccccaagctacccgtcttaccctatatgcagctattttacgatatgacaaaccacttcttgcccgcggaagaactggaacatagcttaggtgagtcagcagcgcagggtgcagccggggttgttctgtgggtttcctggttatcaacttcgacaaaggaaagctgtcaagcaatcaaggaatacgtagatactacgttgggcccttcaatcttgaacgtcacatctggtgccaggttgtgttctcaggtattgtgcagtggccacgggagatgcgcgagaagaccttcatatcctaaagccaggctgattctaaattcaacgtcgttctccatcaagccaacacccggtggtggtccattgacacttcaaggcgctttgagtttggaagacagattacgtatggcagttgagtttgaatgcagatgttacagaggatggagagggacacgttgtgagcagtggggcatgtggtaa

SEQ ID NO: 51 - DNA, 1137 bp, Apis mellifera
atgcagttttccacagttgcttccgtagctttcgtagctttagcaaattttgtcgcagctttgttgttgggcttcgttcagtctactccagataataataagactgtgcgtgaatttaacgtgtattggaacgttcctacattcatgtgccataagtacgggttgagattcgaagaggtgagcgagaagtatggcattttgcaaaattggatggacaaattcagaggagaggaaattgcgatcttatacgaccccggcatgttccccgcactgttgaaagaccccaatgggaatgtagtagctaggaatggcggagtgccccagcttggcaatttaactaaacatttacaggtgttcagggaccacctaatcaatcagattccagataaaagctttcccggagttggtgtaatagatttcgagtcatggagacctattttcagacagaactgggcctctttgcaaccgtacaagaagctatcagtcgaagtcgtacgtcgtgagcacccattctgggatgatcagagagtggagcaagaggcgaaaagaagatttgaaaagtacggccagcttttcatggaagagaccttgaaagccgcaaaacgtatgcgtcctgcagccaattggggctactacgcttacccttattgctacaacctgaccccaaaccagccctcagctcaatgcgaggctacaacaatgcaggagaacgacaagatgtcatggttattcgagtccgaggacgtcttactgcccagcgtctatttgaggtggaacctgactagcggagagagggttggtttagttggtggtcgtgtcaaggaagccctgagaatagctcgtcagatgaccacctctaggaagaaggtcctaccatactactggtacaaataccaggataggagagacacggacctaagtagggctgatcttgaggcgactctacgtaaaatcacggacttaggagccgacggtttcataatctggggatcttccgatgacatcaacaccaaggcaaaatgtttgcagttcagagaatatttgaataacgagttgggcccggcagtgaagagaatagccttaaacaataatgcgaatgacaggctaacagtggatgtgtctgttgaccaggtttaa

SEQ ID NO: 52 - DNA, 1305 bp, Macaca mulatta
atgcaattcagtactgtcgctagcgtcgcctttgtggctcttgcaaatttcgtcgctgcttcacgtgggcctttattgccgaacagaccatttgcaacggtatggaatgctaacacccaatggtgtttagagaggcactcagtagacgtcgatgtctccatatttgatgttgtagcaaaccccggccagacttttcgtgggccggacatgactattttctattccagtcagctaggtacttacccttattataccccgacgggtgaacctgtctttggtggccttccacagaacgcatcacttatagcgcacttggctaggactttccaggatatattggcagctataccagcccccgacttttctgggctagccgtaattgactgggaagcatggagaccgagatgggcatttaactgggacacgaaagacatttacaggcaaaggtcccgtgccttagtccaagcacagcatcccgactggccagttactcaagttgaggctgtggcacaagaccagttccagggtgctgcgagggcatggatggcaggaaccttgcaattaggccgtgctttaagaccaaggggactatggggcttctacggttttcccgattgctacaattacgatttcctatctcctaattacacagggcaatgcccatcagggatcagagcacaaaatgaccagttaggatggttgtggggtcagagtcgtgcattatatcccagtatctacatgccggctgttttggaaggaacaggcaaatcccaaatgtacgtgcagcacagggtcgcggaagcctttagggttgctgttgcagcaggcgacccaaacttacccgtcctaccgtacgtccagattttctacgatatgaccaaccactttcttccattggacgagttagagcatagtcttggagaatcagcggctcagggcgcagcaggagtcgtgttatgggtgtcatgggagaatactagaacaaaagaatcttgccaggcaattaaagaatatatggacactaccttggggccttttattctgaacgtcacttcgggtgcgttgttatgctcgcaagccttatgttcaagccatggtcgttgtgtccgtcgtccctcacatccaaaagccttgctaatccttaatcccgcaagctttagtattcagttgacaccagatggtggtcccttgtcgttaaggggagcattgtcacttgaggaccaggcacaaatggctgttgaattcaaatgcaggtgctaccccgggtggcaaggcccttggtgtgagcagaagagtatgtggtaa

SEQ ID NO: 53 - DNA, 1083 bp, Vespa magnifica
atgcaattttctacagtggcaagtgttgcattcgttgcactagccaactttgtggcggcagatagctgtgggtcaaactgcgaaaagagtgagagaccgaaaagggtcttcaacatttactggaacgtacctacattcatgtgtcaccagtacggactatactttgacgaggtcacgaattttaatataaagcacaacagcaaagacaattttcaaggggacaagatcgcgatcttttatgaccccggggagtttcccgctctgctgccactaaactatggtaagtacaagatcaggaatggtggtgttccacaagagggtaacatcaccatccatctgcagagattcatagagtacctagataagacctatccgaaccgtaacttttcaggcatcggtgtgatcgatttcgagaggtggagaccaattttcagacagaattggggtaatatgaagatttacaagaacttctccatcgatcttgtgcgtaaagagcatcctttctggaataagaaaatgatcgagttggaagcttctaaaagattcgagaaatacgcccgtctgttcatggaagaaacattaaagttggctaagaaaactagaaaacaggccgattggggctactacggttacccctattgcttcaacatgtctcctactaatttcgttcctgactgcgatgtcacagctagggatgagaacaacgagatgtcttggttgtttaacaaccagaatgtcctattaccaagtgtatacattaggagagagctaactcctgaccagaggattgggcttgtacaggggagagtgaaggaagctgtgagaatttcaaataaactgaagcactcacctaaagtcttcagctattggtggtatgtttaccaagacgagaccaacaccttcttaacggagaccgacgtcaagaagacgtttcaggagattgtgatcaacggtggagatgggattataatctggggttcgtcctctgatgtaaacagcttgtccaagtgtacgaggttaagggagtacctattgacagtcttgggaccaattgctgttaacgtgactgaagcagtaaactaa

SEQ ID NO: 54 - DNA, 1407 bp, Cupiennius salei
atgcaattcagcactgttgcatcagttgcatttgtcgccctggcgaattttgtagccgcgttcaagatttactggaacgtcccaacttttcagtgcacgcataactacaaaatcgattatgtcaaattgttgtccacttacgggatacaggtcaatgatggcggtaagtttcaaggaaaccaagtgactatcttttatgaaacccagttgggtttgtatccacgaatcctaaaatctggtaaaatggaaaacggcggaatccctcaacgcggtaactttgagaaacacctagaaaaggcaagcacggacctccagaaagtgatcccttggaaagagtttagcggattaggtgtgatagattgggaggcttggagacccacatgggaatttaactgggaaccgttgaggatatatcaaaccgaatcaattaagagagctaaagaactacaccctaccgcaaacgattccgcagtaaaagaaattgcagagcggcaatgggaagattcagccaagttatacatgttagaaacactgcggctggcaaagaaacttcgacctcaagcgccttggtgttactacttatttcctgattgctataattacgtcggaaagaaaccaaaagatttccaatgtagtgcctcgatacgtaaaggtaacgataagctaagctggttgtggaaagattctacggcattgtgtccatcgatatacgtatatgaatcacaattagacaggtattcttttgaacaaaggacatggcgcgacaatgagaaacttcgggaagcgttgcgtgtagccacgagaacctctaaaatatacccatacgttaactatttcgataaggagcttataccggagcaagaagtatggagaatgcttgcgcaggcagctgctgtcggtggcagtggtgcggtaatttggggctcatctgctgcagttgcatctgaagagttatgtaaatctttaaaacagtatattattgaaacgcttgggccggcggcagagaaggtggcttggcgtagtgacttatgcagcaaagaaatttgtaataatcagggtcgctgcacattcccggacgatgattatgcaaacgcatggaaattatttacagatgatactgttaagttttatgctggtaatattacatgtaggtgctccgagaattattctggtcgtttctgcgaaaagaagaatggatccgccatttctcaaatcactgacggtcaaatccaagctactaccactgctaccaccgaagctaccaccactgctgccccatcttccaccgttgaaactgtttctccatccagcaccgaaactatctctcaacaaactgaaaatggtgctgctaaggccgctgtcggtatgggtgccggtgctctagctgctgctgctatgttgttataa

SEQ ID NO: 55 - DNA, 1428 bp, Tityus serrulatus
atgcaattctctactgtcgcttccgttgctttcgtcgctttggctaactttgttgccgctgctgattttaaagtttactgggaagtgccttccttcctttgttctaaacgttttaaaattaatgtaacggaagttttaacaagtcacgagattcttgtcaatcagggtgagagtttcaacggtgacaagatagtaatcttttacgaaaaccaattggggaagtacccgcatattgactcaaacaatgtggagatcaatggaggaatacttcaagtagccgatttggcgaagcatttgaaagtagccaaggataatatcactaaattcgtcccgaatcctaatttcaacggtgtcggagtgatcgactgggaagcttggcggccatcatgggaatttaactggggtaagttaaaagtatataaagaaaagagcattgacttggtcaagtcgaaacatccggagtggccctccgacagggttgaaaaggttgctaaagaggagtgggaggagagtgccaaagaatggatggtgaagaccctgaagttagcacaggaaatgcgaccgaacgcagtttggtgctattatctattccctgactgctacaattatttcggtaaggatcaaccctctcaattcagctgctcgtctcgaattcagaaggaaaattctcgtctttcttggctctggaatcaatcaacagccatttgcctaagcatttatatccaggaatcccatgttaccaaatataatatgtcccagcggacatggtggatcgatgcgagattaagagaagcaattcgagtcagcgaacacagaccaaacatacccatctacccttacattaattatattctacctggaactaatcaaactgtaccagcaatggactttaaaaggacactgggtcaaatagctagcctcggcctagatggtgctttgttatggggatctagctatcatgttttaacagaatctcaatgcaaaatcacttctgattatgtgaaatcagtgattgctcctaccgtggctactgtcgttctcaatacaaacagatgctcacagataatttgtaagggtcgcggcaactgtgtttggcctgaagaaccatttagttcttggaaatacttagttgaccccaaaatgccagtgttcaagccaaccaacatccactgtaaatgtaaaggttacctaggtagatactgtgagatcccaaagggatccgccatttctcaaatcactgacggtcaaatccaagctactaccactgctaccaccgaagctaccaccactgctgccccatcttccaccgttgaaactgtttctccatccagcaccgaaactatctctcaacaaactgaaaatggtgctgctaaggccgctgtcggtatgggtgccggtgctctagctgctgctgctatgttgttataa

SEQ ID NO: 56 - DNA, 1518 bp, Bos taurus
atgcaattttctactgttgcttctgtcgctttcgtagcactggccaattttgtagctgctggaagccgtgaccccgttgtcccaaaccagcctttcacaacaatatggaacgcaaatacggagtggtgcatgaagaagcatggagtggacgtcgatatttcaatcttcgacgtggtcactaatccaggacagacatttagaggtccaaatatgacaatattttacagttcacaattggggacctacccttactacacatccgcgggtgagccagtattcggtggtctaccccagaacgcaagtttgaatgcccacttggctaggacatttcaagatattctggcagccatgccagagcccaggttctcaggtttggctgtcatagactgggaagcgtggagacctcgttgggcatttaattgggatacgaaagacatatacaggcagaggtccagagcattggttcagaaacagcacccggattggctggcgccacgtgttgaggccgcagcgcaagaccagttcgagggcgctgctgaggagtggatggccggcacacttaaattagggcaggcgcttcgtcctcaaggcttgtggggtttctataacttcccggagtgttacaattatgacttcaaatctccgaactacacaggaaggtgccctttaaacatctgtgcgcagaacgaccagttaggttggctatggggtcaaagtcgtgcactatatccatcaatctacttgccggcggctttggaaggcacgaagaagactcagatgttcgttcagcaccgtgtagcagaggcatttagggtggcggcaggcgctggtgaccccaagctacccgtcttaccctatatgcagctattttacgatatgacaaaccacttcttgcccgcggaagaactggaacatagcttaggtgagtcagcagcgcagggtgcagccggggttgttctgtgggtttcctggttatcaacttcgacaaaggaaagctgtcaagcaatcaaggaatacgtagatactacgttgggcccttcaatcttgaacgtcacatctggtgccaggttgtgttctcaggtattgtgcagtggccacgggagatgcgcgagaagaccttcatatcctaaagccaggctgattctaaattcaacgtcgttctccatcaagccaacacccggtggtggtccattgacacttcaaggcgctttgagtttggaagacagattacgtatggcagttgagtttgaatgcagatgttacagaggatggagagggacacgttgtgagcagtggggcatgtggggatccgccatttctcaaatcactgacggtcaaatccaagctactaccactgctaccaccgaagctaccaccactgctgccccatcttccaccgttgaaactgtttctccatccagcaccgaaactatctctcaacaaactgaaaatggtgctgctaaggccgctgtcggtatgggtgccggtgctctagctgctgctgctatgttgttataa

SEQ ID NO: 57 - DNA, 1347 bp, Apis mellifera
atgcagttttccacagttgcttccgtagctttcgtagctttagcaaattttgtcgcagctttgttgttgggcttcgttcagtctactccagataataataagactgtgcgtgaatttaacgtgtattggaacgttcctacattcatgtgccataagtacgggttgagattcgaagaggtgagcgagaagtatggcattttgcaaaattggatggacaaattcagaggagaggaaattgcgatcttatacgaccccggcatgttccccgcactgttgaaagaccccaatgggaatgtagtagctaggaatggcggagtgccccagcttggcaatttaactaaacatttacaggtgttcagggaccacctaatcaatcagattccagataaaagctttcccggagttggtgtaatagatttcgagtcatggagacctattttcagacagaactgggcctctttgcaaccgtacaagaagctatcagtcgaagtcgtacgtcgtgagcacccattctgggatgatcagagagtggagcaagaggcgaaaagaagatttgaaaagtacggccagcttttcatggaagagaccttgaaagccgcaaaacgtatgcgtcctgcagccaattggggctactacgcttacccttattgctacaacctgaccccaaaccagccctcagctcaatgcgaggctacaacaatgcaggagaacgacaagatgtcatggttattcgagtccgaggacgtcttactgcccagcgtctatttgaggtggaacctgactagcggagagagggttggtttagttggtggtcgtgtcaaggaagccctgagaatagctcgtcagatgaccacctctaggaagaaggtcctaccatactactggtacaaataccaggataggagagacacggacctaagtagggctgatcttgaggcgactctacgtaaaatcacggacttaggagccgacggtttcataatctggggatcttccgatgacatcaacaccaaggcaaaatgtttgcagttcagagaatatttgaataacgagttgggcccggcagtgaagagaatagccttaaacaataatgcgaatgacaggctaacagtggatgtgtctgttgaccaggttggatccgccatttctcaaatcactgacggtcaaatccaagctactaccactgctaccaccgaagctaccaccactgctgccccatcttccaccgttgaaactgtttctccatccagcaccgaaactatctctcaacaaactgaaaatggtgctgctaaggccgctgtcggtatgggtgccggtgctctagctgctgctgctatgttgttataa

SEQ ID NO: 58 - DNA, 1515 bp, Macaca mulatta
atgcaattcagtactgtcgctagcgtcgcctttgtggctcttgcaaatttcgtcgctgcttcacgtgggcctttattgccgaacagaccatttgcaacggtatggaatgctaacacccaatggtgtttagagaggcactcagtagacgtcgatgtctccatatttgatgttgtagcaaaccccggccagacttttcgtgggccggacatgactattttctattccagtcagctaggtacttacccttattataccccgacgggtgaacctgtctttggtggccttccacagaacgcatcacttatagcgcacttggctaggactttccaggatatattggcagctataccagcccccgacttttctgggctagccgtaattgactgggaagcatggagaccgagatgggcatttaactgggacacgaaagacatttacaggcaaaggtcccgtgccttagtccaagcacagcatcccgactggccagttactcaagttgaggctgtggcacaagaccagttccagggtgctgcgagggcatggatggcaggaaccttgcaattaggccgtgctttaagaccaaggggactatggggcttctacggttttcccgattgctacaattacgatttcctatctcctaattacacagggcaatgcccatcagggatcagagcacaaaatgaccagttaggatggttgtggggtcagagtcgtgcattatatcccagtatctacatgccggctgttttggaaggaacaggcaaatcccaaatgtacgtgcagcacagggtcgcggaagcctttagggttgctgttgcagcaggcgacccaaacttacccgtcctaccgtacgtccagattttctacgatatgaccaaccactttcttccattggacgagttagagcatagtcttggagaatcagcggctcagggcgcagcaggagtcgtgttatgggtgtcatgggagaatactagaacaaaagaatcttgccaggcaattaaagaatatatggacactaccttggggccttttattctgaacgtcacttcgggtgcgttgttatgctcgcaagccttatgttcaagccatggtcgttgtgtccgtcgtccctcacatccaaaagccttgctaatccttaatcccgcaagctttagtattcagttgacaccagatggtggtcccttgtcgttaaggggagcattgtcacttgaggaccaggcacaaatggctgttgaattcaaatgcaggtgctaccccgggtggcaaggcccttggtgtgagcagaagagtatgtggggatccgccatttctcaaatcactgacggtcaaatccaagctactaccactgctaccaccgaagctaccaccactgctgccccatcttccaccgttgaaactgtttctccatccagcaccgaaactatctctcaacaaactgaaaatggtgctgctaaggccgctgtcggtatgggtgccggtgctctagctgctgctgctatgttgttataa

SEQ ID NO: 59 - DNA, 1293 bp, Vespa magnifica
atgcaattttctacagtggcaagtgttgcattcgttgcactagccaactttgtggcggcagatagctgtgggtcaaactgcgaaaagagtgagagaccgaaaagggtcttcaacatttactggaacgtacctacattcatgtgtcaccagtacggactatactttgacgaggtcacgaattttaatataaagcacaacagcaaagacaattttcaaggggacaagatcgcgatcttttatgaccccggggagtttcccgctctgctgccactaaactatggtaagtacaagatcaggaatggtggtgttccacaagagggtaacatcaccatccatctgcagagattcatagagtacctagataagacctatccgaaccgtaacttttcaggcatcggtgtgatcgatttcgagaggtggagaccaattttcagacagaattggggtaatatgaagatttacaagaacttctccatcgatcttgtgcgtaaagagcatcctttctggaataagaaaatgatcgagttggaagcttctaaaagattcgagaaatacgcccgtctgttcatggaagaaacattaaagttggctaagaaaactagaaaacaggccgattggggctactacggttacccctattgcttcaacatgtctcctactaatttcgttcctgactgcgatgtcacagctagggatgagaacaacgagatgtcttggttgtttaacaaccagaatgtcctattaccaagtgtatacattaggagagagctaactcctgaccagaggattgggcttgtacaggggagagtgaaggaagctgtgagaatttcaaataaactgaagcactcacctaaagtcttcagctattggtggtatgtttaccaagacgagaccaacaccttcttaacggagaccgacgtcaagaagacgtttcaggagattgtgatcaacggtggagatgggattataatctggggttcgtcctctgatgtaaacagcttgtccaagtgtacgaggttaagggagtacctattgacagtcttgggaccaattgctgttaacgtgactgaagcagtaaacggatccgccatttctcaaatcactgacggtcaaatccaagctactaccactgctaccaccgaagctaccaccactgctgccccatcttccaccgttgaaactgtttctccatccagcaccgaaactatctctcaacaaactgaaaatggtgctgctaaggccgctgtcggtatgggtgccggtgctctagctgctgctgctatgttgttataa

SEQ ID NO: 60 - protein, 398 aa, Cupiennius salei
MQFSTVASVAFVALANFVAAFKIYWNVPTFQCTHNYKIDYVKLLSTYGIQVNDGGKFQGNQVTIFYETQLGLYPRILKSGKMENGGIPQRGNFEKHLEKASTDLQKVIPWKEFSGLGVIDWEAWRPTWEFNWEPLRIYQTESIKRAKELHPTANDSAVKEIAERQWEDSAKLYMLETLRLAKKLRPQAPWCYYLFPDCYNYVGKKPKDFQCSASIRKGNDKLSWLWKDSTALCPSIYVYESQLDRYSFEQRTWRDNEKLREALRVATRTSKIYPYVNYFDKELIPEQEVWRMLAQAAAVGGSGAVIWGSSAAVASEELCKSLKQYIIETLGPAAEKVAWRSDLCSKEICNNQGRCTFPDDDYANAWKLFTDDTVKFYAGNITCRCSENYSGRFCEKKN

SEQ ID NO: 61 - protein, 405 aa, Tityus serrulatus
MQFSTVASVAFVALANFVAAADFKVYWEVPSFLCSKRFKINVTEVLTSHEILVNQGESFNGDKIVIFYENQLGKYPHIDSNNVEINGGILQVADLAKHLKVAKDNITKFVPNPNFNGVGVIDWEAWRPSWEFNWGKLKVYKEKSIDLVKSKHPEWPSDRVEKVAKEEWEESAKEWMVKTLKLAQEMRPNAVWCYYLFPDCYNYFGKDQPSQFSCSSRIQKENSRLSWLWNQSTAICLSIYIQESHVTKYNMSQRTWWIDARLREAIRVSEHRPNIPIYPYINYILPGTNQTVPAMDFKRTLGQIASLGLDGALLWGSSYHVLTESQCKITSDYVKSVIAPTVATVVLNTNRCSQIICKGRGNCVWPEEPFSSWKYLVDPKMPVFKPTNIHCKCKGYLGRYCEIPK

SEQ ID NO: 62 - protein, 435 aa, Bos taurus
MQFSTVASVAFVALANFVAAGSRDPVVPNQPFTTIWNANTEWCMKKHGVDVDISIFDVVTNPGQTFRGPNMTIFYSSQLGTYPYYTSAGEPVFGGLPQNASLNAHLARTFQDILAAMPEPRFSGLAVIDWEAWRPRWAFNWDTKDIYRQRSRALVQKQHPDWLAPRVEAAAQDQFEGAAEEWMAGTLKLGQALRPQGLWGFYNFPECYNYDFKSPNYTGRCPLNICAQNDQLGWLWGQSRALYPSIYLPAALEGTKKTQMFVQHRVAEAFRVAAGAGDPKLPVLPYMQLFYDMTNHFLPAEELEHSLGESAAQGAAGVVLWVSWLSTSTKESCQAIKEYVDTTLGPSILNVTSGARLCSQVLCSGHGRCARRPSYPKARLILNSTSFSIKPTPGGGPLTLQGALSLEDRLRMAVEFECRCYRGWRGTRCEQWGMW

SEQ ID NO: 63 - protein, 378 aa, Apis mellifera
MQFSTVASVAFVALANFVAALLLGFVQSTPDNNKTVREFNVYWNVPTFMCHKYGLRFEEVSEKYGILQNWMDKFRGEEIAILYDPGMFPALLKDPNGNVVARNGGVPQLGNLTKHLQVFRDHLINQIPDKSFPGVGVIDFESWRPIFRQNWASLQPYKKLSVEVVRREHPFWDDQRVEQEAKRRFEKYGQLFMEETLKAAKRMRPAANWGYYAYPYCYNLTPNQPSAQCEATTMQENDKMSWLFESEDVLLPSVYLRWNLTSGERVGLVGGRVKEALRIARQMTTSRKKVLPYYWYKYQDRRDTDLSRADLEATLRKITDLGADGFIIWGSSDDINTKAKCLQFREYLNNELGPAVKRIALNNNANDRLTVDVSVDQV

SEQ ID NO: 64 - protein, 434 aa, Macaca mulatta
MQFSTVASVAFVALANFVAASRGPLLPNRPFATVWNANTQWCLERHSVDVDVSIFDVVANPGQTFRGPDMTIFYSSQLGTYPYYTPTGEPVFGGLPQNASLIAHLARTFQDILAAIPAPDFSGLAVIDWEAWRPRWAFNWDTKDIYRQRSRALVQAQHPDWPVTQVEAVAQDQFQGAARAWMAGTLQLGRALRPRGLWGFYGFPDCYNYDFLSPNYTGQCPSGIRAQNDQLGWLWGQSRALYPSIYMPAVLEGTGKSQMYVQHRVAEAFRVAVAAGDPNLPVLPYVQIFYDMTNHFLPLDELEHSLGESAAQGAAGVVLWVSWENTRTKESCQAIKEYMDTTLGPFILNVTSGALLCSQALCSSHGRCVRRPSHPKALLILNPASFSIQLTPDGGPLSLRGALSLEDQAQMAVEFKCRCYPGWQGPWCEQKSMW

SEQ ID NO: 65 - protein, 360 aa, Vespa magnifica
MQFSTVASVAFVALANFVAADSCGSNCEKSERPKRVFNIYWNVPTFMCHQYGLYFDEVTNFNIKHNSKDNFQGDKIAIFYDPGEFPALLPLNYGKYKIRNGGVPQEGNITIHLQRFIEYLDKTYPNRNFSGIGVIDFERWRPIFRQNWGNMKIYKNFSIDLVRKEHPFWNKKMIELEASKRFEKYARLFMEETLKLAKKTRKQADWGYYGYPYCFNMSPTNFVPDCDVTARDENNEMSWLFNNQNVLLPSVYIRRELTPDQRIGLVQGRVKEAVRISNKLKHSPKVFSYWWYVYQDETNTFLTETDVKKTFQEIVINGGDGIIIWGSSSDVNSLSKCTRLREYLLTVLGPIAVNVTEAVN

SEQ ID NO: 66 - protein, 468 aa, Cupiennius salei
MQFSTVASVAFVALANFVAAFKIYWNVPTFQCTHNYKIDYVKLLSTYGIQVNDGGKFQGNQVTIFYETQLGLYPRILKSGKMENGGIPQRGNFEKHLEKASTDLQKVIPWKEFSGLGVIDWEAWRPTWEFNWEPLRIYQTESIKRAKELHPTANDSAVKEIAERQWEDSAKLYMLETLRLAKKLRPQAPWCYYLFPDCYNYVGKKPKDFQCSASIRKGNDKLSWLWKDSTALCPSIYVYESQLDRYSFEQRTWRDNEKLREALRVATRTSKIYPYVNYFDKELIPEQEVWRMLAQAAAVGGSGAVIWGSSAAVASEELCKSLKQYIIETLGPAAEKVAWRSDLCSKEICNNQGRCTFPDDDYANAWKLFTDDTVKFYAGNITCRCSENYSGRFCEKKNGSAISQITDGQIQATTTATTEATTTAAPSSTVETVSPSSTETISQQTENGAAKAAVGMGAGALAAAAMLL

SEQ ID NO: 67 - protein, 475 aa, Tityus serrulatus
MQFSTVASVAFVALANFVAAADFKVYWEVPSFLCSKRFKINVTEVLTSHEILVNQGESFNGDKIVIFYENQLGKYPHIDSNNVEINGGILQVADLAKHLKVAKDNITKFVPNPNFNGVGVIDWEAWRPSWEFNWGKLKVYKEKSIDLVKSKHPEWPSDRVEKVAKEEWEESAKEWMVKTLKLAQEMRPNAVWCYYLFPDCYNYFGKDQPSQFSCSSRIQKENSRLSWLWNQSTAICLSIYIQESHVTKYNMSQRTWWIDARLREAIRVSEHRPNIPIYPYINYILPGTNQTVPAMDFKRTLGQIASLGLDGALLWGSSYHVLTESQCKITSDYVKSVIAPTVATVVLNTNRCSQIICKGRGNCVWPEEPFSSWKYLVDPKMPVFKPTNIHCKCKGYLGRYCEIPKGSAISQITDGQIQATTTATTEATTTAAPSSTVETVSPSSTETISQQTENGAAKAAVGMGAGALAAAAMLL

SEQ ID NO: 68 - protein, 448 aa, Bos taurus
MQFSTVASVAFVALANFVAALLLGFVQSTPDNNKTVREFNVYWNVPTFMCHKYGLRFEEVSEKYGILQNWMDKFRGEEIAILYDPGMFPALLKDPNGNVVARNGGVPQLGNLTKHLQVFRDHLINQIPDKSFPGVGVIDFESWRPIFRQNWASLQPYKKLSVEVVRREHPFWDDQRVEQEAKRRFEKYGQLFMEETLKAAKRMRPAANWGYYAYPYCYNLTPNQPSAQCEATTMQENDKMSWLFESEDVLLPSVYLRWNLTSGERVGLVGGRVKEALRIARQMTTSRKKVLPYYWYKYQDRRDTDLSRADLEATLRKITDLGADGFIIWGSSDDINTKAKCLQFREYLNNELGPAVKRIALNNNANDRLTVDVSVDQVGSAISQITDGQIQATTTATTEATTTAAPSSTVETVSPSSTETISQQTENGAAKAAVGMGAGALAAAAMLL

SEQ ID NO: 69 - protein, 448 aa, Apis mellifera; identical to SEQ ID NO: 68

SEQ ID NO: 70 - protein, 504 aa, Macaca mulatta
MQFSTVASVAFVALANFVAASRGPLLPNRPFATVWNANTQWCLERHSVDVDVSIFDVVANPGQTFRGPDMTIFYSSQLGTYPYYTPTGEPVFGGLPQNASLIAHLARTFQDILAAIPAPDFSGLAVIDWEAWRPRWAFNWDTKDIYRQRSRALVQAQHPDWPVTQVEAVAQDQFQGAARAWMAGTLQLGRALRPRGLWGFYGFPDCYNYDFLSPNYTGQCPSGIRAQNDQLGWLWGQSRALYPSIYMPAVLEGTGKSQMYVQHRVAEAFRVAVAAGDPNLPVLPYVQIFYDMTNHFLPLDELEHSLGESAAQGAAGVVLWVSWENTRTKESCQAIKEYMDTTLGPFILNVTSGALLCSQALCSSHGRCVRRPSHPKALLILNPASFSIQLTPDGGPLSLRGALSLEDQAQMAVEFKCRCYPGWQGPWCEQKSMWGSAISQITDGQIQATTTATTEATTTAAPSSTVETVSPSSTETISQQTENGAAKAAVGMGAGALAAAAMLL

SEQ ID NO: 71 - protein, 360 aa, Vespa magnifica
DNFQGDKIAIFYDPGEFPALLPLNYGKYKIRNGGVPQEGNITIHLQRFIEYLDKTYPNRNFSGIGVIDFERWRPIFRQNWGNMKIYKNFSIDLVRKEHPFWNKKMIELEASKRFEKYARLFMEETLKLAKKTRKQADWGYYGYPYCFNMSPTNFVPDCDVTARDENNEMSWLFNNQNVLLPSVYIRRELTPDQRIGLVQGRVKEAVRISNKLKHSPKVFSYWWYVYQDETNTFLTETDVKKTFQEIVINGGDGIIIWGSSSDVNSLSKCTRLREYLLTVLGPIAVNVTEAVNAISQITDGQIQATTTATTEATTTAAPSSTVETVSPSSTETISQQTENGAAKAAVGMGAGALAAAAMLL

SEQ ID NO: 72 - DNA, 2154 bp, Saccharomyces cerevisiae
atgtgtggtatctttggttactgcaattatctagtggaaagatccagaggagaaattatcgacaccttagtggatggtttacaaagattagaatatagaggctatgattccaccggtattgctatcgatggtgacgaagctgattctactttcatctataagcaaatcggtaaagtgagtgctttgaaagaggagattactaagcaaaatccgaacagagacgttacttttgtctctcattgtggtattgcgcatactagatgggctactcacggtcgaccagaacaagttaactgtcaccctcaaagatctgacccagaagaccaatttgtggtcgttcataatggtatcatcacaaattttagagaactgaagactcttttaattaacaaaggttataaattcgaaagtgataccgataccgagtgtattgctaaactatatttgcatttatacaatacaaatttacaaaatgggcatgacttagatttccacgaattaaccaagctagttcttttagaactagaaggttcatacgggttattatgtaaatcttgtcactatcctaatgaggttatcgccactagaaaagggtcccctttactgattggtgtcaaatctgaaaaaaaactaaaagtcgacttcgtggatgtggaatttcccgaagaaaacgctggtcaaccggaaattccattgaaatctaacaacaaatcatttggcttgggcccaaagaaagctcgtgaatttgaagctggttcccaaaatgccaatttactaccaattgccgccaatgaatttaacttgagacattctcaatccagggctttcctatcagaagatggatctccaacaccggtggaattttttgtttcttcggatgcggcatctgttgttaaacataccaagaaggtgctatttttagaagatgacgatttggctcatatttacgatggtgagttacatattcatagatctagaagagaagtaggcgcatcaatgacaaggtccattcaaactttagagatggagttagctcagatcatgaagggcccttacgaccattttatgcaaaaggaaatctatgagcaaccagaatctactttcaatactatgagaggtagaatcgactatgaaaataataaagtgatattgggtggtttaaaggcatggttaccagttgtcagaagagcacggagactgatcatgatcgcatgcggtacttcttatcattcatgtttggctactcgtgctatcttcgaagaattatcagatatcccagttagtgtggaattagcgtctgactttctggacagaaaatgccctgtcttcagagacgatgtatgcgtgtttgtttcacaaagtggtgaaactgcggataccatgctggctctaaattattgtttagaaagaggagccttaactgtcggaattgttaacagtgttggttcttctatctctcgtgtcacccactgtggtgttcatattaacgctggtcctgaaattggtgttgcctctacaaaagcttatacttcccagtatattgccttagtgatgtttgctctatcgctgtcagatgaccgtgtatcgaaaatagacagaagaattgaaatcattcaaggcttgaagttaatcccgggccaaattaagcaggtattaaagctggaaccaagaataaaaaagctctgtgcgactgaattaaaggatcaaaaatctctattgttattgggtagaggttaccaatttgctgctgctctggaaggtgctttgaagatcaaagaaatttcttatatgcattctgaaggtgttttggcaggtgagttgaagcacggtgtcttggccttggtggacgaaaacttgccaatcattgcttttggtaccagagactctctattccctaaagtagtttcctctattgagcaagttactgtaagaaagggccatccaattattatttgtaacgaaaatgatgaagtgtgggcgcaaaaatctaaatcaatcgacctgcaaaccttagaagttccacaaactgttgattgtttacaaggtctaattaatattattccattacaactaatgtcatattggttggctgttaataaagggattgatgttgattttccaagaaacttggctaaatctgttaccgtcgaataa

SEQ ID NO: 73 - DNA, 1728 bp, Chlorella virus
atacagaagctagagtaccgcgggtacgattcgtgtggaatagcctatacagacggaggcgccattgaacggatcaggtcaatagatgggatcgacgacttaaggaagaaaacaataacagagtcttccccggtagctatcgctcattcaaggtggagtaccactgggattccaagtgttgtgaacgcccacccgcacatctctcggggcacgtctggatgcgagtcgcgcattgcagtcgtacacaatggcattattgaaaattaccagcagatccgcaagtacttgataaatcttgggtatactttcgattcacagaccgacacggaagtcattgctcatctaatagactcgcagtataacgggaacatcctgcacaccgtccaaatggcagttaagcacctcaaaggaagctacgccatagcagtcatgtgccataaggagtccgggaagatcgtggtggcaaaacagaagagtccgttggtattgggtataggaagcgacggagcttattatatcgcatcggacgtattggctcttcccactaacaaagtggtctacataagtgacggattctctgctgagttaagtcctggatcgatgactatctatgatctggatggcaataaggtagaatacgaggtggaagacgttgaaatggagcagacgtcgatgtctttagataactttgatcactacatgatcaaagagatcaatgaacagccgatatcaattctcaatactattaagaacaaaggattctatgcagagatatttggtgatctagcacatgaaatatttcagaagatcgacaatatattgattctagcctgcggaacttcgtatcatgcaggtctcgtcgggaagcaatggatagagaccatctcacgcatcccggtggatgttcatattgcaagcgagtatgagccgacaattccacgagccaatacacttgtgataacgattagtcaaagcggcgagactgcggatacaatcgcagcccttcaacgagcgcaaaacgcaggaatgatatacactttgtgtatctgcaacagccccaagtcaactctagtgcgcgagtcgataatgaaatacataaccaaatgcggttcagaagtttctgttgccagtactaaagcatttacgtcgcaacttgtggtgttgtatatgcttgcgaacgttcttgcgaacaaaactgatgatctgctaggggatcttcctcaagctatcgagagagtaatctgtcttactaatgacgaaatgaagcgttgggcggatgaaatttgtactgccaaaagtgccatcttcttagggagaggactgaacgcacctgtagcatttgagggcgcgctaaagttgaaggaaatctcatatatccatgccgagggtttcctcggtggagagttgaagcatgggccgctggctttgttggacgataagatccctgtaatcgtgactgttgctgaccacgcttatttggatcacattaaggctaacatcgacgaggtattggcacgaaacgttaccgtatacgcgatcgtcgatcagtatgttaatattgaaccacaggagcgactgcatgtagtaaaagtgccgtttgtgagtaaagagttttctcccatcatccacacaattccgatgcaattgttatcgtattatgtcgcgatcaagctgggcaagaacgttgacaagccacgtaacctggcgaaaagtgttacaacattctaa

SEQ ID NO: 74 - DNA, 1788 bp, Chlorella virus
atgtgtggcatctttggagcagtgtcaaacaacaactctatcgaggtgtcaatcaagggtattcagaagctagaatatcgtgggtatgattcgtgcggtattgcgtatacagatgggggtgcgattgagcgtatacgttctattgacggcattgacgatctgcgtaagaaaacaatcacagaatcatcaccagtggccattgctcactcgcggtggagcaccactggaattccatcagtggtgaacgcacatcctcatatttctcgcggaaccagtgggtgtgagtctcgtatcgcggtagtccacaacggtatcattgaaaactatcagcagatccgaaaatatctcatcaatcttggttatacgtttgatagtcaaacggacacagaggtcattgcgcatttgattgattctcagtacaatgggaatatcttgcacaccgtccaaatggctgtcaagcacctgaagggctcttatgccattgcagttatgtgtcataaagagtctggtaaaatagtcgtggcgaaacagaagtcacccctcgtacttggaatcggctcagatggtgcttactacatcgcttcggacgtgctggcgctgccgacaaataaagttgtttatatttcagacggtttctccgcagaactatctccagggagtatgaccatttacgatcttgatggaaataaagtagaatatgaagtagaggacgttgaaatggaacaaactagtatgtctctcgataactttgatcattacatgattaaggaaattaatgagcaaccaatcagtattctaaacactataaaaaataaagggttctatgcagaaatattcggtgatttggctcatgaaatcttccaaaaaatagacaacatcctgatactggcttgtggtacaagttatcacgccggtcttgtaggaaaacagtggatagagaccatctctagaatccccgtggatgttcacatcgcgagtgaatacgaacctactattccgagagcgaacacattggtaatcactatttcacagtcgggtgaaactgcggacacgatagcggctttgcaacgggcccaaaacgccgggatgatttatacattgtgtatttgcaattcaccaaagagcactcttgttcgtgagagcattatgaagtacatcacgaaatgtggttctgaggtgtcagtggcatcaacgaaggcgtttacttctcagctcgtagtactgtacatgctggcaaacgtattggcaaataaaaccgatgatttgctgggagacctcccacaggcaatagaacgggtaatttgtttgacaaatgacgaaatgaaacgatgggccgacgaaatttgcactgcgaaatccgcgatcttcctgggaagaggactaaacgcaccagttgcctttgagggagcgttgaagctcaaagaaatctcttacattcatgcagagggcttcctgggaggtgagttgaaacacggccccctcgcactccttgatgacaagattcctgttatcgtaaccgtagcagatcatgcttatttggaccatatcaaagcaaatatcgacgaagtgcttgcgaggaacgttacggtatacgccatagtagaccagtatgtgaacatcgagccccaggaacgccttcacgtcgtcaaggttccgtttgtatccaaagaattttctccgataattcacactatcccgatgcaactgctttcgtattacgtggcaattaagcttggaaagaacgttgacaaaccaaggaatcttgcaaaatccgtgactaccttttaa

SEQ ID NO: 75 - protein, 717 aa, Saccharomyces cerevisiae
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 76 - protein, 595 aa, Chlorella virus
MCGIFGAVSNNNSIEVSIKGIQKLEYRGYDSCGIAYTDGGAIERIRSIDGIDDLRKKTITESSPVAIAHSRWSTTGIPSVVNAHPHISRGTSGCESRIAVVHNGIIENYQQIRKYLINLGYTFDSQTDTEVIAHLIDSQYNGNILHTVQMAVKHLKGSYAIAVMCHKESGKIVVAKQKSPLVLGIGSDGAYYIASDVLALPTNKVVYISDGFSAELSPGSMTIYDLDGNKVEYEVEDVEMEQTSMSLDNFDHYMIKEINEQPISILNTIKNKGFYAEIFGDLAHEIFQKIDNILILACGTSYHAGLVGKQWIETISRIPVDVHIASEYEPTIPRANTLVITISQSGETADTIAALQRAQNAGMIYTLCICNSPKSTLVRESIMKYITKCGSEVSVASTKAFTSQLVVLYMLANVLANKTDDLLGDLPQAIERVICLTNDEMKRWADEICTAKSAIFLGRGLNAPVAFEGALKLKEISYIHAEGFLGGELKHGPLALLDDKIPVIVTVADHAYLDHIKANIDEVLARNVTVYAIVDQYVNIEPQERLHVVKVPFVSKEFSPIIHTIPMQLLSYYVAIKLGKNVDKPRNLAKSVTTF

SEQ ID NO: 77 - DNA, 1434 bp, Saccharomyces cerevisiae
atgactgacacaaaacagctattcattgaagccggacaaagtcaacttttccacaattgggaaagcttgtctcgcaaagaccaagaagaattgctttcaaacctggagcaaatatcttccaagaggtcccctgcaaaactactggaagactgtcaaaatgctattaaattctcactagctaactcttctaaggatactggcgtcgaaatttcaccattgccccctacttcgtacgagtcgcttattggcaacagtaagaaagaaaatgaatactggcgtttaggccttgaagctattggcaagggtgaagtcgcagtgattttaatggctggcggacaaggtacgcggttaggatcctctcaaccaaagggctgttacgacattggattgccttctaagaaatctctttttcaaattcaagctgaaaagttgatcaggttgcaagatatggtaaaggacaaaaaggtagaaattccttggtatattatgacatcaggccccactagagcggctactgaggcatactttcaagaacacaattattttggcttgaataaagaacaaattacgttcttcaaccagggaaccctgcctgcctttgatttaaccgggaagcatttcctaatgaaagacccagtaaacctatctcaatcaccagatggaaatggtggactctaccgtgccatcaaggaaaacaagttgaacgaagactttgataggagaggaatcaagcatgtttacatgtactgtgtcgataatgtcctatctaaaatcgcagaccctgtatttattggttttgccatcaagcatggcttcgaactggccaccaaagccgttagaaagagagatgcgcatgaatcagttgggttaattgctactaaaaacgagaaaccatgtgtcatagaatattctgaaatttccaatgaattggctgaagcaaaggataaagatggcttattaaaactacgcgcaggcaacattgtaaatcattattacctagtggatttactaaaacgtgatttggatcagtggtgtgagaatatgccatatcacattgcgaagaagaaaattccagcttatgatagtgttaccggcaagtacactaagcctaccgaaccaaacggtataaaattagagcaattcatatttgatgtctttgacactgtaccactgaacaagtttgggtgcttagaagtagatagatgcaaagaattttcacctttaaaaaacggtcctggttctaagaacgataatcctgagaccagcagactagcatatttgaaactaggaacctcgtggttggaagatgcaggcgctattgtaaaagatggggtactagtcgaagtttccagcaaattgagttatgcaggtgaaaatctatcccagttcaaaggtaaagtctttgacagaagtggtatagtattagaaaaataa

SEQ ID NO: 78 - protein, 477 aa, Saccharomyces cerevis
MTDTKQLFIEAGQSQLFHNWESLSRKDQEELLSNLEQISSKRSPAKLLEDCQNAIKFSLANSSKDTGVEISPLPPTSYESLIGNSKKENEYWRLGLEAIGKGEVAVILMAGGQGTRLGSSQPKGCYDIGLPSKKSLFQIQAEKLIRLQDMVKDKKVEIPWYIMTSGPTRAATEAYFQEHNYFGLNKEQITFFNQGTLPAFDLTGKHFLMKDPVNLSQSPDGNGGLYRAIKENKLNEDFDRRGIKHVYMYCVDNVLSKIADPVFIGFAIKHGFELATKAVRKRDAHESVGLIATKNEKPCVIEYSEISNELAEAKDKDGLLKLRAGNIVNHYYLVDLLKRDLDQWCENMPYHIAKKKIPAYDSVTGKYTKPTEPNGIKLEQFIFDVFDTVPLNKFGCLEVDRCKEFSPLKNGPGSKNDNPETSRLAYLKLGTSWLEDAGAIVKDGVLVEVSSKLSYAGENLSQFKGKVFDRSGIVLEK

SEQ ID NO: 79 - DNA, 1713 bp, Saccharomyces cerevisiae
atgtcacttctaatagattctgtaccaacagttgcttataaggaccaaaaaccgggtacttcaggtttacgtaagaagaccaaggttttcatggatgagcctcattatactgagaacttcattcaagcaacaatgcaatctatccctaatggctcagagggaaccactttagttgttggaggagatggtcgtttctacaacgatgttatcatgaacaagattgccgcagtaggtgctgcaaacggtgtcagaaagttagtcattggtcaaggcggtttactttcaacaccagctgcttctcatataattagaacatacgaggaaaagtgtaccggtggtggtatcatattaactgcctcacacaacccaggcggtccagagaatgatttaggtatcaagtataatttacctaatggtgggccagctccagagagtgtcactaacgctatctgggaagcgtctaaaaaattaactcactataaaattataaagaacttccccaagttgaatttgaacaagcttggtaaaaaccaaaaatatggcccattgttagtggacataattgatcctgccaaagcatacgttcaatttctgaaggaaatttttgattttgacttaattaaaagcttcttagcgaaacagcgcaaagacaaagggtggaagttgttgtttgactccttaaatggtattacaggaccatatggtaaggctatatttgttgatgaatttggtttaccggcagaggaagttcttcaaaattggcaccctttacctgatttcggcggtttacatcccgatccgaatctaacctatgcacgaactcttgttgacagggttgaccgcgaaaaaattgcctttggagcagcctccgatggtgatggtgataggaatatgatttacggttatggccctgctttcgtttcgccaggtgattctgttgccattattgccgaatatgcacccgaaattccatacttcgccaaacaaggtatttatggcttggcacgttcatttcctacatcctcagccattgatcgtgttgcagcaaaaaagggattaagatgttacgaagttccaaccggctggaaattcttctgtgccttatttgatgctaaaaagctatcaatctgtggtgaagaatccttcggtacaggttccaatcatatcagagaaaaggacggtctatgggccattattgcttggttaaatatcttggctatctaccataggcgtaaccctgaaaaggaagcttcgatcaaaactattcaggacgaattttggaacgagtatggccgtactttcttcacaagatacgattacgaacatatcgaatgcgagcaggccgaaaaagttgtagctcttttgagtgaatttgtatcaaggccaaacgtttgtggctcccacttcccagctgatgagtctttaaccgttatcgattgtggtgatttttcgtatagagatctagatggctccatctctgaaaatcaaggccttttcgtaaagttttcgaatgggactaaatttgttttgaggttatccggcacaggcagttctggtgcaacaataagattatacgtagaaaagtatactgataaaaaggagaactatggccaaacagctgacgtcttcttgaaacccgtcatcaactccattgtaaaattcttaagatttaaagaaattttaggaacagacgaaccaacagtccgcacatag

SEQ ID NO: 80 - protein, 570 aa, Saccharomyces cerevisiae
MSLLIDSVPTVAYKDQKPGTSGLRKKTKVFMDEPHYTENFIQATMQSIPNGSEGTTLVVGGDGRFYNDVIMNKIAAVGAANGVRKLVIGQGGLLSTPAASHIIRTYEEKCTGGGIILTASHNPGGPENDLGIKYNLPNGGPAPESVTNAIWEASKKLTHYKIIKNFPKLNLNKLGKNQKYGPLLVDIIDPAKAYVQFLKEIFDFDLIKSFLAKQRKDKGWKLLFDSLNGITGPYGKAIFVDEFGLPAEEVLQNWHPLPDFGGLHPDPNLTYARTLVDRVDREKIAFGAASDGDGDRNMIYGYGPAFVSPGDSVAIIAEYAPEIPYFAKQGIYGLARSFPTSSAIDRVAAKKGLRCYEVPTGWKFFCALFDAKKLSICGEESFGTGSNHIREKDGLWAIIAWLNILAIYHRRNPEKEASIKTIQDEFWNEYGRTFFTRYDYEHIECEQAEKVVALLSEFVSRPNVCGSHFPADESLTVIDCGDFSYRDLDGSISENQGLFVKFSNGTKFVLRLSGTGSSGATIRLYVEKYTDKKENYGQTADVFLKPVINSIVKFLRFKEILGTDEPTVRT

SEQ ID NO: 81 - DNA, 1500 bp, Saccharomyces cerevisiae
atgtccactaagaagcacaccaaaacacattccacttatgcattcgagagcaacacaaacagcgttgctgcctcacaaatgagaaacgccttaaacaagttggcggactctagtaaacttgacgatgctgctcgcgctaagtttgagaacgaactggattcgtttttcacgcttttcaggagatatttggtagagaagtcttctagaaccaccttggaatgggacaagatcaagtctcccaacccggatgaagtggttaagtatgaaattatttctcagcagcccgagaatgtctcaaacctttccaaattggctgttttgaagttgaacggtgggctgggtacctccatgggctgcgttggccctaaatctgttattgaagtgagagagggaaacacctttttggatttgtctgttcgtcaaattgaatacttgaacagacagtacgatagcgacgtgccattgttattgatgaattctttcaacactgacaaggatacggaacacttgattaagaagtattccgctaacagaatcagaatcagatctttcaatcaatccaggttcccaagagtctacaaggattctttattgcctgtccccaccgaatacgattctccactggatgcttggtatccaccaggtcacggtgatttgtttgaatctttacacgtatctggtgaactggatgccttaattgcccaaggaagagaaatattatttgtttctaacggtgacaacttgggtgctaccgtcgacttaaaaattttaaaccacatgatcgagactggtgccgaatatataatggaattgactgataagaccagagccgatgttaaaggtggtactttgatttcttacgatggtcaagtccgtttattggaagtcgcccaagttccaaaagaacacattgacgaattcaaaaatatcagaaagtttaccaacttcaacacgaataacttatggatcaatctgaaagcagtaaagaggttgatcgaatcgagcaatttggagatggaaatcattccaaaccaaaaaactataacaagagacggtcatgaaattaatgtcttacaattagaaaccgcttgtggtgctgctatcaggcattttgatggtgctcacggtgttgtcgttccaagatcaagattcttgcctgtcaagacctgttccgatttgttgctggttaaatcagatctattccgtctggaacacggttctttgaagttagacccatcccgttttggtccaaacccattaatcaagttgggctcgcatttcaaaaaggtttctggttttaacgcaagaatccctcacatcccaaaaatcgtcgagctagatcatttgaccatcactggtaacgtctttttaggtaaagatgtcactttgaggggtactgtcatcatcgtttgctccgacggtcataaaatcgatattccaaacggctccatattggaaaatgttgtcgttactggtaatttgcaaatcttggaacattga

SEQ ID NO: 82 - protein, 499 aa, Saccharomyces cerevisiae
MSTKKHTKTHSTYAFESNTNSVAASQMRNALNKLADSSKLDDAARAKFENELDSFFTLFRRYLVEKSSRTTLEWDKIKSPNPDEVVKYEIISQQPENVSNLSKLAVLKLNGGLGTSMGCVGPKSVIEVREGNTFLDLSVRQIEYLNRQYDSDVPLLLMNSFNTDKDTEHLIKKYSANRIRIRSFNQSRFPRVYKDSLLPVPTEYDSPLDAWYPPGHGDLFESLHVSGELDALIAQGREILFVSNGDNLGATVDLKILNHMIETGAEYIMELTDKTRADVKGGTLISYDGQVRLLEVAQVPKEHIDEFKNIRKFTNFNTNNLWINLKAVKRLIESSNLEMEIIPNQKTITRDGHEINVLQLETACGAAIRHFDGAHGVVVPRSRFLPVKTCSDLLLVKSDLFRLEHGSLKLDPSRFGPNPLIKLGSHFKKVSGFNARIPHIPKIVELDHLTITGNVFLGKDVTLRGTVIIVCSDGHKIDIPNGSILENVVVTGNLQILEH

SEQ ID NO: 83 - DNA, 480 bp, Saccharomyces cerevisiae
atgagcttacccgatggattttatataaggcgaatggaagagggggatttggaacaggtcactgagacgctaaaggttttgaccaccgtgggcactattacccccgaatccttcagcaaactcataaaatactggaatgaagccacagtatggaatgataacgaagataaaaaaataatgcaatataaccccatggtgattgtggacaagcgcaccgagacggttgccgctacggggaatatcatcatcgaaagaaagatcattcatgaactggggctatgtggccacatcgaggacattgcagtaaactccaagtatcagggccaaggtttgggcaagctcttgattgatcaattggtaactatcggctttgactacggttgttataagattattttagattgcgatgagaaaaatgtcaaattctatgaaaaatgtgggtttagcaacgcaggcgtggaaatgcaaattagaaaatag

SEQ ID NO: 84 - protein, 159 aa, Saccharomyces cerevisiae
MSLPDGFYIRRMEEGDLEQVTETLKVLTTVGTITPESFSKLIKYWNEATVWNDNEDKKIMQYNPMVIVDKRTETVAATGNIIIERKIIHELGLCGHIEDIAVNSKYQGQGLGKLLIDQLVTIGFDYGCYKIILDCDEKNVKFYEKCGFSNAGVEMQIRK

SEQ ID NO: 85 - DNA, 1674 bp, Saccharomyces cerevisiae
atgaaggttgattacgagcaattgtgcaaactctacgatgacacgtgccgcacaaagaatgtgcagttcagttacggtacggccggattcagaacgctggccaagaatttggatacggtgatgttcagtactggtatactggcggttctcaggtcgctgaagcttcagggtcagtatgtgggggtgatgatcacggcgtcgcacaacccataccaggacaacggggtcaagatcgtggaaccagacggatcgatgcttttggccacatgggagccatatgccatgcagttggccaatgcggcctcttttgccactaattttgaagaatttcgtgttgagttggccaagctgattgaacacgaaaagattgatttgaatacaaccgtcgtgcctcacatcgtggttgggagagactctagggaaagtagtccatacttgctgcgctgcttgacttcctccatggccagcgtcttccacgcgcaagttttggacctaggctgtgtcactacgcctcaattgcattacattactgatttgtccaacaggcggaaactggaaggagacacagcgccagttgccacagaacaggactactattcgttctttataggagccttcaacgagctcttcgccacgtatcagctggagaagaggctgtctgtcccaaaattgttcatagacacagccaatggtatcggtggtccacagttgaaaaaactactggcctccgaagattgggacgtgccagcggagcaagttgaggtaatcaacgacaggtccgatgttccagaactgttgaattttgaatgcggtgcggattatgtgaagactaaccagagattacccaagggtctttctccatcctcgtttgattcgctatattgctcctttgatggtgacgcagacagggttgtgttctactatgtcgactcaggatcaaaatttcatttgttggatggtgacaaaatttccactttgtttgcaaagttcttgtctaaacaactagaattggcacacctagaacattctttgaagattggtgttgtgcaaactgcctatgcaaacggcagttccaccgcttacataaaaaatacgttgcactgtcccgtgtcttgcactaagacaggtgttaaacacttgcatcatgaagctgccactcagtacgatattggcatttatttcgaagcaaatggacatggtacgattatattcagcgaaaaatttcatcgaactatcaaatctgaattatccaagtccaagttaaatggtgatacgttagctttgagaactttgaagtgtttctctgaattgattaatcagaccgtgggagatgctatttcagacatgcttgctgtccttgctactttggcgattttgaaaatgtcgccaatggattgggatgaagagtatactgatttgcccaacaagctggttaagtgcatcgttcctgataggtcaattttccaaaccacggaccaggaaagaaaattgctcaatccagtggggttgcaagacaagatagatcttgtggtagccaagtatcccatgggaagaagctttgtcagagccagtggtacggaggatgcggtgagggtttatgcggaatgtaaggactcctctaagttaggtcaattttgtgacgaagtggtggagcacgttaaggcatctgcttga

SEQ ID NO: 86 - protein, 557 aa, Saccharomyces cerevisiae
MKVDYEQLCKLYDDTCRTKNVQFSYGTAGFRTLAKNLDTVMFSTGILAVLRSLKLQGQYVGVMITASHNPYQDNGVKIVEPDGSMLLATWEPYAMQLANAASFATNFEEFRVELAKLIEHEKIDLNTTVVPHIVVGRDSRESSPYLLRCLTSSMASVFHAQVLDLGCVTTPQLHYITDLSNRRKLEGDTAPVATEQDYYSFFIGAFNELFATYQLEKRLSVPKLFIDTANGIGGPQLKKLLASEDWDVPAEQVEVINDRSDVPELLNFECGADYVKTNQRLPKGLSPSSFDSLYCSFDGDADRVVFYYVDSGSKFHLLDGDKISTLFAKFLSKQLELAHLEHSLKIGVVQTAYANGSSTAYIKNTLHCPVSCTKTGVKHLHHEAATQYDIGIYFEANGHGTIIFSEKFHRTIKSELSKSKLNGDTLALRTLKCFSELINQTVGDAISDMLAVLATLAILKMSPMDWDEEYTDLPNKLVKCIVPDRSIFQTTDQERKLLNPVGLQDKIDLVVAKYPMGRSFVRASGTEDAVRVYAECKDSSKLGQFCDEVVEHVKASA

SEQ ID NO: 87 - DNA, 514 bp, Artificial Sequence
  Synthetic nucleic acid sequence of promoter pTDH3
aggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 88 - DNA, 585 bp, Artificial Sequence
  Synthetic nucleic acid sequence of promoter pTDH3.Sk
caattcatcggcccttttagcggctacccgcgccatctaaatgatagggcgggtgacactatggtaaatcccataattaggtgtctgggtgagtggttctgatgccggcatccactaaatatattggagcccattttttacgcgggcttccagaaaaaaagagaatcccagcaccaaaaggtggttctcttcaccaaccatcagatcataggtccacaaccacacataacaggggcacaaaaaggcaaaaaacggacataacctcaatggagtgatgcaaattgactggagcaaaagctgacacaaggcattgattgacctacgcatgtatctgtattcttttcttacaccttctattaccttctaactctttgggttggaaaaaactgaaaaaaaaggttgggacctggttcccccaagttgtccccctacttggttattaaatatataaagacagcaagtgttgattataatcttgtaaatctatagttcttaatctatacttctatttatattttaaattagtctttttatttccaagtccccaagaacttagtttcgaataaacacacacaaataaacaca

SEQ ID NO: 89 - DNA, 700 bp, Artificial Sequence
  Synthetic nucleic acid sequence of promoter pTDH3-1.sba
gcagcgcttcttccgctctagtttttatagttattattactaccaccttaaaaatacgtaaatactcaaaatagtagtgatattcccaaccttattcatccaaggcacatcatcatcatcagccattcatctttcacctgccattagtaacccgtcttctcattgagcgggttacggcagccacaggccacattccgaatgtctgggtgagcggtcccttttccagcatccactaaatatctccgatcccgctttttaatctggcttcctgaaaaaaagagaatcccagcaccaaaaaatggctctcttcaccaaccatcagatcataggtcccattctcttaccgcaaccgtacagaacaggggaaaacgggtacaacctcaatggagtgatgcaaactgactggagcaaaaagctgacacaaggcaatcgacctacgtgtctgtctattttctcacaccttctattaccttctaactctctgggttggaaaaaactgaaaaaaaggttgagaccagtttccacaaatcatccccctgtttgattaataaatatataaagacgacaactatcgatcataaactcataaaactataactcctttacacttcttattttatagttattctattttaattcttattgattttaaaaccccaagaacttagtttcgaaaacacacacacacaaacaattaaaa

SEQ ID NO: 90 - DNA, 602 bp, Artificial Sequence
  Synthetic nucleic acid sequence of promoter pTDH3.Sar
gagctaaatatcagcccttcgggtcctgcctgctacccggtcctgttcgaataaaaacgcgggtaacacgacccagtaacacctgtcgttgggtgtctgggtcagaagttctgataccggcttccactaaatagattgggttccgctctttacgctggcttcctgaaaaaagagattccgggcaccaaaaaattggtctctttgccaaccatcagatcataggtccattctcttaccataaccacacaggataggggcaccacaggcgaaaatgggcacaaaatctcaatggagtgatgcaaattagctggaacaaaagctgacacaaggcaattaacctgcgcatgtatccatctccttttcttacaccttctcttacgttctaactgtttgggttggaaaaattaaaaaaaaaaggttgagaccagtttccccaaatcgtccccctacttgattcataaatatataaagacgacaactattgattataatcttgtaaatctataactctttactttctcctatttataatttaacttaatctttttagatttaaaaccccaagaacttagtttcgaacaaacacacacaaataaacaaaa

SEQ ID NO: 91 - DNA, 550 bp, Artificial Sequence
  Synthetic nucleic acid sequence of promoter pENO2
cgctcagcatctgcttcttcccaaagatgaacgcggcgttatgtcactaacgacgtgcaccaacttgcggaaagtggaatcccgttccaaaactggcatccactaattgatacatctacacaccgcacgccttttttctgaagcccactttcgtggactttgccatatgcaaaattcatgaagtgtgataccaagtcagcatacacctcactagggtagtttctttggttgtattgatcatttggttcatcgtggttcattaattttttttctccattgctttctggctttgatcttactatcatttggatttttgtcgaaggttgtagaattgtatgtgacaagtggcaccaagcatatataaaaaaaaaaagcattatcttcctaccagagttgattgttaaaaacgtatttatagcaaacgcaattgtaattaattcttattttgtatcttttcttcccttgtctcaatcttttatttttattttatttttcttttcttagtttctttcataacaccaagcaactaatactataacatacaataata

SEQ ID NO: 92 - DNA, 598 bp, Artificial Sequence
  Synthetic nucleic acid sequence of promoter pTEF3
ggctgataatagcgtataaacaatgcatactttgtacgttcaaaatacaatgcagtagatatatttatgcatattacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa

SEQ ID NO: 93 - DNA, 383 bp, Artificial Sequence
  Synthetic nucleic acid sequence of promoter pTEF1
gtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctacgatacagttctcacatcacatccgaacataaacaacc

SEQ ID NO: 94 - DNA, 383 bp, Artificial Sequence; identical to SEQ ID NO: 93
  Synthetic nucleic acid sequence of promoter pTEF1.ago

SEQ ID NO: 95 - DNA, 498 bp, Artificial Sequence
  Synthetic nucleic acid sequence of promoter pTEF1.Sba
cgccaacaaaccttcgaacactttaattttcgaggaccgcagatcctcacatcacacccacacccaagactgcttcccccacacaccctgcatctgtacactttcttctgctctgtttttctctccggcgttctctcgggtcgcccgcatcgccgcgccggctggaaccgcccacgcaccgcatattgcaaatcgcctgccccctcttgctcctttttgagggcgcgccgttacccgcgcccagggtccggaaaaagaaacaaggctctaccgcgtttctttttccttgtcgaaaaaggcaaaaatgaaaatttttatcacgtttctttttttttgaaaaattttttttttggttttttttctttcgatggcctcccattgatatttaagttaataaatggttttcagttttcaagtttcagtttgtgttcttctttgctaactttcacttacacctcgaaagaaagtatagcaatctaatcttagttttaattacaa

SEQ ID NO: 96 - DNA, 700 bp, Artificial Sequence
  Synthetic nucleic acid sequence of promoter pPDC1
ttatttacctatctctaaacttcaacaccttatatcataactaatatttcttgagataagcacactgcacccataccttccttaaaaacgtagcttccagtttttggtggttccggcttccttcccgattccgcccgctaaacgcatatttttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccctttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttgcaatcagatttgggtttgttccctttattttcatatttcttgtcatattcctttctcaattattattttctactcataacctcacgcaaaataacacagtcaaatcaatcaaa

SEQ ID NO: 97 - DNA, 924 bp, Artificial Sequence
  Synthetic nucleic acid sequence of promoter pCCW12
aactgtaaacaggtacaatgcggacccttttgccacaaaacatacatcattcattgccggaaaaagaaagaagtgaagacagcagtgcagccagccatgttgcgccaatctaattatagatgctggtgccctgaggatgtatctggagccagccatggcatcatgcgctaccgccggatgtaaaatccgacacgcaaaagaaaaccttcgaggttgcgcacttcgcccacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaataaaagccaatcctcgaagcgtgggtggtaggccctggattatcccgtacaagtatttctcaggagtaaaaaaaccgtttgttttggaattccccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaata

SEQ ID NO: 98 - DNA, 976 bp, Artificial Sequence
  Synthetic nucleic acid sequence of promoter pCCW12.Sm
cgttgtgctgtagtgaaggaagactaaaaaggataatcacagttgtaaagaggtataatgcggacccttttgccacaaaacacacatctttcgtttccaaaataagaaagaagaaagcaaaaagattagcagccacgttgctgcgatctaattatagacggtggcgtcatcattctcacccaagattgtgtcttgaacctgccacgggtcctgcgttatcgccggatgtaaaactagacatgcaaaaaaaggaccttccaggtagcgtgctccacaccacccatgaccaccacagttagtccaaaagaggcagcaccacttcccgatgggggaattagattgctgccaccctcacctcactaatgctgcggtgtgcggatatgccctgctatatatagctccgcgtttttgaaccagcacagcgcgagcaccaaaaaggaaaatcgcataggcccagaactgatttcagcacgggctattggcgttgggtttccgttctgggaaaccttcgccgcgtccccctcacaaacctccgcacaagttcgagcaagcgggaaaaaacgaaaaacgccattaatactaaataaagcaaatcctcgaagcgtgggtggcaagcccctggatttttccgcacaagtactcttctcaggagtaaaaaaacccgtttgttttggaattccccatttcgcggccatctacgccggtatcttcgcaatatctatcagcgataactcagcaattttaatattcgtgttgcagtgctgcgatagcgggagtcttgtttgtaacataacggcagaaaggaatgagagaaaattttccattctttggcctccgttcaagtatataaagccggcatgcttggtaatctttctctctcttctgtattgtttctataatacttttatcttctaattattttctgaaaaaaccaagaaattaatcttctgtcattcgcttaaacactatatcaata

SEQ ID NO: 99 - DNA, 750 bp, Artificial Sequence
  Synthetic nucleic acid sequence of promoter pCCW12.Sk
tgtaaaatcctacacgcaaaaaacctcttgggttgcgcgttttccaccacccacgacccgcacaatcaatccaaaaggggcaacgccagttcccaatgcgggaattagctcctcacccccctcacccgctaacgctgcggtgtgcggacacgcagcagtatttatagatcctcgtgttggaaccagcccgcgtgagcaccaaattggaaagtcgcaatgggcccagaaccgctttcagtactgggccattggcgtctagtttccgttttgagcgtccttcgccgcgtctctctgtgaaatccccgcacaagtctgagcaggcaaaaaaaaaaaacgccaccaaaaataaataaagccaatcctcgaaggatgagtaggaaaggaagcccctggttttttcccgcacgaatatttttcaggagtgaaaaaatccgtttgttttggaattccccatttcgcgctcacctacgccggtatctttggaacaactatcagcgataactcagcaaaatttgcatatccgtgctgcaatagtgcgatagtgggattgggagtcttgttgcatcataacggcagaaaggaatgaataaaaattttccgttctttgtctccgttcaagtatataaagtcggcatgcttgattatcttttcttctcttctactacatttctatttcactttctactctattcttccctgaaaaacccaagaaaataatcttctgtcattcgcttaaacaatatatcaaaa

SEQ ID NO: 100 - DNA, 750 bp, Artificial Sequence
  Synthetic nucleic acid sequence of promoter pCCW12.Sba
accatgccacggtgctggccccacttccacccacgacccgcacggttggcccgaaaggggcaacaccagttcccaatacggaaattagtccgccaccaccctcacctcgctagagctgcggtgtgcgggcgtgcatcgctatttatagacatgcctgctggcgtcaccgcgcgcgagcaccaaacaggaaaatcgcactgggcccagaaccacgctatgcgctgggccgatggcgtccggtttccctttgggagccccctgccacgttcgcctaacaaatccccgcaccggcttgagaaaaaagcgaaaagcgaaaaaaaaaaatcaacgccaccaaaattaaaaaaaaagagccatcctcgaagggtgaatagtagcccctgacttttcccgcacagacagacacctttcaggagtgaacaaaaaagcagtttgttttggaattcccccatttcgcggtggcctgcgcaggtatctctgcgtcaactatcagcgataactcagcaaattttgcatattcgtgttgcgatactacgataatgggagtctgtcgcctaataacggcaacaaggaatgagagagaaaaattttcttcattctccagctcccgttcaagtatataaggtcggcatggtcgattgtctttccttctcttcagttacgtctctctatttacattattcttatttttatttaataaaaaccccaagaaattaatcttctgtcattcgcttaaacactatatcaaaa

SEQ ID NO: 101 - DNA, 750 bp, Artificial Sequence
  Synthetic nucleic acid sequence of promoter pCCW12.Sar
aaaaaacaaccttctgccaacctgcgtgcttctcaccacccatgacccacacaattgacccgaatggggcaactccagttcccaatacgggaattaactcgccaccatatttaccgcgttgaagctgtggtgtgcggacactccgtactatttatagacccacgcggtggaaccagcacgcgcgcgcactaaacaggaaaatcgcattgagtccagaaccgccaccagcacttggccattggcgtctaatttccgttttcggcgcccctcaccgcgtccttctaacaaagcgcgcacaagcttgagcaagtgaaaagaaaattaaaaataaaaaaccgccaccaaaacaaataaagcaatcttcgaagtgtgggttggtgggaagcccctggcttttcccgcaccagtcgttttcaggagtaaaaaaatacccgtttgttttggaattccccatttcgcggcgacctgcgccggtatctttgcaacaactatttgcgataactcagcaaaatttgcatattcgtgttgggatattgcgatagtgggagtcttgttgcataataacggtaaaaagaagtgaaggaaaaaaatttgcatcctttagtctcagttcaagtatataaagtcgggatattcaattatctttctttctcttgctcaaaggtttctatattttttttatagtatttcttttgttataaaataccaagaaattaatcttctgtcattcgcttaaatactacatcaata

SEQ ID NO: 102 - DNA, 500 bp, Artificial Sequence
  Synthetic nucleic acid sequence of promoter pNUP57
tcatctgcgcaatgactatcaagaccttctgcaagaatttcaaatctcactgaaaatcttgaccgaaaagtgtcttgaaaacccatcaagcctgcaaaacctatctttgacattagtctccattataaaaacggcatagttgggagaaaactttccatacttcaattgtggactgatataagtattttagttttgcccgcatgatcatcccacatggctacagcagttctctcataggaaaatagtacaatagctacgtgatataatctaaataattgttgccaatgtgtaattatatcattttgaacgtacgcgaaatggattattttcaaaaattttgtttcttgaaatgagtaaaagcaaaagtccaactctccaagtcgatgtaaacaactttttgccaaagggactgaaagactaaatcgaggattatcccgttcaaactattccagaaacgctcgttagtaacaaaagacataccttgttgaccaattgatcac

SEQ ID NO: 103 - DNA, 523 bp, Artificial Sequence
  Synthetic nucleic acid sequence of promoter pCCW10.ago
ggtaccacggcaacctcgttcgctgttcatccccttcgtcacacaggacgttggatgccgtaagcagcgttgcttttgatcctcaggatcggccgggtaacccgcggctgcttctattttagtattcatatctcaagcacatccattccggccgtttgggggcgccgccgcactcgtgtccattcctaccgtggcacttagggctatcctgtcggagcgccccgccgaccgccttatcggcaccaaaagtagaagccccggccccgcgtggctcagactcaccatcggtgctatttacttttcgatcagatcgcggcgcgcggtggccggcatttccggaagcggccacggagcagaggtggcgcattcgaatcgcatacgtcttcgccacgccggaaaaaaaattttcggctatataaggagaggcggccgtcttgctgcaggcagtttcactttctctaaaaccaaagaacatcgatttctttagtcactcgcttccttacaccgaactcgaggcggccgc

SEQ ID NO: 104 - DNA, 943 bp, Artificial Sequence
  Synthetic nucleic acid sequence of promoter pCWP2
ctagcctcccctttttattttgtgcggtcaccgcaagggacaaagcttttcttagaaaaccgtctgagaagcataacgtacgccatcccctagacatattaataatgctacagatactatgctgctcgtctttttttgacgacccttttattgcaatgtgcaactaatggcaaacaaccacatagtatcacagtattacattgcctccaccgatgcggatgttagggcgccaagtctgtcatgaagcatgttcctgtcataatcttgtatgcaaaataccgcgttctgcgccactgatatgctaggcagcagcaacctatgcagaagattgcttttcccacgcctgttttacgtctccagggcacttgaaacaatgcagcgatcgccgccacaacacgccaaagagaagcgaaagtgggcctgggcggcctcagtttcggcagaggtaaacaacacgaactgaactgccttagctccgaagggcaattccacaggcactccgcggggcccggccaaggcccaaaaggcgtggaatatgcgcgttttggggccataacacccagtaccacggccggaacgggccatataataagtttttcactctcaagaatggtaaacgtaaataggaacatcccactaccctagaaattgcggaaatttcgcgcttatcattagaaaatctggaaccgtcctttttcctctttcttgcatttccctttccgtattattgccattctttaactgcatttggggaaccgtagaccaaaagccaaacagagaaatgtaacgttctaaaaaaaaaacaacgaaaaaattgaaaaataagatacaataatcgtatataaatcaggcttcttgttcatcattttcaattctcttcttgccatcccttttcctatctttgttcttttcttctcataatcaagaataaataacttcatca

SEQ ID NO: 105 - DNA, 650 bp, Artificial Sequence
  Synthetic nucleic acid sequence of promoter pFBA1
acgcaagccctaagaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaa

SEQ ID NO: 106 - DNA, 996 bp, Artificial Sequence
  Synthetic nucleic acid sequence of promoter pCCW120.Sm
cgttgtgctgtagtgaaggaagactaaaaaggataatcacagttgtaaagaggtataatgcggacccttttgccacaaaacacacatctttcgtttccaaaataagaaagaagaaagcaaaaagattagcagccacgttgctgcgatctaattatagacggtggcgtcatcattctcacccaagattgtgtcttgaacctgccacgggtcctgcgttatcgccggatgtaaaactagacatgcaaaaaaaggaccttccaggtagcgtgctccacaccacccatgaccaccacagttagtccaaaagaggcagcaccacttcccgatgggggaattagattgctgccaccctcacctcactaatgctgcggtgtgcggatatgccctgctatatatagctccgcgtttttgaaccagcacagcgcgagcaccaaaaaggaaaatcgcataggcccagaactgatttcagcacgggctattggcgttgggtttccgttctgggaaaccttcgccgcgtccccctcacaaacctccgcacaagttcgagcaagcgggaaaaaacgaaaaacgccattaatactaaataaagcaaatcctcgaagcgtgggtggcaagcccctggatttttccgcacaagtactcttctcaggagtaaaaaaacccgtttgttttggaattccccatttcgcggccatctacgccggtatcttcgcaatatctatcagcgataactcagcaattttaatattcgtgttgcagtgctgcgatagcgggagtcttgtttgtaacataacggcagaaaggaatgagagaaaattttccattctttggcctccgttcaagtatataaagccggcatgcttggtaatctttctctctcttctgtattgtttctataatacttttatcttctaattattttctgaaaaaaccaagaaattaatcttctgtcattcgcttaaacactatatcaatactcgaaatccaaggtggtgt

SEQ ID NO: 107 - DNA, 505 bp, Artificial Sequence
  Synthetic nucleic acid sequence of promoter pCUP1
cggcaaacttcaacgatttctatgatgcattttataattagtaagccgatcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcacataaa

SEQ ID NO: 108 - DNA, 510 bp, Artificial Sequence
  Synthetic nucleic acid sequence of promoter pMET6
ccacaggaaatatttcacgtgacttacaaacagagtcgtacgtcaggaccggagtcaggtgaaaaaatgtgggccggtaaagggaaaaaaccagaaacgggactactatcgaactcgtttagtcgcgaacgtgcaaaaggccaatatttttcgctagagtcatcgcagtcatggcagctctttcgctctatctcccggtcgcaaaactgtggtagtcatagctcgttctgctcaattgagaactgtgaatgtgaatatggaacaaatgcgatagatgcactaatttaagggaagctagctagttttcccaactgcgaaagaaaaaaaggaaagaaaaaaaaattctatataagtgatagatatttccatctttactagcattagtttctcttttacgtattcaatatttttgttaaactcttcctttatcataaaaaagcaagcatctaagagcattgacaacactctaagaaacaaaataccaatataatttcaaagtacatatcaaaa

SEQ ID NO: 109 - DNA, 686 bp, Artificial Sequence
  Synthetic nucleic acid sequence of promoter pMET25
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcattattttttgctttttctcttgaggtcacatgatcgcaaaatggcaaatggcacgtgaagctgtcgatattggggaactgtggtggttggcaaatgactaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 110 - DNA, 363 bp, Artificial Sequence
  Synthetic nucleic acid sequence of promoter pSAM1
gaaacggacgtaagacggaaatagaatttgaagataaagttatatatcactacacacgaatactttcttttttttttttcacaggaaaactgtggtggcgcccttgcctactagtgcatttcttttttcgggttcttgtctcgacgaaattttagcctcatcgtagtttttcactctggtatcgatgaaaaagggaagagtaaaaagttttccgtttagtacttaatgggattggtttgggacgtatatatcgactggtgttgtctgttattcatcgttgtttttcggttagcttcgaaaaaaaaatagagtaaaaaccaggaatttaccctaaaaacaagaaaaaataagataaacgaaaat

SEQ ID NO: 111 - DNA, 300 bp, Artificial Sequence
  Synthetic nucleic acid sequence of terminator tTPI1
gattaatataattatataaaaatattatcttcttttctttatatctagtgttatgtaaaataaattgatgactacggaaagcttttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattactttgtctctaaatccttgtaaaatgtgtacgatctctatatgggttactca

SEQ ID NO: 112 - DNA, 299 bp, Artificial Sequence
  Synthetic nucleic acid sequence of terminator tMET25
gtgtgcgtaatgagttgtaaaattatgtataaacctactttctctcacaagtactatacttttataaaacgaactttattgaaatgaatatcctttttttcccttgttacatgtcgtgactcgtactttgaacctaaattgttctaacatcaaagaacagtgttaattcgcagtcgagaagaaaaatatggtgaacaagactcatctacttcatgagactactttacgcctcctataaagctgtcacactggataaatttattgtaggaccaagttacaaaagaggatgatggaggttt

SEQ ID NO: 113 - DNA, 300 bp, Artificial Sequence
  Synthetic nucleic acid sequence of terminator tDIT1
taaagtaagagcgctacattggtctacctttttgttcttttacttaaacattagttagttcgttttctttttctcatttttttatgtttcccccccaaagttctgattttataatattttatttcacacaattccatttaacagagggggaatagattctttagcttagaaaattagtgatcaatatatatttgcctttcttttcatcttttcagtgatattaatggtttcgagacactgcaatggccctagttgtctaagaggatagatgttactgtcaaagatgatattttgaatttc

SEQ ID NO: 114 - DNA, 300 bp, Artificial Sequence
  Synthetic nucleic acid sequence of terminator tRPL3
gaagttttgttagaaaataaatcattttttaattgagcattcttattcctattttatttaaatagttttatgtattgttagctacatacaacagtttaaatcaaattttctttttcccaagtccaaaatggaggtttattttgatgacccgcatgcgattatgttttgaaagtataagactacatacatgtacatatatttaaacatgtaaacccgtccattatattgcttactttcttcttttttgccgttttgacttggacctctggtttgctatttccttacaatctttgctacaat

SEQ ID NO: 115 - DNA, 304 bp, Artificial Sequence
  Synthetic nucleic acid sequence of terminator tRPL3.sm
gaagtttttaaagcattttttagacacttctcatttttctaagtttttttaaaatagttttatgtatttactacgtatcacaatttgaaataattcatcttcccaaaaaactaagatttttatccttgtcacgatccgtaaccagtttataatattttagagcttatacacgtacgtatacacacgtgtcggtacatgagaattacgttcaaaattattcactttttttttctctgccgttttacttttgaactctgtctcgctatttccttacaatcttcgctacaataccacttgcccttgg

SEQ ID NO: 116 - DNA, 299 bp, Artificial Sequence
  Synthetic nucleic acid sequence of terminator tRPL3.sba
gaagttttctgacaaaaacataacgttttttccaatcatttcttatttttccggtttatttaaatagtttttatgtactattatacgtatgactatttaacttaaattcttcctcccaagaaatctcccaagtttttcattatcatggcatacaccactatcagttacaaaatggtagctcaaccatatatatatctctatatacacatataaatgcaaacaggtccaagtcaccgctcactgcagtttcttttgccgttttgacttcgatctctgcttggctattttctcacaatcct

SEQ ID NO: 117 - DNA, 300 bp, Artificial Sequence
  Synthetic nucleic acid sequence of terminator tRPL41B
gcggattgagagcaaatcgttaagttcaggtcaagtaaaaattgatttcgaaaactaatttctcttatacaatcctttgattggaccgtcatcctttcgaatataagattttgttaagaatattttagacagagatctactttatatttaatatctagatattacataatttcctctctaataaaatatcattaataaaataaaaatgaagcgatttgattttgtgttgtcaacttagtttgccgctatgcctcttgggtaatgctattattgaatcgaagggctttattatattaccct

SEQ ID NO: 118 - DNA, 301 bp, Artificial Sequence
  Synthetic nucleic acid sequence of terminator tRPL41B.Sba
gcggattgcgagtgattttttgagtacagaccaagtaaaattgatctatgcataatcttttctcatccacaatccctagaatggaccgtcatcctttctaaaatgttttattaagaatatttaaagtggaagtcaggtttatatctatcaattaaatattacataatttccccttaataaaatactgttaataaaataacgaaacgttcatagtttttcataaatagtcctgatgcttactgcaccggaacgtactgttttagtgtattgctcccttcattgccatttttcctagttattg

SEQ ID NO: 119 - DNA, 300 bp, Artificial Sequence
  Synthetic nucleic acid sequence of terminator tRPL15A
gctggttgatggaaaatataattttattgggcaaacttttgtttatctgatgtgttttatactattatctttttaattaatgattctatatacaaacctgtatattttttctttaaccaatttttttttttatagacctagagctgtacttttattctgctatcaagcaaacccctaccccctcttctcaatcctcccctcaggcagaacttatctacctgtatcaaggagcggacgagggagtcctaattgttctacgtataccaatgctagcagcttacataggtggtggcactacca

SEQ ID NO: 120 - DNA, 299 bp, Artificial Sequence
  Synthetic nucleic acid sequence of terminator tRPL15A.Sm
gctaattagaaccatatatacatcgataaaaacacttgaatgtatttttctttttcttcttattttttcattttccttcgctttctattagcgtgcgaatctaaagtgtgcagcgctatctttgactctttttttcctttagtgggaacgaacaatattaatcatgccaagttaagtcattgaagaagtcagtagtatgttattcggagagaagaccctgctatgtatctcagcctacccaaaaaatgaattgactcctctcataacttgttaatattcggatgagcacatcacgttcg

SEQ ID NO: 121 - DNA, 300 bp, Artificial Sequence
  Synthetic nucleic acid sequence of terminator tRPL15A.sba
gctggctgatgaaaaatatagttctgttgggcaagcatttgtttacctagcatctcttttatactattattatctttatatttgatgattttatatacaagttgtataccttttctttaaccaatttttttttttctaatggtgcacctagaagtacattttttctcaccaatagatagtcaagatactcccagcctctatggcgttaccacggagccgacaagggaaagctgcttatcttacatatgcagatgccaaggcccgttacaggccgccttattgatgtttagaaatagcttc

SEQ ID NO: 122 - DNA, 300 bp, Artificial Sequence
  Synthetic nucleic acid sequence of terminator tIDP1
tcgaatttacgtagcccaatctaccacttttttttttcattttttaaagtgttatacttagttatgctctaggataatgaactacttttttttttttttttttactgttatcataaatatatataccttattgttgtttgcaaccgtcggttaattccttatcaaggttccccaagttcggatcattaccatcaatttccaacattttcatgagttcttcttcttcattaccgtgttttagggggctgttcgcacttctaatagggctatcaccaagctgttctaattcgtccaaaagtt

SEQ ID NO: 123 - DNA, 290 bp, Artificial Sequence
  Synthetic nucleic acid sequence of terminator tIDP1.Sba
tctgatctttggaatgctccatttacttttttatttcatatttaggtacgatatatagttacgcttgagaactatgaactaataacttcctttttctctaatacatacttacttcagctgatggttgcaaacaacaataaaattccttgtcgaggttccctaaattaggatcattgccatcgacttctaacattttcatgagatcctcctcttcgttcccattctttaaaagactgtttccacttctaatagggttatcgcccaattgttctagttcgtctagcaattcg

SEQ ID NO: 124 - DNA, 300 bp, Artificial Sequence
  Synthetic nucleic acid sequence of terminator tTEF1.sba
ggagattgataggacttttctagttgcatatcttttatttttaaatcttatctattagttaattttttgtaatttatccttatatatatagtttggttattctaaaacatcatttcagtatctaaaacctctcttattcattacctttttatttaatggtttttgctacaggcaaaaatttaatggtttttgctacaggcaaaaatcccgccgtggacttattccacgttaactcggttacagggtcatgaaccattttgtcaattatcgaaataacttcttcaaaagtccctcttactt

SEQ ID NO: 125 - DNA, 60 bp, Artificial Sequence
  Synthetic nucleic acid sequence of the secretion sequence added in 5a
atgcaatttagcacagtcgcatcagtagccttcgttgccttggccaacttcgtggcagca

SEQ ID NO: 126 - protein, 20 aa, Artificial Sequence
  Synthetic amino acid sequence of the secretion sequence added in N-ter
MQFSTVASVAFVALANFVAA

SEQ ID NO: 127 - DNA, 213 bp, Artificial Sequence
  Synthetic nucleic acid sequence of the anchoring sequence added in 3a
ggatccgccatttctcaaatcactgacggtcaaatccaagctactaccactgctaccaccgaagctaccaccactgctgccccatcttccaccgttgaaactgtttctccatccagcaccgaaactatctctcaacaaactgaaaatggtgctgctaaggccgctgtcggtatgggtgccggtgctctagctgctgctgctatgttgttataa

SEQ ID NO: 128 - protein, 68 aa, Artificial Sequence
  Synthetic amino acid sequence of the anchoring sequence added in C-ter
AISQITDGQIQATTTATTEATTTAAPSSTVETVSPSSTETISQQTENGAAKAAVGMGAGALAAAAMLL